(19) United States
(12) Patent Application Publication
(10) Pub. No.: US 2010/0105607 A1
(43) Pub. Date: Apr. 29, 2010

(54) NOVEL SEMI-SYNTHETIC GLYCOPEPTIDES AS ANTIBACTERIAL AGENTS

(75) Inventors: Daniel Chu, Santa Clara, CA (US); Tao Ye, Hung Hom (CN); Bing Wang, San Jose, CA (US)

Correspondence Address: WILSON, SONSINI, GOODRICH & ROSATI, 650 PAGE MILL ROAD, PALO ALTO, CA, 94304-1050, US

(73) Assignee: LEAD Therapeutics, Inc., San Bruno, CA (US)

(21) Appl. No.: 12/603,435

(22) Filed: Oct. 21, 2009

Related U.S. Application Data
(60) Provisional application No. 61/108,446, filed on Oct. 24, 2008.
(60) Provisional application No. 61/110,447, filed on Oct. 31, 2008.

(51) Int. Cl. A61K 38/08; C07K 9/00; A61P 31/04

(52) U.S. Cl. 514/8; 530/322

(57) ABSTRACT

Semi-synthetic glycopeptides having antibacterial activity are described, in particular, the semi-synthetic glycopeptides described herein are made by chemical modification of the a glycopeptide (Compound A, Compound B, Compound H or Compound C) or the monosaccharide made by hydrolyzing the disaccharide moiety of the amino acid-4 of the parent glycopeptide in acidic medium to give the amino acid-4 monosaccharide; conversion of the monosaccharide to the amino-sugar derivative; acylation of the amino substituent on the amino acid-4 amino-substituted sugar moiety on these scaffolds with certain acyl groups; conversion of the amide group in amino acid-3 on these scaffolds to various acylamide, acylsulfonamide, acylsulfonylurea derivatives; aminomethylation with substituent containing sulfonamide or acylsulfonamide group on amino acid-7 through Mannich reaction; and conversion of the acid moiety on the macrocyclic ring of these scaffolds to certain substituted amides. Also provided are methods for the synthesis of the compounds, pharmaceutical compositions containing the compounds, and methods of use of the compounds for the treatment and/or prophylaxis of diseases, especially bacterial infections.

### CROSS REFERENCE

This application claims priority to U.S. Provisional Application Nos. 61/108,446, filed Oct. 24, 2008, and 61/110,447, filed Oct. 31, 2008, both of which are incorporated by reference in their entirety.

### FIELD OF THE INVENTION

Described herein are semi-synthetic glycopeptides having antibacterial activity, to pharmaceutical compositions comprising these compounds, and to a medical method of treatment.

### BACKGROUND OF THE INVENTION

The emergence of drug resistant bacterial strains has highlighted the need for synthesizing and identifying antibiotics with improved activity. Naturally occurring and semi-synthetic glycopeptide antibiotics used to combat bacterial infections include compounds such as vancomycin, desmethylvancomycin, eremomycin, teicoplanin (complex of five compounds), dalbavancin, oritavancin, telavancin, and A82846B (LY264826) having structures A, B, C, D, E, F, G and H:

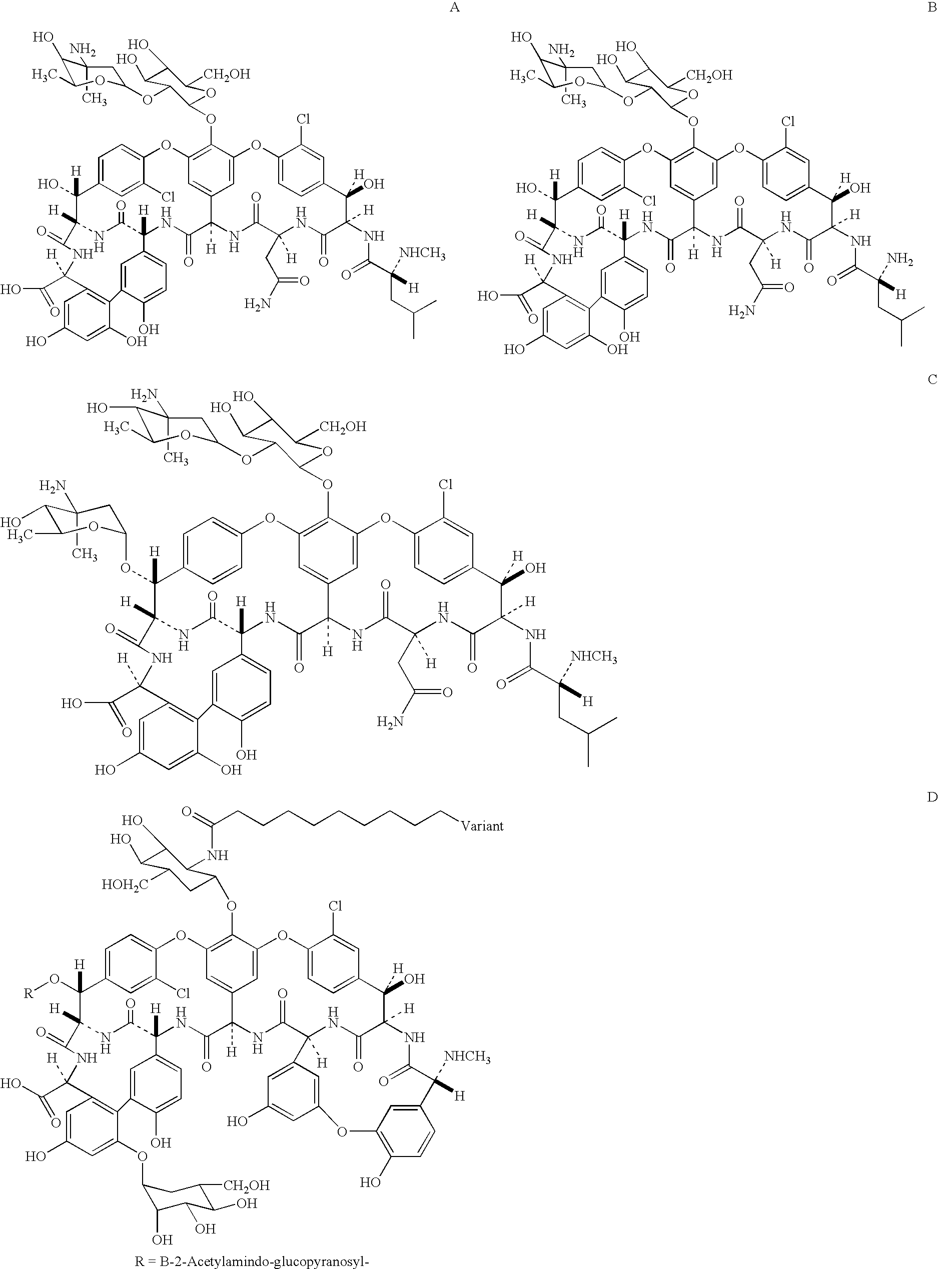

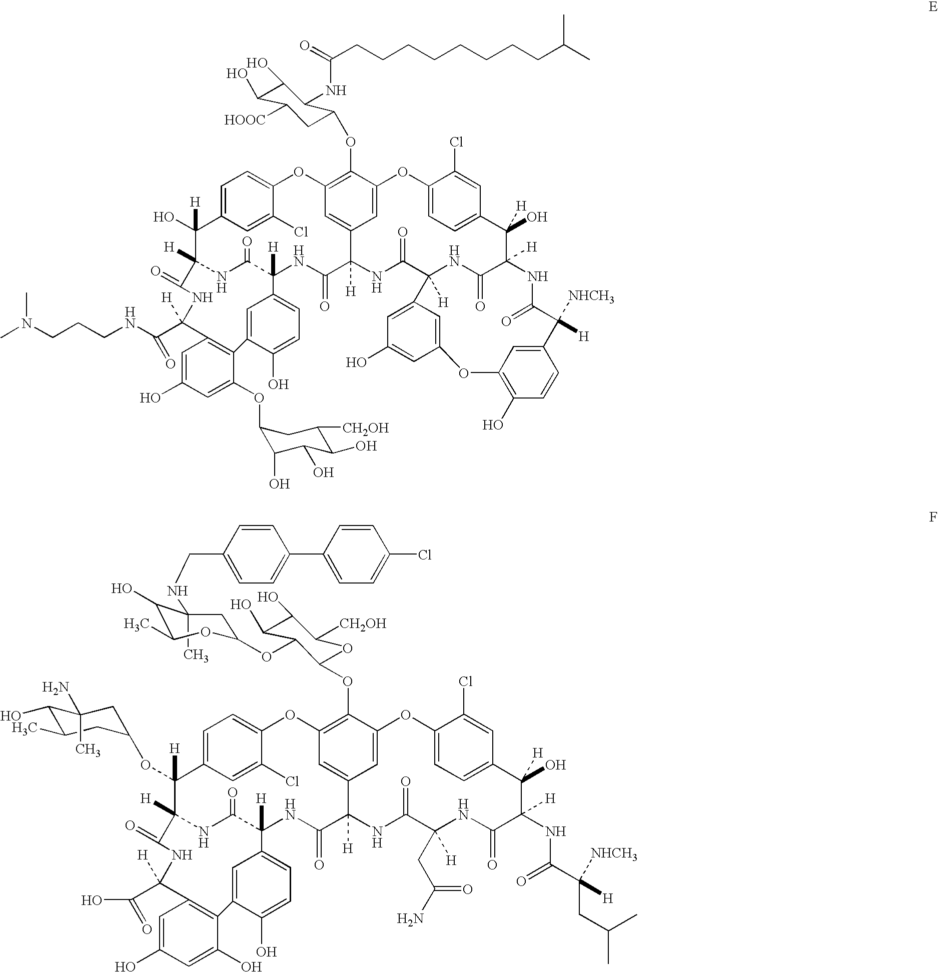

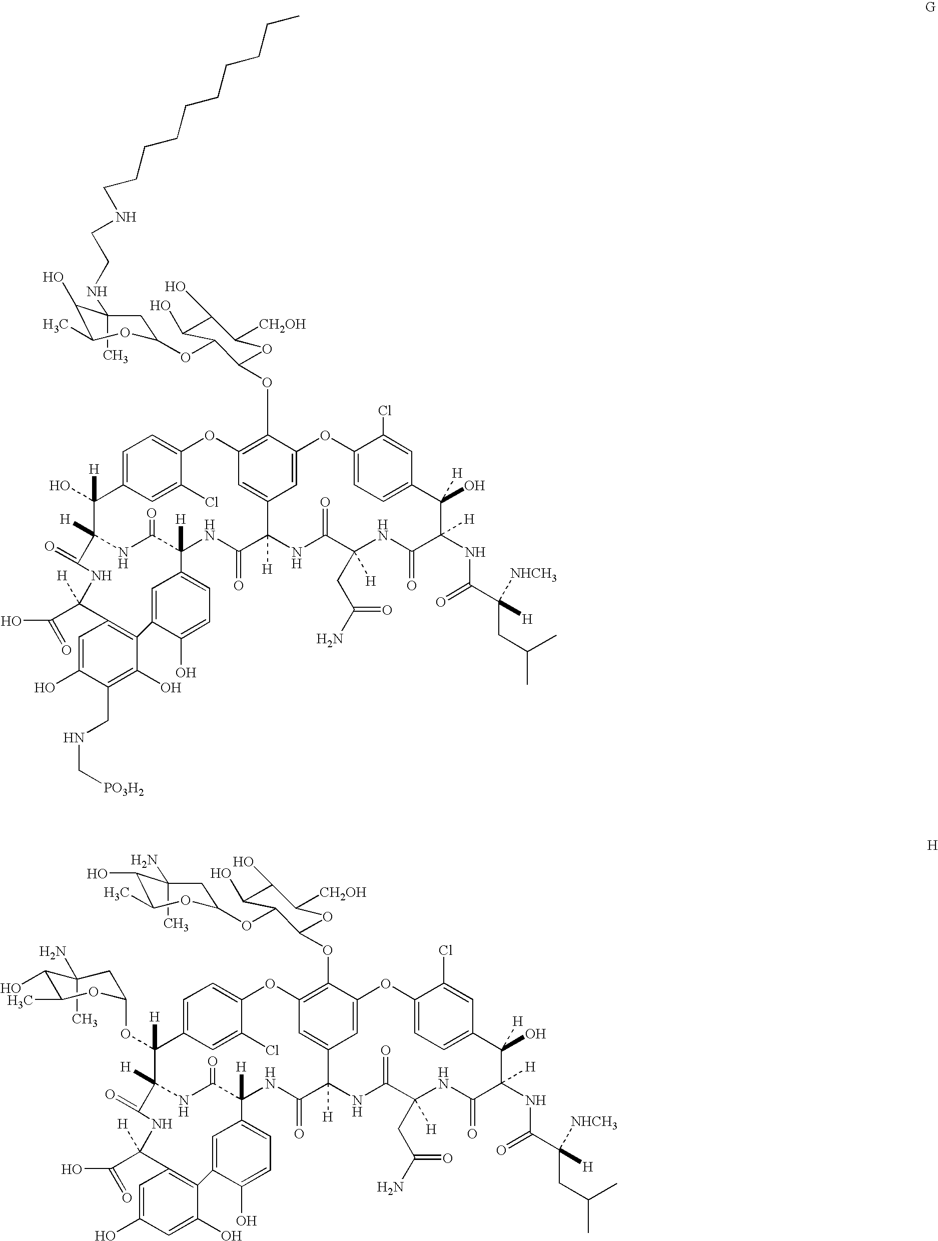

These compounds are used to treat and prevent bacterial infection, but as with other antibacterial agents, bacterial strains having resistance or insufficient susceptibility to these compounds have been identified, and these compounds have been found to have limited effect against certain bacterial infections e.g., against pulmonary S. aureus infections caused by Compound A intermediate-resistant S. aureus or infections due to Compound A resistant-enterococci.

### SUMMARY OF THE INVENTION

Described herein are semi-synthetic glycopeptides that have antibacterial activity. Also provided are methods for synthesis of the compounds, pharmaceutical compositions containing the compounds, and methods of use of the compounds for the treatment and/or prophylaxis of diseases, especially bacterial infections.

In one aspect described herein are compounds formed by modification of Compound A, Compound B, Compound C or Compound H scaffolds to provide novel semi-synthetic glycopeptides that have antibacterial activity, as well as their pharmaceutical acceptable salts, esters, solvates, alkylated quaternary ammonium salts, stereoisomers, tautomers or prodrugs thereof, and which are used, in some embodiments, as antibacterial agents for treatment of bacterial infections with superior microbiology and pharmacokinetic properties than currently available glycopeptide antibacterial agents.

In one aspect described herein are compounds having a structure selected from the group consisting of Formulas (I-V):

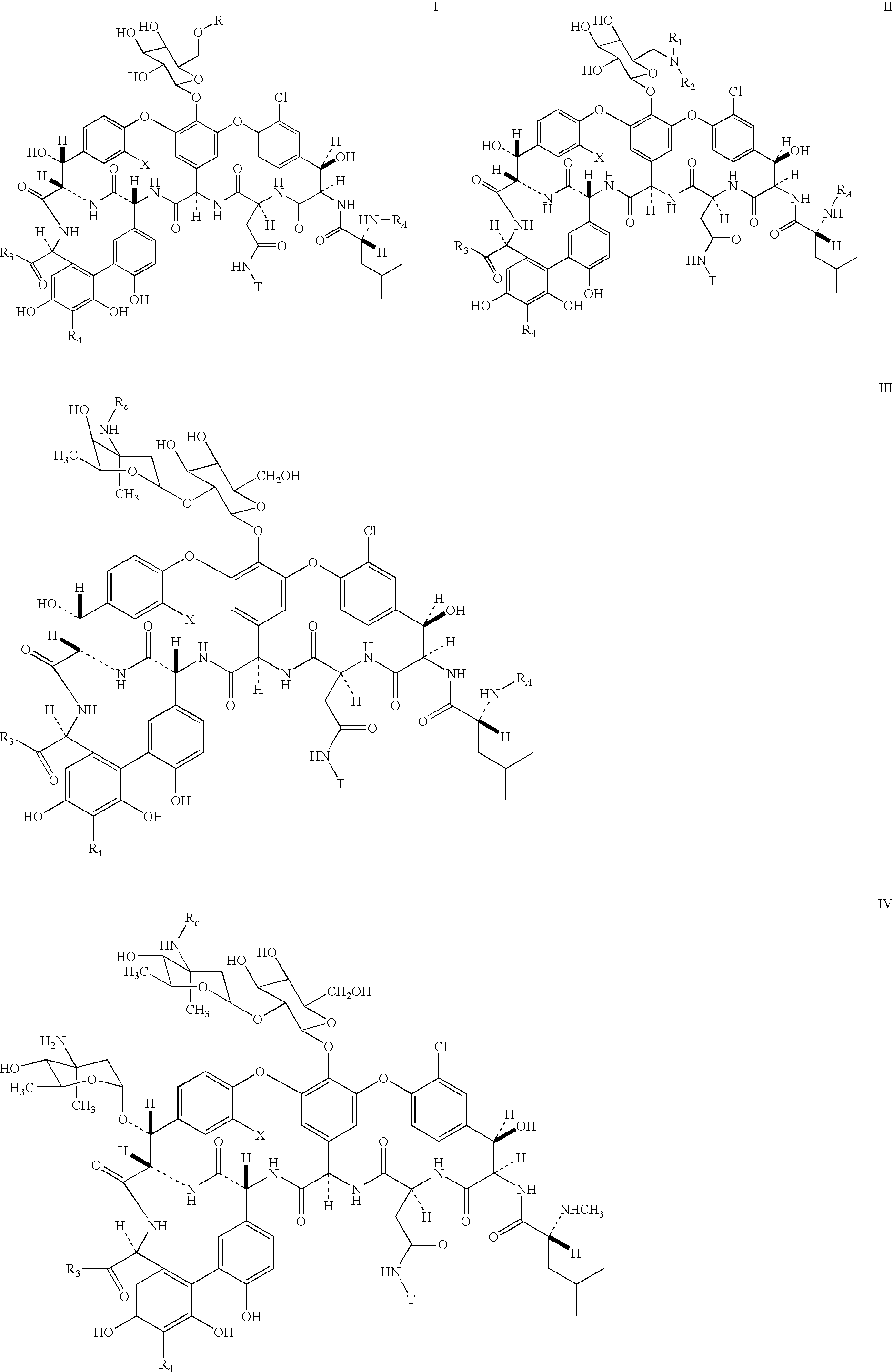

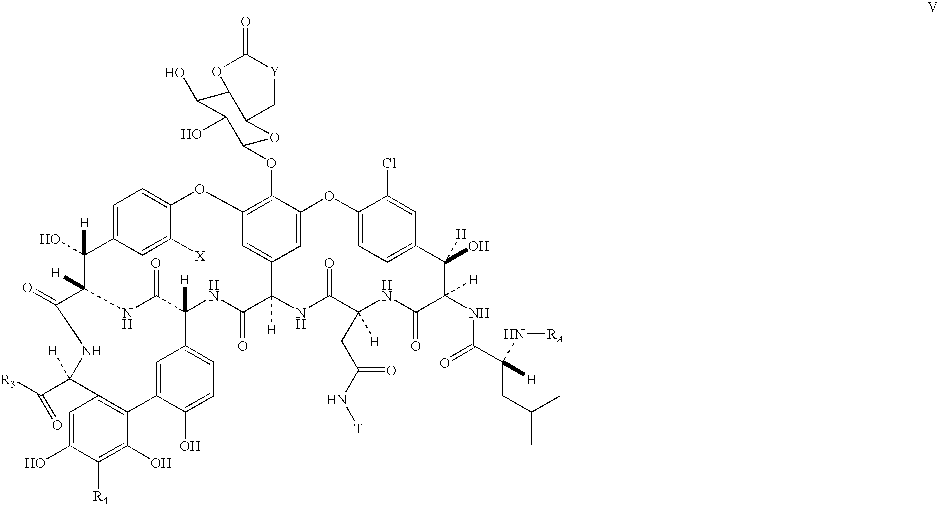

wherein,
R_{A} is selected from the group consisting of
   a) hydrogen,
   b) methyl,
   c) C₂-C₁₂-alkyl;
R₁ and R₂ are each independently selected from the group consisting of
   a) hydrogen,
   b) C₁-C₁₂-alkyl,
   c) C₁-C₁₂-alkyl substituted with one or more substituents selected from the group consisting of
      (a) halogen,
      (b) hydroxy,
      (c) C₁-C₁₂-alkoxy,
      (d) C₁-C₃-alkoxy-C₁-C₃-alkoxy,
      (e) amino,
      (f) C₁-C₁₂-alkylamino,
      (g) C₁-C₁₂-dialkylamino,
      (h) alkenyl,
      (i) alkynyl,
      (j) C₁-C₁₂-thioalkoxy,
   d) C₁-C₁₂-alkyl substituted with aryl,
   e) C₁-C₁₂-alkyl substituted with substituted aryl,
   f) C₁-C₁₂-alkyl substituted with heteroaryl,
   g) C₁-C₁₂-alkyl substituted with substituted heteroaryl,
   h) cycloalkyl,
   i) cycloalkenyl,
   j) heterocycloalkyl,
      or
      R₁ and R₂ taken together with the atom to which they are attached form a substituted heteroaryl or 3-10 membered heterocycloalkyl ring which optionally contains one or two hetero functionalities selected from the group consisting of —O—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, —S—, and S(O)ₙ— wherein n is 1 or 2 and the 3-10 membered heterocycloalkyl ring is optionally substituted with one or more substituents independently selected from the group consisting of
      (a) halogen,
      (b) hydroxyl,
      (c) C₁-C₃-alkoxy,
      (d) C₁-C₃-alkoxy-C₁-C₃-alkoxy,
      (e) oxo,
      (f) C₁-C₃-alkyl,
      (g) C₁-C₃haloalkyl,
      (h) C₁-C₃-alkoxy-C₁-C₃-alkyl,
   and
   k) C(═O)R₇,
   l) C(═O)CHR₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
      or
      R₉ and R₁₀ taken together with the atom to which they are attached form a 3-10 membered heterocycloalkyl ring which is optionally substituted with one or more substituents independently selected from the group consisting of
      (a) halogen,
      (b) hydroxyl,
      (c) C₁-C₃-alkoxy,
      (d) C₁-C₃-alkoxy-C₁-C₃-alkoxy,
      (e) oxo,
      (f) C₁-C₃-alkyl,
      (g) C₁-C₃haloalkyl,
      (h) C₁-C₃-alkoxy-C₁-C₃-alkyl;
R₇ is selected from the group consisting of
   a) hydrogen,
   b) C₁-C₁₂-alkyl,
   c) C₁-C₁₂-alkyl substituted with one or more substituents selected from the group consisting of
      (a) halogen,
      (b) hydroxy,
      (c) C₁-C₁₂-alkoxy,
      (d) C₁-C₃-alkoxy-C₁-C₃-alkoxy,
      (e) amino,
      (f) C₁-C₁₂-alkylamino,
      (g) C₁-C₁₂-dialkylamino,
      (h) alkenyl,
      (i) alkynyl,
      (j) C₁-C₁₂-thioalkoxy,
   d) C₁-C₁₂-alkyl substituted with aryl,
   e) C₁-C₁₂-alkyl substituted with substituted aryl,
   f) C₁-C₁₂-alkyl substituted with heteroaryl,
   g) C₁-C₁₂-alkyl substituted with substituted heteroaryl,
   h) cycloalkyl,
   i) cycloalkenyl,
   j) heterocycloalkyl,
   k) C₁-C₁₂-alkylamino,
   l) amino,
   m) amino-cycloalkyl;
X is selected from the group consisting of
   (1) hydrogen,
   (2) chlorine;
Y is selected from the group consisting of
   (1) oxygen,
   (2) NR₁, wherein R₁ is as previously defined;
T is selected from the group consisting of
   (1) —SO₂R_{B},
   (2) —COR_{B},
   (3) —CONHSO₂R_{B};
R is selected from the group consisting of
   (1) hydrogen,
   (2) cycloalkyl,
   (3) cycloalkenyl,
   (4) C₁-C₁₂-alkyl,
   (5) C₁-C₁₂-alkyl substituted with one or more substituents selected from the group consisting of
      (a) halogen,
      (b) hydroxy,
      (c) C₁-C₁₂-alkoxy,
      (d) C₁-C₃-alkoxy-C₁-C₃-alkoxy,
      (e) —COOR₅ wherein R₅ is hydrogen or loweralkyl,
      (f) —C(O)NR₅R₆ wherein R₅ is as previously defined and R₆ is hydrogen or loweralkyl,
      (g) amino,
      (h) —NR₅R₆ wherein R₅ and R₆ are as previously defined,
         or
         R₅ and R₆ taken together with the atom to which they are attached form a 3-10 membered heterocycloalkyl ring which is optionally substituted with one or more substituents independently selected from the group consisting of
         (i) halogen.
         (ii) hydroxy,
         (iii) C₁-C₃-alkoxy,
         (iv) C₁-C₃-alkoxy-C₁-C₃-alkoxy,
         (v) oxo,
         (vi) C₁-C₁₂-alkyl,
         (vii) C₁-C₁₂haloalkyl,
         and
         (viii) C₁-C₃-alkoxy-C₁-C₁₂-alkyl,
      (i) aryl,
      (j) substituted aryl,
      (k) heteroaryl,
      (l) substituted heteroaryl,
      (m) mercapto,
      (n) C₁-C₁₂-thioalkoxy,
   (6) C(═O)OR₁₁, wherein R₁₁ is hydrogen, loweralkyl, substituted loweralkyl, aryl, substituted aryl, heteroaryl or substituted heteroaryl,
   (7) C(═O)NR₁₁R₁₂, wherein R₁₁ is as previously defined and R₁₂ is hydrogen, loweralkyl, substituted loweralkyl, aryl, substituted aryl, heteroaryl or substituted heteroaryl,
      or
      R₁₁ and R₁₂ together with the atom to which they are attached form a 3-10 membered heterocycloalkyl ring, which is optionally substituted with one or more substituents independently selected from the group consisting of
         (a) halogen,
         (b) hydroxy,
         (c) C₁-C₃-alkoxy,
         (d) C₁-C₃-alkoxy-C₁-C₃-alkoxy,
         (e) oxo,
         (f) C₁-C₁₂-alkyl,
         (g) substituted loweralkyl,
         (h) C₁-C₁₂haloalkyl,
         (i) amino,
         (j) alkylamino,
         (k) dialkylamino
         and
         (l) C₁-C₃-alkoxy-C₁-C₁₂-alkyl,
      or
      R and its connected oxygen atom taken together is halogen;
R₃ is selected from the group consisting of
   (1) OH,
   (2) 1-adamantanamino,
   (3) 2-adamantanamino,
   (4) 3-amino-1-adamantanamino,
   (5) 1-amino-3-adamantanamino,
   (6) 3-loweralkylamino-1-adamantanamino,
   (7) 1-loweralkylamino-3-adamantanamino,
   (8) amino
   (9) NR₁₃R₁₄ wherein R₁₃ and R₁₄ are each independently selected from the group consisting of hydrogen, loweralkyl, substituted loweralkyl, cycloalkyl, substituted cycloalkyl, aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy
   or
R₁₃ and R₁₄ together with the atom to which they are attached form a 3-10 membered heterocycloalkyl ring, which is optionally substituted with one or more substituents independently selected from the group consisting of
   (a) halogen.
   (b) hydroxy,
   (c) C₁-C₃-alkoxy,
   (d) C₁-C₃-alkoxy-C₁-C₃-alkoxy,
   (e) oxo,
   (f) C₁-C₁₂-alkyl,
   (g) substituted loweralkyl,
   (h) C₁-C₁₂haloalkyl,
   (i) amino,
   (j) alkylamino,
   (k) dialkylamino
   and
   (l) C₁-C₃-alkoxy-C₁-C₁₂-alkyl;
R₄ is selected from the group consisting of
   (1) CH₂NH—CHR₁₅—(CH₂)ₘ—NHSO₂R_{B}, wherein m is 1 to 6 and R₁₅ is H or loweralkyl,
   (2) CH₂NH—CHR₁₅—(CH₂)ₚ—CONHSO₂R_{B}, wherein p is 0 to 6 and R₁₅ is H or loweralkyl,
   (3) CH₂NH—CHR₁₅—(CH₂)ₚ—COOH, wherein p is 0 to 6 and R₁₅ is H or loweralkyl,
   (4) CH₂NR_{D}—CHR₁₅—(CH₂)_{q}—NR_{E}SO₂R_{B}, wherein q is 2 to 4 and R₁₅ is H or loweralkyl, R_{D} and R_{E} together represents a —CH₂—,
   (5) H,
   (6) CH₂NHCH₂PO₃H₂,
   (7) aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy;
R_{B} is selected from the group consisting of
   a) aryl,
   b) C₁-C₁₂-alkyl,
   c) C₁-C₁₂-alkyl substituted with one or more substituents selected from the group consisting of
      (a) halogen,
      (b) hydroxy,
      (c) C₁-C₁₂-alkoxy,
      (d) C₁-C₃-alkoxy-C₁-C₃-alkoxy,
      (e) amino,
      (f) C₁-C₁₂-alkylamino,
      (g) C₁-C₁₂-dialkylamino,
      (h) alkenyl,
      (i) alkynyl,
      (j) C₁-C₁₂-thioalkoxy,
   d) C₁-C₁₂-alkyl substituted with aryl,
   e) C₁-C₁₂-alkyl substituted with substituted aryl,
   f) C₁-C₁₂-alkyl substituted with heteroaryl,
   g) C₁-C₁₂-alkyl substituted with substituted heteroaryl,
   h) cycloalkyl,
   i) heteroaryl,
   j) heterocycloalkyl,
   k) aryl substituted with one or more substituents selected from the group consisting of
      (a) halogen,
      (b) hydroxy,
      (c) C₁-C₁₂-alkoxy,
      (d) C₁-C₆-alkoxy-C₁-C₆-alkoxy,
      (e) amino,
      (f) amino-C₁-C₆-alkoxy,
      (g) C₁-C₁₂-alkylamino,
      (h) C₁-C₁₂-alkylamino-C₁-C₆-alkoxy,
      (i) C₁-C₁₂-dialkylamino,
      (j) C₁-C₁₂-dialkylamino-C₁-C₆-alkoxy,
      (k) alkenyl,
      (l) alkynyl,
      (m) C₁-C₁₂-thioalkoxy,
      (n) C₁-C₁₂-alkyl,
      (o) C₁-C₁₂-substituted alkyl,
      (p) C₁-C₁₂-alkoxy-morpholino,
      (q) C₁-C₁₂-alkoxy-C₁-C₁₂-dialkoxyamino,
      (r) C₁-C₁₂-alkoxy-NHSO₂ C₁-C₆-alkyl,
      (s) C₁-C₁₂-alkoxy-NHCO C₁-C₆-alkyl;
   l) heteroaryl substituted with one or more substituents selected from the group consisting of
      (a) halogen,
      (b) hydroxy,
      (c) C₁-C₁₂-alkoxy,
      (d) C₁-C₆-alkoxy-C₁-C₆-alkoxy,
      (e) amino,
      (f) amino-C₁-C₆-alkoxy,
      (g) C₁-C₁₂-alkylamino,
      (h) C₁-C₁₂-alkylamino-C₁-C₆-alkoxy,
      (i) C₁-C₁₂-dialkylamino,
      (j) C₁-C₁₂-dialkylamino-C₁-C₆-alkoxy,
      (k) alkenyl,
      (l) alkynyl,
      (m) C₁-C₁₂-thioalkoxy,
      (n) C₁-C₁₂-alkyl,
      (o) C₁-C₁₂-substituted alkyl;
R_{C} is each independently selected from the group consisting of
   a) hydrogen,
   b) C₁-C₁₂-alkyl,
   c) C₁-C₁₂-alkyl substituted with one or more substituents selected from the group consisting of
      (a) halogen,
      (b) hydroxy,
      (c) C₁-C₁₂-alkoxy,
      (d) C₁-C₃-alkoxy-C₁-C₃-alkoxy,
      (e) amino,
      (f) C₁-C₁₂-alkylamino,
      (g) C₁-C₁₂-dialkylamino,
      (h) alkenyl,
      (i) alkynyl,
      (j) C₁-C₁₂-thioalkoxy,
   d) C₁-C₁₂-alkyl substituted with aryl,
   e) C₁-C₁₂-alkyl substituted with substituted aryl,
   f) C₁-C₁₂-alkyl substituted with heteroaryl,
   g) C₁-C₁₂-alkyl substituted with substituted heteroaryl,
   h) cycloalkyl,
   i) cycloalkenyl,
   j) heterocycloalkyl,
   k) C(═O)R₇ wherein R₇ is previously defined,
   l) C(═O)CHR₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
      or
      R₉ and R₁₀ taken together with the atom to which they are attached form a 3-10 membered heterocycloalkyl ring which is optionally substituted with one or more substituents independently selected from the group consisting of
      (a) halogen,
      (b) hydroxyl,
      (c) C₁-C₃-alkoxy,
      (d) C₁-C₃-alkoxy-C₁-C₃-alkoxy,
      (e) oxo,
      (f) C₁-C₃-alkyl,
      (g) C₁-C₃haloalkyl,
      (h) C₁-C₃-alkoxy-C₁-C₃-alkyl;
or a pharmaceutically acceptable salt, ester, solvate, alkylated quaternary ammonium salt, stereoisomer, tautomer or prodrug thereof.

In a further embodiment, the compound has the structure of Formula I

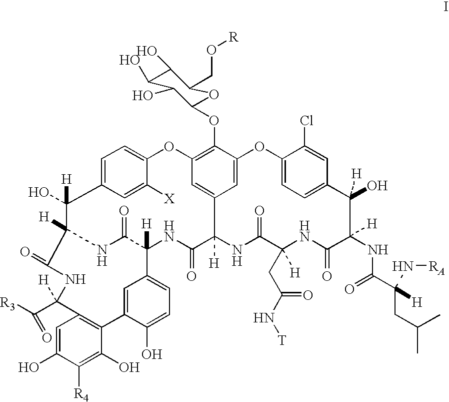

or a pharmaceutically acceptable salt, ester, solvate, alkylated quaternary ammonium salt, stereoisomer, tautomer or prodrug thereof, wherein R, etc. have the meanings as defined herein.

In a further embodiment, the compound has the structure of Formula II

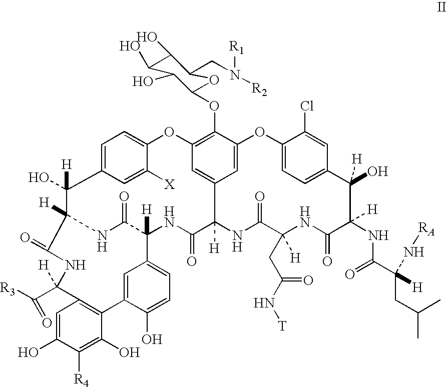

or a pharmaceutically acceptable salt, ester, solvate, alkylated quaternary ammonium salt, stereoisomer, tautomer or prodrug thereof, wherein R₁, etc. have the meanings as defined herein.

In a further embodiment, the compound has the structure of Formula III

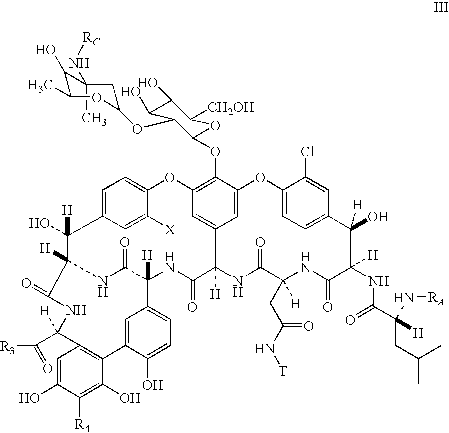

or a pharmaceutically acceptable salt, ester, solvate, alkylated quaternary ammonium salt, stereoisomer, tautomer or prodrug thereof, wherein R_{A}, etc. have the meanings as defined herein.

In a further embodiment, the compound has the structure of Formula IV

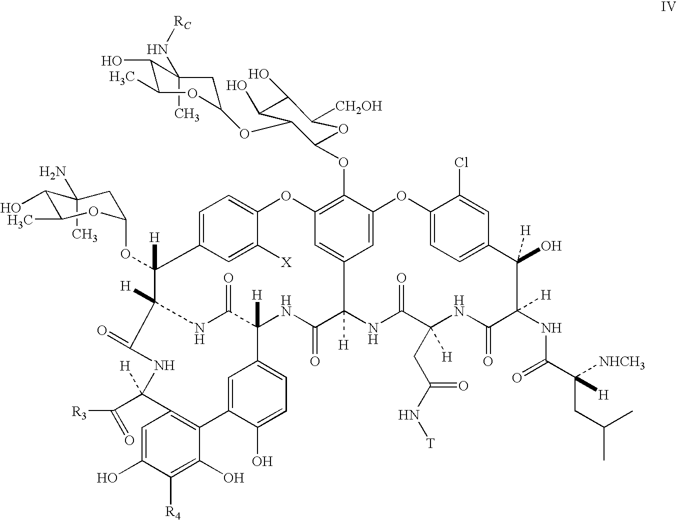

or a pharmaceutically acceptable salt, ester, solvate, alkylated quaternary ammonium salt, stereoisomer, tautomer or prodrug thereof, wherein R_{C}, etc. have the meanings as defined herein.

In a further embodiment, the compound has the structure of Formula V

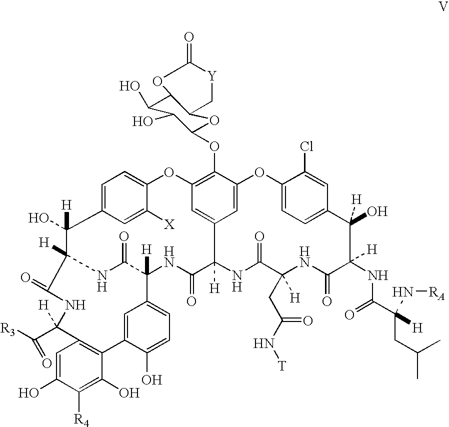

or a pharmaceutically acceptable salt, ester, solvate, alkylated quaternary ammonium salt, stereoisomer, tautomer or prodrug thereof, wherein R_{A}, etc. have the meanings as defined herein.

In a further embodiment of any of the above structures, R_{A} is methyl and R₄ is hydrogen. In another embodiment, R_{A} is hydrogen and R₄ is hydrogen. In another embodiment, X is hydrogen and R₄ is hydrogen. In another embodiment, X is chlorine and R₄ is hydrogen. In another embodiment, R_{A} is methyl and R₄ is CH₂NHCH₂PO₃H₂. In another embodiment, R_{A} is hydrogen and R₄ is CH₂NHCH₂PO₃H₂. In another embodiment, R_{A} is hydrogen and R₄ is CH₂NH—CHR₁₅—(CH₂)ₘ—NHSO₂R_{B}, wherein m is 1 to 6 and R₁₅ is H or loweralkyl. In another embodiment, R_{A} is methyl and R₄ is CH₂NH—CHR₁₅—(CH₂)ₘ—NHSO₂R_{B}, wherein m is 1 to 6 and R₁₅ is H or loweralkyl. In another embodiment, R_{A} is hydrogen and R₄ is CH₂NH—CHR₁₅—(CH₂)ₚ—CONHSO₂R_{B}, wherein p is 0 to 6 and R₁₅ is H or loweralkyl. In another embodiment, R_{A} is methyl and R₄ is CH₂NH—CHR₁₅—(CH₂)ₚ—CONHSO₂R_{B}, wherein p is 0 to 6 and R₁₅ is H or loweralkyl. In another embodiment, R_{A} is hydrogen and R₄ is CH₂NR_{D}—CHR₁₅—(CH₂)_{q}—NR_{E}SO₂R_{E}, wherein q is 2 to 4 and R₁₅ is H or loweralkyl, R_{D} and R_{E} together represents —CH₂—. In another embodiment, R_{A} is methyl and R₄ is CH₂NR_{D}—CHR₁₅—(CH₂)_{q}—NR_{E}SO₂R_{B}, wherein q is 2 to 4 and R₁₅ is H or loweralkyl, R_{D} and R_{E} together represents —CH₂—.

In a further embodiment of any of the aforementioned embodiments, R₃ is selected from the group consisting of
(1) OH,
(2) 1-adamantanamino,
(3) 2-adamantanamino,
(4) 3-amino-1-adamantanamino,
(5) 1-amino-3-adamantanamino,
(6) 3-loweralkylamino-1-adamantanamino,
(7) 1-loweralkylamino-3-adamantanamino,
(8) amino
(9) NR₁₃R₁₄ wherein R₁₃ and R₁₄ are each independently selected from the group consisting of hydrogen, loweralkyl, substituted loweralkyl, cycloalkyl, substituted cycloalkyl, aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy
or
R₁₃ and R₁₄ together with the atom to which they are attached form a 3-10 membered heterocycloalkyl ring, which is optionally substituted with one or more substituents independently selected from the group consisting of
   (a) halogen,
   (b) hydroxy,
   (c) C₁-C₃-alkoxy,
   (d) C₁-C₃-alkoxy-C₁-C₃-alkoxy,
   (e) oxo,
   (f) C₁-C₁₂-alkyl,
   (g) substituted loweralkyl,
   (h) C₁-C₁₂haloalkyl,
   (i) amino,
   (j) alkylamino,
   (k) dialkylamino and
   (l) C₁-C₃-alkoxy-C₁-C₁₂-alkyl.

In a further embodiment, R₃ is OH. In another embodiment, R₃ is 2-adamantanamino. In another embodiment, R₃ is dimethylamino. In a further embodiment, R₃ is diethylamino. In another embodiment, R₃ is dimethylaminoethylamino. In another embodiment, R₃ is N-methylpiperazino.

In a further embodiment of any of the aforementioned embodiments, R₁ and R₂ are each independently selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
   or
   R₁ and R₂ taken together with the atom to which they are attached form a substituted heteroaryl or 3-10 membered heterocycloalkyl ring which optionally contains one or two hetero functionalities selected from the group consisting of —O—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring optionally be substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) C₁-C₃haloalkyl,
   (h) C₁-C₃-alkoxy-C₁-C₃-alkyl,
and
k) C(═O)R₇,
l) C(═O)CHR₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl, or
   R₉ and R₁₀ taken together with the atom to which they are attached form a 3-10 membered heterocycloalkyl ring which is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) C₁-C₃haloalkyl,
   (h) C₁-C₃-alkoxy-C₁-C₃-alkyl.

In a further embodiment of any of the aforementioned embodiments, R₁ and R₂ are hydrogen. In another embodiment, R₁ is C₁-C₁₂-alkyl and R₂ is hydrogen. In another embodiment, R₁ is C₁-C₁₂-alkyl substituted with aryl or substituted aryl and R₂ is hydrogen. In another embodiment, R₁ is C(═O)C₁-C₁₂-alkyl and R₂ is hydrogen. In another embodiment, R₁ is C(═O)CH₂NHC₁-C₁₂-alkyl and R₂ is hydrogen. In another embodiment, R₁ is C₁-C₁₂-alkyl substituted C₁-C₁₂-alkoxy and R₂ is hydrogen. In another embodiment, R₁ is C₁-C₁₂-alkyl substituted C₁-C₁₂-thioalkoxy and R₂ is hydrogen. In another embodiment, R₁ is C₁-C₁₂-alkyl substituted C₁-C₁₂-alkylamino and R₂ is hydrogen.

In a further embodiment of any of the aforementioned embodiments, R is selected from the group consisting of
(1) hydrogen,
(2) cycloalkyl,
(3) cycloalkenyl,
(4) C₁-C₁₂-alkyl,
(5) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) —COOR₅ wherein R₅ is hydrogen or loweralkyl,
   (f) —C(O)NR₅R₆ wherein R₅ is as previously defined and R₆ is hydrogen or loweralkyl,
   (g) amino,
   (h) —NR₅R₆ wherein R₅ and R₆ are as previously defined,
      or
      R₅ and R₆ taken together with the atom to which they are attached form a 3-10 membered heterocycloalkyl ring which is optionally substituted with one or more substituents independently selected from the group consisting of
      (i) halogen,
      (ii) hydroxy,
      (iii) C₁-C₃-alkoxy,
      (iv) C₁-C₃-alkoxy-C₁-C₃-alkoxy,
      (v) oxo,
      (vi) C₁-C₁₂-alkyl,
      (vii) C₁-C₁₂haloalkyl,
      and
      (viii) C₁-C₃-alkoxy-C₁-C₁₂-alkyl,
   (i) aryl,
   (j) substituted aryl,
   (k) heteroaryl,
   (l) substituted heteroaryl,
   (m) mercapto,
   (n) C₁-C₁₂-thioalkoxy,
(6) C(═O)OR₁₁, wherein R₁₁ is hydrogen, loweralkyl, substituted loweralkyl, aryl, substituted aryl, heteroaryl or substituted heteroaryl,
(7) C(═O)NR₁₁R₁₂, wherein R₁₁ is as previously defined and R₁₂ is hydrogen, loweralkyl, substituted loweralkyl, aryl, substituted aryl, heteroaryl or substituted heteroaryl,
   or
   R₁₁ and R₁₂ together with the atom to which they are attached form a 3-10 membered heterocycloalkyl ring, which is optionally substituted with one or more substituents independently selected from the group consisting of
      (a) halogen,
      (b) hydroxy,
      (c) C₁-C₃-alkoxy,
      (d) C₁-C₃-alkoxy-C₁-C₃-alkoxy,
      (e) oxo,
      (f) C₁-C₁₂-alkyl,
      (g) substituted loweralkyl,
      (h) C₁-C₁₂haloalkyl,
      (i) amino,
      (j) alkylamino,
      (k) dialkylamino
      and
      (l) C₁-C₃-alkoxy-C₁-C₁₂-alkyl,
   or
   R and its connected oxygen atom taken together is halogen.

In a further embodiment of any of the aforementioned embodiments, R is hydrogen. In another embodiment, R is C₁-C₁₂-alkyl. In another embodiment, R is C₁-C₁₂-alkyl substituted with aryl or substituted aryl. In another embodiment, R is C(═O)NHC₁-C₁₂-alkyl. In another embodiment, R is C(═O)NHC₁-C₁₂-alkyl substituted with aryl or substituted aryl. In another embodiment, R is C(═O)OC₁-C₁₂-alkyl. In another embodiment, R is C(═O)NHC₁-C₁₂-alkyl substituted with heteroaryl or substituted heteroaryl.

In a further embodiment of any of the aforementioned embodiments, R_{B} is selected from the group consisting of
a) aryl,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) heteroaryl,
j) heterocycloalkyl,
k) aryl substituted with one or more substituents selected from the group consisting of
   (a) halogen,
   (b) hydroxy,
   (c) C₁-C₁₂-alkoxy,
   (d) C₁-C₆-alkoxy-C₁-C₆-alkoxy,
   (e) amino,
   (f) amino-C₁-C₆-alkoxy
   (g) C₁-C₁₂-alkylamino,
   (h) C₁-C₁₂-alkylamino-C₁-C₆-alkoxy,
   (i) C₁-C₁₂-dialkylamino,
   (j) C₁-C₁₂-dialkylamino-C₁-C₆-alkoxy,
   (k) alkenyl,
   (l) alkynyl,
   (m) C₁-C₁₂-thioalkoxy,
   (n) C₁-C₁₂-alkyl,
   (o) C₁-C₁₂-substituted alkyl,
   (p) C₁-C₁₂-alkoxy-morpholino,
   (q) C₁-C₁₂-alkoxy-C₁-C₁₂-dialkoxyamino,
   (r) C₁-C₁₂-alkoxy-NHSO₂ C₁-C₆-alkyl,
   (s) C₁-C₁₂-alkoxy-NHCO C₁-C₆-alkyl;
l) heteroaryl substituted with one or more substituents selected from the group consisting of
   (a) halogen,
   (b) hydroxy,
   (c) C₁-C₁₂-alkoxy,
   (d) C₁-C₆-alkoxy-C₁-C₆-alkoxy,
   (e) amino,
   (f) amino-C₁-C₆-alkoxy,
   (g) C₁-C₁₂-alkylamino,
   (h) C₁-C₁₂-alkylamino-C₁-C₆-alkoxy,
   (i) C₁-C₁₂-dialkylamino,
   (j) C₁-C₁₂-dialkylamino-C₁-C₆-alkoxy,
   (k) alkenyl,
   (l) alkynyl,
   (m) C₁-C₁₂-thioalkoxy,
   (n) C₁-C₁₂-alkyl,
   (o) C₁-C₁₂-substituted alkyl;

In a further embodiment of any of the aforementioned embodiments, R_{B} is C₁-C₁₂-alkyl. In another embodiment, R_{B} is C₁-C₁₂-alkyl substituted with aryl or substituted aryl. In another embodiment, R_{B} is C₁-C₁₂-alkyl substituted with heteroaryl or substituted heteroaryl. In another embodiment, R_{B} is aryl substituted with C₁-C₁₂-alkyl. In another embodiment, R_{B} is aryl substituted with halogen. In another embodiment, R_{B} is aryl substituted with substituted C₁-C₁₂-alkyl. In another embodiment, R_{B} is C₁-C₁₂-alkyl substituted with alkoxy. In another embodiment, R_{B} is C₁-C₁₂-alkyl substituted with halogen. In another embodiment, R_{B} is aryl substituted with C₁-C₁₂-alkoxy. In another embodiment, R_{B} is aryl substituted with C₁-C₆-alkoxy-C₁-C₆-alkoxy. In another embodiment, R_{B} is aryl substituted with amino-C₁-C₆-alkoxy. In another embodiment, R_{B} is aryl substituted with C₁-C₁₂-alkylamino-C₁-C₆-alkoxy. In another embodiment, R_{B} is C₁-C₁₂-alkyl substituted with heteroaryl or substituted heteroaryl. In another embodiment, R_{B} is heteroaryl substituted with C₁-C₁₂-alkyl. In another embodiment, R_{B} is heteroaryl substituted with halogen. In another embodiment, R_{B} is heteroaryl substituted with C₁-C₁₂-alkyl. In another embodiment, R_{B} is heteroaryl substituted with substituted C₁-C₁₂-alkyl. In another embodiment, R_{B} is heteroaryl substituted with C₁-C₁₂-alkoxy. In another embodiment, R_{B} is heteroaryl substituted with C₁-C₆-alkoxy-C₁-C₆-alkoxy. In another embodiment, R_{B} is heteroaryl substituted with amino-C₁-C₆-alkoxy. In another embodiment, R_{B} is heteroaryl substituted with C₁-C₁₂-alkylamino-C₁-C₆-alkoxy.

In a further embodiment of any of the aforementioned embodiments, R_{C} is each selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C(═O)R₇ wherein R₇ is previously defined,
l) C(═O)CHR₈NR₉R₁₀ wherein R₈, R₉ and R₁₀ are independently selected from a group consisting of hydrogen, loweralkyl, substituted loweralkyl, aryl, substituted aryl, heteroaryl or substituted heteroaryl,
   or
   R₉ and R₁₀ taken together with the atom to which they are attached form a 3-10 membered heterocycloalkyl ring which is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) C₁-C₃haloalkyl,
   (h) C₁-C₃-alkoxy-C₁-C₃-alkyl.

In a further embodiment, R_{C} is hydrogen. In another embodiment, R_{C} is C₁-C₁₂-alkyl. In another embodiment, R_{C} is C₁-C₁₂-alkyl substituted with aryl or substituted aryl. In another embodiment, R_{C} is C₁-C₁₂-alkyl substituted with heteroaryl or substituted heteroaryl. In another embodiment, R_{C} is C₁-C₁₂-alkyl substituted with halogen. In another embodiment, R_{C} is C(═O)C₁-C₁₂-alkyl. In another embodiment, R_{C} is C(═O)CH₂NH C₁-C₂-alkyl. In another embodiment, R_{C} is C₁-C₁₂-alkyl substituted C₁-C₁₂-alkoxy. In another embodiment, R_{C} is C₁-C₁₂-alkyl substituted C₁-C₁₂-thioalkoxy. In another embodiment, R_{C} is C₁-C₁₂-alkyl substituted C₁-C₁₂-alkylamino.

In a further embodiment of any of the above structures, Y is oxygen and R₄ is hydrogen. In another embodiment, Y is NH and R₄ is hydrogen. In another embodiment, Y is oxygen and R₄ is CH₂NHCH₂PO₃H₂. In another embodiment, Y is NH and R₄ is CH₂NHCH₂PO₃H₂.

In a further embodiment, T is —SO₂R_{B} and R₄ is hydrogen. In another embodiment, T is —COR_{B} and R₄ is hydrogen. In another embodiment, T is —CONHSO₂R_{B} and R₄ is hydrogen. In another embodiment, T is —SO₂R_{B} and R₄ is CH₂NHCH₂PO₃H₂. In another embodiment, T is —COR_{B} and R₄ is CH₂NHCH₂PO₃H₂. In another embodiment, T is —CONHSO₂R_{B} and R₄ is CH₂NHCH₂PO₃H₂.

In a further embodiment of any of the aforementioned embodiments, R₁ is hydrogen and R₂ is COCHR₈NHR₁₆ wherein R₁₆ is substituted arylalkyl and R₈ is as previously defined.

In another aspect are compounds selected from Compound (23), Compound (24), Compound (26), Compound (27), Compound (28), Compound (29), Compound (30), Compound (31), Compound (32), Compound (33), Compound (34), Compound (35), Compound (36), Compound (37), Compound (38), Compound (39), Compound (40), Compound (41), Compound (42), Compound (43), Compound (44), Compound (45), Compound (46), Compound (47), Compound (48), Compound (49), Compound (50), Compound (51), Compound (52), Compound (53), Compound (54), Compound (55), Compound (56), Compound (57), Compound (58), Compound (59), Compound (60), Compound (61), Compound (62), Compound (64), Compound (64), Compound (65), Compound (66), Compound (67), Compound (68), Compound (69), Compound (98), Compound (99), Compound (100), Compound (101), Compound (102), Compound (103), Compound (104), Compound (105), Compound (106), Compound (107), Compound (115), Compound (116), Compound (117), Compound (118), Compound (119), Compound (120), Compound (121), Compound (122), Compound (123), Compound (124), Compound (125), Compound (126), Compound (127), Compound (143), Compound (144), Compound (145), Compound (150), Compound (151), Compound (152), Compound (153), Compound (155), Compound (156), Compound (157), Compound (158), Compound (159), Compound (160), Compound (161), Compound (162), Compound (163), Compound (164), Compound (165) and Compound (166).

In another aspect are pharmaceutical compositions comprising a therapeutically effective amount of any of the aforementioned compounds, together with a pharmaceutically acceptable carrier or excipient thereof.

In another aspect are methods of treating a mammal in need of such treatment comprising administering to the mammal an antibacterial effective amount of any of the aforementioned compounds together with a pharmaceutically acceptable carrier or excipient thereof.

In another aspect, described herein is the use of a compound described herein in the manufacture of a medicament for the treatment of a bacterial-related disease or condition.

In another aspect, described herein are articles of manufacture, comprising packaging material, a compound of any of Formula I, Formula II, Formula III, Formula IV, or Formula V, which is effective for treatment, prevention or amelioration of one or more symptoms of a bacterial-mediated disease or condition, within the packaging material, and a label that indicates that the compound or composition, or pharmaceutically acceptable salt, pharmaceutically acceptable N-oxide, pharmaceutically acceptable acyl glucuride metabolite, pharmaceutically acceptable prodrug, or pharmaceutically acceptable solvate thereof, is used for treatment, prevention or amelioration of one or more symptoms of a bacterial-mediated disease or condition, are provided.

In another aspect are methods of making a compound of Formulas I-V, comprising:
modifying a compound from the group consisting of Formulas i, ii, iii, iv and v,

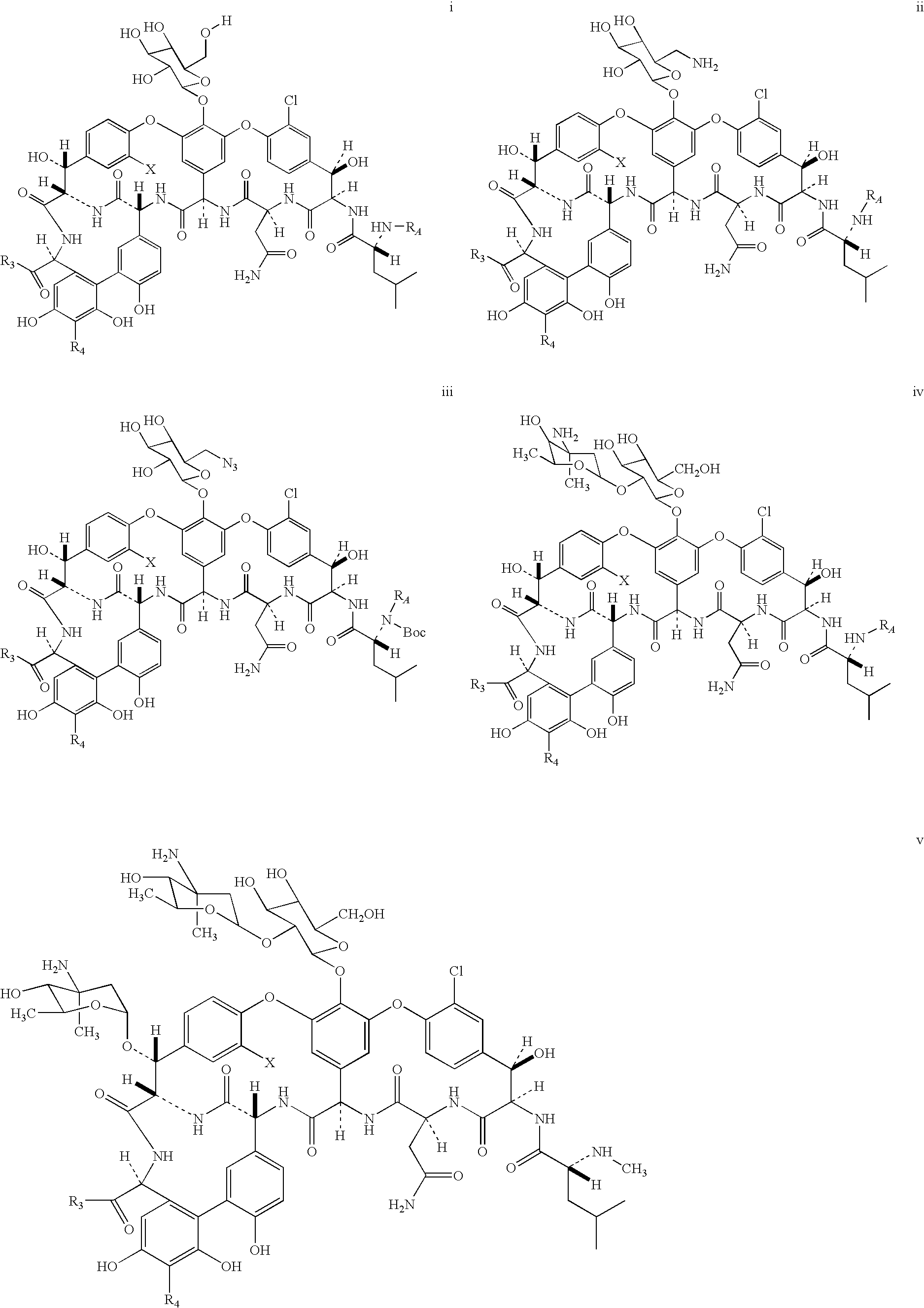

wherein R_{A} is hydrogen or methyl, X is chlorine or hydrogen, R₃ is OH or alkoxy, 2-adamantanamino, or loweralkylamino as defined herein, R₄ is hydrogen or properly protected CH₂NHCH₂PO₃H₂, or Boc-aminoloweralkyl as defined herein, by a technique selected from the group consisting of,
   (a) protecting the amino group with 9-fluorenylmethoxycarbonyl (Fmoc) or tert-butoxycarbonyl (Boc), or other appropriate nitrogen protecting groups.
   (b) acylating the primary amide group of the 3^{rd} amino acid asparagine with an R_{B}SO₂Cl, R_{B}COOH with a coupling reagent, or R_{B}SO₂—NCO group in the presence of a base such as triethylamine and the like,
   (c) removing the amino protecting group. Boc protecting group is removed with mild acid such as trifluoroacetic acid and Fmoc group is removed with base such as diethylamine and the like.
   (d) if the R₃ is alkoxy, removal of the alkoxy group by mild base or acid hydrolysis to give the carboxylic acid derivative,
   (e) reducing the azide function to an amine,
   (f) alkylating the primary alcohol of the mono-sugar or the amino substituent on the amino-substituted sugar moiety of the 4^{th} amino acid of the compound with an alkyl halide having structure R-J where J is a halogen, R₁-J where J is a halogen, R₂-J where J is a halogen or R_{C}-J where J is a halogen
   (g) acylating the primary alcohol of the mono-sugar or the amino substituent on the amino-substituted sugar moiety of the 4^{th} amino acid of the compound with an acyl group having the structure, C(═O)R₇,
   (h) acylating the primary alcohol of the mono-sugar or the amino substituent on the amino-substituted sugar moiety of the 4^{th} amino acid of the compound with an acyl group having the structure, C(═O)CHR₈NR₉R₁₀,
   (i) reacting the amino substituent on the amino-substituted sugar moiety of the 4^{th} amino acid of the compound with an aldehyde or ketone followed by reductive amination of the resulting imine,
   (j) converting the acid moiety on the macrocyclic ring of the compound with substituted amide as defined by R₃,
   (k) phosgene reaction on primary alcohol or primary amine of the mono-sugar moiety of the 4^{th} amino acid of the compound with the adjacent hydroxyl group,
   (l) Mannich reaction on the 7^{th} amino acid of the compound where R₄ is hydrogen with NH₂—CHR₁₅—(CH₂)ₘ—NHSO₂R_{B}, NHR_{D}—CHR₁₅—(CH₂)_{q}—NR_{E}SO₂R_{B}, or NH₂—CHR₁₅—(CH₂)ₚ—CONHSO₂R_{B} in the presence of aqueous formaldehyde in acetonitrile and water or other suitable organic solvent,
   (m) a combination of (a), (b) and (c),
   (n) a combination of (a), (b), (c) and (d),
   (o) a combination of (a), (b), (d), (j) and (c),
   (p) a combination of (a), (b), (f), and (c),
   (q) a combination of (a), (b), (g) and (c),
   (r) a combination of (a), (b), (h) and (c),
   (s) a combination of (a), (b), (i) and (c),
   (t) a combination of (a), (b), (e) and (c),
   (u) a combination of (a), (b), (e), (d) and (c),
   (v) a combination of (a), (b), (d), (j), (e) and (c),
   (w) a combination of (a), (b), (d), (e) and (c),
   (x) a combination of (a), (b), (d), (j), (e), (f) and (c),
   (y) a combination of (a), (b), (d), (j), (e), (g) and (c),
   (z) a combination of (a), (b), (d), (j), (e), (h) and (c),
   (aa) a combination of (a), (b), (d), (j), (e), (i) and (c),
   (bb) a combination of (a), (b), (d), (e), (f) and (c),
   (cc) a combination of (a), (b), (d), (e), (g) and (c),
   (dd) a combination of (a), (b), (d), (e), (h) and (c),
   (ee) a combination of (a), (b), (d), (e), (i) and (c),
   (ff) a combination of (a), (b), (k), and (c),
   (gg) a combination of (a), (b), (k), (d), (j) and (c),
   (hh) a combination of (a), (b), (e), (k), and (c),
   (ii) a combination of (a), (b), (e), (k), (d), (j) and (c),
   (jj) a combination of (a), (l), and (c),
   (kk) a combination of (a), (j), (l), and (c),
   (ll) a combination of (j), (a), (l), and (c),
to form a compound having a formula selected from the group consisting of:

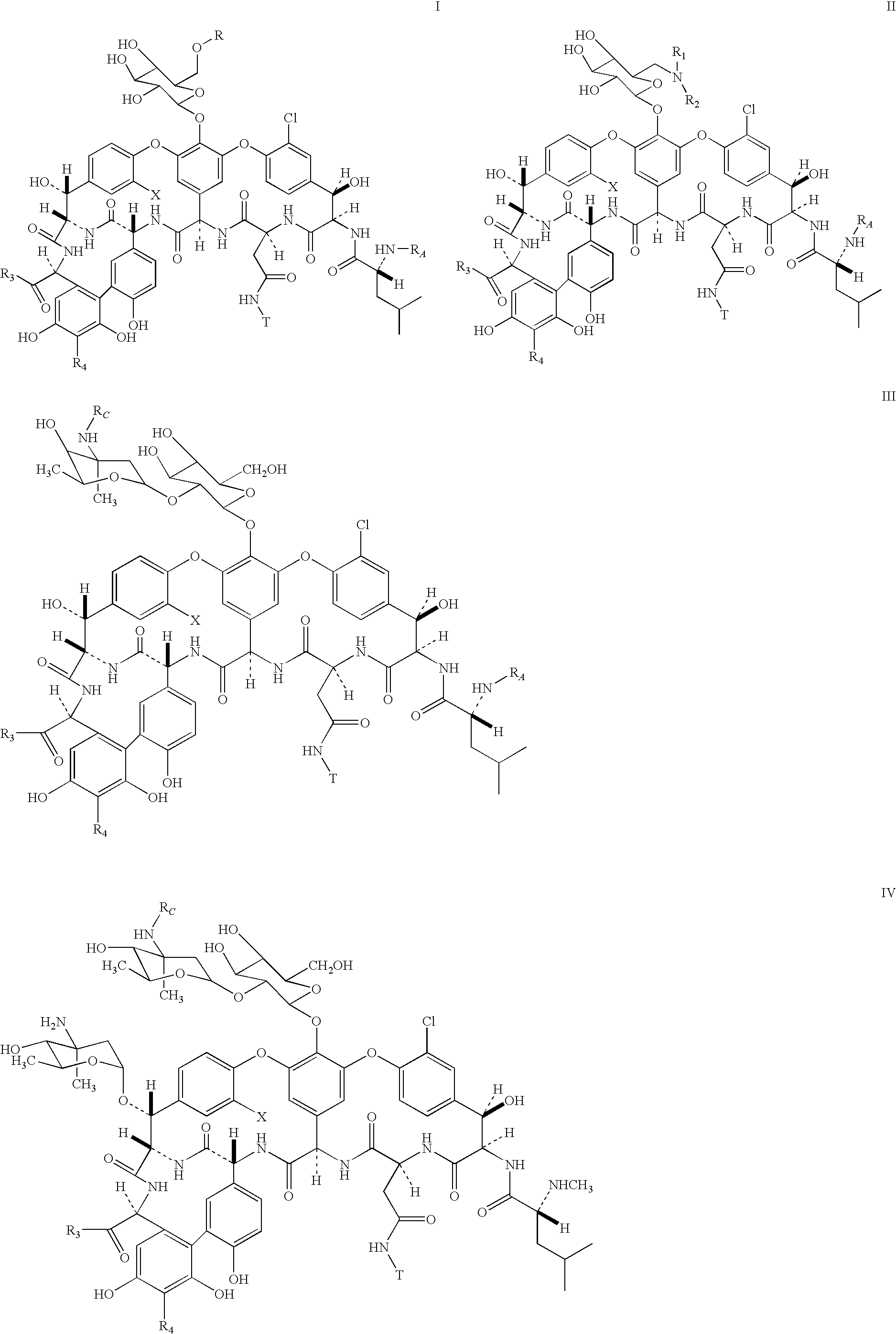

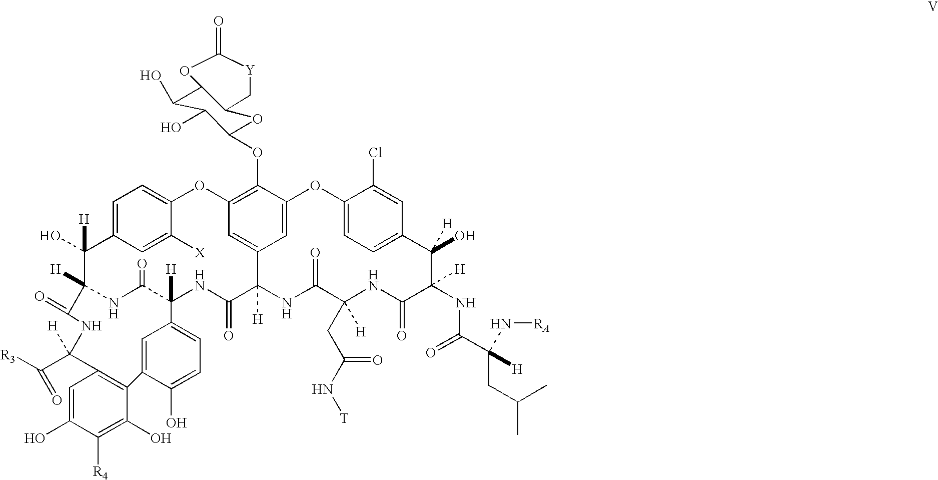

wherein R, R₁, R₂, R₃, R₄, R_{A}, X, Y, and T are as defined herein.

### DETAILED DESCRIPTION

The materials and associated techniques and apparatuses described herein will now be described with reference to several embodiments. Important properties and characteristics of the described embodiments are illustrated in the structures in the text. While the compositions, compounds and methods described herein are described in conjunction with these embodiments, it should be understood that the compositions, compounds and methods described herein are not to be limited to these embodiments. On the contrary, the compositions, compounds and methods described herein cover alternatives, modifications, and equivalents as are included within the spirit and scope of the appended claims. In the following description, numerous specific details are set forth in order to provide a thorough understanding of the compositions, compounds and methods described herein. The compositions, compounds and methods described herein are optionally practiced without some or all of these specific details.

Described herein are novel semi-synthetic glycopeptides that have antibacterial activity. The semi-synthetic glycopeptides described herein are either modification of natural glycopeptides or based on hydrolysis of the disaccharide moiety of the amino acid-4 of the parent glycopeptide to monosaccharide; conversion of the monosaccharide to the amino-sugar; acylation of the amino substituent on the amino-substituted sugar moiety on these natural or semisynthetic scaffolds with certain acyl groups; and conversion of the acid moiety on the macrocyclic ring of these scaffolds to certain substituted amides. Key reaction is acylation of properly protected intermediate compound on the primary amide group of the 3^{rd} amino acid asparagine with an R_{B}SO₂Cl, R_{B}COOH with a coupling reagent, or R_{B}SO₂—NCO group in the presence of a base such as triethylamine or Mannich reaction on the 7^{th} amino acid of the properly protected compound where R₄ is hydrogen with NH₂—CHR₁₅—(CH₂)ₘ—NHSO₂R_{B}, NHR_{D}—CHR₁₅—(CH₂)_{q}—NR_{E}SO₂R_{B}, or NH₂—CHR₁₅—(CH₂)ₚ—CONHSO₂R_{B} in the presence of aqueous formaldehyde in acetonitrile and water or other suitable organic solvent. Also provided are methods for synthesis of the compounds, pharmaceutical compositions containing the compounds, and methods of use of the compounds for the treatment and/or prophylaxis of diseases, especially bacterial infections.

### Compounds

Described herein are compounds having a structure selected from the group consisting of Formulas I, II, III, IV, and V,

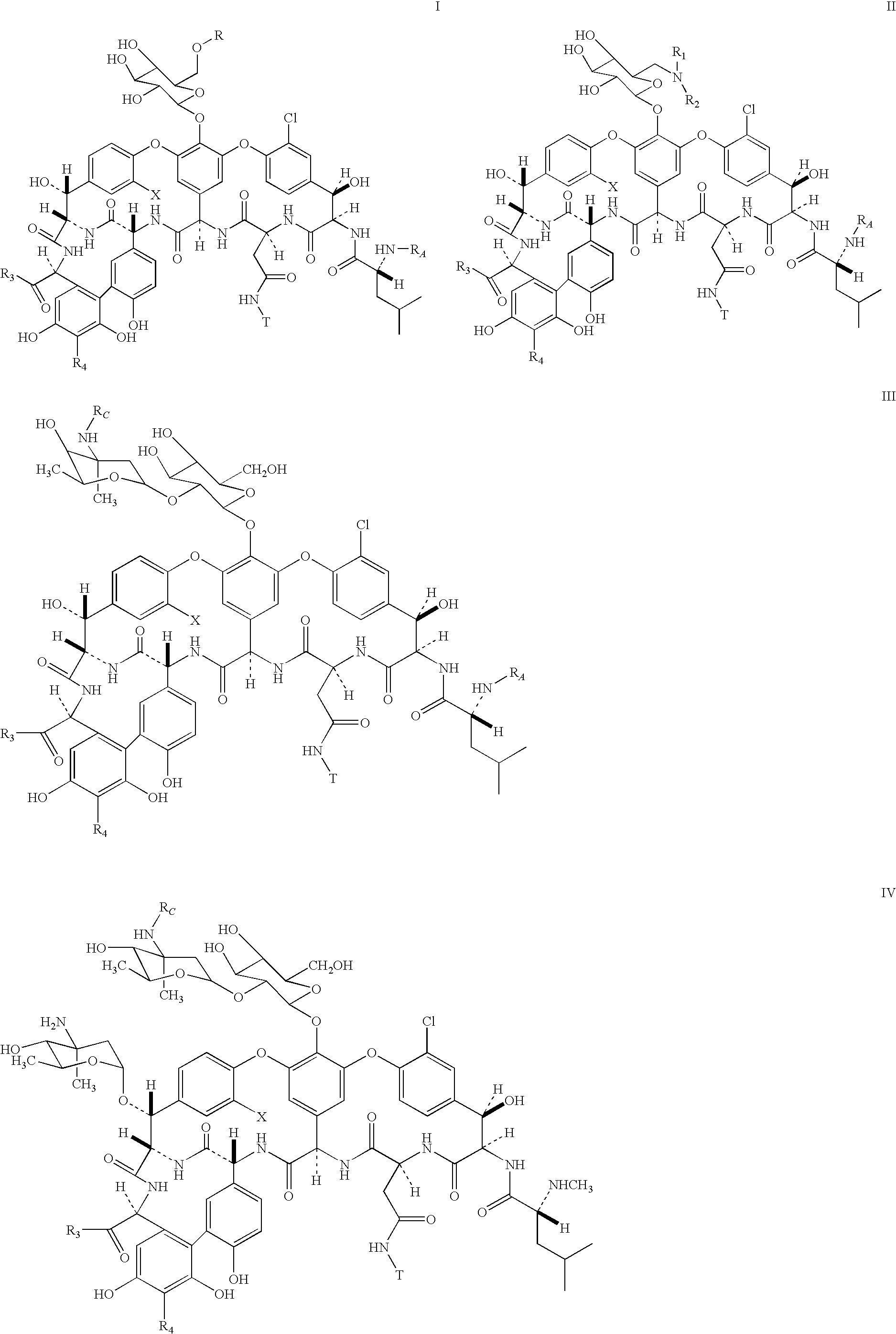

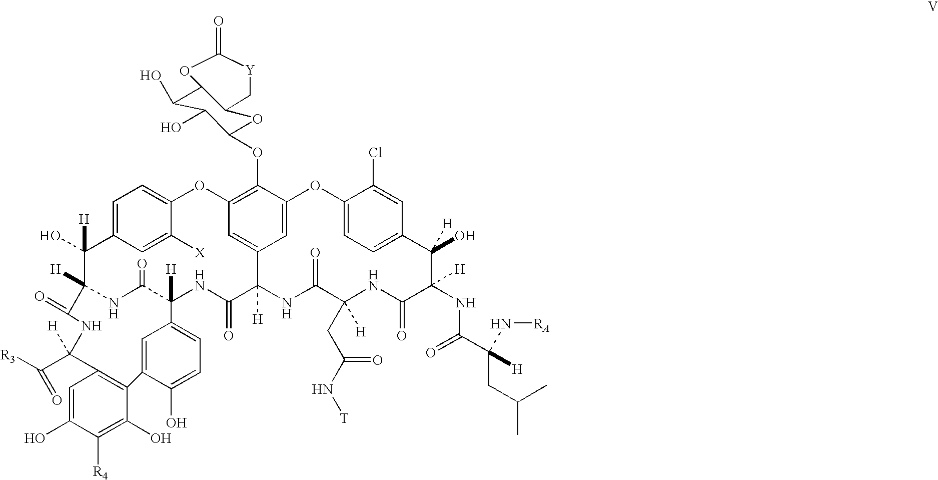

wherein,
R_{A} is selected from the group consisting of
   a) hydrogen,
   b) methyl,
   c) C₂-C₁₂-alkyl;
R₁ and R₂ are each independently selected from the group consisting of
   a) hydrogen,
   b) C₁-C₁₂-alkyl,
   c) C₁-C₁₂-alkyl substituted with one or more substituents selected from the group consisting of
      (a) halogen,
      (b) hydroxy,
      (c) C₁-C₁₂-alkoxy,
      (d) C₁-C₃-alkoxy-C₁-C₃-alkoxy,
      (e) amino,
      (f) C₁-C₁₂-alkylamino,
      (g) C₁-C₁₂-dialkylamino,
      (h) alkenyl,
      (i) alkynyl,
      (j) C₁-C₁₂-thioalkoxy,
   d) C₁-C₁₂-alkyl substituted with aryl,
   e) C₁-C₁₂-alkyl substituted with substituted aryl,
   f) C₁-C₁₂-alkyl substituted with heteroaryl,
   g) C₁-C₁₂-alkyl substituted with substituted heteroaryl,
   h) cycloalkyl,
   i) cycloalkenyl,
   j) heterocycloalkyl,
      or
      R₁ and R₂ taken together with the atom to which they are attached form a substituted heteroaryl or 3-10 membered heterocycloalkyl ring which optionally contains one or two hetero functionalities selected from the group consisting of —O—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring optionally be substituted with one or more substituents independently selected from the group consisting of
      (a) halogen,
      (b) hydroxyl,
      (c) C₁-C₃-alkoxy,
      (d) C₁-C₃-alkoxy-C₁-C₃-alkoxy,
      (e) oxo,
      (f) C₁-C₃-alkyl,
      (g) C₁-C₃haloalkyl,
      (h) C₁-C₃-alkoxy-C₁-C₃-alkyl,
   and
   k) C(═O)R₇,
   l) C(═O)CHR₈NR₉R₁₀ wherein R₈, R₉ and R₁₀ are independently selected from a group consisting of hydrogen, loweralkyl, substituted loweralkyl, aryl, substituted aryl, heteroaryl or substituted heteroaryl,
      or
      R₉ and R₁₀ taken together with the atom to which they are attached form a 3-10 membered heterocycloalkyl ring which is optionally substituted with one or more substituents independently selected from the group consisting of
      (a) halogen,
      (b) hydroxyl,
      (c) C₁-C₃-alkoxy,
      (d) C₁-C₃-alkoxy-C₁-C₃-alkoxy,
      (e) oxo,
      (f) C₁-C₃-alkyl,
      (g) C₁-C₃-haloalkyl,
      (h) C₁-C₃-alkoxy-C₁-C₃-alkyl;
R₇ is selected from the group consisting of
   a) hydrogen,
   b) C₁-C₁₂-alkyl,
   c) C₁-C₁₂-alkyl substituted with one or more substituents selected from the group consisting of
      (a) halogen,
      (b) hydroxy,
      (c) C₁-C₁₂-alkoxy,
      (d) C₁-C₃-alkoxy-C₁-C₃-alkoxy,
      (e) amino,
      (f) C₁-C₁₂-alkylamino,
      (g) C₁-C₁₂-dialkylamino,
      (h) alkenyl,
      (i) alkynyl,
      (j) C₁-C₁₂-thioalkoxy,
   d) C₁-C₁₂-alkyl substituted with aryl,
   e) C₁-C₁₂-alkyl substituted with substituted aryl,
   f) C₁-C₁₂-alkyl substituted with heteroaryl,
   g) C₁-C₁₂-alkyl substituted with substituted heteroaryl,
   h) cycloalkyl,
   i) cycloalkenyl,
   j) heterocycloalkyl,
   k) C₁-C₁₂-alkylamino,
   l) amino,
   m) amino-cycloalkyl;
X is selected from the group consisting of
   (1) hydrogen,
   (2) chlorine;
Y is selected from the group consisting of
   (1) oxygen,
   (2) NR₁, wherein R₁ is as previously defined;
T is selected from the group consisting of
   (1) —SO₂R_{B},
   (2) —COR_{B},
   (3) —CONHSO₂R_{B};
R is selected from the group consisting of
   (1) hydrogen,
   (2) cycloalkyl,
   (3) cycloalkenyl,
   (4) C₁-C₁₂-alkyl,
   (5) C₁-C₁₂-alkyl substituted with one or more substituents selected from the group consisting of
      (a) halogen,
      (b) hydroxy,
      (c) C₁-C₁₂-alkoxy,
      (d) C₁-C₃-alkoxy-C₁-C₃-alkoxy,
      (e) —COOR₅ wherein R₅ is hydrogen or loweralkyl,
      (f) —C(O)NR₅R₆ wherein R₅ is as previously defined and R₆ is hydrogen or loweralkyl,
      (g) amino,
      (h) —NR₅R₆ wherein R₅ and R₆ are as previously defined,
         or
         R₅ and R₆ taken together with the atom to which they are attached from a 3-10 membered heterocycloalkyl ring which is optionally substituted with one or more substituents independently selected from the group consisting of
         (i) halogen,
         (ii) hydroxy,
         (iii) C₁-C₃-alkoxy,
         (iv) C₁-C₃-alkoxy-C₁-C₃-alkoxy,
         (v) oxo,
         (vi) C₁-C₁₂-alkyl,
         (vii) C₁-C₁₂haloalkyl,
         and
         (viii) C₁-C₃-alkoxy-C₁-C₁₂-alkyl,
      (i) aryl,
      (j) substituted aryl,
      (k) heteroaryl,
      (l) substituted heteroaryl,
      (m) mercapto,
      (n) C₁-C₁₂-thioalkoxy,
   (6) C(═O)OR₁₁, wherein R₁₁ is hydrogen, loweralkyl, substituted loweralkyl, aryl, substituted aryl, heteroaryl or substituted heteroaryl,
   (7) C(═O)NR₁₁R₁₂, wherein R₁₁ is as previously defined and R₁₂ is hydrogen, loweralkyl, substituted loweralkyl, aryl, substituted aryl, heteroaryl or substituted heteroaryl,
      or
      R₁₁ and R₁₂ together with the atom to which they are attached form a 3-10 membered heterocycloalkyl ring, which is optionally substituted with one or more substituents independently selected from the group consisting of
         (a) halogen,
         (b) hydroxy,
         (c) C₁-C₃-alkoxy,
         (d) C₁-C₃-alkoxy-C₁-C₃-alkoxy,
         (e) oxo,
         (f) C₁-C₁₂-alkyl,
         (g) substituted loweralkyl,
         (h) C₁-C₁₂haloalkyl,
         (i) amino,
         (j) alkylamino,
         (k) dialkylamino
         and
         (l) C₁-C₃-alkoxy-C₁-C₁₂-alkyl,
      or
      R and its connected oxygen atom taken together is halogen;
R₃ is selected from the group consisting of
   (1) OH,
   (2) 1-adamantanamino,
   (3) 2-adamantanamino,
   (4) 3-amino-1-adamantanamino,
   (5) 1-amino-3-adamantanamino,
   (6) 3-loweralkylamino-1-adamantanamino,
   (7) 1-loweralkylamino-3-adamantanamino,
   (8) amino
   (9) NR₁₃R₁₄ wherein R₁₃ and R₁₄ are each independently selected from the group consisting of hydrogen, loweralkyl, substituted loweralkyl, cycloalkyl, substituted cycloalkyl, aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy
   or
R₁₃ and R₁₄ together with the atom to which they are attached form a 3-10 membered heterocycloalkyl ring, which is optionally substituted with one or more substituents independently selected from the group consisting of
   (a) halogen,
   (b) hydroxy,
   (c) C₁-C₃-alkoxy,
   (d) C₁-C₃-alkoxy-C₁-C₃-alkoxy,
   (e) oxo,
   (f) C₁-C₁₂-alkyl,
   (g) substituted loweralkyl,
   (h) C₁-C₁₂haloalkyl,
   (i) amino,
   (j) alkylamino,
   (k) dialkylamino
   and
   (l) C₁-C₃-alkoxy-C₁-C₁₂-alkyl;
R₄ is selected from the group consisting of
   (1) CH₂NH—CHR₁₅—(CH₂)ₘ—NHSO₂R_{B}, wherein m is 1 to 6 and R₁₅ is H or loweralkyl,
   (2) CH₂NH—CHR₁₅—(CH₂)ₚ—CONHSO₂R_{B}, wherein p is 0 to 6 and R₁₅ is H or loweralkyl,
   (3) CH₂NH—CHR₁₅—(CH₂)ₚ—COOH, wherein p is 0 to 6 and R₁₅ is H or loweralkyl,
   (4) CH₂NR_{D}—CHR₁₅—(CH₂)_{q}—NR_{E}SO₂R_{B}, wherein q is 2 to 4 and R₁₅ is H or loweralkyl, R_{D} and R_{E} together represents —CH₂—,
   (5) H,
   (6) CH₂NHCH₂PO₃H₂,
   (7) aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy,
with the proviso that when T is hydrogen, R₄ of structures II, III, and IV cannot be selected from the group consisting of H, CH₂NHCH₂PO₃H₂, aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy;
R_{B} is selected from the group consisting of
   a) aryl,
   b) C₁-C₁₂-alkyl,
   c) C₁-C₁₂-alkyl substituted with one or more substituents selected from the group consisting of
      (a) halogen,
      (b) hydroxy,
      (c) C₁-C₁₂-alkoxy,
      (d) C₁-C₃-alkoxy-C₁-C₃-alkoxy,
      (e) amino,
      (f) C₁-C₁₂-alkylamino,
      (g) C₁-C₁₂-dialkylamino,
      (h) alkenyl,
      (i) alkynyl,
      (j) C₁-C₁₂-thioalkoxy,
   d) C₁-C₁₂-alkyl substituted with aryl,
   e) C₁-C₁₂-alkyl substituted with substituted aryl,
   f) C₁-C₁₂-alkyl substituted with heteroaryl,
   g) C₁-C₁₂-alkyl substituted with substituted heteroaryl,
   h) cycloalkyl,
   i) heteroaryl,
   j) heterocycloalkyl,
   k) aryl substituted with one or more substituents selected from the group consisting of
      (a) halogen,
      (b) hydroxy,
      (c) C₁-C₁₂-alkoxy,
      (d) C₁-C₆-alkoxy-C₁-C₆-alkoxy,
      (e) amino,
      (f) amino-C₁-C₆-alkoxy
      (g) C₁-C₁₂-alkylamino,
      (h) C₁-C₁₂-alkylamino-C₁-C₆-alkoxy,
      (i) C₁-C₁₂-dialkylamino,
      (j) C₁-C₁₂-dialkylamino-C₁-C₆-alkoxy,
      (k) alkenyl,
      (l) alkynyl,
      (m) C₁-C₁₂-thioalkoxy,
      (n) C₁-C₁₂-alkyl,
      (o) C₁-C₁₂-substituted alkyl,
      (p) C₁-C₁₂-alkoxy-morpholino,
      (q) C₁-C₁₂-alkoxy-C₁-C₁₂-dialkoxyamino,
      (r) C₁-C₁₂-alkoxy-NHSO₂C₁-C₆-alkyl,
      (s) C₁-C₁₂-alkoxy-NHCOC₁-C₆-alkyl;
   l) heteroaryl substituted with one or more substituents selected from the group consisting of
      (a) halogen,
      (b) hydroxy,
      (c) C₁-C₁₂-alkoxy,
      (d) C₁-C₆-alkoxy-C₁-C₆-alkoxy,
      (e) amino,
      (f) amino-C₁-C₆-alkoxy
      (g) C₁-C₁₂-alkylamino,
      (h) C₁-C₁₂-alkylamino-C₁-C₆-alkoxy,
      (i) C₁-C₁₂-dialkylamino,
      (j) C₁-C₁₂-dialkylamino-C₁-C₆-alkoxy,
      (k) alkenyl,
      (l) alkynyl,
      (m) C₁-C₁₂-thioalkoxy,
      (n) C₁-C₁₂-alkyl,
      (o)C₁-C₁₂-substituted alkyl;
R_{C} is each selected from the group consisting of
   a) hydrogen,
   b) C₁-C₁₂-alkyl,
   c) C₁-C₁₂-alkyl substituted with one or more substituents selected from the group consisting of
      (a) halogen,
      (b) hydroxy,
      (c) C₁-C₁₂-alkoxy,
      (d) C₁-C₃-alkoxy-C₁-C₃-alkoxy,
      (e) amino,
      (f) C₁-C₁₂-alkylamino,
      (g) C₁-C₁₂-dialkylamino,
      (h) alkenyl,
      (i) alkynyl,
      (j) C₁-C₁₂-thioalkoxy,
   d) C₁-C₁₂-alkyl substituted with aryl,
   e) C₁-C₁₂-alkyl substituted with substituted aryl,
   f) C₁-C₁₂-alkyl substituted with heteroaryl,
   g) C₁-C₁₂-alkyl substituted with substituted heteroaryl,
   h) cycloalkyl,
   i) cycloalkenyl,
   j) heterocycloalkyl,
   k) C(═O)R₇ wherein R₇ is previously defined,
   l) C(═O)CHR₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
      or
      R₈ and R₁ taken together with the atom to which they are attached form a 3-10 membered heterocycloalkyl ring which is optionally substituted with one or more substituents independently selected from the group consisting of
      (a) halogen,
      (b) hydroxyl,
      (c) C₁-C₃-alkoxy,
      (d) C₁-C₃-alkoxy-C₁-C₃-alkoxy,
      (e) oxo,
      (f) C₁-C₃-alkyl,
      (g) C₁-C₃haloalkyl,
      (h) C₁-C₃-alkoxy-C₁-C₃-alkyl;
or a pharmaceutically acceptable salt, ester, solvate, alkylated quaternary ammonium salt, stereoisomer, tautomer or prodrug thereof.

Also provided herein are pharmaceutical compositions which comprise a therapeutically effective amount of a compound as defined above in combination with a pharmaceutically acceptable carrier.

According to the methods of treatment provided herein, bacterial infections are treated or prevented in a patient such as a human or lower mammal by administering to the patient a therapeutically effective amount of a compound provided herein, in such amounts and for such time as is necessary to achieve the desired result.

In a further aspect are provided processes and intermediates for the preparation of semi-synthetic glycopeptides of Formulas I, II, III, IV, and V above.

In a further embodiment of any of the above structures, R_{A} is methyl and R₄ is hydrogen. In another embodiment, R_{A} is hydrogen and R₄ is hydrogen. In another embodiment, X is hydrogen and R₄ is hydrogen. In another embodiment, X is chlorine and R₄ is hydrogen. In another embodiment, R_{A} is methyl and R₄ is CH₂NHCH₂PO₃H₂. In another embodiment, R_{A} is hydrogen and R₄ is CH₂NHCH₂PO₃H₂. In another embodiment, R_{A} is hydrogen and R₄ is CH₂NH—CHR₁₅—(CH₂)ₘ—NHSO₂R_{B}, wherein m is 1 to 6 and R₁₅ is H or loweralkyl. In another embodiment, R_{A} is hydrogen and R₄ is CH₂NR_{D}—CHR₁₅—(CH₂)_{q}—NR_{E}SO₂R_{B}, wherein q is 2 to 4 and R₁₅ is H or loweralkyl, R_{D} and R_{E} together represents —CH₂—. In another embodiment, R_{A} is hydrogen and R₄ is CH₂NH—CHR₁₅—(CH₂)ₚ—CONHSO₂R_{B}, wherein p is 0 to 6 and R₁₅ is H or loweralkyl. In another embodiment, R_{A} is hydrogen and R₄ is CH₂NH—CHR₁₅—(CH₂)ₚ—COOH, wherein p is 0 to 6 and R₁₅ is H or loweralkyl. In another embodiment, R_{A} is methyl and R₄ is CH₂NH—CHR₁₅—(CH₂)ₘ—NHSO₂R_{B}, wherein m is 1 to 6 and R₁₅ is H or loweralkyl. In another embodiment, R_{A} is methyl and R₄ is CH₂NH—CHR₁₅—(CH₂)ₚ—CONHSO₂R_{B}, wherein p is 0 to 6 and R₁₅ is H or loweralkyl. In another embodiment, R_{A} is methyl and R₄ is CH₂NH—CHR₁₅—(CH₂)ₚ—COOH, wherein p is 0 to 6 and R₁₅ is H or loweralkyl.

In a further embodiment of any of the aforementioned embodiments, R₃ is selected from the group consisting of
(1) OH,
(2) 1-adamantanamino,
(3) 2-adamantanamino,
(4) 3-amino-1-adamantanamino,
(5) 1-amino-3-adamantanamino,
(6) 3-loweralkylamino-1-adamantanamino,
(7) 1-loweralkylamino-3-adamantanamino,
(8) amino
(9) NR₁₃R₁₄ wherein R₁₃ and R₁₄ are each independently selected from the group consisting of hydrogen, loweralkyl, substituted loweralkyl, cycloalkyl, substituted cycloalkyl, aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy
or
R₁₃ and R₁₄ together with the atom to which they are attached form a 3-10 membered heterocycloalkyl ring, which is optionally substituted with one or more substituents independently selected from the group consisting of
   (a) halogen,
   (b) hydroxy,
   (c) C₁-C₃-alkoxy,
   (d) C₁-C₃-alkoxy-C₁-C₃-alkoxy,
   (e) oxo,
   (f) C₁-C₁₂-alkyl,
   (g) substituted loweralkyl,
   (h) C₁-C₁₂haloalkyl,
   (i) amino,
   (j) alkylamino,
   (k) dialkylamino
   and
   (l) C₁-C₃-alkoxy-C₁-C₁₂-alkyl.

In a further embodiment, R₃ is OH. In another embodiment, R₃ is 2-adamantanamino. In another embodiment, R₃ is dimethylamino. In another embodiment, R₃ is diethylamino. In another embodiment, R₃ is dimethylaminoethylamino. In another embodiment, R₃ is N-methylpiperazino.

In a further embodiment of any of the aforementioned embodiments, R₁ and R₂ are each independently selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
   or
   R₁ and R₂ taken together with the atom to which they are attached form a substituted heteroaryl or 3-10 membered heterocycloalkyl ring which optionally contains one or two hetero functionalities selected from the group consisting of —O—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or S(O)ₙ— wherein n is 1 or 2 and the 3-10 membered heterocycloalkyl ring optionally be substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) C₁-C₃haloalkyl,
   (h) C₁-C₃-alkoxy-C₁-C₃-alkyl,
and
k) C(═O)R₇,
l) C(═O)CHR₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl, or
   R₉ and R₁₀ taken together with the atom to which they are attached form a 3-10 membered heterocycloalkyl ring which is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) C₁-C₃haloalkyl,
   (h) C₁-C₃-alkoxy-C₁-C₃-alkyl.

In a further embodiment of any of the aforementioned embodiments, R₁ and R₂ are hydrogen. In another embodiment, R₁ is C₁-C₁₂-alkyl and R₂ is hydrogen. In another embodiment, R₁ is C₁-C₁₂-alkyl substituted with aryl or substituted aryl and R₂ is hydrogen. In another embodiment, R₁ is C(═O)C₁-C₁₂-alkyl and R₂ is hydrogen. In another embodiment, R₁ is C(═O)CH₂NH C₁-C₁₂-alkyl and R₂ is hydrogen. In another embodiment, R₁ is C₁-C₁₂-alkyl substituted C₁-C₁₂-alkoxy and R₂ is hydrogen. In another embodiment, R₁ is C₁-C₁₂-alkyl substituted C₁-C₁₂-thioalkoxy and R₂ is hydrogen. In another embodiment, R₁ is C₁-C₁₂-alkyl substituted C₁-C₁₂-alkylamino and R₂ is hydrogen.

In a further embodiment of any of the aforementioned embodiments, R is selected from the group consisting of
(1) hydrogen,
(2) cycloalkyl,
(3) cycloalkenyl,
(4) C₁-C₁₂-alkyl,
(5) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) —COOR₅ wherein R₅ is hydrogen or loweralkyl,
   (f) —C(O)NR₅R₆ wherein R₅ is as previously defined and R₆ is hydrogen or loweralkyl,
   (g) amino,
   (h) —NR₅R₆ wherein R₅ and R₆ are as previously defined,
      or
      R₅ and R₆ taken together with the atom to which they are attached form a 3-10 membered heterocycloalkyl ring which is optionally substituted with one or more substituents independently selected from the group consisting of
      (i) halogen.
      (ii) hydroxy,
      (iii) C₁-C₃-alkoxy,
      (iv) C₁-C₃-alkoxy-C₁-C₃-alkoxy,
      (v) oxo,
      (vi) C₁-C₁₂-alkyl,
      (vii) C₁-C₁₂haloalkyl,
      and
      (viii) C₁-C₃-alkoxy-C₁-C₁₂-alkyl,
   (i) aryl,
   (j) substituted aryl,
   (k) heteroaryl,
   (l) substituted heteroaryl,
   (m) mercapto,
   (n) C₁-C₁₂-thioalkoxy,
(6) C(═O)OR₁₁, wherein R₁₁ is hydrogen, loweralkyl, substituted loweralkyl, aryl, substituted aryl, heteroaryl or substituted heteroaryl,
(7) C(═O)NR₁₁R₁₂, wherein R₁₁ is as previously defined and R₁₂ is hydrogen, loweralkyl, substituted loweralkyl, aryl, substituted aryl, heteroaryl or substituted heteroaryl,
   or
   R₁₁ and R₁₂ together with the atom to which they are attached form a 3-10 membered heterocycloalkyl ring, which is optionally substituted with one or more substituents independently selected from the group consisting of
      (a) halogen,
      (b) hydroxy,
      (c) C₁-C₃-alkoxy,
      (d) C₁-C₃-alkoxy-C₁-C₃-alkoxy,
      (e) oxo,
      (f) C₁-C₁₂-alkyl,
      (g) substituted loweralkyl,
      (h) C₁-C₁₂haloalkyl,
      (i) amino,
      (j) alkylamino,
      (k) dialkylamino
      and
      (l) C₁-C₃-alkoxy-C₁-C₁₂-alkyl,
   or
   R and its connected oxygen atom taken together is halogen.

In a further embodiment of any of the aforementioned embodiments, R is hydrogen. In another embodiment, R is C₁-C₁₂-alkyl. In another embodiment, R is C₁-C₁₂-alkyl substituted with aryl or substituted aryl. In another embodiment, R is C(═O)NHC₁-C₁₂-alkyl. In another embodiment, R is C(═O)NHC₁-C₁₂-alkyl substituted with aryl or substituted aryl. In another embodiment, R is C(═O)OC₁-C₁₂-alkyl. In another embodiment, R is C(═O)NHC₁-C₁₂-alkyl substituted with heteroaryl or substituted heteroaryl.

In a further embodiment of any of the aforementioned embodiments, R_{B} is selected from the group consisting of
a) aryl,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) heteroaryl,
j) heterocycloalkyl,
k) aryl substituted with one or more substituents selected from the group consisting of
   (a) halogen,
   (b) hydroxy,
   (c) C₁-C₁₂-alkoxy,
   (d) C₁-C₆-alkoxy-C₁-C₆-alkoxy,
   (e) amino,
   (f) amino-C₁-C₆-alkoxy
   (g) C₁-C₁₂-alkylamino,
   (h) C₁-C₁₂-alkylamino-C₁-C₆-alkoxy,
   (i) C₁-C₁₂-dialkylamino,
   (j) C₁-C₁₂-dialkylamino-C₁-C₆-alkoxy,
   (k) alkenyl,
   (l) alkynyl,
   (m) C₁-C₁₂-thioalkoxy,
   (n) C₁-C₁₂-alkyl,
l) heteroaryl substituted with one or more substituents selected from the group consisting of
   (a) halogen,
   (b) hydroxy,
   (c) C₁-C₁₂-alkoxy,
   (d) C₁-C₆-alkoxy-C₁-C₆-alkoxy,
   (e) amino,
   (f) amino-C₁-C₆-alkoxy,
   (g) C₁-C₁₂-alkylamino,
   (h) C₁-C₁₂-alkylamino-C₁-C₆-alkoxy,
   (i) C₁-C₁₂-dialkylamino,
   (j) C₁-C₁₂-dialkylamino-C₁-C₆-alkoxy,
   (k) alkenyl,
   (l) alkynyl,
   (m) C₁-C₁₂-thioalkoxy,
   (n) C₁-C₁₂-alkyl;

In a further embodiment of any of the aforementioned embodiments, R_{B} is C₁-C₁₂-alkyl. In another embodiment, R_{B} is C₁-C₁₂-alkyl substituted with aryl or substituted aryl. In another embodiment, R_{B} is C₁-C₁₂-alkyl substituted with heteroaryl or substituted heteroaryl. In another embodiment, R_{B} is aryl substituted with C₁-C₁₂-alkyl. In another embodiment, R_{B} is aryl substituted with halogen. In another embodiment, R_{B} is aryl substituted with substituted C₁-C₁₂-alkyl. In another embodiment, R_{B} is C₁-C₁₂-alkyl substituted with alkoxy. In another embodiment, R_{B} is C₁-C₁₂-alkyl substituted with halogen. In another embodiment, R_{B} is aryl substituted with C₁-C₁₂-alkoxy. In another embodiment, R_{B} is aryl substituted with C₁-C₆-alkoxy-C₁-C₆-alkoxy. In another embodiment, R_{B} is aryl substituted with amino-C₁-C₆-alkoxy. In another embodiment, R_{B} is aryl substituted with C₁-C₁₂-alkylamino-C₁-C₆-alkoxy. In another embodiment, R_{B} is C₁-C₁₂-alkyl substituted with heteroaryl or substituted heteroaryl. In another embodiment, R_{B} is heteroaryl substituted with C₁-C₁₂-alkyl. In another embodiment, R_{B} is heteroaryl substituted with halogen. In another embodiment, R_{B} is heteroaryl substituted with C₁-C₁₂-alkyl. In another embodiment, R_{B} is heteroaryl substituted with substituted C₁-C₁₂-alkyl. In another embodiment, R_{B} is heteroaryl substituted with C₁-C₁₂-alkoxy. In another embodiment, R_{B} is heteroaryl substituted with C₁-C₆-alkoxy-C₁-C₆-alkoxy. In another embodiment, R_{B} is heteroaryl substituted with amino-C₁-C₆-alkoxy. In another embodiment, R_{B} is heteroaryl substituted with C₁-C₁₂-alkylamino-C₁-C₆-alkoxy.

In a further embodiment of any of the aforementioned embodiments, R_{C} is each selected from the group consisting of
a) hydrogen,
b) C₁-C₁₂-alkyl,
c) C₁-C₁₂-alkyl substituted with one or more substituents selected from the group consisting of
   (a) halogen,
   (b) hydroxy,
   (c) C₁-C₁₂-alkoxy,
   (d) C₁-C₃-alkoxy-C₁-C₃-alkoxy,
   (e) amino,
   (f) C₁-C₁₂-alkylamino,
   (g) C₁-C₁₂-dialkylamino,
   (h) alkenyl,
   (i) alkynyl,
   (j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C(═O)R₇ wherein R₇ is previously defined,
l) C(═O)CHR₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
   or
   R₉ and R₁₀ taken together with the atom to which they are attached form a 3-10 membered heterocycloalkyl ring which is optionally substituted with one or more substituents independently selected from the group consisting of
   (a) halogen,
   (b) hydroxyl,
   (c) C₁-C₃-alkoxy,
   (d) C₁-C₃-alkoxy-C₁-C₃-alkoxy,
   (e) oxo,
   (f) C₁-C₃-alkyl,
   (g) C₁-C₃haloalkyl,
   (h) C₁-C₃-alkoxy-C₁-C₃-alkyl.

In a further embodiment of any of the aforementioned embodiments, R_{C} is hydrogen. In another embodiment, R_{C} is C₁-C₁₂-alkyl. In another embodiment, R_{C} is C₁-C₁₂-alkyl substituted with aryl or substituted aryl. In another embodiment, R_{C} is C₁-C₁₂-alkyl substituted with heteroaryl or substituted heteroaryl. In another embodiment, R_{C} is C₁-C₁₂-alkyl substituted with halogen. In another embodiment, R_{C} is C(═O)C₁-C₁₂-alkyl. In another embodiment, R_{C} is C(═O)CH₂NH C₁-C₂-alkyl. In another embodiment, R_{C} is C₁-C₁₂-alkyl substituted C₁-C₁₂-alkoxy. In another embodiment, R_{C} is C₁-C₁₂-alkyl substituted C₁-C₁₂-thioalkoxy. In another embodiment, R_{C} is C₁-C₁₂-alkyl substituted C₁-C₁₂-alkylamino.

In a further embodiment of any of the above structures, Y is oxygen and R₄ is hydrogen. In another embodiment, Y is NH and R₄ is hydrogen. In another embodiment, Y is oxygen and R₄ is CH₂NHCH₂PO₃H₂. In another embodiment, Y is NH and R₄ is CH₂NHCH₂PO₃H₂.

In a further embodiment, T is —SO₂R_{B} and R₄ is hydrogen. In another embodiment, T is —COR_{B} and R₄ is hydrogen. In another embodiment, T is —CONHSO₂R_{B} and R₄ is hydrogen. In another embodiment, T is —SO₂R_{B} and R₄ is CH₂NHCH₂PO₃H₂. In another embodiment, T is —COR_{B} and R₄ is CH₂NHCH₂PO₃H₂. In another embodiment, T is —CONHSO₂R_{B} and R₄ is CH₂NHCH₂COOH. In another embodiment, T is —SO₂R_{B} and R₄ is CH₂NHCH₂COOH. In another embodiment, T is —COR_{B} and R₄ is CH₂NHCH₂COOH. In another embodiment, T is —CONHSO₂R_{B} and R₄ is CH₂NHCH₂COOH.

In a further embodiment of any of the aforementioned embodiments, R₁ is hydrogen and R₂ is COCHR₈NHR₁₆ wherein R₁₆ is substituted arylalkyl and R₈ is as previously defined.

In another embodiment are provided intermediate compounds of Formulas i, ii, iii, iv, and wherein R_{A} is hydrogen or methyl, X is chlorine or hydrogen, and R₄ is hydrogen, CH₂NHCH₂PO₃H₂, or aminoloweralkyl, R₃ is alkoxy or 2-adamantylamino for the synthesis of antibacterial agents of Formulas I-V.

### DEFINITIONS

Unless otherwise noted, terminology used herein should be given its normal meaning as understood by one of skill in the field.

The term “alkyl” as used herein refers to saturated, straight- or branched-chain hydrocarbon radicals derived from a hydrocarbon moiety containing between one and twenty carbon atoms by removal of a single hydrogen atom.

The term substituted alkyl as used herein refers to alkyl substituted by one, two or three groups consisting of halogen, alkoxy, amino, alkylamino, dialkylamino, hydroxy, aryl, heteroaryl, alkenyl or alkynyl groups.

The term “alkenyl” as used herein refers to unsaturated, straight- or branched-chain hydrocarbon radicals derived from a hydrocarbon moiety containing between two and twenty carbon atoms by removal of a single hydrogen atom.

The term “cycloalkyl” as used herein refers to a monovalent group derived from a monocyclic or bicyclic saturated carbocyclic ring compound containing between three and twenty carbon atoms by removal of a single hydrogen atom.

The term substituted cycloalkyl as used herein refers to cycloalkyl substituted by one, two or three groups consisting of halogen, alkoxy, amino, alkylamino, dialkylamino, hydroxy, aryl, heteroaryl, alkenyl or alkynyl groups.

The term “cycloalkenyl” as used herein refers to a monovalent group derived from a monocyclic or bicyclic unsaturated carbocyclic ring compound containing between three and twenty carbon atoms by removal of a single hydrogen atom.

The terms “C₁-C₃-alkyl”, “C₁-C₆-alkyl”, and “C₁-C₁₂-alkyl” as used herein refer to saturated, straight- or branched-chain hydrocarbon radicals derived from a hydrocarbon moiety containing between one and three, one and six, and one and twelve carbon atoms, respectively, by removal of a single hydrogen atom. Examples of C₁-C₃-alkyl radicals include methyl, ethyl, propyl and isopropyl. Examples of C₁-C₆-alkyl radicals include, but not limited to, methyl, ethyl, propyl, isopropyl, n-butyl, tert-butyl, neopentyl and n-hexyl. Examples of C₁-C₁₂-alkyl radicals include, but not limited to, methyl, ethyl, propyl, isopropyl, n-butyl, tert-butyl, neopentyl, n-hexyl. N-heptyl, n-octyl, n-nonyl, n-decyl, n-undecyl and n-docecyl.

The term loweralkyl as used herein refers to C₁-C₁₂-alkyl as defined above.

The term substituted loweralkyl as used herein refers to C₁-C₁₂-alkyl substituted by one, two or three groups consisting of halogen, alkoxy, amino, alkylamino, dialkylamino, hydroxy, aryl, heteroaryl, alkenyl or alkynyl groups.

The term “C₃-C₁₂-cycloalkyl” denoted a monovalent group derived from a monocyclic or bicyclic saturated carbocyclic ring compound by removal of a single hydrogen atom. Examples include cyclopropyl, cyclobutyl, cyclopentyl, cyclohexyl, bicyclo[2.2.1]heptyl, and bicyclo[2.2.2]octyl.

The terms “C₁-C₃-alkoxy”, “C₁-C₆-alkoxy” as used herein refers to the C₁-C₃-alkyl group and C₁-C₆-alkyl group, as previously defined, attached to the parent molecular moiety through an oxygen atom. Examples of C₁-C₆-alkoxy radicals include, but not limited to, methoxy, ethoxy, propoxy, isopropoxy, n-butoxy, tert-butoxy, neopentoxy and n-hexoxy.

The term “loweralkylamino” as used herein refers to C₁-C₁₂-alkyl groups, as previously defined, attached to the parent molecular moiety through a nitrogen atom. Examples of loweralkylamino include, but are not limited to methylamino, dimethylamino, ethylamino, diethylamino, propylamino and decylamino.

The term “oxo” denotes a group wherein two hydrogen atoms on a single carbon atom in an alkyl group as defined above are replaced with a single oxygen atom (i.e. a carbonyl group).

The term “aryl” as used herein refers to a mono- or bicyclic carbocylic ring system having one or two aromatic rings including, but not limited to, phenyl, naphthyl, tetrahydronaphthyl, indanyl, indenyl and the like and is optionally un-substituted or substituted (including bicyclic aryl groups) with one, two or three substituents independently selected from loweralkyl, substituted loweralkyl, haloalkyl, C₁-C₁₂-alkoxy, thioalkoxy, C₁-C₁₂-thioalkoxy, aryloxy, amino, alkylamino, dialkylamino, acylamino, cyano, hydroxy, halogen, mercapto, nitro, carboxaldehyde, carboxy, alkoxycarbonyl and carboxamide. In addition, substituted aryl groups include tetrafluorophenyl and pentafluorophenyl.

The term “substituted aryl” as used herein refers to a mono- or bicyclic carbocylic ring system having one or two aromatic rings including, but not limited to, phenyl, naphthyl, tetrahydronaphthyl, indanyl, indenyl and the like substituted (including bicyclic aryl groups) with one, two or three substituents independently selected from loweralkyl, substituted loweralkyl, haloalkyl, C₁-C₁₂-alkoxy, thioalkoxy, C₁-C₁₂-thioalkoxy, alkoxyalkylalkoxy, aryloxy, amino, aminoalkyl, aminoalkylalkoxy, alkylamino, alkylaminoalkyl, alkylaminoalkylalkoxy, dialkylamino, dialkylaminoalkyl, dialkylaminoalkylalkoxy, acylamino, cyano, hydroxy, halogen, mercapto, nitro, carboxaldehyde, carboxy, alkoxycarbonyl, aryl, heteroaryl, heterocycloaryl and carboxamide. In addition, substituted aryl groups include tetrafluorophenyl and pentafluorophenyl.

The term “arylalkyl” as used herein refers to an aryl group as defined above attached to the parent molecular moiety through an alkyl group wherein the alkyl group is of one to twelve carbon atoms.

The term “substituted arylalkyl” as used herein refers to a substituted aryl group as defined above attached to the parent molecular moiety through an alkyl group wherein the alkyl group is of one to twelve carbon atoms.

The term “alkylaryl” as used herein refers to an alkyl group as defined above attached to the parent molecular moiety through an aryl group.

The term “halo” and “halogen” as used herein refer to an atom selected from fluorine, chlorine, bromine and iodine.

The term “alkylamino” refers to a group having the structure —NHR′ wherein R′ is alkyl, as previously defined. Examples of alkylamino include methylamino, ethylamino, iso-propylamino, and the like.

The term “dialkylamino” refers to a group having the structure —NHR′R″ wherein R′ and R″ are independently selected from alkyl, as previously defined. Additionally, R′ and R″ taken together optionally be —(CH₂)ₖ— where k is an integer of from 2 to 6. Examples of dialkylamino include dimethylamino, diethylamino, methylpropylamino, piperidino, and the like.

The term “haloalkyl” denotes an alkyl group, as defined above, having one, two or three halogen atoms attached thereto and is exemplified by such group as chloromethyl, bromoethyl, trifluoromethyl, and the like.

The term “alkoxycarbonyl” represents as ester group; i.e. an alkoxy group, attached to the parent molecular moiety through a carbonyl group such as methoxycarbonyl, ethoxycarbonyl, and the like.

The term “thioalkoxy” refers to an alkyl group previously defined attached to the parent molecular moiety through a sulfur atom.

The term “carboxaldehyde” as used herein refers to a group of formula —CHO.

The term “carboxy” as used herein refers to a group of formula —CO₂H.

The term “carboxamide” as used herein refers to a group of formula —CONHR′R″ wherein R′ and R″ are independently selected from hydrogen, alkyl, substituted loweralkyl, or R′ and R″ taken together optionally be —(CH₂)ₖ— where k is an integer of from 2 to 6.

The term “heteroaryl”, as used herein, refers to a cyclic or bicyclic aromatic radical having from five to ten ring atoms in each ring of which at least one atom of the cyclic or bicyclic ring is selected from optionally substituted S, O, and N; zero, one or two ring atoms are additional heteroatoms independently selected from optionally substituted S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, naphthyridinyl; and the like.

The term “substituted heteroaryl” as used herein refers to a cyclic or bicyclic aromatic radical having from five to ten ring atoms in each ring of which at least one atom of the cyclic or bicyclic ring is selected from optionally substituted S, O, and N; zero, one or two ring atoms are additional heteroatoms independently selected from optionally substituted S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, naphthyridinyl; and the like substituted with one, two or three substituents independently selected from loweralkyl, substituted loweralkyl, haloalkyl, C₁-C₁₂-alkoxy, thioalkoxy, C₁-C₁₂-thioalkoxy, alkoxyalkylalkoxy, aryloxy, amino, aminoalkyl, aminoalkylalkoxy, alkylamino, alkylaminoalkyl, alkylaminoalkylalkoxy, dialkylamino, dialkylaminoalkyl, dialkylaminoalkylalkoxy, acylamino, cyano, hydroxy, halogen, mercapto, nitro, carboxaldehyde, carboxy, alkoxycarbonyl, aryl, heteroaryl, heterocycloaryl and carboxamide.

The term “heterocycloalkyl” as used herein, refers to a non-aromatic partially unsaturated or fully saturated 3- to 10-membered ring system, which includes single rings of 3 to 8 atoms in size and bi- or tri-cyclic ring systems which includes aromatic six-membered aryl or heteroaryl rings fused to a non-aromatic ring. These heterocycloalkyl rings include those having from one to three heteroatoms independently selected from oxygen, sulfur and nitrogen, in which the nitrogen and sulfur heteroatoms optionally be oxidized and the nitrogen heteroatom optionally be quaternized. Representative heterocycloalkyl rings include, but not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “heteroarylalkyl” as used herein, refers to a heteroaryl group as defined above attached to the parent molecular moiety through an alkylene group wherein the alkylene group is of one to four carbon atoms.

“Protecting group” refers to an easily removable group to protect a functional group, for example, a hydroxyl, ketone or amine, against undesirable reaction during synthetic procedures and to be selectively removable. Examples of such protecting groups are known, cf., for example, T. H. Greene and P. G. M. Wuts, Protective Groups in Organic Synthesis, 2nd edition, John Wiley & Sons, New York (1991). Examples of hydroxy-protecting groups include, but not limited to, methylthiomethyl, tert-dimethylsilyl, tert-butyldiphenylsilyl, ethers such as methoxymethyl, and esters including acetyl, benzoyl, and the like. Examples of ketone protecting groups include, but not limited to, ketals, oximes, O-substituted oximes for example O-benzyl oxime, O-phenylthiomethyl oxime, 1-isopropoxycyclohexyl oxime, and the like. Examples of amine protecting groups include, but are not limited to, tert-butoxycarbonyl (Boc) and carbobenzyloxy (Cbz).

A term “protected-hydroxy” refers to a hydroxy group protected with a hydroxy protecting group, as defined above.

The term amino acid refers to amino acids having D or L stereochemistry, and also refers to synthetic, non-natural amino acids having side chains other than those found in the 20 common amino acids. Non-natural amino acids are commercially available or are optionally prepared according to U.S. Pat. No. 5,488,131 and references therein. Amino acids are optionally further substituted to contain modifications to their amino, carboxy, or side-chain groups. These modifications include the numerous protecting group commonly used in peptide synthesis (T. H. Greene and P. G. M. Wuts, Protective Groups in Organic Synthesis, 2nd edition, John Wiley & Sons, New York, 1991).

The term “substituted heteroaryl” as used herein, refers to a heteroaryl group as defined herein substituted by independent replacement of one, two or three of the hydrogen atoms thereon with Cl, Br, F, I, OH, CN, C₁-C₁₂-alkyl, C₁-C₁₂-alkoxy, C₁-C₁₂-alkoxy substituted with aryl, haloalkyl, thioalkyl, amino, alkylamino, dialkylamino, mercapto, nitro, carboxaldehyde, carboxy, alkoxycarbonyl and carboxamide. In addition, any one substituent is optionally an aryl, heteroaryl, or heterocycloalkyl group.

The term “substituted heterocycloalkyl” as used herein, refers to a heterocycloalkyl group as defined herein substituted by independent replacement of one, two or three of the hydrogen atoms thereon with Cl, Br, F, I, OH, CN, C₁-C₁₂-alkyl, C₁-C₁₂-alkoxy, C₁-C₁₂-alkoxy substituted with aryl, haloalkyl, thioalkyl, amino, alkylamino, dialkylamino, mercapto, nitro, carboxaldehyde, carboxy, alkoxycarbonyl and carboxamide. In addition, any one substituent is optionally aryl, heteroaryl, or heterocycloalkyl group.

The term “stereoisomer” as used herein, refers to either of two forms of a compound having the same molecular formula and having their constituent atoms attached in the same order, but having different arrangement if their atoms in space about an asymmetric center. If asymmetric centers exist in the described compounds, except where otherwise noted, the compounds described herein include the various stereoisomers and mixtures thereof. Accordingly, except where otherwise noted, it is intended that a mixture of stereo-orientations or an individual isomer of assigned or unassigned orientation is present.

The term “tautomer” as used herein refers to either of the two forms of a chemical compound that exhibits tautomerism, which is the ability of certain chemical compounds to exist as a mixture of two interconvertible isomers in equilibrium via proton transfer. The keto and enol forms of carbonyl compounds are examples of tautomers. They are interconvertible in the presence of traces of acids and bases via a resonance stabilized anion, the enolate ion.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. For example, S. M. Berge, et al. describes pharmaceutically acceptable salts in detail in J. Pharmaceutical Sciences, 66: 1-19 (1977), incorporated herein by reference for this purpose. The salts are prepared in situ during the final isolation and purification of the compounds described herein, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term “pharmaceutically acceptable ester” refers to esters which hydrolyze in vivo and include those that break down in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Representative examples of particular esters include, but are not limited to, formates, acetates, propionates, butyrates, acrylates and ethylsuccinates.

The term “solvate” as used herein refers to a compound formed by solvation, the combination of solvent molecules with molecules or ions of solute composed of a compound described herein. The term “pharmaceutically acceptable solvate” refers to those solvates which are, within the scope of sound medical judgment, suitable for use in contact with the tissues of humans and lover animals without undue toxicity, irritation, allergic response and the like, and are commensurate with a reasonable benefit/risk ratio.

The term “alkylated quaternary ammonium salt” as used herein refers to a compound formed by alkylation of the nitrogen atom of the primary, secondary or tertiary amine of the molecule with alkyl halide to form alkyl quaternary ammonium salt.

The term “pharmaceutically acceptable prodrugs” refers to those prodrugs of the compounds described herein which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds described herein. The term “prodrug” refers to compounds that are transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference for this purpose.

### Synthetic Methods

Synthesis of the compounds described herein is broadly summarized as follows. The compounds described herein are made, for example, by chemical modifications of the Compound A, Compound B, Compound H and Compound C scaffolds. In particular, the semi-synthetic glycopeptides described herein are made by chemical modification of Compound A, Compound B, Compound H and Compound C or of the monosaccharide of the about glycopeptides made by subjecting the parent glycopeptide in acidic medium to hydrolyze the disaccharide moiety of the amino acid-4 of the parent glycopeptide to give the monosaccharide; protection of the amino function by t-butoxycarbonyl group, carbobenzyloxy group, allyloxycarbonyl group or 9-fluorenylmethoxycarbonyl group; conversion of the acid moiety on the macrocyclic ring of these scaffolds to certain substituted amides; acylation of properly protected intermediate compound on the primary amide group of the 3^{rd} amino acid asparagine with an R_{B}SO₂Cl, R_{B}COOH with a coupling reagent, or R_{B}SO₂—NCO group in the presence of a base such as triethylamine and removal of the protecting group. In some embodiments, if amino function on the monosaccharide is required, conversion of the monosaccharide to the amino-sugar derivative; acylation of the amino substituent on the amino-substituted sugar moiety on these scaffolds with certain acyl groups; protection of the amino function by t-butoxycarbonyl group, carbobenzyloxy group, allyloxycarbonyl group or 9-fluorenylmethoxycarbonyl group; conversion of the acid moiety on the macrocyclic ring of these scaffolds to certain substituted amides and treatment of the compound with isocyanate. The compounds described herein are made, for example, by coupling the amino-sugar moiety of functionalized or unfunctionalized glycopeptides from the above scaffolds with the appropriate acyl and/or amino groups under amide formation conditions and conversion of the acid moiety on the macrocyclic ring of the resulting glycopeptide derivative to certain substituted amides; or a combination of an alkylation modification of the substituent on the amino-substituted sugar moiety on this scaffold with certain alkyl groups or acylation modification of the amino substituent on the amino-substituted sugar moiety on this scaffold with certain acyl groups, α-amino acid or β-amino acids or derivatives thereof, and conversion of the acid moiety on the macrocyclic ring of this scaffold to certain substituted amides. In another series, the compounds described herein are made, for example, by chemical modifications of the Compound A, Compound B, Compound H and Compound C scaffolds. In particular, the semi-synthetic glycopeptides described herein are made by chemical modification of Compound A, Compound B, Compound H and Compound C or of the monosaccharide of the about glycopeptides made by subjecting the parent glycopeptide in acidic medium to hydrolyze the disaccharide moiety of the amino acid-4 of the parent glycopeptide to give the monosaccharide; protection of the amino function by t-butoxycarbonyl group, carbobenzyloxy group, allyloxycarbonyl group or 9-fluorenylmethoxycarbonyl group; Mannich reaction on the 7^{th} amino acid of the properly protected compound where R₄ is hydrogen with NH₂—CHR₁₅—(CH₂)ₘ—NHSO₂R_{B}, NHR_{D}—CHR₁₅—(CH₂)_{q}—NR_{E}SO₂R_{B}, or NH₂—CHR₁₅—(CH₂)ₚ—CONHSO₂R_{B} in the presence of aqueous formaldehyde in acetonitrile and water or other suitable organic solvent. and removal of the protecting group. In some embodiments, synthesis of compounds also involves the use of protecting or blocking groups in order to maximize yields, minimize unwanted side products, or improve the ease purification.

In particular, the semi-synthetic glycopeptides of the compounds described herein are made, for example, by modifying Compound A, Compound B, Compound H and Compound C scaffolds. The glycopeptide starting material is optionally unsubstituted or substituted at the 7^{th} amino acid at the 4′ position of the phenyl ring with CH₂NHCH₂PO₃H₂, or aminoloweralkyl as defined herein.

Selective hydrolysis of Compound A, Compound B, Compound H or Compound C in which the 7^{th} amino acid at the 4′ position of the phenyl ring substituted with hydrogen, CH₂NHCH₂PO₃H₂, or aminoloweralkyl as defined herein with acid gives the monosaccharide intermediate.

In general, compound of Formulas I-V, described herein are made by modifying a compound from the group consisting of Formulas i, ii, iii, iv and v,

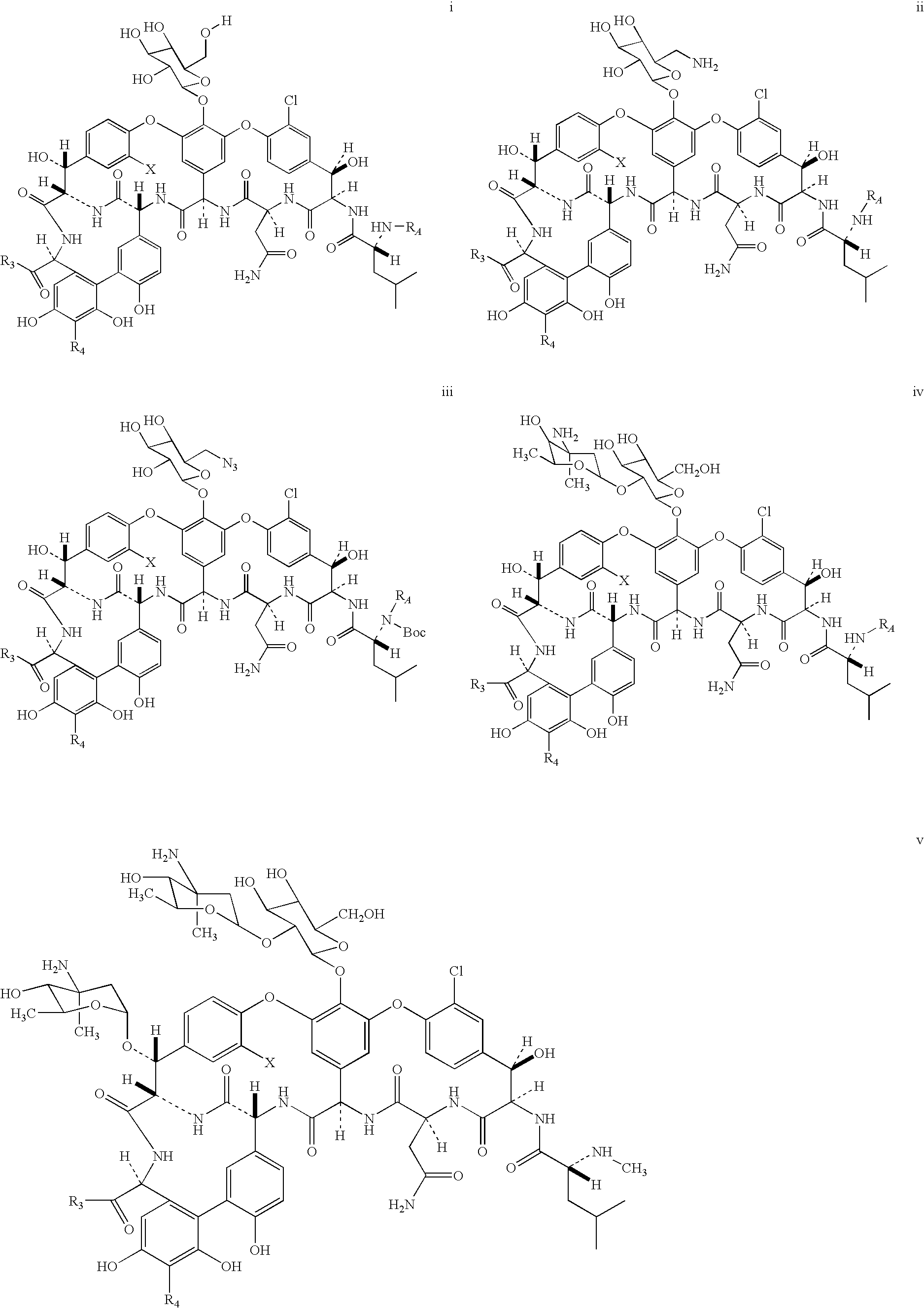

wherein R_{A} is hydrogen or methyl, X is chlorine or hydrogen, R₃ is OH or alkoxy, 2-adamantanamino, or loweralkylamino as defined herein, R₄ is hydrogen or properly protected CH₂NHCH₂PO₃H₂, or Boc-aminoloweralkyl as defined herein, by a technique selected from the group consisting of,
   (a) protecting the amino group with 9-fluorenylmethoxycarbonyl (Fmoc) or tert-butoxycarbonyl (Boc), or other appropriate nitrogen protecting groups.
   (b) acylating the primary amide group of the 3^{rd} amino acid asparagine with an R_{B}SO₂Cl, R_{B}COOH with a coupling reagent, or R_{B}SO₂—NCO group in the presence of a base such as triethylamine and the like,
   (c) removing the amino protecting group. Boc protecting group is removed with mild acid such as trifluoroacetic acid and Fmoc group is removed with base such as diethylamine and the like.
   (d) if the R₃ is alkoxy, removal of the alkoxy group by mild base or acid hydrolysis to give the carboxylic acid derivative,
   (e) reducing the azide function to an amine,
   (f) alkylating the primary alcohol of the mono-sugar or the amino substituent on the amino-substituted sugar moiety of the 4^{th} amino acid of the compound with an alkyl halide having structure R-J where J is a halogen, R₁-J where J is a halogen, R₂-J where J is a halogen or R_{C}-J where J is a halogen
   (g) acylating the primary alcohol of the mono-sugar or the amino substituent on the amino-substituted sugar moiety of the 4^{th} amino acid of the compound with an acyl group having the structure, C(═O)R₇,
   (h) acylating the primary alcohol of the mono-sugar or the amino substituent on the amino-substituted sugar moiety of the 4^{th} amino acid of the compound with an acyl group having the structure, C(═O)CHR₈NR₉R₁₀,
   (i) reacting the amino substituent on the amino-substituted sugar moiety of the 4^{th} amino acid of the compound with an aldehyde or ketone followed by reductive amination of the resulting imine,
   (j) converting the acid moiety on the macrocyclic ring of the compound with substituted amide as defined by R₃,
   (k) phosgene reaction on primary alcohol or primary amine of the mono-sugar moiety of the 4^{th} amino acid of the compound with the adjacent hydroxyl group,
   (l) Mannich reaction on the 7^{th} amino acid of the compound where R₄ is hydrogen with NH₂—CHR₁₅—(CH₂)ₘ—NHSO₂R_{B}, NHR_{D}—CHR₁₅—(CH₂)_{q}—NR_{E}SO₂R_{B}, or NH₂—CHR₁₅—(CH₂)ₚ—CONHSO₂R_{B} in the presence of aqueous formaldehyde in acetonitrile and water or other suitable organic solvent,
   (m) a combination of (a), (b) and (c),
   (n) a combination of (a), (b), (c) and (d),
   (o) a combination of (a), (b), (d), (j) and (c),
   (p) a combination of (a), (b), (f), and (c),
   (q) a combination of (a), (b), (g) and (c),
   (r) a combination of (a), (b), (h) and (c),
   (s) a combination of (a), (b), (i) and (c),
   (t) a combination of (a), (b), (e) and (c),
   (u) a combination of (a), (b), (e), (d) and (c),
   (v) a combination of (a), (b), (d), (j), (e) and (c),
   (w) a combination of (a), (b), (d), (e) and (c),
   (x) a combination of (a), (b), (d), (j), (e), (f) and (c),
   (y) a combination of (a), (b), (d), (j), (e), (g) and (c),
   (z) a combination of (a), (b), (d), (j), (e), (h) and (c),
   (aa) a combination of (a), (b), (d), (j), (e), (i) and (c),
   (bb) a combination of (a), (b), (d), (e), (f) and (c),
   (cc) a combination of (a), (b), (d), (e), (g) and (c),
   (dd) a combination of (a), (b), (d), (e), (h) and (c),
   (ee) a combination of (a), (b), (d), (e), (i) and (c),
   (ff) a combination of (a), (b), (k), and (c),
   (gg) a combination of (a), (b), (k), (d), (j) and (c),
   (hh) a combination of (a), (b), (e), (k), and (c),
   (ii) a combination of (a), (b), (e), (k), (d), (j) and (c),
   (jj) a combination of (a), (l), and (c),
   (kk) a combination of (a), (j), (l), and (c),
   (ll) a combination of (j), (a), (l), and (c),
to form a compound having a formula selected from the group consisting of:

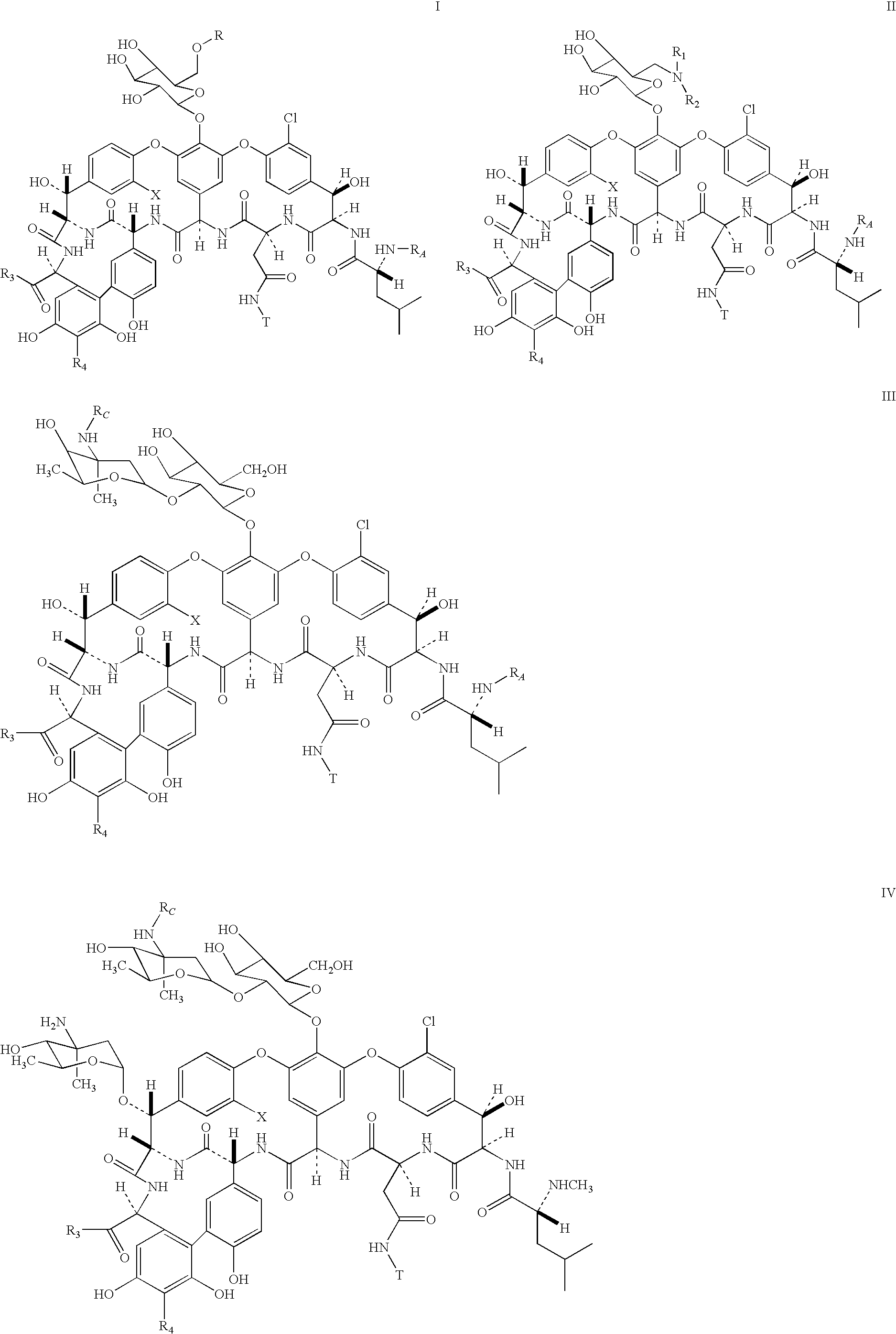

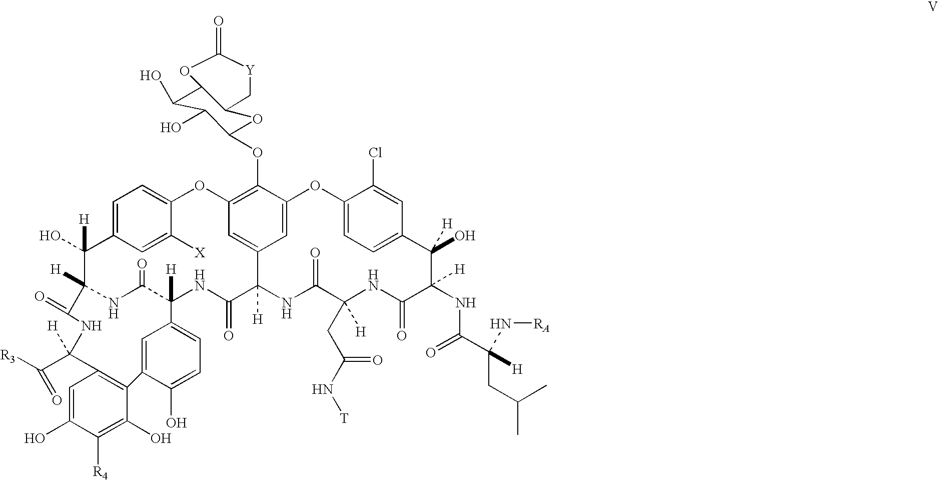

wherein R, R₁, R₂, R₃, R₄, R_{A}, X, Y, and T are as defined herein.

In particular, the semi-synthetic glycopeptides described herein are made, for example, by modifying Compound A, Compound B, Compound H or Compound C scaffolds. These natural glycopeptide starting material is optionally unsubstituted or substituted at R₄ with CH₂NHCH₂PO₃H₂, or aminoloweralkyl as defined herein.

Substitutions at R₄ are introduced, for example, via Mannich reaction wherein the glycopeptide is treated with an amine and formaldehyde under basic conditions (for example, as described in The Journal of Antibiotics, Vol. 50, No. 6, p. 509-513).

### Pharmaceutical Compositions

Pharmaceutical compositions described herein comprise a therapeutically effective amount of a compound described herein formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants are also present in the composition, according to the judgment of the formulator. The pharmaceutical compositions described herein are administered to humans and other animals orally, rectally, parenterally, intracisternally, intravaginally, intraperitoneally, topically (as by powders, ointments, or drops), bucally, or as an oral or nasal spray, or a liquid aerosol or dry powder formulation for inhalation.

Liquid dosage forms for oral administration include pharmaceutically acceptable emulsions, microemulsions, solutions, suspensions, syrups and elixirs. In addition to the active compounds, the liquid dosage forms optionally contain inert diluents such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions optionally also include adjuvants such as wetting agents, emulsifying and suspending agents, sweetening, flavoring, and perfuming agents.

Injectable preparations, for example, sterile injectable aqueous or oleaginous suspensions are formulated using suitable dispersing or wetting agents and suspending agents. The sterile injectable preparation are optionally a sterile injectable solution, suspension or emulsion in a nontoxic parenterally acceptable diluent or solvent, for example, as a solution in 1,3-butanediol. Among the acceptable vehicles and solvents that are optionally employed are water, Ringer's solution, U.S.P. and isotonic sodium chloride solution. In addition, sterile, fixed oils are optionally employed as a solvent or suspending medium. For this purpose any bland fixed oil is optionally employed including synthetic mono- or diglycerides. In addition, fatty acids such as oleic acid are used in the preparation of injectables.

The injectable formulations are sterilized, for example, by filtration through a bacterial-retaining filter, or by incorporating sterilizing agents in the form of sterile solid compositions which is dissolved or dispersed in sterile water or other sterile injectable medium prior to use.

In order to prolong the effect of a drug, it is often desirable to slow the absorption of the drug from subcutaneous or intramuscular injection. This is accomplished, for example, by the use of a liquid suspension of crystalline or amorphous material with poor water solubility. The rate of absorption of the drug then depends upon its rate of dissolution which, in turn, depends upon crystal size and crystalline form. In other embodiments, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release is optionally controlled. Examples of other biodegradable polymers include poly(orthoesters) and poly(anhydrides). Depot injectable formulations are also prepared, for example, by entrapping the drug in liposomes or microemulsions which are compatible with body tissues.

Compositions for rectal or vaginal administration are preferably suppositories which are optionally prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form optionally comprises buffering agents.

Solid compositions of a similar type are optionally employed as fillers in soft and hard-filled gelatin capsules using such excipients as lactose or milk sugar as well as high molecular weight polyethylene glycols and the like.

The solid dosage forms of tablets, dragees, capsules, pills, and granules are prepared, for example, with coatings and shells such as enteric coatings and other documented coatings. They optionally contain opacifying agents and also are of a composition that they release the active ingredient(s) only, or preferentially, in a certain part of the intestinal tract, optionally, in a delayed manner. Examples of embedding compositions which are used include polymeric substances and waxes.

Solid compositions of a similar type are optionally employed as fillers in soft and hard-filled gelatin capsules using such excipients as lactose or milk sugar as well as high molecular weight polyethylene glycols and the like.

The active compounds are optionally in micro-encapsulated form with one or more excipients as noted above. The solid dosage forms of tablets, dragees, capsules, pills, and granules are optionally prepared with coatings and shells such as enteric coatings, release controlling coatings and other documented coatings. In such solid dosage forms the active compound is admixed, for example, with at least one inert diluent such as sucrose, lactose or starch. Such dosage forms optionally comprise additional substances other than inert diluents, e.g., tableting lubricants and other tableting aids such a magnesium stearate and microcrystalline cellulose. In the case of capsules, tablets and pills, the dosage forms optionally comprise buffering agents. They optionally contain opacifying agents and are of a composition that they release the active ingredient(s) only, or preferentially, in a certain part of the intestinal tract, optionally, in a delayed manner. Examples of embedding compositions which are used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as required. Ophthalmic formulations, ear drops, and the like are also contemplated.

The ointments, pastes, creams and gels optionally contain, in addition to an active compound described herein, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions described herein are optionally formulated for delivery as a liquid aerosol or inhalable dry powder. Liquid aerosol formulations are nebulized, for example, predominantly into particle sizes that are delivered to the terminal and respiratory bronchioles where bacteria reside in patients with bronchial infections, such as chronic bronchitis and pneumonia. Pathogenic bacteria are commonly present throughout airways down to bronchi, bronchioli and lung parenchema, particularly in terminal and respiratory bronchioles. During exacerbation of infection, bacteria can also be present in alveoli. Liquid aerosol and inhalable dry powder formulations are preferably delivered throughout the endobronchial tree to the terminal bronchioles and eventually to the parenchymal tissue.

Aerosolized formulations described herein are delivered, for example, using an aerosol forming device, such as a jet, vibrating porous plate or ultrasonic nebulizer, preferably selected to allow the formation of a aerosol particles having with a mass medium average diameter predominantly between 1 to 5μ. Further, the formulation preferably has balanced osmolarity ionic strength and chloride concentration, and the smallest aerosolizable volume able to deliver effective dose of the compounds described herein to the site of the infection. Additionally, the aerosolized formulation preferably does not impair negatively the functionality of the airways and does not cause undesirable side effects.

Aerosolization devices suitable for administration of aerosol formulations described herein include, for example, jet, vibrating porous plate, ultrasonic nebulizers and energized dry powder inhalers, that are able to nebulize the formulation into aerosol particle size predominantly in the size range from 1-5μ. Predominantly in this application means that at least 70% but preferably more than 90% of all generated aerosol particles are within 1-5μ range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AeroNeb™ and AeroDose™ vibrating porous plate nebulizers (AeroGen, Inc., Sunnyvale, Calif.), Sidestream® nebulizers (Medic-Aid Ltd., West Sussex, England), Pari LC® and Pari LC Star® jet nebulizers (Pari Respiratory Equipment, Inc., Richmond, Va.), and Aerosonic™ (DeVilbiss Medizinische Produkte (Deutschland) GmbH, Heiden, Germany) and UltraAire® (Omron Healthcare, Inc., Vernon Hills, Ill.) ultrasonic nebulizers.

Compounds described herein are formulated, for example, for use as topical powders and sprays that contain, in addition to the compounds described herein, excipients such as lactose, talc, silicic acid, aluminum hydroxide, calcium silicates and polyamide powder, or mixtures of these substances. Sprays optionally contain customary propellants such as chlorofluorohydrocarbons.

Transdermal patches have the added advantage of providing controlled delivery of a compound to the body. Such dosage forms made, for example, by dissolving or dispensing the compound in the proper medium. Absorption enhancers are optionally used to increase the flux of the compound across the skin. The rate is controlled, for example, by either providing a rate controlling membrane or by dispersing the compound in a polymer matrix or gel.

According to the methods of treatment described herein, bacterial infections are treated or prevented in a patient such as a human or lower mammal by administering to the patient a therapeutically effective amount of a compound described herein, in such amounts and for such time as is necessary to achieve the desired result. By a “therapeutically effective amount” of a compound described herein is meant a sufficient amount of the compound to treat bacterial infections, at a reasonable benefit/risk ratio applicable to any medical treatment. It will be understood, however, that the total daily usage of the compounds and compositions described herein will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors known in the medical arts.

The total daily dose of the compounds described herein administered to a human or other mammal in single or in divided doses is in amounts, for example, from 0.01 to 50 mg/kg body weight or more usually from 0.1 to 25 mg/kg body weight. Single dose compositions contain, for example, such amounts or submultiples thereof to make up the daily dose. In general, treatment regimens described herein comprise administration to a patient in need of such treatment from about 10 mg to about 2000 mg of the compound(s) described herein per day in single or multiple doses.

### ABBREVIATIONS

In some embodiments, abbreviations have been used in the descriptions of the schemes and the examples that follow are: AcOH for acetic acid; AIBN for azobisisobutyronitrile; nBu for normal butyl; Bu₃SnH for tributyltin hydride; CDI for carbonyldiimidazole; DBU for 1,8-diazabicyclo[5.4.0]undec-7-ene; DCC for dicyclohexyl carbodiimide; DCM for dichloromethane; DEAD for diethylazodicarboxylate; DMF for dimethylformamide; DIEA or DIPEA for N,N-diisopropylethylamine; DMP for 2,2-dimethoxypropane; DMSO for dimethylsulfoxide (or methylsulfoxide); DPPA for diphenylphosphoryl azide; Et₃N for triethylamine; EtOAc for ethyl acetate; Et₂O for diethyl ether; EtOH for ethanol; HOAc for acetic acid; HOSu for N-hydroxysuccinimide; LiHMDS or LiN(TMS)₂ for lithium bis(trimethylsilyl)amide; MCPBA for meta-chloroperbenzoic acid; MeOH for methanol; MsCl for methanesulfonyl chloride; NaHMDS or NaN(TMS)₂ for sodium bis(trimethylsilyl)amide; NMO for N-methylmorpholine N-oxide; SOCl₂ for thionyl chloride; PPTS for pyridium p-toluene sulfonate; Pd(OAc)₂ for palladium (II) acetate; PPh₃ for triphenylphosphine; Py for pyridine; TFA for trifluoroacetic acid; TEA for triethylamine; THF for tetrahydrofuran; TMSCl for trimethylsilyl chloride; TMSCF₃ for trimethyl(trifluoromethyl)-silane; TPP for triphenylphosphine; TPAP for tetra-n-propylammonium perruthenate; DMAP for 4-dimethylamino pyridine; TsOH for p-toluene sulfonic acid; MsOH for methanesulfonic acid; OMs for mesylate, OTs for tosylate; OTf for triflate; Boc for tert-butoxycarbonyl; Fmoc for N-fluorenylmethoxycarbonyl; Su for succinimide; Ph for phenyl; HBPyU for O-benzotriazol-1-yl-N,N,N′,N′-bis(tetramethylene)uronium hexafluorophosphate; PyBOP for benzotriazol-1-yloxytripyrrolidinophosphonium hexafluorophosphate; HATU for N,N,N′,N′-tetramethyl-O-(7-azabenzotriazol-1-yl)uranium hexafluorophosphate.

Methicillin-Resistant Staphylococcus aureus

Staphylococcus aureus (S. aureus), a spherical bacterium, is the most common cause of staph infections. S. aureus has been known to cause a range of illnesses from minor skin infections, such as pimples, impetigo, boils, cellulitis folliculitis, furuncles, carbuncles, scalded skin syndrome, abscesses, to life-threatening diseases such as pneumonia, meningitis, osteomyelitis endocarditis, toxic shock syndrome, and septicemia. Further, S. aureus is one of the most common causes of nosocomial infections, often causing postsurgical wound infections.

Methicillin was introduced in the late 1950s to treat infections caused by penicillin-resistant S. aureus. It has been reported previously that S. aureus isolates had acquired resistance to methicillin (methicillin-resistant S. aureus, MRSA). The methicillin resistance gene (mecA) encodes a methicillin-resistant penicillin-binding protein that is not present in susceptible strains. mecA is carried on a mobile genetic element, the staphylococcal cassette chromosome mec (SCCmec), of which four forms have been described that differ in size and genetic composition. The methicillin-resistant penicillin-binding protein allows for resistance to β-lactam antibiotics and obviates their clinical use during MRSA infections.

In one aspect is a method for treating a subject having a resistant bacterium comprising administering to the subject a compound of Formula (I) or a pharmaceutically acceptable salt, ester, solvate, alkylated quaternary ammonium salt, stereoisomer, tautomer or prodrug thereof. In one embodiment, the bacterium is a Gram-positive bacteria. In another embodiment, the Gram-positive bacterium is S. aureus. In further embodiment, the S. aureus is resistant or refractory to a beta-lactam antibiotic. In yet a further embodiment, the beta-lactam antibiotic belongs to the class of penicillins. In a further embodiment, the beta-lactam antibiotic is methicillin. In yet another embodiment, the subject has a methicillin-resistant S. aureus bacteria. In one embodiment the beta-lactam antibiotic is flucloxacillin. In another embodiment is a method for treating a subject having a dicloxacillin-resistant bacteria comprising administering to the subject a compound of Formula (I) or a pharmaceutically acceptable salt, ester, solvate, alkylated quaternary ammonium salt, stereoisomer, tautomer or prodrug thereof wherein the subject is refractory to dicloxacillin. Also disclosed herein is a method for treating a subject having a methicillin-resistant bacteria comprising administering a compound of Formula (I) or a pharmaceutically acceptable salt, ester, solvate, alkylated quaternary ammonium salt, stereoisomer, tautomer or prodrug thereof wherein the subject has been determined to have a methicillin-resistant bacteria. In one embodiment the subject is screened for methicillin-resistant bacteria. In another embodiment, the subject screening is performed through a nasal culture. In a further embodiment the methicillin-resistant bacteria is detected by swabbing the nostril(s) of the subject and isolating the bacteria. In another embodiment, Real-time PCR and/or Quantitative PCR is employed to determine whether the subject has a methicillin-resistant bacteria.

In one embodiment is a method for treating a subject having a first-generation cephalosporin-resistant bacteria comprising administering a compound of Formula (I) or a pharmaceutically acceptable salt, ester, solvate, alkylated quaternary ammonium salt, stereoisomer, tautomer or prodrug thereof wherein the subject is refractory to a first-generation cephalosporin. In one embodiment, the bacteria is resistant to a first-generation cephalosporin. In a further embodiment, the bacteria is resistant to cefacetrile. In another embodiment, the bacteria is resistant to cefadroxil. In yet another embodiment, the bacteria is resistant to cefalexin. In one embodiment, the bacteria is resistant to cefaloglycin. In another embodiment, the bacteria is resistant to cefalonium. In another embodiment, the bacteria is resistant to cefaloridine. In yet another embodiment, the bacteria is resistant to cefalotin. In a further embodiment, the bacteria is resistant to cefapirin. In yet a further embodiment, the bacteria is resistant to cefatrizine. In one embodiment, the bacteria is resistant to cefazaflur. In another embodiment, the bacteria is resistant to cefazedone. In yet another embodiment, the bacteria is resistant to cefazolin. In a further embodiment, the bacteria is resistant to cefradine. In yet a further embodiment, the bacteria is resistant to cefroxadine. In one embodiment, the bacteria is resistant to ceftezole.

In one embodiment is a method for treating a subject having a second-generation cephalosporin-resistant bacteria comprising administering a compound of Formula (I) or a pharmaceutically acceptable salt, ester, solvate, alkylated quaternary ammonium salt, stereoisomer, tautomer or prodrug thereof wherein the subject is refractory to a second-generation cephalosporin. In another embodiment, the bacteria is resistant to a second-generation cephalosporin. In a further embodiment, the bacteria is resistant to cefaclor. In another embodiment, the bacteria is resistant to cefonicid. In yet another embodiment, the bacteria is resistant to cefprozil. In one embodiment, the bacteria is resistant to cefuroxime. In another embodiment, the bacteria is resistant to cefuzonam. In another embodiment, the bacteria is resistant to cefmetazole. In yet another embodiment, the bacteria is resistant to cefotetan. In a further embodiment, the bacteria is resistant to cefoxitin.

In one embodiment is a method for treating a subject having a third-generation cephalosporin-resistant bacteria comprising administering a compound of Formula (I) or a pharmaceutically acceptable salt, ester, solvate, alkylated quaternary ammonium salt, stereoisomer, tautomer or prodrug thereof wherein the subject is refractory to a third-generation cephalosporin. In another embodiment, the bacteria is resistant to a third-generation cephalosporin. In a further embodiment, the bacteria is resistant to cefcapene. In another embodiment, the bacteria is resistant to cefdaloxime. In yet another embodiment, the bacteria is resistant to cefdinir. In one embodiment, the bacteria is resistant to cefditoren. In another embodiment, the bacteria is resistant to cefixime. In another embodiment, the bacteria is resistant to cefmenoxime. In yet another embodiment, the bacteria is resistant to cefodizime. In a further embodiment, the bacteria is resistant to cefotaxime. In yet a further embodiment, the bacteria is resistant to cefpimizole. In one embodiment, the bacteria is resistant to cefpodoxime. In another embodiment, the bacteria is resistant to cefteram. In yet another embodiment, the bacteria is resistant to ceftibuten. In a further embodiment, the bacteria is resistant to ceftiofur. In yet a further embodiment, the bacteria is resistant to ceftiolene. In one embodiment, the bacteria is resistant to ceftizoxime. In another embodiment, the bacteria is resistant to ceftriaxone. In yet another embodiment, the bacteria is resistant to cefoperazone. In yet a further embodiment, the bacteria is resistant to ceftazidime.

In one embodiment is a method for treating a subject having a fourth-generation cephalosporin-resistant bacteria comprising administering a compound of Formula (I) or a pharmaceutically acceptable salt, ester, solvate, alkylated quaternary ammonium salt, stereoisomer, tautomer or prodrug thereof wherein the subject is refractory to a fourth-generation cephalosporin. In another embodiment, the bacteria is resistant to a fourth-generation cephalosporin. In a further embodiment, the bacteria is resistant to cefclidine. In another embodiment, the bacteria is resistant to cefepime. In yet another embodiment, the bacteria is resistant to cefluprenam. In one embodiment, the bacteria is resistant to cefoselis. In another embodiment, the bacteria is resistant to cefozopran. In another embodiment, the bacteria is resistant to cefpirome. In yet another embodiment, the bacteria is refractory to cefquinome.

In one embodiment is a method for treating a subject having a carbapenem-resistant bacteria comprising administering a compound of Formula (I) or a pharmaceutically acceptable salt, ester, solvate, alkylated quaternary ammonium salt, stereoisomer, tautomer or prodrug thereof wherein the subject is refractory to a carbapenem. In another embodiment, the bacteria is resistant to a carbapenem. In a further embodiment, is a method for treating a subject having a imipenem-resistant bacteria comprising administering a compound of Formula (I) or a pharmaceutically acceptable salt, ester, solvate, alkylated quaternary ammonium salt, stereoisomer, tautomer or prodrug thereof wherein the bacteria is resistant to imipenem. In another embodiment, is a method for treating a subject having a meropenem-resistant bacteria comprising administering a compound of Formula (I) or a pharmaceutically acceptable salt, ester, solvate, alkylated quaternary ammonium salt, stereoisomer, tautomer or prodrug thereof wherein the bacteria is resistant to meropenem. In yet another embodiment, is a method for treating a subject having a ertapenem-resistant bacteria comprising administering a compound of Formula (I) or a pharmaceutically acceptable salt, ester, solvate, alkylated quaternary ammonium salt, stereoisomer, tautomer or prodrug thereof wherein the bacteria is resistant to ertapenem. In one embodiment, is a method for treating a subject having a faropenem-resistant bacteria comprising administering a compound of Formula (I) or a pharmaceutically acceptable salt, ester, solvate, alkylated quaternary ammonium salt, stereoisomer, tautomer or prodrug thereof wherein the bacteria is resistant to faropenem. In another embodiment, is a method for treating a subject having a doripenem-resistant bacteria comprising administering a compound of Formula (I) or a pharmaceutically acceptable salt, ester, solvate, alkylated quaternary ammonium salt, stereoisomer, tautomer or prodrug thereof wherein the bacteria is resistant to doripenem. In another embodiment, is a method for treating a subject having a panipenem-resistant bacteria comprising administering a compound of Formula (I) or a pharmaceutically acceptable salt, ester, solvate, alkylated quaternary ammonium salt, stereoisomer, tautomer or prodrug thereof wherein the bacteria is resistant to panipenem. In yet another embodiment, is a method for treating a subject having a biapenem-resistant bacteria comprising administering a compound of Formula (I) or a pharmaceutically acceptable salt, ester, solvate, alkylated quaternary ammonium salt, stereoisomer, tautomer or prodrug thereof wherein the bacteria is resistant to biapenem.

In one aspect is a method for treating a subject having a resistant bacterium comprising administering to the subject a compound of Formula (II) or a pharmaceutically acceptable salt, ester, solvate, alkylated quaternary ammonium salt, stereoisomer, tautomer or prodrug thereof. In one embodiment, the bacterium is a Gram-positive bacteria. In another embodiment, the Gram-positive bacterium is S. aureus. In further embodiment, the S. aureus is resistant or refractory to a beta-lactam antibiotic. In yet a further embodiment, the beta-lactam antibiotic belongs to the class of penicillins. In a further embodiment, the beta-lactam antibiotic is methicillin. In yet another embodiment, the subject has a methicillin-resistant S. aureus bacteria. In one embodiment the beta-lactam antibiotic is flucloxacillin. In another embodiment is a method for treating a subject having a dicloxacillin-resistant bacteria comprising administering to the subject a compound of Formula (II) or a pharmaceutically acceptable salt, ester, solvate, alkylated quaternary ammonium salt, stereoisomer, tautomer or prodrug thereof wherein the subject is refractory to dicloxacillin. Also disclosed herein is a method for treating a subject having a methicillin-resistant bacteria comprising administering a compound of Formula (II) or a pharmaceutically acceptable salt, ester, solvate, alkylated quaternary ammonium salt, stereoisomer, tautomer or prodrug thereof wherein the subject has been determined to have a methicillin-resistant bacteria. In one embodiment the subject is screened for methicillin-resistant bacteria. In another embodiment, the subject screening is performed through a nasal culture. In a further embodiment the methicillin-resistant bacteria is detected by swabbing the nostril(s) of the subject and isolating the bacteria. In another embodiment, Real-time PCR and/or Quantitative PCR is employed to determine whether the subject has a methicillin-resistant bacteria.

In one embodiment is a method for treating a subject having a first-generation cephalosporin-resistant bacteria comprising administering a compound of Formula (II) or a pharmaceutically acceptable salt, ester, solvate, alkylated quaternary ammonium salt, stereoisomer, tautomer or prodrug thereof wherein the subject is refractory to a first-generation cephalosporin. In one embodiment, the bacteria is resistant to a first-generation cephalosporin. In a further embodiment, the bacteria is resistant to cefacetrile. In another embodiment, the bacteria is resistant to cefadroxil. In yet another embodiment, the bacteria is resistant to cefalexin. In one embodiment, the bacteria is resistant to cefaloglycin. In another embodiment, the bacteria is resistant to cefalonium. In another embodiment, the bacteria is resistant to cefaloridine. In yet another embodiment, the bacteria is resistant to cefalotin. In a further embodiment, the bacteria is resistant to cefapirin. In yet a further embodiment, the bacteria is resistant to cefatrizine. In one embodiment, the bacteria is resistant to cefazaflur. In another embodiment, the bacteria is resistant to cefazedone. In yet another embodiment, the bacteria is resistant to cefazolin. In a further embodiment, the bacteria is resistant to cefradine. In yet a further embodiment, the bacteria is resistant to cefroxadine. In one embodiment, the bacteria is resistant to ceftezole.

In one embodiment is a method for treating a subject having a second-generation cephalosporin-resistant bacteria comprising administering a compound of Formula (II) or a pharmaceutically acceptable salt, ester, solvate, alkylated quaternary ammonium salt, stereoisomer, tautomer or prodrug thereof wherein the subject is refractory to a second-generation cephalosporin. In another embodiment, the bacteria is resistant to a second-generation cephalosporin. In a further embodiment, the bacteria is resistant to cefaclor. In another embodiment, the bacteria is resistant to cefonicid. In yet another embodiment, the bacteria is resistant to cefprozil. In one embodiment, the bacteria is resistant to cefuroxime. In another embodiment, the bacteria is resistant to cefuzonam. In another embodiment, the bacteria is resistant to cefmetazole. In yet another embodiment, the bacteria is resistant to cefotetan. In a further embodiment, the bacteria is resistant to cefoxitin.

In one embodiment is a method for treating a subject having a third-generation cephalosporin-resistant bacteria comprising administering a compound of Formula (II) or a pharmaceutically acceptable salt, ester, solvate, alkylated quaternary ammonium salt, stereoisomer, tautomer or prodrug thereof wherein the subject is refractory to a third-generation cephalosporin. In another embodiment, the bacteria is resistant to a third-generation cephalosporin. In a further embodiment, the bacteria is resistant to cefcapene. In another embodiment, the bacteria is resistant to cefdaloxime. In yet another embodiment, the bacteria is resistant to cefdinir. In one embodiment, the bacteria is resistant to cefditoren. In another embodiment, the bacteria is resistant to cefixime. In another embodiment, the bacteria is resistant to cefmenoxime. In yet another embodiment, the bacteria is resistant to cefodizime. In a further embodiment, the bacteria is resistant to cefotaxime. In yet a further embodiment, the bacteria is resistant to cefpimizole. In one embodiment, the bacteria is resistant to cefpodoxime. In another embodiment, the bacteria is resistant to cefteram. In yet another embodiment, the bacteria is resistant to ceftibuten. In a further embodiment, the bacteria is resistant to ceftiofur. In yet a further embodiment, the bacteria is resistant to ceftiolene. In one embodiment, the bacteria is resistant to ceftizoxime. In another embodiment, the bacteria is resistant to ceftriaxone. In yet another embodiment, the bacteria is resistant to cefoperazone. In yet a further embodiment, the bacteria is resistant to ceftazidime.

In one embodiment is a method for treating a subject having a fourth-generation cephalosporin-resistant bacteria comprising administering a compound of Formula (II) or a pharmaceutically acceptable salt, ester, solvate, alkylated quaternary ammonium salt, stereoisomer, tautomer or prodrug thereof wherein the subject is refractory to a fourth-generation cephalosporin. In another embodiment, the bacteria is resistant to a fourth-generation cephalosporin. In a further embodiment, the bacteria is resistant to cefclidine. In another embodiment, the bacteria is resistant to cefepime. In yet another embodiment, the bacteria is resistant to cefluprenam. In one embodiment, the bacteria is resistant to cefoselis. In another embodiment, the bacteria is resistant to cefozopran. In another embodiment, the bacteria is resistant to cefpirome. In yet another embodiment, the bacteria is refractory to cefquinome.

In one embodiment is a method for treating a subject having a carbapenem-resistant bacteria comprising administering a compound of Formula (II) or a pharmaceutically acceptable salt, ester, solvate, alkylated quaternary ammonium salt, stereoisomer, tautomer or prodrug thereof wherein the subject is refractory to a carbapenem. In another embodiment, the bacteria is resistant to a carbapenem. In a further embodiment, is a method for treating a subject having a imipenem-resistant bacteria comprising administering a compound of Formula (II) or a pharmaceutically acceptable salt, ester, solvate, alkylated quaternary ammonium salt, stereoisomer, tautomer or prodrug thereof wherein the bacteria is resistant to imipenem. In another embodiment, is a method for treating a subject having a meropenem-resistant bacteria comprising administering a compound of Formula (II) or a pharmaceutically acceptable salt, ester, solvate, alkylated quaternary ammonium salt, stereoisomer, tautomer or prodrug thereof wherein the bacteria is resistant to meropenem. In yet another embodiment, is a method for treating a subject having a ertapenem-resistant bacteria comprising administering a compound of Formula (II) or a pharmaceutically acceptable salt, ester, solvate, alkylated quaternary ammonium salt, stereoisomer, tautomer or prodrug thereof wherein the bacteria is resistant to ertapenem. In one embodiment, is a method for treating a subject having a faropenem-resistant bacteria comprising administering a compound of Formula (II) or a pharmaceutically acceptable salt, ester, solvate, alkylated quaternary ammonium salt, stereoisomer, tautomer or prodrug thereof wherein the bacteria is resistant to faropenem. In another embodiment, is a method for treating a subject having a doripenem-resistant bacteria comprising administering a compound of Formula (II) or a pharmaceutically acceptable salt, ester, solvate, alkylated quaternary ammonium salt, stereoisomer, tautomer or prodrug thereof wherein the bacteria is resistant to doripenem. In another embodiment, is a method for treating a subject having a panipenem-resistant bacteria comprising administering a compound of Formula (II) or a pharmaceutically acceptable salt, ester, solvate, alkylated quaternary ammonium salt, stereoisomer, tautomer or prodrug thereof wherein the bacteria is resistant to panipenem. In yet another embodiment, is a method for treating a subject having a biapenem-resistant bacteria comprising administering a compound of Formula (II) or a pharmaceutically acceptable salt, ester, solvate, alkylated quaternary ammonium salt, stereoisomer, tautomer or prodrug thereof wherein the bacteria is resistant to biapenem.

In one aspect is a method for treating a subject having a resistant bacterium comprising administering to the subject a compound of Formula (III) or a pharmaceutically acceptable salt, ester, solvate, alkylated quaternary ammonium salt, stereoisomer, tautomer or prodrug thereof. In one embodiment, the bacterium is a Gram-positive bacteria. In another embodiment, the Gram-positive bacterium is S. aureus. In further embodiment, the S. aureus is resistant or refractory to a beta-lactam antibiotic. In yet a further embodiment, the beta-lactam antibiotic belongs to the class of penicillins. In a further embodiment, the beta-lactam antibiotic is methicillin. In yet another embodiment, the subject has a methicillin-resistant S. aureus bacteria. In one embodiment the beta-lactam antibiotic is flucloxacillin. In another embodiment is a method for treating a subject having a dicloxacillin-resistant bacteria comprising administering to the subject a compound of Formula (III) or a pharmaceutically acceptable salt, ester, solvate, alkylated quaternary ammonium salt, stereoisomer, tautomer or prodrug thereof wherein the subject is refractory to dicloxacillin. Also disclosed herein is a method for treating a subject having a methicillin-resistant bacteria comprising administering a compound of Formula (III) or a pharmaceutically acceptable salt, ester, solvate, alkylated quaternary ammonium salt, stereoisomer, tautomer or prodrug thereof wherein the subject has been determined to have a methicillin-resistant bacteria. In one embodiment the subject is screened for methicillin-resistant bacteria. In another embodiment, the subject screening is performed through a nasal culture. In a further embodiment the methicillin-resistant bacteria is detected by swabbing the nostril(s) of the subject and isolating the bacteria. In another embodiment, Real-time PCR and/or Quantitative PCR is employed to determine whether the subject has a methicillin-resistant bacteria.

In one embodiment is a method for treating a subject having a first-generation cephalosporin-resistant bacteria comprising administering a compound of Formula (III) or a pharmaceutically acceptable salt, ester, solvate, alkylated quaternary ammonium salt, stereoisomer, tautomer or prodrug thereof wherein the subject is refractory to a first-generation cephalosporin. In one embodiment, the bacteria is resistant to a first-generation cephalosporin. In a further embodiment, the bacteria is resistant to cefacetrile. In another embodiment, the bacteria is resistant to cefadroxil. In yet another embodiment, the bacteria is resistant to cefalexin. In one embodiment, the bacteria is resistant to cefaloglycin. In another embodiment, the bacteria is resistant to cefalonium. In another embodiment, the bacteria is resistant to cefaloridine. In yet another embodiment, the bacteria is resistant to cefalotin. In a further embodiment, the bacteria is resistant to cefapirin. In yet a further embodiment, the bacteria is resistant to cefatrizine. In one embodiment, the bacteria is resistant to cefazaflur. In another embodiment, the bacteria is resistant to cefazedone. In yet another embodiment, the bacteria is resistant to cefazolin. In a further embodiment, the bacteria is resistant to cefradine. In yet a further embodiment, the bacteria is resistant to cefroxadine. In one embodiment, the bacteria is resistant to ceftezole.

In one embodiment is a method for treating a subject having a second-generation cephalosporin-resistant bacteria comprising administering a compound of Formula (III) or a pharmaceutically acceptable salt, ester, solvate, alkylated quaternary ammonium salt, stereoisomer, tautomer or prodrug thereof wherein the subject is refractory to a second-generation cephalosporin. In another embodiment, the bacteria is resistant to a second-generation cephalosporin. In a further embodiment, the bacteria is resistant to cefaclor. In another embodiment, the bacteria is resistant to cefonicid. In yet another embodiment, the bacteria is resistant to cefprozil. In one embodiment, the bacteria is resistant to cefuroxime. In another embodiment, the bacteria is resistant to cefuzonam. In another embodiment, the bacteria is resistant to cefmetazole. In yet another embodiment, the bacteria is resistant to cefotetan. In a further embodiment, the bacteria is resistant to cefoxitin.

In one embodiment is a method for treating a subject having a third-generation cephalosporin-resistant bacteria comprising administering a compound of Formula (III) or a pharmaceutically acceptable salt, ester, solvate, alkylated quaternary ammonium salt, stereoisomer, tautomer or prodrug thereof wherein the subject is refractory to a third-generation cephalosporin. In another embodiment, the bacteria is resistant to a third-generation cephalosporin. In a further embodiment, the bacteria is resistant to cefcapene. In another embodiment, the bacteria is resistant to cefdaloxime. In yet another embodiment, the bacteria is resistant to cefdinir. In one embodiment, the bacteria is resistant to cefditoren. In another embodiment, the bacteria is resistant to cefixime. In another embodiment, the bacteria is resistant to cefmenoxime. In yet another embodiment, the bacteria is resistant to cefodizime. In a further embodiment, the bacteria is resistant to cefotaxime. In yet a further embodiment, the bacteria is resistant to cefpimizole. In one embodiment, the bacteria is resistant to cefpodoxime. In another embodiment, the bacteria is resistant to cefteram. In yet another embodiment, the bacteria is resistant to ceftibuten. In a further embodiment, the bacteria is resistant to ceftiofur. In yet a further embodiment, the bacteria is resistant to ceftiolene. In one embodiment, the bacteria is resistant to ceftizoxime. In another embodiment, the bacteria is resistant to ceftriaxone. In yet another embodiment, the bacteria is resistant to cefoperazone. In yet a further embodiment, the bacteria is resistant to ceftazidime.

In one embodiment is a method for treating a subject having a fourth-generation cephalosporin-resistant bacteria comprising administering a compound of Formula (III) or a pharmaceutically acceptable salt, ester, solvate, alkylated quaternary ammonium salt, stereoisomer, tautomer or prodrug thereof wherein the subject is refractory to a fourth-generation cephalosporin. In another embodiment, the bacteria is resistant to a fourth-generation cephalosporin. In a further embodiment, the bacteria is resistant to cefclidine. In another embodiment, the bacteria is resistant to cefepime. In yet another embodiment, the bacteria is resistant to cefluprenam. In one embodiment, the bacteria is resistant to cefoselis. In another embodiment, the bacteria is resistant to cefozopran. In another embodiment, the bacteria is resistant to cefpirome. In yet another embodiment, the bacteria is refractory to cefquinome.

In one embodiment is a method for treating a subject having a carbapenem-resistant bacteria comprising administering a compound of Formula (III) or a pharmaceutically acceptable salt, ester, solvate, alkylated quaternary ammonium salt, stereoisomer, tautomer or prodrug thereof wherein the subject is refractory to a carbapenem. In another embodiment, the bacteria is resistant to a carbapenem. In a further embodiment, is a method for treating a subject having a imipenem-resistant bacteria comprising administering a compound of Formula (III) or a pharmaceutically acceptable salt, ester, solvate, alkylated quaternary ammonium salt, stereoisomer, tautomer or prodrug thereof wherein the bacteria is resistant to imipenem. In another embodiment, is a method for treating a subject having a meropenem-resistant bacteria comprising administering a compound of Formula (III) or a pharmaceutically acceptable salt, ester, solvate, alkylated quaternary ammonium salt, stereoisomer, tautomer or prodrug thereof wherein the bacteria is resistant to meropenem. In yet another embodiment, is a method for treating a subject having a ertapenem-resistant bacteria comprising administering a compound of Formula (III) or a pharmaceutically acceptable salt, ester, solvate, alkylated quaternary ammonium salt, stereoisomer, tautomer or prodrug thereof wherein the bacteria is resistant to ertapenem. In one embodiment, is a method for treating a subject having a faropenem-resistant bacteria comprising administering a compound of Formula (III) or a pharmaceutically acceptable salt, ester, solvate, alkylated quaternary ammonium salt, stereoisomer, tautomer or prodrug thereof wherein the bacteria is resistant to faropenem. In another embodiment, is a method for treating a subject having a doripenem-resistant bacteria comprising administering a compound of Formula (III) or a pharmaceutically acceptable salt, ester, solvate, alkylated quaternary ammonium salt, stereoisomer, tautomer or prodrug thereof wherein the bacteria is resistant to doripenem. In another embodiment, is a method for treating a subject having a panipenem-resistant bacteria comprising administering a compound of Formula (III) or a pharmaceutically acceptable salt, ester, solvate, alkylated quaternary ammonium salt, stereoisomer, tautomer or prodrug thereof wherein the bacteria is resistant to panipenem. In yet another embodiment, is a method for treating a subject having a biapenem-resistant bacteria comprising administering a compound of Formula (III) or a pharmaceutically acceptable salt, ester, solvate, alkylated quaternary ammonium salt, stereoisomer, tautomer or prodrug thereof wherein the bacteria is resistant to biapenem.

In one aspect is a method for treating a subject having a resistant bacterium comprising administering to the subject a compound of Formula (IV) or a pharmaceutically acceptable salt, ester, solvate, alkylated quaternary ammonium salt, stereoisomer, tautomer or prodrug thereof. In one embodiment, the bacterium is a Gram-positive bacteria. In another embodiment, the Gram-positive bacterium is S. aureus. In further embodiment, the S. aureus is resistant or refractory to a beta-lactam antibiotic. In yet a further embodiment, the beta-lactam antibiotic belongs to the class of penicillins. In a further embodiment, the beta-lactam antibiotic is methicillin. In yet another embodiment, the subject has a methicillin-resistant S. aureus bacteria. In one embodiment the beta-lactam antibiotic is flucloxacillin. In another embodiment is a method for treating a subject having a dicloxacillin-resistant bacteria comprising administering to the subject a compound of Formula (IV) or a pharmaceutically acceptable salt, ester, solvate, alkylated quaternary ammonium salt, stereoisomer, tautomer or prodrug thereof wherein the subject is refractory to dicloxacillin. Also disclosed herein is a method for treating a subject having a methicillin-resistant bacteria comprising administering a compound of Formula (IV) or a pharmaceutically acceptable salt, ester, solvate, alkylated quaternary ammonium salt, stereoisomer, tautomer or prodrug thereof wherein the subject has been determined to have a methicillin-resistant bacteria. In one embodiment the subject is screened for methicillin-resistant bacteria. In another embodiment, the subject screening is performed through a nasal culture. In a further embodiment the methicillin-resistant bacteria is detected by swabbing the nostril(s) of the subject and isolating the bacteria. In another embodiment, Real-time PCR and/or Quantitative PCR is employed to determine whether the subject has a methicillin-resistant bacteria.

In one embodiment is a method for treating a subject having a first-generation cephalosporin-resistant bacteria comprising administering a compound of Formula (IV) or a pharmaceutically acceptable salt, ester, solvate, alkylated quaternary ammonium salt, stereoisomer, tautomer or prodrug thereof wherein the subject is refractory to a first-generation cephalosporin. In one embodiment, the bacteria is resistant to a first-generation cephalosporin. In a further embodiment, the bacteria is resistant to cefacetrile. In another embodiment, the bacteria is resistant to cefadroxil. In yet another embodiment, the bacteria is resistant to cefalexin. In one embodiment, the bacteria is resistant to cefaloglycin. In another embodiment, the bacteria is resistant to cefalonium. In another embodiment, the bacteria is resistant to cefaloridine. In yet another embodiment, the bacteria is resistant to cefalotin. In a further embodiment, the bacteria is resistant to cefapirin. In yet a further embodiment, the bacteria is resistant to cefatrizine. In one embodiment, the bacteria is resistant to cefazaflur. In another embodiment, the bacteria is resistant to cefazedone. In yet another embodiment, the bacteria is resistant to cefazolin. In a further embodiment, the bacteria is resistant to cefradine. In yet a further embodiment, the bacteria is resistant to cefroxadine. In one embodiment, the bacteria is resistant to ceftezole.

In one embodiment is a method for treating a subject having a second-generation cephalosporin-resistant bacteria comprising administering a compound of Formula (IV) or a pharmaceutically acceptable salt, ester, solvate, alkylated quaternary ammonium salt, stereoisomer, tautomer or prodrug thereof wherein the subject is refractory to a second-generation cephalosporin. In another embodiment, the bacteria is resistant to a second-generation cephalosporin. In a further embodiment, the bacteria is resistant to cefaclor. In another embodiment, the bacteria is resistant to cefonicid. In yet another embodiment, the bacteria is resistant to cefprozil. In one embodiment, the bacteria is resistant to cefuroxime. In another embodiment, the bacteria is resistant to cefuzonam. In another embodiment, the bacteria is resistant to cefmetazole. In yet another embodiment, the bacteria is resistant to cefotetan. In a further embodiment, the bacteria is resistant to cefoxitin.

In one embodiment is a method for treating a subject having a third-generation cephalosporin-resistant bacteria comprising administering a compound of Formula (IV) or a pharmaceutically acceptable salt, ester, solvate, alkylated quaternary ammonium salt, stereoisomer, tautomer or prodrug thereof wherein the subject is refractory to a third-generation cephalosporin. In another embodiment, the bacteria is resistant to a third-generation cephalosporin. In a further embodiment, the bacteria is resistant to cefcapene. In another embodiment, the bacteria is resistant to cefdaloxime. In yet another embodiment, the bacteria is resistant to cefdinir. In one embodiment, the bacteria is resistant to cefditoren. In another embodiment, the bacteria is resistant to cefixime. In another embodiment, the bacteria is resistant to cefmenoxime. In yet another embodiment, the bacteria is resistant to cefodizime. In a further embodiment, the bacteria is resistant to cefotaxime. In yet a further embodiment, the bacteria is resistant to cefpimizole. In one embodiment, the bacteria is resistant to cefpodoxime. In another embodiment, the bacteria is resistant to cefteram. In yet another embodiment, the bacteria is resistant to ceftibuten. In a further embodiment, the bacteria is resistant to ceftiofur. In yet a further embodiment, the bacteria is resistant to ceftiolene. In one embodiment, the bacteria is resistant to ceftizoxime. In another embodiment, the bacteria is resistant to ceftriaxone. In yet another embodiment, the bacteria is resistant to cefoperazone. In yet a further embodiment, the bacteria is resistant to ceftazidime.

In one embodiment is a method for treating a subject having a fourth-generation cephalosporin-resistant bacteria comprising administering a compound of Formula (IV) or a pharmaceutically acceptable salt, ester, solvate, alkylated quaternary ammonium salt, stereoisomer, tautomer or prodrug thereof wherein the subject is refractory to a fourth-generation cephalosporin. In another embodiment, the bacteria is resistant to a fourth-generation cephalosporin. In a further embodiment, the bacteria is resistant to cefclidine. In another embodiment, the bacteria is resistant to cefepime. In yet another embodiment, the bacteria is resistant to cefluprenam. In one embodiment, the bacteria is resistant to cefoselis. In another embodiment, the bacteria is resistant to cefozopran. In another embodiment, the bacteria is resistant to cefpirome. In yet another embodiment, the bacteria is refractory to cefquinome.

In one embodiment is a method for treating a subject having a carbapenem-resistant bacteria comprising administering a compound of Formula (IV) or a pharmaceutically acceptable salt, ester, solvate, alkylated quaternary ammonium salt, stereoisomer, tautomer or prodrug thereof wherein the subject is refractory to a carbapenem. In another embodiment, the bacteria is resistant to a carbapenem. In a further embodiment, is a method for treating a subject having a imipenem-resistant bacteria comprising administering a compound of Formula (IV) or a pharmaceutically acceptable salt, ester, solvate, alkylated quaternary ammonium salt, stereoisomer, tautomer or prodrug thereof wherein the bacteria is resistant to imipenem. In another embodiment, is a method for treating a subject having a meropenem-resistant bacteria comprising administering a compound of Formula (IV) or a pharmaceutically acceptable salt, ester, solvate, alkylated quaternary ammonium salt, stereoisomer, tautomer or prodrug thereof wherein the bacteria is resistant to meropenem. In yet another embodiment, is a method for treating a subject having a ertapenem-resistant bacteria comprising administering a compound of Formula (IV) or a pharmaceutically acceptable salt, ester, solvate, alkylated quaternary ammonium salt, stereoisomer, tautomer or prodrug thereof wherein the bacteria is resistant to ertapenem. In one embodiment, is a method for treating a subject having a faropenem-resistant bacteria comprising administering a compound of Formula (IV) or a pharmaceutically acceptable salt, ester, solvate, alkylated quaternary ammonium salt, stereoisomer, tautomer or prodrug thereof wherein the bacteria is resistant to faropenem. In another embodiment, is a method for treating a subject having a doripenem-resistant bacteria comprising administering a compound of Formula (IV) or a pharmaceutically acceptable salt, ester, solvate, alkylated quaternary ammonium salt, stereoisomer, tautomer or prodrug thereof wherein the bacteria is resistant to doripenem. In another embodiment, is a method for treating a subject having a panipenem-resistant bacteria comprising administering a compound of Formula (IV) or a pharmaceutically acceptable salt, ester, solvate, alkylated quaternary ammonium salt, stereoisomer, tautomer or prodrug thereof wherein the bacteria is resistant to panipenem. In yet another embodiment, is a method for treating a subject having a biapenem-resistant bacteria comprising administering a compound of Formula (IV) or a pharmaceutically acceptable salt, ester, solvate, alkylated quaternary ammonium salt, stereoisomer, tautomer or prodrug thereof wherein the bacteria is resistant to biapenem.

In one aspect is a method for treating a subject having a resistant bacterium comprising administering to the subject a compound of Formula (V) or a pharmaceutically acceptable salt, ester, solvate, alkylated quaternary ammonium salt, stereoisomer, tautomer or prodrug thereof. In one embodiment, the bacterium is a Gram-positive bacteria. In another embodiment, the Gram-positive bacterium is S. aureus. In further embodiment, the S. aureus is resistant or refractory to a beta-lactam antibiotic. In yet a further embodiment, the beta-lactam antibiotic belongs to the class of penicillins. In a further embodiment, the beta-lactam antibiotic is methicillin. In yet another embodiment, the subject has a methicillin-resistant S. aureus bacteria. In one embodiment the beta-lactam antibiotic is flucloxacillin. In another embodiment is a method for treating a subject having a dicloxacillin-resistant bacteria comprising administering to the subject a compound of Formula (V) or a pharmaceutically acceptable salt, ester, solvate, alkylated quaternary ammonium salt, stereoisomer, tautomer or prodrug thereof wherein the subject is refractory to dicloxacillin. Also disclosed herein is a method for treating a subject having a methicillin-resistant bacteria comprising administering a compound of Formula (V) or a pharmaceutically acceptable salt, ester, solvate, alkylated quaternary ammonium salt, stereoisomer, tautomer or prodrug thereof wherein the subject has been determined to have a methicillin-resistant bacteria. In one embodiment the subject is screened for methicillin-resistant bacteria. In another embodiment, the subject screening is performed through a nasal culture. In a further embodiment the methicillin-resistant bacteria is detected by swabbing the nostril(s) of the subject and isolating the bacteria. In another embodiment, Real-time PCR and/or Quantitative PCR is employed to determine whether the subject has a methicillin-resistant bacteria.

In one embodiment is a method for treating a subject having a first-generation cephalosporin-resistant bacteria comprising administering a compound of Formula (V) or a pharmaceutically acceptable salt, ester, solvate, alkylated quaternary ammonium salt, stereoisomer, tautomer or prodrug thereof wherein the subject is refractory to a first-generation cephalosporin. In one embodiment, the bacteria is resistant to a first-generation cephalosporin. In a further embodiment, the bacteria is resistant to cefacetrile. In another embodiment, the bacteria is resistant to cefadroxil. In yet another embodiment, the bacteria is resistant to cefalexin. In one embodiment, the bacteria is resistant to cefaloglycin. In another embodiment, the bacteria is resistant to cefalonium. In another embodiment, the bacteria is resistant to cefaloridine. In yet another embodiment, the bacteria is resistant to cefalotin. In a further embodiment, the bacteria is resistant to cefapirin. In yet a further embodiment, the bacteria is resistant to cefatrizine. In one embodiment, the bacteria is resistant to cefazaflur. In another embodiment, the bacteria is resistant to cefazedone. In yet another embodiment, the bacteria is resistant to cefazolin. In a further embodiment, the bacteria is resistant to cefradine. In yet a further embodiment, the bacteria is resistant to cefroxadine. In one embodiment, the bacteria is resistant to ceftezole.

In one embodiment is a method for treating a subject having a second-generation cephalosporin-resistant bacteria comprising administering a compound of Formula (V) or a pharmaceutically acceptable salt, ester, solvate, alkylated quaternary ammonium salt, stereoisomer, tautomer or prodrug thereof wherein the subject is refractory to a second-generation cephalosporin. In another embodiment, the bacteria is resistant to a second-generation cephalosporin. In a further embodiment, the bacteria is resistant to cefaclor. In another embodiment, the bacteria is resistant to cefonicid. In yet another embodiment, the bacteria is resistant to cefprozil. In one embodiment, the bacteria is resistant to cefuroxime. In another embodiment, the bacteria is resistant to cefuzonam. In another embodiment, the bacteria is resistant to cefmetazole. In yet another embodiment, the bacteria is resistant to cefotetan. In a further embodiment, the bacteria is resistant to cefoxitin.

In one embodiment is a method for treating a subject having a third-generation cephalosporin-resistant bacteria comprising administering a compound of Formula (V) or a pharmaceutically acceptable salt, ester, solvate, alkylated quaternary ammonium salt, stereoisomer, tautomer or prodrug thereof wherein the subject is refractory to a third-generation cephalosporin. In another embodiment, the bacteria is resistant to a third-generation cephalosporin. In a further embodiment, the bacteria is resistant to cefcapene. In another embodiment, the bacteria is resistant to cefdaloxime. In yet another embodiment, the bacteria is resistant to cefdinir. In one embodiment, the bacteria is resistant to cefditoren. In another embodiment, the bacteria is resistant to cefixime. In another embodiment, the bacteria is resistant to cefmenoxime. In yet another embodiment, the bacteria is resistant to cefodizime. In a further embodiment, the bacteria is resistant to cefotaxime. In yet a further embodiment, the bacteria is resistant to cefpimizole. In one embodiment, the bacteria is resistant to cefpodoxime. In another embodiment, the bacteria is resistant to cefteram. In yet another embodiment, the bacteria is resistant to ceftibuten. In a further embodiment, the bacteria is resistant to ceftiofur. In yet a further embodiment, the bacteria is resistant to ceftiolene. In one embodiment, the bacteria is resistant to ceftizoxime. In another embodiment, the bacteria is resistant to ceftriaxone. In yet another embodiment, the bacteria is resistant to cefoperazone. In yet a further embodiment, the bacteria is resistant to ceftazidime.

In one embodiment is a method for treating a subject having a fourth-generation cephalosporin-resistant bacteria comprising administering a compound of Formula (V) or a pharmaceutically acceptable salt, ester, solvate, alkylated quaternary ammonium salt, stereoisomer, tautomer or prodrug thereof wherein the subject is refractory to a fourth-generation cephalosporin. In another embodiment, the bacteria is resistant to a fourth-generation cephalosporin. In a further embodiment, the bacteria is resistant to cefclidine. In another embodiment, the bacteria is resistant to cefepime. In yet another embodiment, the bacteria is resistant to cefluprenam. In one embodiment, the bacteria is resistant to cefoselis. In another embodiment, the bacteria is resistant to cefozopran. In another embodiment, the bacteria is resistant to cefpirome. In yet another embodiment, the bacteria is refractory to cefquinome.

In one embodiment is a method for treating a subject having a carbapenem-resistant bacteria comprising administering a compound of Formula (V) or a pharmaceutically acceptable salt, ester, solvate, alkylated quaternary ammonium salt, stereoisomer, tautomer or prodrug thereof wherein the subject is refractory to a carbapenem. In another embodiment, the bacteria is resistant to a carbapenem. In a further embodiment, is a method for treating a subject having a imipenem-resistant bacteria comprising administering a compound of Formula (V) or a pharmaceutically acceptable salt, ester, solvate, alkylated quaternary ammonium salt, stereoisomer, tautomer or prodrug thereof wherein the bacteria is resistant to imipenem. In another embodiment, is a method for treating a subject having a meropenem-resistant bacteria comprising administering a compound of Formula (V) or a pharmaceutically acceptable salt, ester, solvate, alkylated quaternary ammonium salt, stereoisomer, tautomer or prodrug thereof wherein the bacteria is resistant to meropenem. In yet another embodiment, is a method for treating a subject having a ertapenem-resistant bacteria comprising administering a compound of Formula (V) or a pharmaceutically acceptable salt, ester, solvate, alkylated quaternary ammonium salt, stereoisomer, tautomer or prodrug thereof wherein the bacteria is resistant to ertapenem. In one embodiment, is a method for treating a subject having a faropenem-resistant bacteria comprising administering a compound of Formula (V) or a pharmaceutically acceptable salt, ester, solvate, alkylated quaternary ammonium salt, stereoisomer, tautomer or prodrug thereof wherein the bacteria is resistant to faropenem. In another embodiment, is a method for treating a subject having a doripenem-resistant bacteria comprising administering a compound of Formula (V) or a pharmaceutically acceptable salt, ester, solvate, alkylated quaternary ammonium salt, stereoisomer, tautomer or prodrug thereof wherein the bacteria is resistant to doripenem. In another embodiment, is a method for treating a subject having a panipenem-resistant bacteria comprising administering a compound of Formula (V) or a pharmaceutically acceptable salt, ester, solvate, alkylated quaternary ammonium salt, stereoisomer, tautomer or prodrug thereof wherein the bacteria is resistant to panipenem. In yet another embodiment, is a method for treating a subject having a biapenem-resistant bacteria comprising administering a compound of Formula (V) or a pharmaceutically acceptable salt, ester, solvate, alkylated quaternary ammonium salt, stereoisomer, tautomer or prodrug thereof wherein the bacteria is resistant to biapenem.

Vancomycin-Intermediate and Vancomycin-Resistant Staphylococcus aureus

Vancomycin-intermediate Staphylococcus aureus and vancomycin-resistant staphylococcus aureus are specific types of antimicrobial-resistant Staph bacteria that are refractory to vancomycin treatment. S. aureus isolates for which vancomycin MICs are 4-8 μg/mL are classified as vancomycin-intermediate and isolates for which vancomycin MICs are ≧16 μg/mL are classified as vancomycin-resistant (Clinical and Laboratory Standards Institute/NCCLS. Performance Standards for Antimicrobial Susceptibility Testing. Sixteenth informational supplement. M100-S16. Wayne, Pa.: CLSI, 2006).

As used herein, the term “minimum inhibitory concentration” (MIC) refers to the lowest concentration of an antibiotic that is needed to inhibit growth of a bacterial isolate in vitro. A common method for determining the MIC of an antibiotic is to prepare several tubes containing serial dilutions of the antibiotic, that are then inoculated with the bacterial isolate of interest. The MIC of an antibiotic is determined from the tube with the lowest concentration that shows no turbidity (no growth).

In one aspect is a method of treating a subject having a bacterial infection comprising administering to the subject a compound of Formula (I) or a pharmaceutically acceptable salt, ester, solvate, alkylated quaternary ammonium salt, stereoisomer, tautomer or prodrug thereof wherein the bacterial infection comprises a vancomycin-intermediate Staphylococcus aureus bacterium. In one embodiment, is a method of treating a subject having a bacterial infection comprising administering to the subject a compound of Formula (I) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of between about 4 to about 8 μg/mL. In another embodiment, is a method of treating a subject having a bacterial infection comprising administering to the subject a compound of Formula (I) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 4 μg/mL. In yet another embodiment, is a method of treating a subject having a bacterial infection comprising administering to the subject a compound of Formula (I) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 5 μg/mL. In a further embodiment, is a method of treating a subject having a bacterial infection comprising administering to the subject a compound of Formula (I) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 6 μg/mL. In yet a further embodiment, is a method of treating a subject having a bacterial infection comprising administering to the subject a compound of Formula (I) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 7 μg/mL. In one embodiment, is a method of treating a subject having a bacterial infection comprising administering to the subject a compound of Formula (I) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 8 μg/mL.

In another aspect is a method of treating a subject having a bacterial infection comprising administering to the subject a compound of Formula (I) or a pharmaceutically acceptable salt, ester, solvate, alkylated quaternary ammonium salt, stereoisomer, tautomer or prodrug thereof wherein the bacterial infection comprises a vancomycin-resistant Staphylococcus aureus bacterium. In one embodiment, the vancomycin-resistant Staphylococcus aureus bacterium has a MIC of between about 16 μg/mL. In another embodiment, the vancomycin-resistant Staphylococcus aureus bacterium has a MIC of about ≧16 μg/mL. In yet another embodiment, the vancomycin-resistant Staphylococcus aureus bacterium has a MIC of about 20 μg/mL. In a further embodiment, the vancomycin-resistant Staphylococcus aureus bacterium has a MIC of about 25 μg/mL.

In one aspect is a method of treating a subject having a bacterial infection comprising administering to the subject a compound of Formula (II) or a pharmaceutically acceptable salt, ester, solvate, alkylated quaternary ammonium salt, stereoisomer, tautomer or prodrug thereof wherein the bacterial infection comprises a vancomycin-intermediate Staphylococcus aureus bacterium. In one embodiment, is a method of treating a subject having a bacterial infection comprising administering to the subject a compound of Formula (II) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of between about 4 to about 8 μg/mL. In another embodiment, is a method of treating a subject having a bacterial infection comprising administering to the subject a compound of Formula (II) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 4 μg/mL. In yet another embodiment, is a method of treating a subject having a bacterial infection comprising administering to the subject a compound of Formula (II) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 5 μg/mL. In a further embodiment, is a method of treating a subject having a bacterial infection comprising administering to the subject a compound of Formula (II) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 6 μg/mL. In yet a further embodiment, is a method of treating a subject having a bacterial infection comprising administering to the subject a compound of Formula (II) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 7 μg/mL. In one embodiment, is a method of treating a subject having a bacterial infection comprising administering to the subject a compound of Formula (II) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 8 μg/mL.

In another aspect is a method of treating a subject having a bacterial infection comprising administering to the subject a compound of Formula (II) or a pharmaceutically acceptable salt, ester, solvate, alkylated quaternary ammonium salt, stereoisomer, tautomer or prodrug thereof wherein the bacterial infection comprises a vancomycin-resistant Staphylococcus aureus bacterium. In one embodiment, the vancomycin-resistant Staphylococcus aureus bacterium has a MIC of between about 16 μg/mL. In another embodiment, the vancomycin-resistant Staphylococcus aureus bacterium has a MIC of about ≧16 μg/mL. In yet another embodiment, the vancomycin-resistant Staphylococcus aureus bacterium has a MIC of about 20 μg/mL. In a further embodiment, the vancomycin-resistant Staphylococcus aureus bacterium has a MIC of about 25 μg/mL.

In one aspect is a method of treating a subject having a bacterial infection comprising administering to the subject a compound of Formula (III) or a pharmaceutically acceptable salt, ester, solvate, alkylated quaternary ammonium salt, stereoisomer, tautomer or prodrug thereof wherein the bacterial infection comprises a vancomycin-intermediate Staphylococcus aureus bacterium. In one embodiment, is a method of treating a subject having a bacterial infection comprising administering to the subject a compound of Formula (III) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of between about 4 to about 8 μg/mL. In another embodiment, is a method of treating a subject having a bacterial infection comprising administering to the subject a compound of Formula (III) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 4 μg/mL. In yet another embodiment, is a method of treating a subject having a bacterial infection comprising administering to the subject a compound of Formula (III) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 5 μg/mL. In a further embodiment, is a method of treating a subject having a bacterial infection comprising administering to the subject a compound of Formula (III) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 6 μg/mL. In yet a further embodiment, is a method of treating a subject having a bacterial infection comprising administering to the subject a compound of Formula (III) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 7 μg/mL. In one embodiment, is a method of treating a subject having a bacterial infection comprising administering to the subject a compound of Formula (III) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 8 μg/mL.

In another aspect is a method of treating a subject having a bacterial infection comprising administering to the subject a compound of Formula (III) or a pharmaceutically acceptable salt, ester, solvate, alkylated quaternary ammonium salt, stereoisomer, tautomer or prodrug thereof wherein the bacterial infection comprises a vancomycin-resistant Staphylococcus aureus bacterium. In one embodiment, the vancomycin-resistant Staphylococcus aureus bacterium has a MIC of between about 16 μg/mL. In another embodiment, the vancomycin-resistant Staphylococcus aureus bacterium has a MIC of about ≧16 μg/mL. In yet another embodiment, the vancomycin-resistant Staphylococcus aureus bacterium has a MIC of about 20 μg/mL. In a further embodiment, the vancomycin-resistant Staphylococcus aureus bacterium has a MIC of about 25 μg/mL.

In one aspect is a method of treating a subject having a bacterial infection comprising administering to the subject a compound of Formula (IV) or a pharmaceutically acceptable salt, ester, solvate, alkylated quaternary ammonium salt, stereoisomer, tautomer or prodrug thereof wherein the bacterial infection comprises a vancomycin-intermediate Staphylococcus aureus bacterium. In one embodiment, is a method of treating a subject having a bacterial infection comprising administering to the subject a compound of Formula (IV) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of between about 4 to about 8 μg/mL. In another embodiment, is a method of treating a subject having a bacterial infection comprising administering to the subject a compound of Formula (IV) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 4 μg/mL. In yet another embodiment, is a method of treating a subject having a bacterial infection comprising administering to the subject a compound of Formula (IV) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 5 μg/mL. In a further embodiment, is a method of treating a subject having a bacterial infection comprising administering to the subject a compound of Formula (IV) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 6 μg/mL. In yet a further embodiment, is a method of treating a subject having a bacterial infection comprising administering to the subject a compound of Formula (IV) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 7 μg/mL. In one embodiment, is a method of treating a subject having a bacterial infection comprising administering to the subject a compound of Formula (IV) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 8 μg/mL.

In another aspect is a method of treating a subject having a bacterial infection comprising administering to the subject a compound of Formula (IV) or a pharmaceutically acceptable salt, ester, solvate, alkylated quaternary ammonium salt, stereoisomer, tautomer or prodrug thereof wherein the bacterial infection comprises a vancomycin-resistant Staphylococcus aureus bacterium. In one embodiment, the vancomycin-resistant Staphylococcus aureus bacterium has a MIC of between about 16 μg/mL. In another embodiment, the vancomycin-resistant Staphylococcus aureus bacterium has a MIC of about ≧16 μg/mL. In yet another embodiment, the vancomycin-resistant Staphylococcus aureus bacterium has a MIC of about 20 μg/mL. In a further embodiment, the vancomycin-resistant Staphylococcus aureus bacterium has a MIC of about 25 μg/mL.

In one aspect is a method of treating a subject having a bacterial infection comprising administering to the subject a compound of Formula (V) or a pharmaceutically acceptable salt, ester, solvate, alkylated quaternary ammonium salt, stereoisomer, tautomer or prodrug thereof wherein the bacterial infection comprises a vancomycin-intermediate Staphylococcus aureus bacterium. In one embodiment, is a method of treating a subject having a bacterial infection comprising administering to the subject a compound of Formula (V) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of between about 4 to about 8 μg/mL. In another embodiment, is a method of treating a subject having a bacterial infection comprising administering to the subject a compound of Formula (V) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 4 μg/mL. In yet another embodiment, is a method of treating a subject having a bacterial infection comprising administering to the subject a compound of Formula (V) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 5 μg/mL. In a further embodiment, is a method of treating a subject having a bacterial infection comprising administering to the subject a compound of Formula (V) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 6 μg/mL. In yet a further embodiment, is a method of treating a subject having a bacterial infection comprising administering to the subject a compound of Formula (V) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 7 μg/mL. In one embodiment, is a method of treating a subject having a bacterial infection comprising administering to the subject a compound of Formula (V) or a pharmaceutically acceptable salt, ester, solvate, alkylated quaternary ammonium salt, stereoisomer, tautomer or prodrug thereof wherein the vancomycin-intermediate Staphylococcus aureus bacterium has a MIC of about 8 μg/mL.

In another aspect is a method of treating a subject having a bacterial infection comprising administering to the subject a compound of Formula (V) or a pharmaceutically acceptable salt, ester, solvate, alkylated quaternary ammonium salt, stereoisomer, tautomer or prodrug thereof wherein the bacterial infection comprises a vancomycin-resistant Staphylococcus aureus bacterium. In one embodiment, the vancomycin-resistant Staphylococcus aureus bacterium has a MIC of between about 16 μg/mL. In another embodiment, the vancomycin-resistant Staphylococcus aureus bacterium has a MIC of about ≧16 μg/mL. In yet another embodiment, the vancomycin-resistant Staphylococcus aureus bacterium has a MIC of about 20 μg/mL. In a further embodiment, the vancomycin-resistant Staphylococcus aureus bacterium has a MIC of about 25 μg/mL.

### Vancomycin-Resistant Enterococci

Enterococci are bacteria that are normally present in the human intestines and in the female genital tract and are often found in the environment. These bacteria sometimes cause infections. In some cases, enterococci have become resistant to vancomycin (also known as vancomycin-resistant enterococci or VRE.) Common forms of resistance to vancomycin occur in enterococcal strains that involve the acquisition of a set of genes encoding proteins that direct peptidoglycan precursors to incorporate D-Ala-D-Lac instead of D-Ala-D-Ala. The six different types of vancomycin resistance shown by enterococcus are: Van-A, Van-B, Van-C, Van-D, Van-E and Van-F. In some cases, Van-A VRE is resistant to both vancomycin and teicoplanin, while in other cases, Van-B VRE is resistant to vancomycin but sensitive to teicoplanin; in further cases Van-C is partly resistant to vancomycin, and sensitive to teicoplanin.

In one aspect, is a method of treating a subject having a vancomycin-resistant enterococci comprising administering to the subject a compound of Formula (I) or a pharmaceutically acceptable salt, ester, solvate, alkylated quaternary ammonium salt, stereoisomer, tautomer or prodrug thereof wherein the enterococci has developed resistance to vancomycin. In one embodiment, the subject has been previously treated with vancomycin for a sustained period of time. In another embodiment, the subject has been hospitalized. In yet another embodiment, the subject has a weakened immune system such as patients in Intensive Care Units or in cancer or transplant wards. In a further embodiment, the subject has undergone surgical procedures such as, for example, abdominal or chest surgery. In yet a further embodiment, the subject has been colonized with VRE. In one embodiment, the subject has a medical device such that an infection has developed. In another embodiment, the medical device is a urinary catheter or central intravenous (IV) catheter.

In another embodiment, is a method of treating a subject having a vancomycin-resistant enterococci comprising administering to the subject a compound of Formula (I) or a pharmaceutically acceptable salt, ester, solvate, alkylated quaternary ammonium salt, stereoisomer, tautomer or prodrug thereof wherein the enterococcus has Van-A resistance.

In another embodiment, is a method of treating a subject having a vancomycin-resistant enterococci comprising administering to the subject a compound of Formula (I) or a pharmaceutically acceptable salt, ester, solvate, alkylated quaternary ammonium salt, stereoisomer, tautomer or prodrug thereof wherein the enterococcus has Van-B resistance.

In another embodiment, is a method of treating a subject having a vancomycin-resistant enterococci comprising administering to the subject a compound of Formula (I) or a pharmaceutically acceptable salt, ester, solvate, alkylated quaternary ammonium salt, stereoisomer, tautomer or prodrug thereof wherein the enterococcus has Van-C resistance.

In one aspect, is a method of treating a subject having a vancomycin-resistant enterococci comprising administering to the subject a compound of Formula (II) or a pharmaceutically acceptable salt, ester, solvate, alkylated quaternary ammonium salt, stereoisomer, tautomer or prodrug thereof wherein the enterococci has developed resistance to vancomycin. In one embodiment, the subject has been previously treated with vancomycin for a sustained period of time. In another embodiment, the subject has been hospitalized. In yet another embodiment, the subject has a weakened immune system such as patients in Intensive Care Units or in cancer or transplant wards. In a further embodiment, the subject has undergone surgical procedures such as, for example, abdominal or chest surgery. In yet a further embodiment, the subject has been colonized with VRE. In one embodiment, the subject has a medical device such that an infection has developed. In another embodiment, the medical device is a urinary catheter or central intravenous (IV) catheter.

In another embodiment, is a method of treating a subject having a vancomycin-resistant enterococci comprising administering to the subject a compound of Formula (II) or a pharmaceutically acceptable salt, ester, solvate, alkylated quaternary ammonium salt, stereoisomer, tautomer or prodrug thereof wherein the enterococcus has Van-A resistance.

In another embodiment, is a method of treating a subject having a vancomycin-resistant enterococci comprising administering to the subject a compound of Formula (II) or a pharmaceutically acceptable salt, ester, solvate, alkylated quaternary ammonium salt, stereoisomer, tautomer or prodrug thereof wherein the enterococcus has Van-B resistance.

In another embodiment, is a method of treating a subject having a vancomycin-resistant enterococci comprising administering to the subject a compound of Formula (II) or a pharmaceutically acceptable salt, ester, solvate, alkylated quaternary ammonium salt, stereoisomer, tautomer or prodrug thereof wherein the enterococcus has Van-C resistance.

In one aspect, is a method of treating a subject having a vancomycin-resistant enterococci comprising administering to the subject a compound of Formula (III) or a pharmaceutically acceptable salt, ester, solvate, alkylated quaternary ammonium salt, stereoisomer, tautomer or prodrug thereof wherein the enterococci has developed resistance to vancomycin. In one embodiment, the subject has been previously treated with vancomycin for a sustained period of time. In another embodiment, the subject has been hospitalized. In yet another embodiment, the subject has a weakened immune system such as patients in Intensive Care Units or in cancer or transplant wards. In a further embodiment, the subject has undergone surgical procedures such as, for example, abdominal or chest surgery. In yet a further embodiment, the subject has been colonized with VRE. In one embodiment, the subject has a medical device such that an infection has developed. In another embodiment, the medical device is a urinary catheter or central intravenous (IV) catheter.

In another embodiment, is a method of treating a subject having a vancomycin-resistant enterococci comprising administering to the subject a compound of Formula (III) or a pharmaceutically acceptable salt, ester, solvate, alkylated quaternary ammonium salt, stereoisomer, tautomer or prodrug thereof wherein the enterococcus has Van-A resistance.

In another embodiment, is a method of treating a subject having a vancomycin-resistant enterococci comprising administering to the subject a compound of Formula (III) or a pharmaceutically acceptable salt, ester, solvate, alkylated quaternary ammonium salt, stereoisomer, tautomer or prodrug thereof wherein the enterococcus has Van-B resistance.

In another embodiment, is a method of treating a subject having a vancomycin-resistant enterococci comprising administering to the subject a compound of Formula (III) or a pharmaceutically acceptable salt, ester, solvate, alkylated quaternary ammonium salt, stereoisomer, tautomer or prodrug thereof wherein the enterococcus has Van-C resistance.

In one aspect, is a method of treating a subject having a vancomycin-resistant enterococci comprising administering to the subject a compound of Formula (IV) or a pharmaceutically acceptable salt, ester, solvate, alkylated quaternary ammonium salt, stereoisomer, tautomer or prodrug thereof wherein the enterococci has developed resistance to vancomycin. In one embodiment, the subject has been previously treated with vancomycin for a sustained period of time. In another embodiment, the subject has been hospitalized. In yet another embodiment, the subject has a weakened immune system such as patients in Intensive Care Units or in cancer or transplant wards. In a further embodiment, the subject has undergone surgical procedures such as, for example, abdominal or chest surgery. In yet a further embodiment, the subject has been colonized with VRE. In one embodiment, the subject has a medical device such that an infection has developed. In another embodiment, the medical device is a urinary catheter or central intravenous (IV) catheter.

In another embodiment, is a method of treating a subject having a vancomycin-resistant enterococci comprising administering to the subject a compound of Formula (IV) or a pharmaceutically acceptable salt, ester, solvate, alkylated quaternary ammonium salt, stereoisomer, tautomer or prodrug thereof wherein the enterococcus has Van-A resistance.

In another embodiment, is a method of treating a subject having a vancomycin-resistant enterococci comprising administering to the subject a compound of Formula (IV) or a pharmaceutically acceptable salt, ester, solvate, alkylated quaternary ammonium salt, stereoisomer, tautomer or prodrug thereof wherein the enterococcus has Van-B resistance.

In another embodiment, is a method of treating a subject having a vancomycin-resistant enterococci comprising administering to the subject a compound of Formula (IV) or a pharmaceutically acceptable salt, ester, solvate, alkylated quaternary ammonium salt, stereoisomer, tautomer or prodrug thereof wherein the enterococcus has Van-C resistance.

In one aspect, is a method of treating a subject having a vancomycin-resistant enterococci comprising administering to the subject a compound of Formula (V) or a pharmaceutically acceptable salt, ester, solvate, alkylated quaternary ammonium salt, stereoisomer, tautomer or prodrug thereof wherein the enterococci has developed resistance to vancomycin. In one embodiment, the subject has been previously treated with vancomycin for a sustained period of time. In another embodiment, the subject has been hospitalized. In yet another embodiment, the subject has a weakened immune system such as patients in Intensive Care Units or in cancer or transplant wards. In a further embodiment, the subject has undergone surgical procedures such as, for example, abdominal or chest surgery. In yet a further embodiment, the subject has been colonized with VRE. In one embodiment, the subject has a medical device such that an infection has developed. In another embodiment, the medical device is a urinary catheter or central intravenous (IV) catheter.

In another embodiment, is a method of treating a subject having a vancomycin-resistant enterococci comprising administering to the subject a compound of Formula (V) or a pharmaceutically acceptable salt, ester, solvate, alkylated quaternary ammonium salt, stereoisomer, tautomer or prodrug thereof wherein the enterococcus has Van-A resistance.

In another embodiment, is a method of treating a subject having a vancomycin-resistant enterococci comprising administering to the subject a compound of Formula (V) or a pharmaceutically acceptable salt, ester, solvate, alkylated quaternary ammonium salt, stereoisomer, tautomer or prodrug thereof wherein the enterococcus has Van-B resistance.

In another embodiment, is a method of treating a subject having a vancomycin-resistant enterococci comprising administering to the subject a compound of Formula (V) or a pharmaceutically acceptable salt, ester, solvate, alkylated quaternary ammonium salt, stereoisomer, tautomer or prodrug thereof wherein the enterococcus has Van-C resistance.

### EXAMPLES

The following examples provide details concerning the synthesis, properties and activities and applications of semi-synthetic glycopeptides described herein. It should be understood the following is representative only.

### Example 1

### Synthesis of Compound (1)

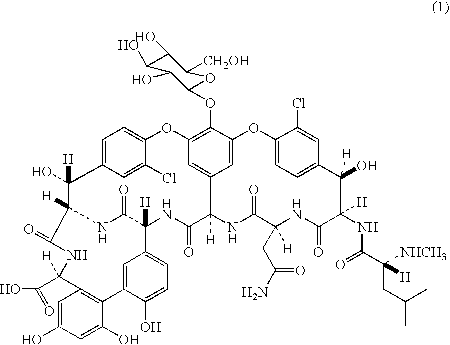

Vancomycin (30 g) was added slowly to a mixture solution (300 ml, TFA:H₂O=9:1) at 10° C. Then the reaction mixture was stirred at 10° C. for 2 hrs (with reaction progress checked by HPLC). The reaction mixture was quenched to 1500 ml cold diethyl ether, the precipitate was filtered and washed by ether several times, dried under vacuum. The crude product was purified by reverse phase column (MeCN:H₂O=10%˜20%) to afford Compound (1) as a white solid. (yield=45%).

### Example 2

### Synthesis of Compound (2)

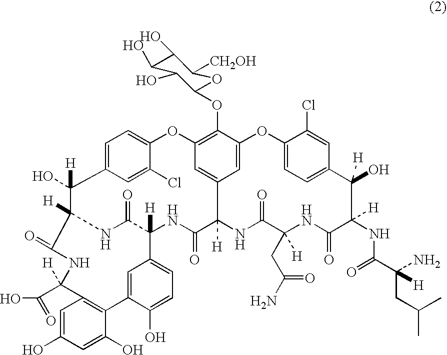

Using a procedure similar to the preparation of Compound (1), and replacing vancomycin with desmethylvancomycin, Compound (2) is made.

### Example 3

### Synthesis of Compound (3)

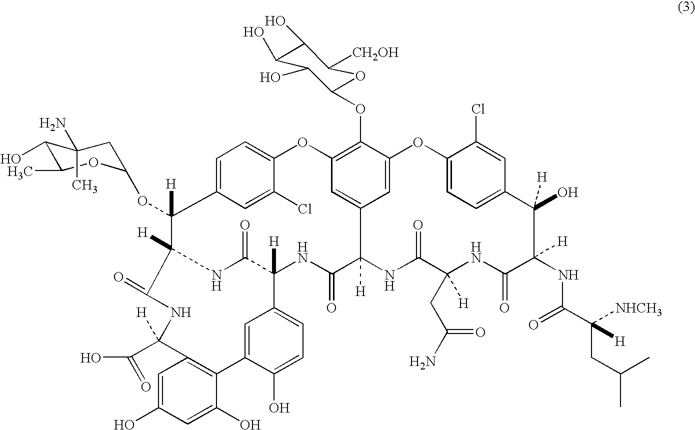

Using a procedure similar to the preparation of Compound (1), and replacing vancomycin with LY264826, Compound (3) is made.

### Example 4

### Synthesis of Compound (4)

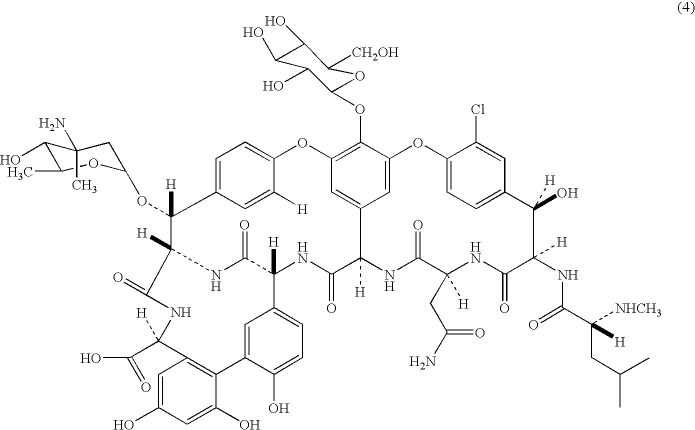

Using a procedure similar to the preparation of Compound (1), and replacing vancomycin with eremomycin, Compound (4) is made.

### Example 5

### Synthesis of Compound (5)

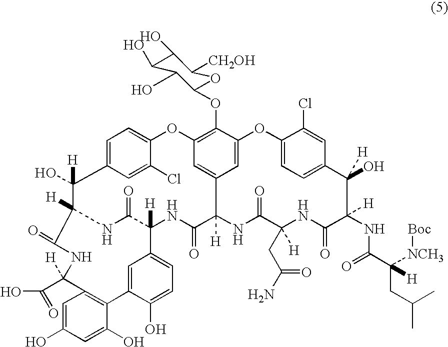

Compound (1) (5.0 g, 3.72 mmol) was dissolved in THF/H₂O (35 ml/35 ml). TEA (0.77 ml, 5.58 mmol) was then added. The reaction mixture was cooled down to 15° C. and then (Boc)₂O (0.89 g, 4.08 mmol) was added slowly. After the addition, the reaction mixture was allowed to be stirred at 15° C. for 7 hrs. It was concentrated and the crude was purified by reverse phase column (MeCN:H₂O=1:5-3:10). 3 g of Compound (5) was obtained as a white solid (yield=60%).

### Example 6

### Synthesis of Compound (6)

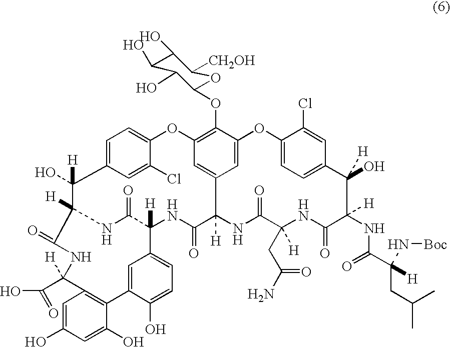

Using a procedure similar to the preparation of Compound (5), and replacing Compound (1) with Compound (2), Compound (6) is made.

### Example 7

### Synthesis of Compound (7)

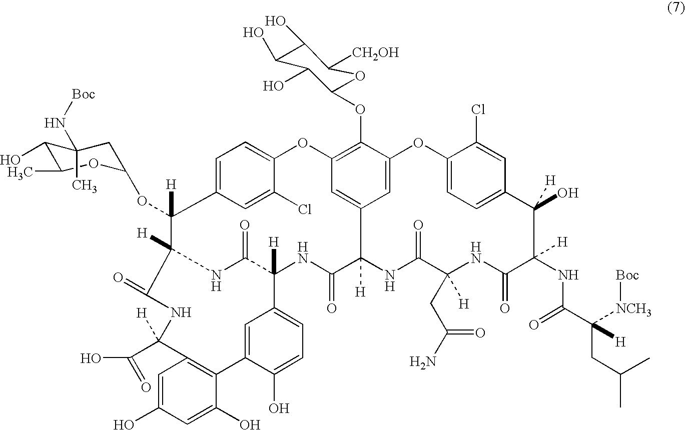

Using a procedure similar to the preparation of Compound (5), and replacing Compound (1) with Compound (3), Compound (7) is made.

### Example 8

### Synthesis of Compound (8)

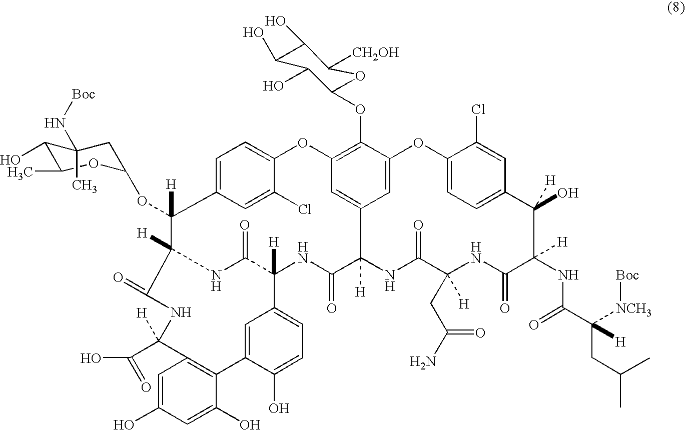

Using a procedure similar to the preparation of Compound (5), and replacing Compound (1) with Compound (4), Compound (8) is made.

### Example 9

### Synthesis of Compound (91)

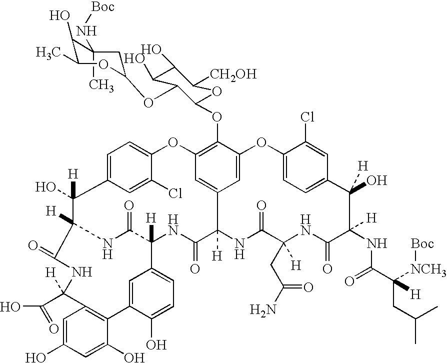

Using a procedure similar to the preparation of Compound (5), and replacing Compound (1) with vancomycin, Compound (9) is made.

### Example 10

### Synthesis of Compound (10)

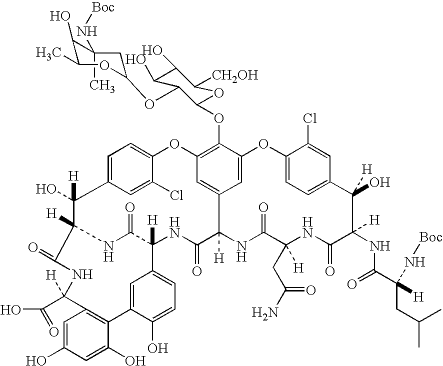

Using a procedure similar to the preparation of Compound (5), and replacing Compound (1) with desmethylvancomycin Compound (10) is made.

### Example 11

### Synthesis of Compound (11)

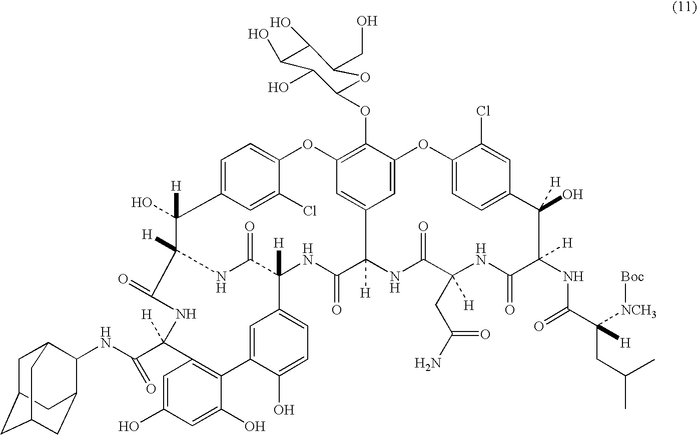

Compound (5) (1 g, 0.712 mmol) and 2-adamantylamine hydrochloride (0.4 g, 2.1 mmol) were dissolved in anhydrous DMSO (12 ml). DIEA was added the solution to adjust the pH of reaction mixture to 8. HATU (0.3 g, 0.789 mmol) was then added in the presence of DIEA. Stirring was continued for about 1 hr, checking the progress of the reaction to completion by TLC. The resulting mixture was then added to 120 ml of water and filtered. The cake was washed for two times with water and dried in vacuum. Purification by running a normal phase silica column (MeOH:CH₂Cl₂=1:7-1:3) gave the Compound (11) as white solid (850 mg, yield=77%).

### Example 12

### Synthesis of Compound (12)

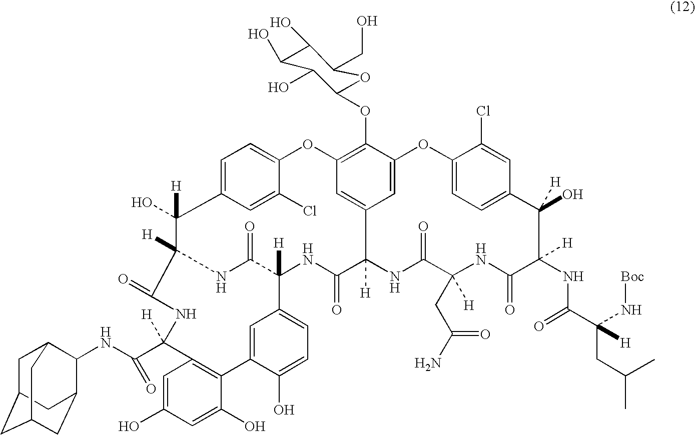

Using a procedure similar to the preparation of Compound (11), and replacing Compound (5) with Compound (6), Compound (12) is made.

### Example 13

### Synthesis of Compound (13)

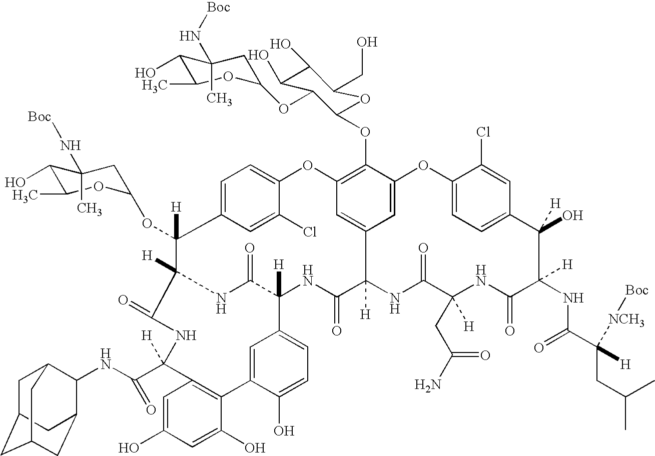

Using a procedure similar to the preparation of Compound (11), and replacing Compound (5) with Compound (7), Compound (13) is made.

### Example 14

### Synthesis of Compound (14)

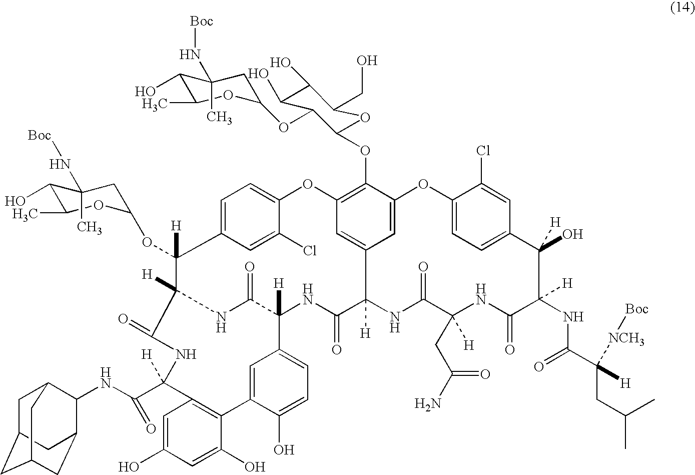

Using a procedure similar to the preparation of Compound (11), and replacing Compound (5) with Compound (8), Compound (14) is made.

### Example 15

### Synthesis of Compound (15)

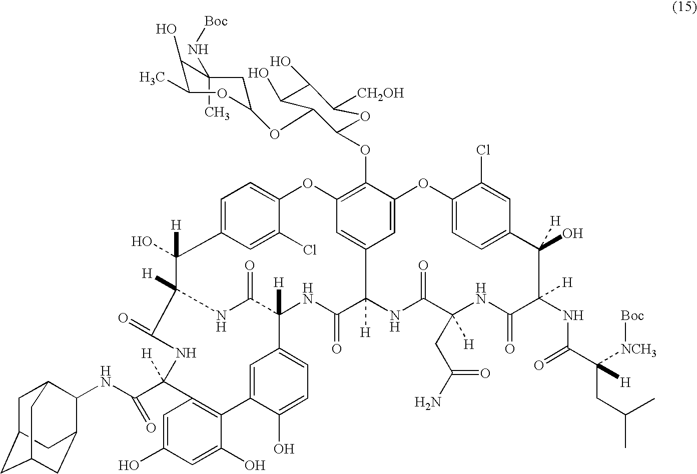

Using a procedure similar to the preparation of Compound (11), and replacing Compound (5) with Compound (9), Compound (15) is made.

### Example 16

### Synthesis of Compound (16)

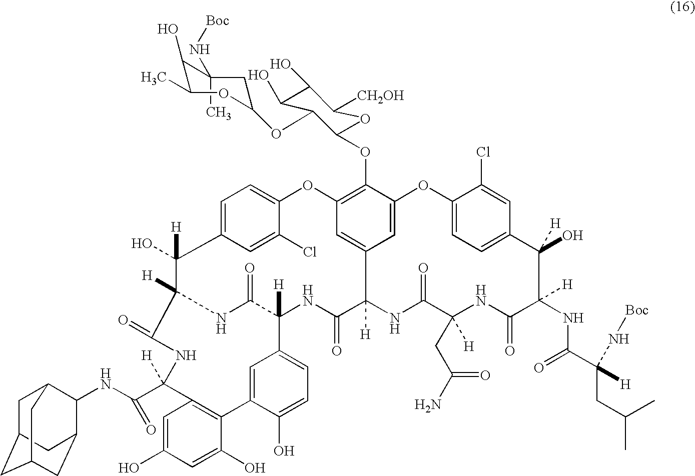

Using a procedure similar to the preparation of Compound (11), and replacing Compound (5) with Compound (10), Compound (16) is made.

### Example 17

### Synthesis of Compound (17)

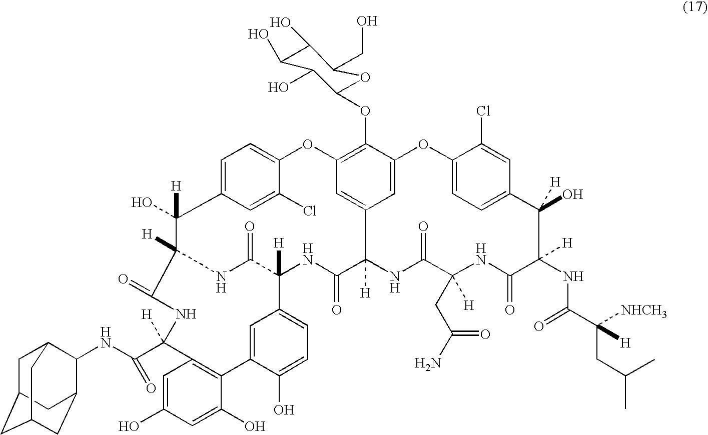

To a suspension of Compound (11) (380 mg) in CH₂Cl₂ (4 ml) at 0° C., was added TFA (0.5 ml) dropwise. The reaction mixture was stirred at 0° C. for 1 hour and then at room temperature for another hour. The reaction was follow by HPLC until the analysis showed no starting material present. Ether (30 ml) was added and the forming solid was collected and washed with ether twice. The collected white solid was dried and purified by preparative HPLC to yield Compound (17) as TFA salt.

### Example 18

### Synthesis of Compound (18)

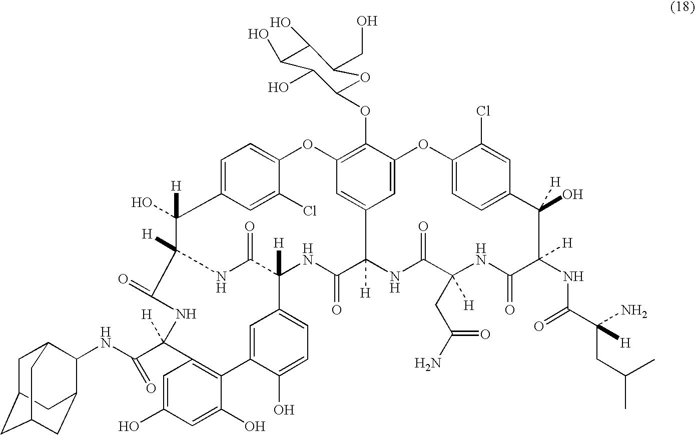

Using a procedure similar to the preparation of Compound (17), and replacing Compound (11) with Compound (12), Compound (18) as TFA salt is made.

### Example 19

### Synthesis of Compound (19)

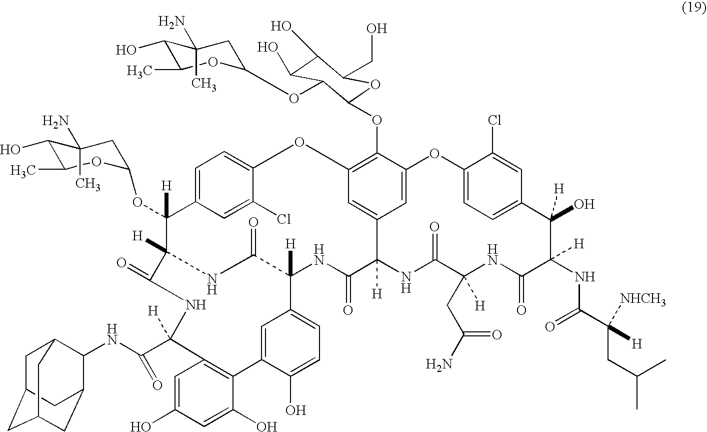

Using a procedure similar to the preparation of Compound (17), and replacing Compound (11) with Compound (13), Compound (19) as TFA salt is made.

### Example 20

### Synthesis of Compound (20)

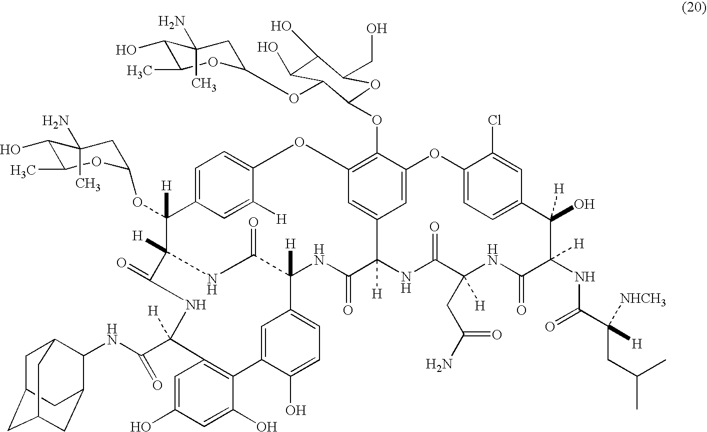

Using a procedure similar to the preparation of Compound (17), and replacing Compound (11) with Compound (14), Compound (20) as TFA salt is made.

### Example 21

### Synthesis of Compound (21)

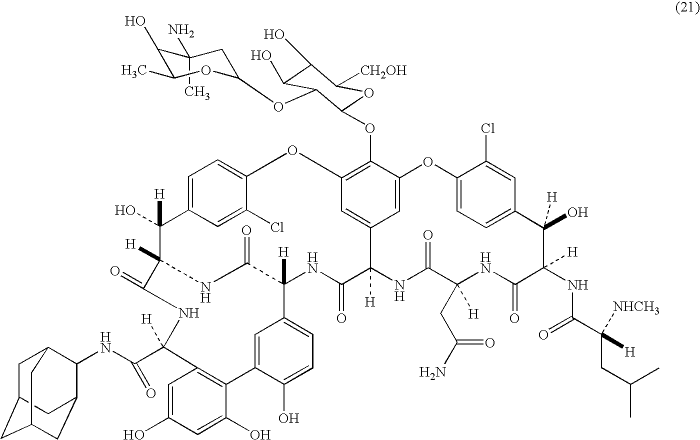

Using a procedure similar to the preparation of Compound (17), and replacing Compound (11) with Compound (15), Compound (21) as TFA salt is made.

### Example 22

### Alternate Synthesis of Compound (21)

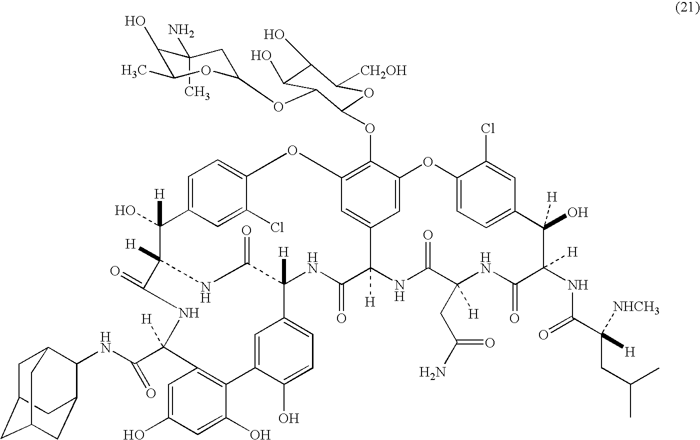

To a solution of vancomycin hydrochloride (100.0 g) in DMSO (800 mL) was added 2-adamantylamine hydrochloride (20.0 g), DIPEA (35.0 g) and HATU (28.1 g) with stirring at ambient temperature. The reaction mixture was stirred overnight. Analytical HPLC showed the reaction completed. DMSO was removed under vacuum. The residue was subjected to purification by reverse phase silica gel column chromatography (C18 silica gel, CH₃CN—H₂O: 5%-30%). The collected fraction was condensed to give Compound (21) (45 g) as white powder.

### Example 23

### Synthesis of Compound (22)

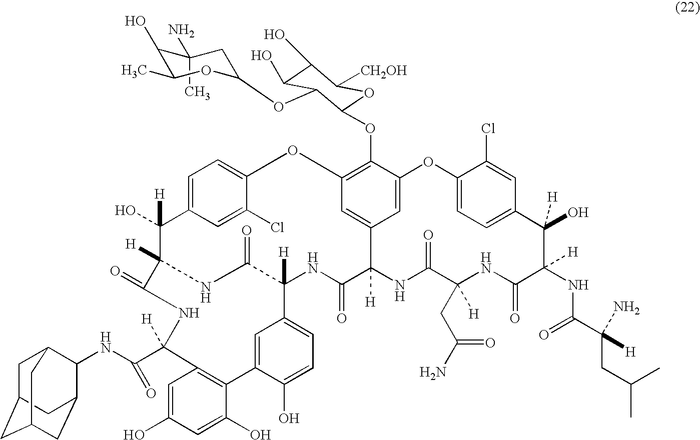

Using a procedure similar to the preparation of Compound (17), and replacing Compound (11) with Compound (16), Compound (22) as TFA salt is made.

### Example 24

### 4-(pentyloxy)benzene-1-sulfonyl chloride

A. A mixture of phenol (28.2, 0.3 mol, 1 eq.), potassium carbonate (63 g, 1.5 eq.) and 1-bromopentane (47.6 g) in acetone (200 mL) was stirred at reflux overnight. The solid was filtrated away. The filtrate was condensed under reduced pressure. The residue was purified by flash silica gel column chromatography (300-400 mesh, eluent: hexanes) to give pentyloxybenzene (45 g, 90%) as colorless oil.

B. To a solution of pentyloxybenzene (41 g, 1 eq) in CH₂Cl₂ (300 mL) was added dropwise a solution of chlorosulfonic acid (70 g, 2 eq.) in CH₂Cl₂ (50 mL) at −10° C. with stirring. After completion monitored by TLC (EtOAc/Hexanes=1/10), the reaction mixture was poured into ice-water mixture (500 g). The organic layer was partitioned. The water layer was extracted with CH₂Cl₂. The combined organic phase was washed with brine, dried over Na₂SO₄, condensed under reduced pressure and purified by silica gel column chromatography (300-400 mesh, eluent: hexanes) to give 4-(pentyloxy)benzene-1-sulfonyl chloride (42 g, 60%) as yellowish oil.

### Example 25

### 4-butoxybenzene-1-sulfonyl chloride

To a mixture of 40% aqueous hydrobromic acid (76 g) and n-butanol (18.5 g) was added condensed sulfuric acid (12.5 g) at 0° C. with stirring. The reaction mixture was heated at reflux for 5 h and then applied to atmospheric distillation. The collected liquid was dissolved into ether and washed with diluted sodium bicarbonate. The ether layer was dried and condensed to give 1-bromobutane (24 g, 69%) as colorless oil. ¹H NMR (400 M) 7.9 (2H, d, Ar), 7.0 (2H, d, Ar), 4.0 (2H, t, —CH₂—), 1.8 (2H, m), 1.4 (4H, m), 0.8 (3H). A mixture of phenol (17.3 g), potassium carbonate (48 g) and 1-bromobutane (24 g) in acetone was stirred at reflux overnight. The solid was filtrated away. The filtrate was condensed under reduced pressure. The residue was purified by flash silica gel column chromatography to give butoxybenzene (25 g, 90%) as colorless oil. To a solution of butoxybenzene (25 g) in CH₂Cl₂ (200 mL) was added dropwise a solution of chlorosulfonic acid (40 g) in CH₂Cl₂ (100 mL) at −10° C. with stirring. After completion monitoring by TLC (EtOAc/Hexanes=1/10), the reaction mixture was poured into ice-water mixture (300 g). The organic layer was partitioned. The water layer was extracted with CH₂Cl₂. The combined organic phase was washed with brine, dried over Na₂SO₄, condensed under reduced pressure and purified by silica gel column chromatography (300-400 mesh, eluent: hexanes) to give 4-butoxybenzene-1-sulfonyl chloride (19 g, 46%) as yellowish oil.

### Example 26

### 4-(hexyloxy)benzene-1-sulfonyl chloride

Using a procedure similar to Example 25, using hexanol instead of n-butanol, 4-(hexyloxy)benzene-1-sulfonyl chloride is made.

### Example 27

### 4-propoxybenzene-1-sulfonyl chloride

Using a procedure similar to Example 25, using propan-ol instead of n-butanol, 4-propoxybenzene-1-sulfonyl chloride is made.

### Example 28

### (3-methoxypropoxy)benzene

To a suspension of phenol (4.7 g, 63.2 mmol) and K₂CO₃ powder (26 g, 186 mmol) in N,N-dimethylformamide (100 ml) with stirring was added 1,3-dibromopropane (20 ml, 186 mmol) at room temperature. The reaction mixture was heated at 50° C. for 5 h, and monitored by TLC till the phenol was disappeared. The reaction mixture was quenched by adding water (200 ml) and extracted with dichloromethane. The combined organic phase was washed with brine, dried over Na₂SO₄, condensed by rotary evaporator to give a mixture of (3-bromopropoxy)benzene and some elimination products. (3-Bromopropoxy)benzene (6.2 g, 48%) was obtained by vacuum distillation. ¹H NMR (CDCl₃, 400 MHz) 2.3 (2H), 3.6 (2H), 4.1 (2H), 6.85-6.95 (3H), 7.3 (2H); ¹³C NMR (CDCl₃, 400 MHz) 59, 67, 71, 114, 120, 129, 158). To a solution of NaOMe in methanol (in situ prepared from 1.1 g of sodium dissolved into 200 ml of MeOH) was added dropwise a solution of (3-bromopropoxy)benzene (9.2 g, 42.8 mmol) in 20 ml of MeOH. After completion of the addition, the reaction mixture was stirred overnight at reflux till the starting material was disappeared. The volatile solvents were removed by rotary evaporator under vacuum. (3-Methoxypropoxy)benzene (4.7 g, 66%) was obtained by vacuum distillation. ¹H NMR (CDCl₃, 400 MHz) 3.45 (3H), 3.75 (2H), 4.12 (2H), 6.95 (2H), 7.3 (2H); ¹³C NMR (CDCl₃, 400 MHz) δ59, 67, 71, 114, 120, 129, 158. is made.

### Example 29

### (3-ethoxypropoxy)benzene

To a solution of NaOEt (in situ prepared from 1.1 g of sodium dissolved into 200 ml of EtOH), was added dropwise a solution of (3-bromopropoxy)benzene (9.2 g, 42.7 mmol) in 20 ml of EtOH. After completion of the addition, the reaction mixture was stirred overnight at reflux till the starting material was disappeared. The volatile solvents were removed by rotary evaporator under vacuum. (3-Ethoxypropoxy)benzene (4.7 g, 60%) was isolated by vacuum distillation. ¹H NMR (CDCl₃, 400 MHz) 1.25 (3H), 2.1 (2H), 3.55 (2H), 3.7 (2H), 4.2 (2H), 4.15 (2H), 6.95-7.05 (4H), 7.3-7.4 (2H), ¹³C NMR (CDCl₃, 400 MHz) 15.2, 29.9, 64.3, 65.2, 66.3, 114.5, 120.6, 129.4, 159.0.

### Example 30

### (3-propoxypropoxy)benzene

Using a procedure similar to Example 29, using n propanol instead of ethanol, (3-propoxypropoxy)benzene is made. ¹H NMR (CDCl₃, 400 MHz) 0.8 (3H), 1.4-1.6 (2H), 1.9-2.0 (2H), 3.2-3.3 (2H), 3.5-3.6 (2H), 3.9-4.1 (2H), 6.7-6.9 (2H), 7.1-7.2 (2H).

### Example 31

### 4-(3-propoxypropoxy)benzene-1-sulfonyl chloride

To a solution of (3-propoxypropoxy)benzene (1.52 g, 10 mmol) in 40 ml of tetrahydrofuran (THF), was added dropwise a solution of chlorosulphonic acid (4.0 g, 35 mmol) in dichloromethane at 0° C. with rapid stirring under nitrogen. The reaction mixture was stirred at room temperature for 45 min and was poured into cooled water. Extraction, washing, drying and condensation gave a syrup, which was isolated by flash column chromatography on silica gel to provide 4-(3-propoxypropoxy)benzene-1-sulfonyl chloride (1 g, 37%). ¹H NMR (CDCl₃, 400 MHz) 0.8 (3H), 1.5-1.6 (2H), 2.0-2.1 (2H), 3.25-3.35 (2H), 3.5-3.6 (2H), 4.1-4.2 (2H), 6.9-7.0 (3H), 7.8-7.9 (2H).

### Example 32

### 4-(3-methoxypropoxy)benzene-1-sulfonyl chloride

Using a procedure similar to Example 31 using (3-methoxypropoxy)benzene instead of (3-propoxypropoxy)benzene, 4-(3-methoxypropoxy)benzene-1-sulfonyl chloride is made.

### Example 33

### 4-(3-ethoxypropoxy)benzene-1-sulfonyl chloride

Using a procedure similar to Example 31 using 3-ethoxypropoxybenzene instead of (3-propoxypropoxy)benzene, 4-(3-ethoxypropoxy)benzene-1-sulfonyl chloride is made.

### Example 34

### (2-ethoxyethoxy)benzene

To a 500 ml of round-bottom flask was added sequentially EtOH (150 ml), phenol (15.4 g), K₂CO₃ (10.7 g), and ethylene oxide (10 ml) at 0° C. with stirring. The resulting mixture was warmed to room temperature and stirred overnight. The reaction was monitored by GC-MS and 200 ml of dichloromethane was added to the reaction mixture when the reaction was completed. The crude product was passed through a pad of celite to remove insoluble solids. The organic phase was concentrated under vacuum. The residue was isolated by distillation under reduce pressure to afford the 2-phenoxyethanol (17.75 g) as an oil. To a solution of 2-phenoxyethanol (7 g) in 40 ml of THF was added powdered NaH (2.7 g, 60% in mineral oil) at room temperature with stirring. The resulting mixture was stirred at room temperature for 1 hr then bromoethane (4 ml) was added and stirred for an additional 2 hr. The reaction was monitored by TLC (hexanes:EtOAc=10:1). 10 ml of ice-water was added into the reaction mixture. The organic layer was separated. The aqueous layer was extracted with EtOAc (2×20 mL). The combined organic phases were washed with brine, dried on Na₂SO₄, condensed under reduce pressure. The residue was purified by flash column chromatography on silica gel (eluent: hexane) to afford the (2-ethoxyethoxy)benzene (6.68 g, 79%) as an oil.

### Example 35

### (2-propoxyethoxy)benzene

Using a procedure similar to the preparation of (2-ethoxyethoxy)benzene as in Example 34 and replacing bromoethane with 1-bromopropane, (2-propoxyethoxy)benzene is made.

### Example 36

### 4-(2-ethoxyethoxy)benzene-1-sulfonyl chloride

Using a procedure similar to Example 24A, on the preparation of 4-(pentyloxy)benzene-1-sulfonyl chloride, and replacing pentyloxybenzene with (2-ethoxyethoxy)benzene, 4-(2-ethoxyethoxy)benzene-1-sulfonyl chloride is made.

### Example 37

### 4-(2-prooxyethoxy)benzene-1-sulfonyl chloride

Using a procedure similar to Example 24A, on the preparation of 4-(pentyloxy)benzene-1-sulfonyl chloride, and replacing pentyloxybenzene with (2-prooxyethoxy)benzene, 4-(2-ethoxyethoxy)benzene-1-sulfonyl chloride is made.

### Example 38

### N-(2-aminoethyl)-4-(pentyloxy)benzenesulfonamide

To a solution of ethane-1,2-diamine (8.5 g) in CH₂Cl₂ (50 mL) was slowly added dropwise a solution of 4-butoxybenzene-1-sulfonyl chloride (4 g) in DCM (10 mL) with stirring at 0° C. After being stirred at rt for 3.5 h, the reaction mixture was washed with saturated NH₄Cl. The organic layer was dried over Na₂SO₄, condensed under reduced pressure and purified by silica gel column chromatography to give N-(2-aminoethyl)-4-(pentyloxy)benzenesulfonamide (4 g) as a white powder.

### Example 39

### N-(6-aminohexyl)-4-(pentyloxy)benzenesulfonamide

To a solution of hexane-1,6-diamine (8.8 g) in CH₂Cl₂ (40 mL) was slowly added dropwise a solution of 4-butoxybenzene-1-sulfonyl chloride (2 g) in DCM (20 mL) with stirring at 0° C. After being stirred at rt for 4 h, the reaction mixture was washed with saturated NH₄Cl. The organic layer was dried over Na₂SO₄, condensed under reduced pressure and purified by silica gel column chromatography to give N-(6-aminohexyl)-4-(pentyloxy)benzenesulfonamide (2 g) as a white made.

### Example 40

### 4-amino-N-(4-(pentyloxy)phenylsulfonyl)butanamide hydrochloride

A solution of 4-aminobutanoic acid (10.3 g) in THF (100 mL) was mixed with a solution K₂CO₃ (6.9 g) in water (50 mL). Di-tent-butyl dicarbonate (26 g) was added dropwise into the mixture at room temperature with stirring. The resulting mixture was stirred at room temperature until the starting material was completely consumed by TLC monitoring. THF was removed under reduced pressure and the remaining aqueous phase was adjusted to pH=4-5 by using aqueous KHSO₄ (1 N) at 0° C. Extracted with EtOAc, washed with brine, dried on MgSO₄ and condensed to afford 4-(tert-butoxycarbonylamino)butanoic acid (19.6 g, 96%). DCC (1.59 g) and HOSu (0.88 g) was added into a solution of 4-(tert-butoxycarbonyl)butanoic acid (1.737 g) in DMF (20 ml) at 0° C. with stirring. The mixture was allowed to warm to room temperature and stirred overnight. The starting material was disappeared by TLC monitoring. The resulted solid was filtered away and washed with a little of DMF. To the filtration containing 2,5-dioxopyrrolidin-1-yl-4-(tert-butoxycarbonylamino)butanoate was added 4-(pentyloxy)benzenesulfonamide (2 g) and K₂CO₃ (2.12 g) at room temperature with stirring. The mixture was stirred until the starting material was disappeared by TLC monitoring. The resulted solid was removed by filtration under vacuum. The filtrate was poured into water (20 ml) and the organic layer was partitioned. The aqueous layer was extracted with EtOAc. The combined organic layers were washed with water and brine, and then dried over anhydrous Na₂SO. Upon concentration under reduced pressure, it gave tert-butyl 4-oxo-4-(4-(pentyloxy)phenylsulfonamide)butylcarbamate as a syrup. This syrup was dissolved into 10 ml of EtOAc and was treated with in situ HCl (prepared from 10 ml of acetyl chloride dissolved into methanol) at 0° C. On completion of the reaction, the solvent was removed under reduce pressure. The residue was dissolved in 20 ml of water. The aqueous solution was washed with EtOAc (2×10 ml) and was adjusted to pH=8 with saturated aqueous K₂CO₃. Extracted with EtOAc, washed with brine, dried on Na₂SO₄ and concentrated under reduced pressure to provide 4-amino-N-(4-(pentyloxy)phenylsulfonyl)butanamide hydrochloride (500 mg) as a white solid.

### Example 41

### 2-amino-N-(4-(pentyloxy)phenylsulfonyl)acetamide hydrochloride

DCC (5 g, 28.5 mmol) and HOSu (3.83 g, 30 mmol) was added into a solution of 2-(tert-butoxycarbonylamino)acetic acid (5 g, 28.5 mmol) in DMF (65 ml) under nitrogen at room temperature. The reaction mixture was stirred for 12 h. DMF was removed under reduced pressure by oil pump. The residue was treated with dichloromethane. The insoluble solid was filtered away by filtration under reduced pressure. The filtrate was concentrated by rotary evaporator to provide 6.8 g of 2,5-dioxopyrrolidin-1-yl 2-(tert-butoxycarbonylamino)acetate in 87% yield as a white powder. A mixture of 2,5-dioxopyrrolidin-1-yl 2-(tert-butoxycarbonylamino)acetate (2.58 g, 9.6 mmol), 4-(pentyloxy)benzenesulfonamide (2.4 g, 9.6 mmol) and solid K₂CO₃ (2.65 g, 19.2 mmol) in DMF (50 ml) was stirred at room temperature for 2 hr. After completion of the reaction, the reaction mixture was poured into an ice-water. Extracted with EtOAc (2×50 ml), washed with brine (2×50 ml), dried on Na₂SO₄ and condensed under reduced pressure to give 8 g of tert-butyl 2-oxo-2-(4-(pentyloxy)phenylsulfonamido)ethylcarbamate in 87% yield as a white powder. tent-Butyl 2-oxo-2-(4-(pentyloxy)phenylsulfonamido)ethylcarbamate (4 g, 10 mmol) was treated with 4 N of HCl in methanol and stirred at room temperature for 30 min. On completion of the reaction, the solid 2-amino-N-(4-(pentyloxy)phenylsulfonyl)acetamide hydrochloride (2 g, 90%) was collected by filtration under reduced pressure.

### Example 42

### 2-(hexyloxy)acetic acid

Sodium hydride (16.8 g, 3.5 eq.) was added under nitrogen into a mixture of 1-hexanol (22.5 g, 1.1 eq) and THF (100 mL) at 0° C. with rapidly stirring. On completion of the addition, the reaction mixture was allowed to warm to room temperature and stirred for an additional 4 h. A solution of the 2-bromoacetic acid (1 eq.) in THF was added dropwise to the reaction mixture at 0° C. The reaction mixture was allowed to warm to room temperature and stirred at reflux overnight. The volatile solvents were removed away by rotary evaporator under reduced pressure. The residue was washed with hexanes and then was dissolved into H₂O and adjusted to pH=4 by adding aq. HCl (2 N). Extraction, drying and condensation gave 2-(hexyloxy)acetic acid (28 g) as an colorless oil in 88% yield. ¹H NMR (400 MHz, CDCl₃): 0.7 (3H) 1.1-1.3 (6H), 1.6 (2H), 3.4 (2H), 4 (2H), 10.5 (1H); ¹³C NMR (400 MHz, CDCl₃): 13, 22, 25, 29, 31, 67, 71, 175.

### Example 43

### 2,5-dioxopyrrolidin-1-yl 2-(trimethylsilyl)ethyl carbonate

To a solution of triphosgene (25 g, 0.5 eq.) in THF (200 mL) was added dropwise a mixture of 2-(trimethylsilyl)ethanol (20 g, 1 eq.) and TEA (25 mL, 1.1 eq.) in THF at 0° C. After being stirred at rt for 2 hr, a solution of HOSu (1 eq.) in THF (50 mL) was added into the reaction mixture at 0° C. The resulting mixture was stirred overnight. The resulted solid was removed away by filtration under reduced pressure. The filtrate was treated by chromatographic column on silica gel eluted by CH₂Cl₂. The collected fractions was condensed to 10 mL of volume and poured into pure hexanes. The precipitated solid 2,5-dioxopyrrolidin-1-yl 2-(trimethylsilyl)ethyl carbonate was collected in 78% yield by filtration under reduced pressure. ¹H NMR (400 M) 4.4 (d, 2H), 2.9 (s, 4H), 2.2 (d, 2H), 0.1 (s, 1H); ¹³C NMR 172, 154, 74, 28, 19, 0.

### Example 44

### 4,5-dimethoxy-2-nitrobenzyl carbonochloridate

To a stirred solution of bis(trichloromethyl)carbonate (BTC, triphosgene) (14 g, 47.2 mol) and (4,5-dimethoxy-2-nitrophenyl)methanol (10 g, 47 mmol) in 200 ml THF was added dropwise a solution of TEA (6.5 ml) in THF (150 ml) at 0° C. On completion of the addition, the mixture was stirred at room temperature overnight. The resulting solid was filtered away. The filtrate was concentrated to give crude 4,5-dimethoxy-2-nitrobenzyl carbonochloridate, which was purified by re-crystallization in benzene to give 4,5-dimethoxy-2-nitrobenzyl carbonochloridate (10.8 g) as a yellowish solid. ¹H NMR (400 MHz, CDCl₃): δ4 (s, 6H), 5.7 (s, 2H) 6.9 (s, 1H), 7.7 (s, 1H).

### Example 45

### N-(3-aminopropyl)-4-(pentyloxy)benzenesulfonamide

Using a procedure similar to the preparation of N-(6-aminohexyl)-4-(pentyloxy)benzenesulfonamide as in Example 39 and replacing hexane-1,6-diamine with propane-1,3-diamine, the title compound, N-(3-aminopropyl)-4-(pentyloxy)benzenesulfonamide is made.

### Example 46

### N-(4-aminobutyl)-4-(pentyloxy)benzenesulfonamide

Using a procedure similar to the preparation of N-(6-aminohexyl)-4-(pentyloxy)benzenesulfonamide as in Example 39 and replacing hexane-1,6-diamine with butane-1,4-diamine, the title compound, N-(4-aminobutyl)-4-(pentyloxy)benzenesulfonamide is made.

### Example 47

### N-(5-aminopentyl)-4-(pentyloxy)benzenesulfonamide

Using a procedure similar to the preparation of N-(6-aminohexyl)-4-(pentyloxy)benzenesulfonamide as in Example 39 and replacing hexane-1,6-diamine with pentane-1,4-diamine, the title compound, N-(5-aminopentyl)-4-(pentyloxy)benzenesulfonamide is made.

### Example 48

### Synthesis of Compound (23)

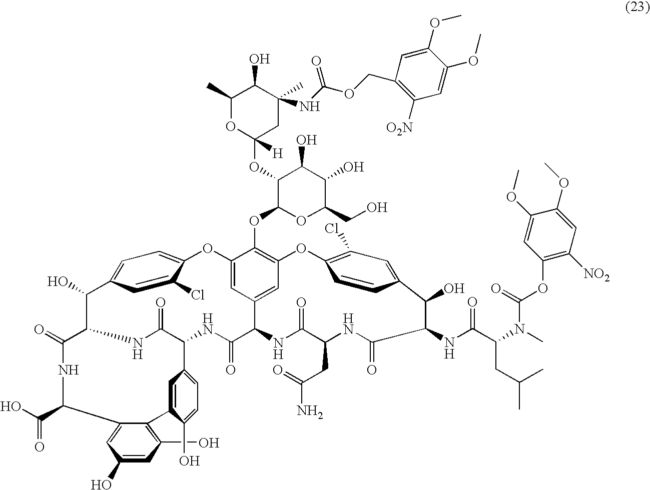

To a solution of vancomycin hydrochloride (5 g) in THF (30 ml) and H₂O (20 ml) was added NaHCO₃ (1.5 g), followed by 4,5-dimethoxy-2-nitrobenzyl carbonochloridate (14 g) at room temperature. The resulted mixture was stirred at room temperature for 2 h. The solvents were removed under vacuum. The resulting solid was separated by column chromatography on silica gel (300 mesh, eluent: CH₂Cl₂/MeOH=2/1). Compound (23), (2.6 g) was obtained in 50% yield.

### Example 49

### Synthesis of Compound (24)

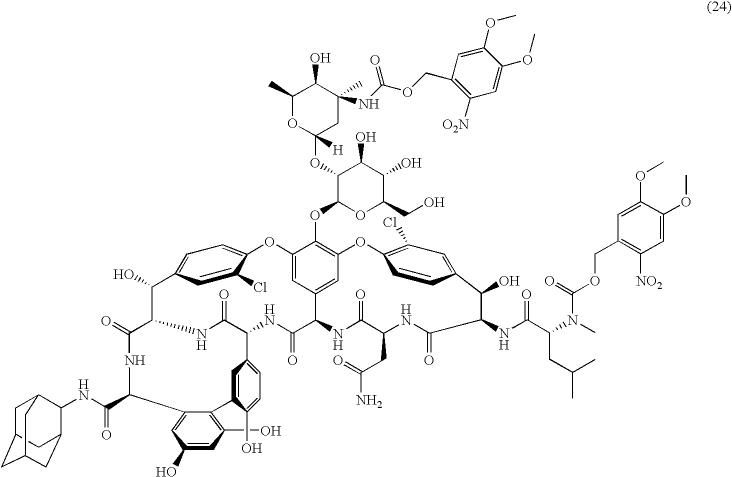

Using a procedure similar to the preparation of Compound (23), as in Example 48 and replacing vancomycin hydrochloride with Compound (21), Compound (24), is made.

### Example 50

### Synthesis of Compound (25)

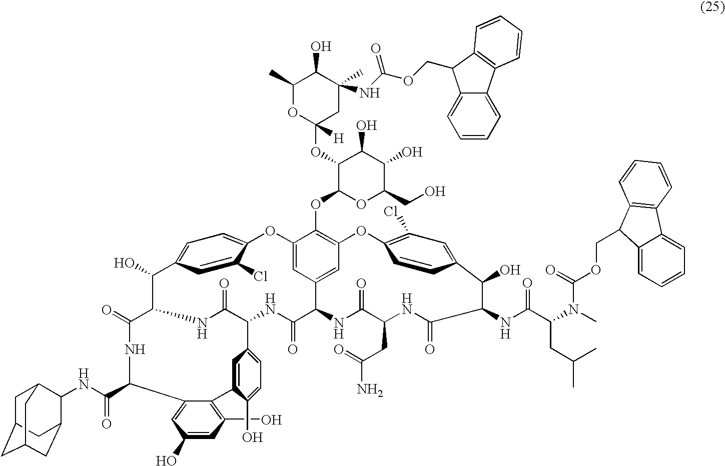

To a solution of Compound (21) (35.0 g) in 1,4-dioxane (50 mL) and water (50 mL) was added Fmoc-OSu (9-fluorenylmethyloxycarbonyl-O-succinimide) (11.0 g) with stirring at room temperature. After the reaction mixture was stirred at ambient temperature for 2 hr, the solvent was removed under reduced pressure. The resulting solid was collected by filtration under vacuum and was purified by silica gel column chromatography (silica gel, MeOH—CH2Cl2: 10%-20%) to give Compound (25), (20 g) as white solid.

### Example 51

### Synthesis of Compound (26)

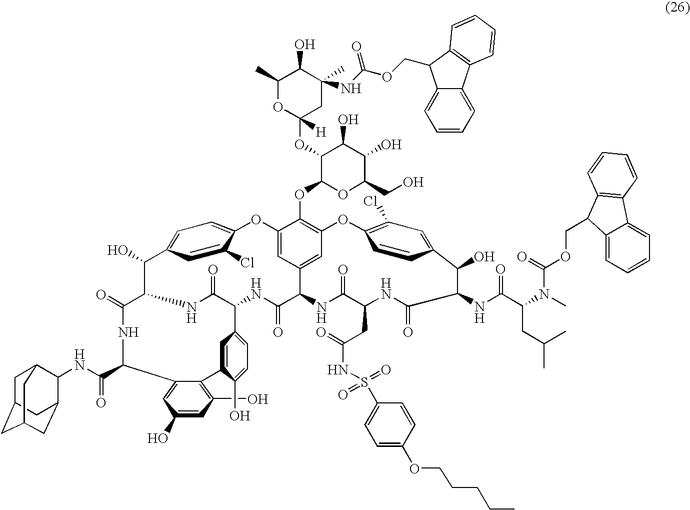

Under argon atmosphere, to a solution of Compound (25), (1 g) in dry DMF (10 mL) was added freshly distilled triethylamine (55 mg) and a solution of p-pentyloxybenzenesulfonyl chloride (170 mg) in DMF (1 mL) at ambient temperature with stirring. The reaction mixture was stirred for an additional two hours and then was monitored by analytical HPLC. After completion, the mixture was poured into ether (100 mL). The precipitated white solid was collected by filtration under reduced pressure giving Compound (26).

### Example 52

### Synthesis of Compound (27)

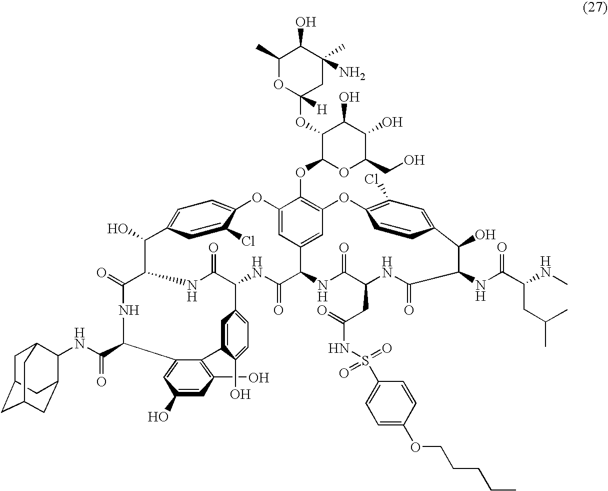

Compound (26) obtained from Example 51 was dissolved into DMF (9 mL) and then diethylamine (3 eq.) was added at ambient temperature. After being stirred at room temperature for 2 hr, the reaction mixture was poured into ether. The formed solid was applied on preparative HPLC to give Compound (27) (50 mg) as white powder. ESIMS m/z at 1809.3 (90%), 1206.6 (100%), 905.3 (40%). ESI-MS m/z Calcd for C₈₇H₁₀₄Cl₂N₁₀O₂₆S [M+H]⁺ 1809.8. Found: 1809.3

### Example 53

### Synthesis of Compound (28), (29), (30), (31), (32), (33), (34), (35), (36), (37), (38), (39), & (40)

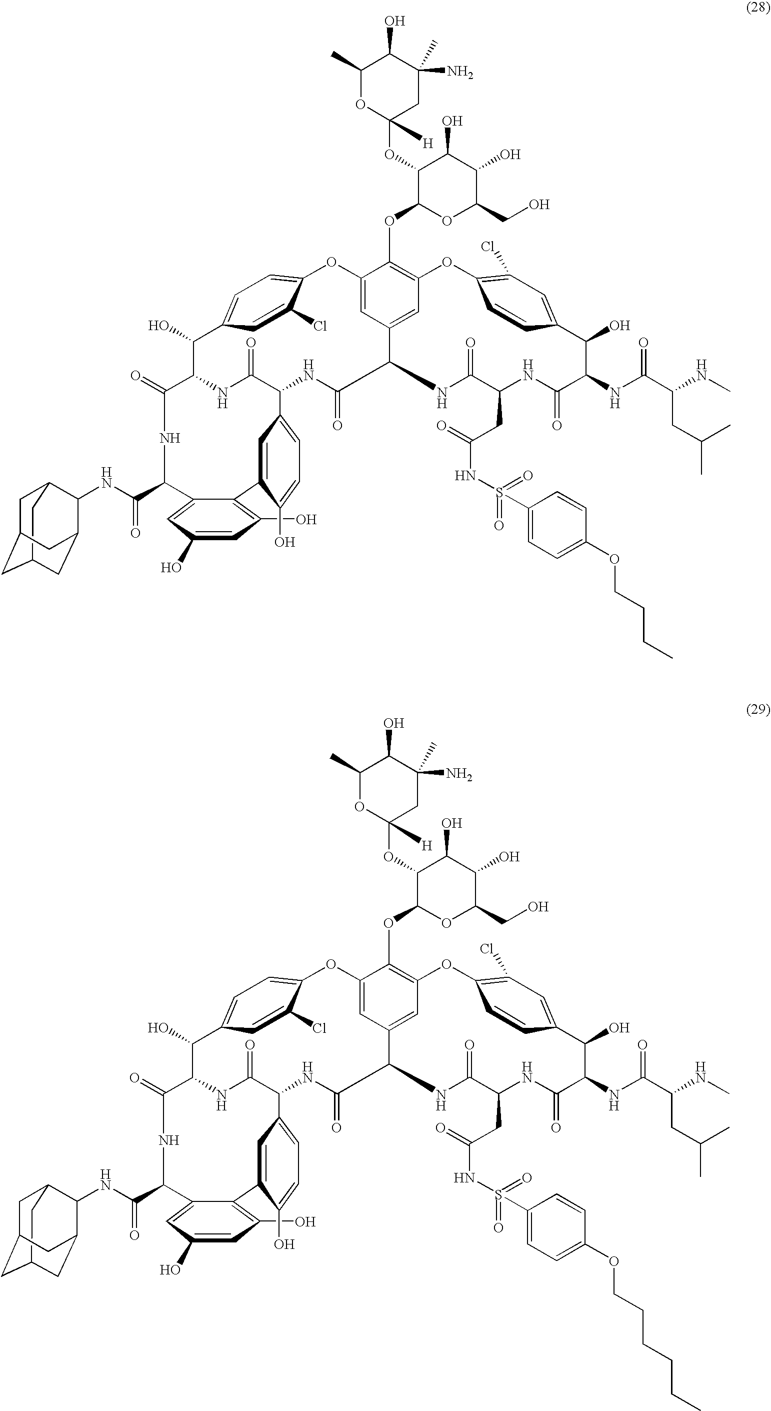

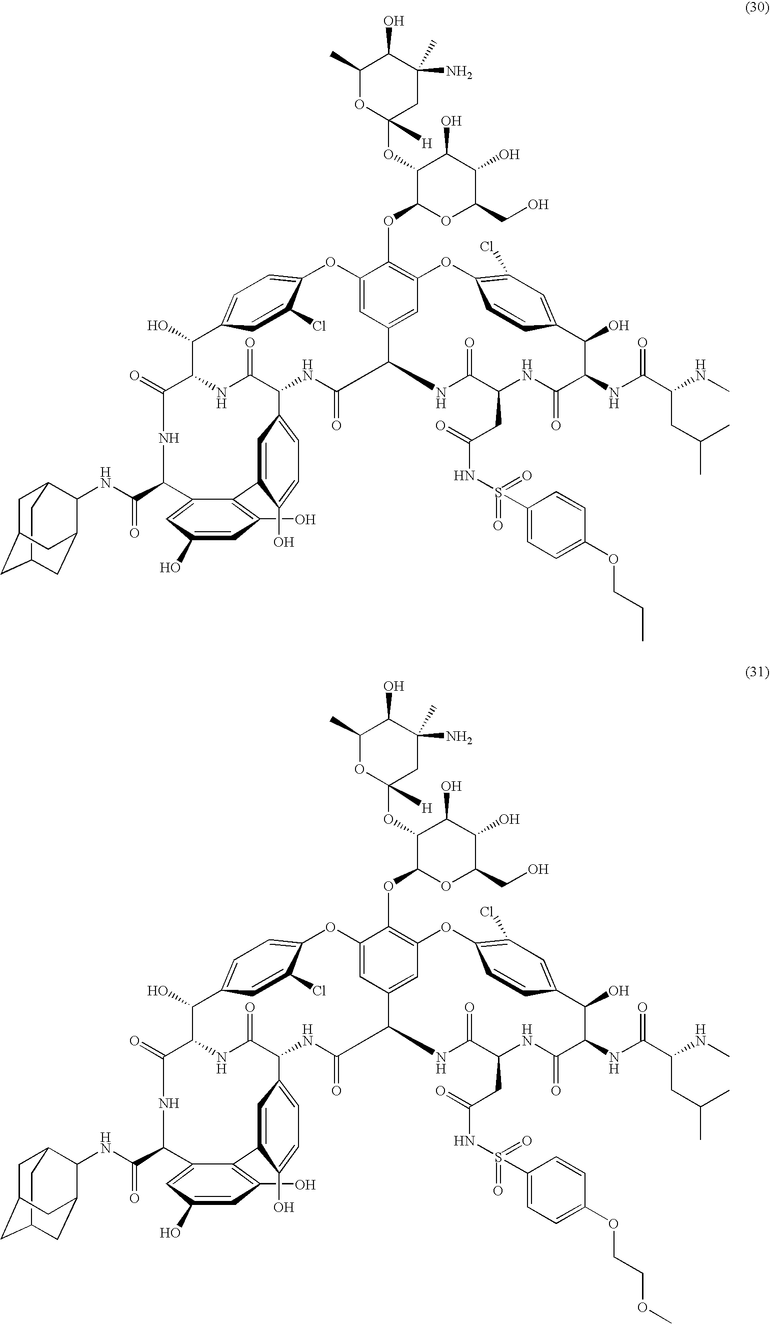

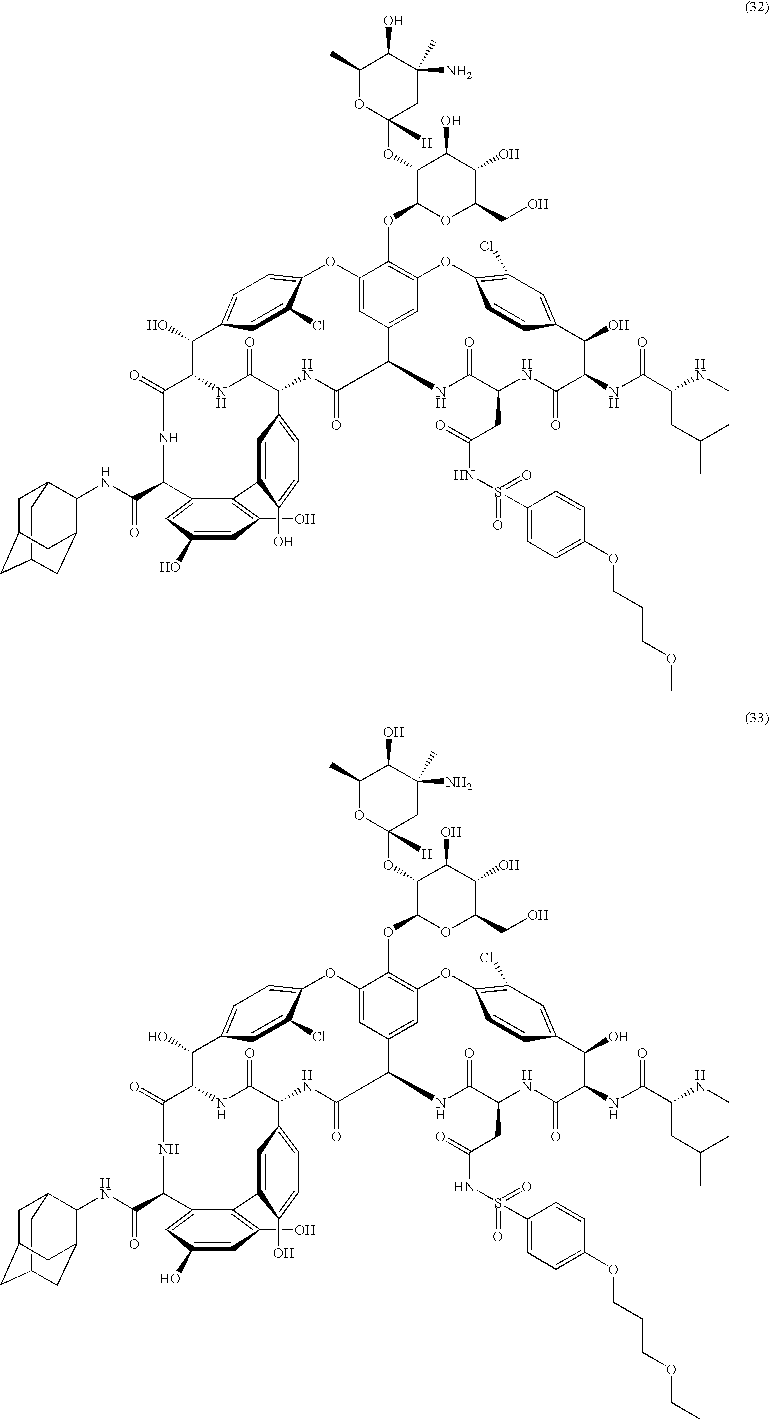

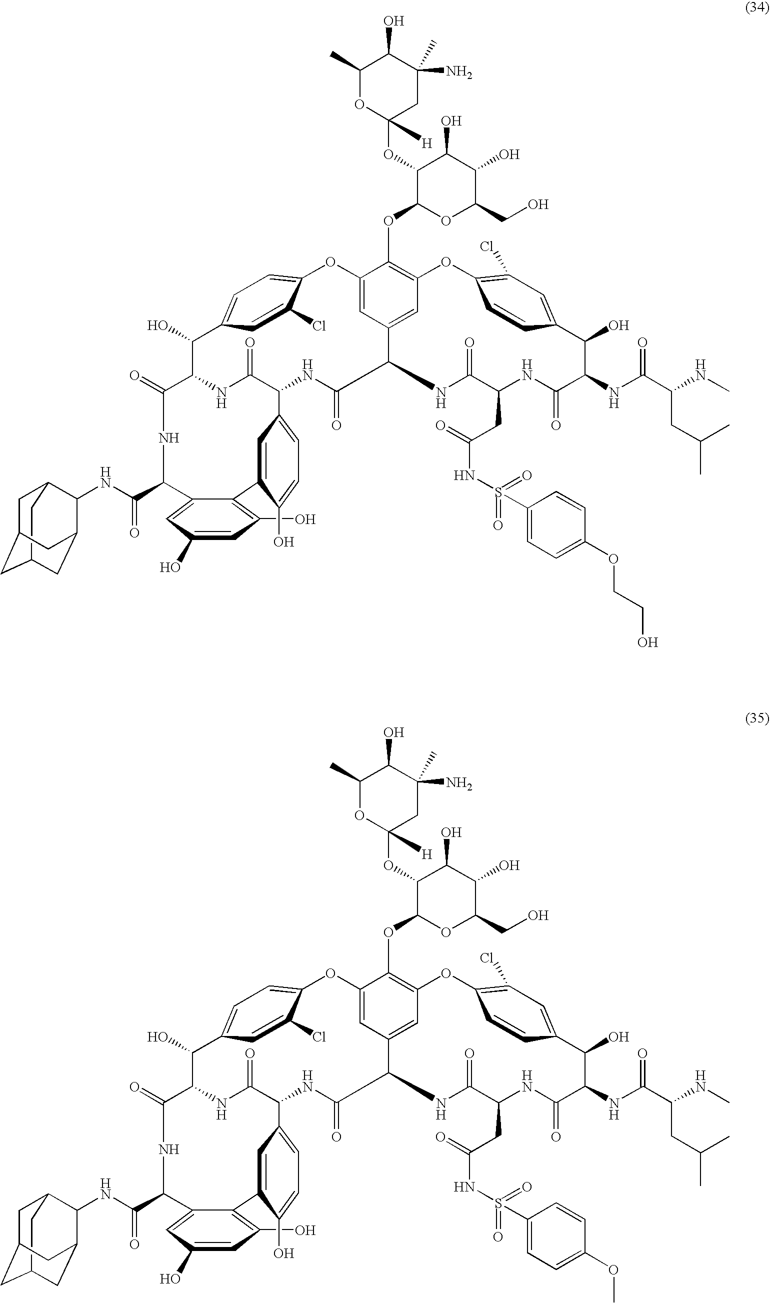

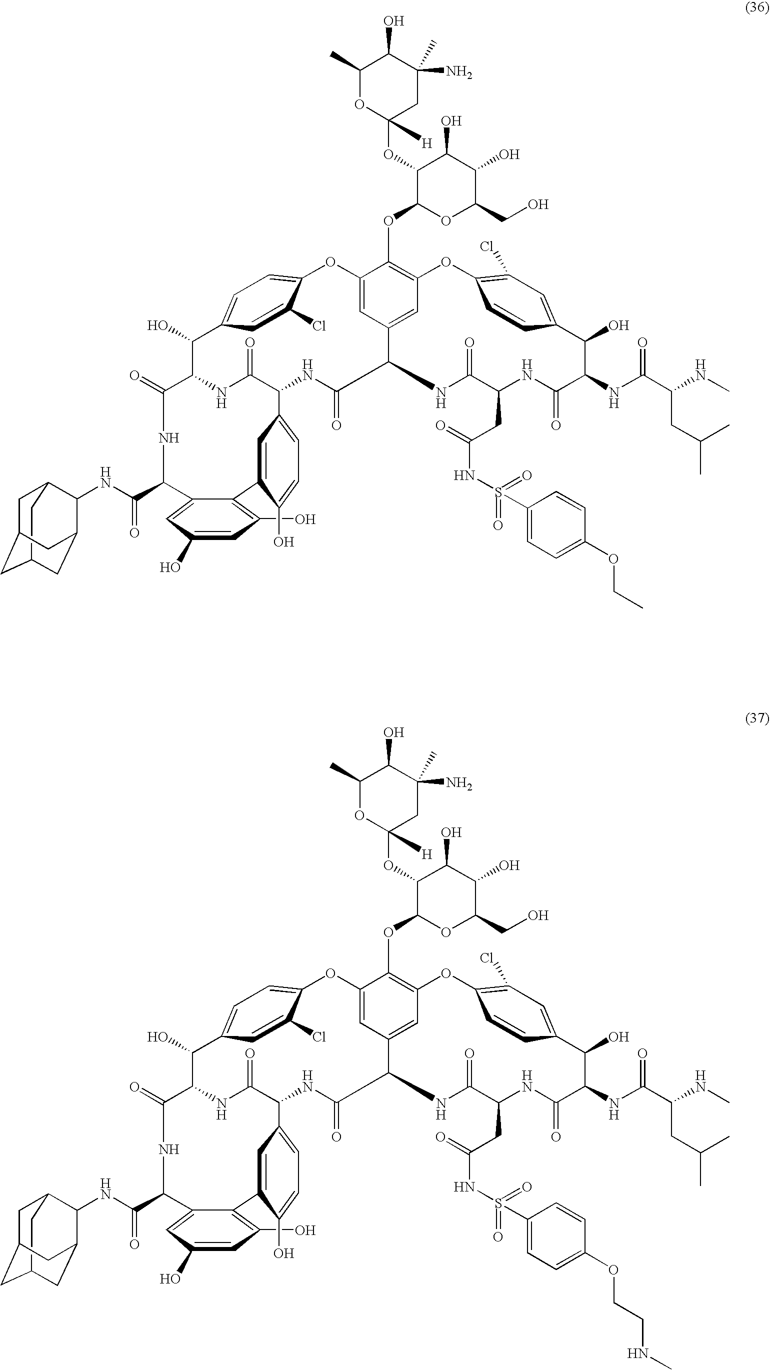

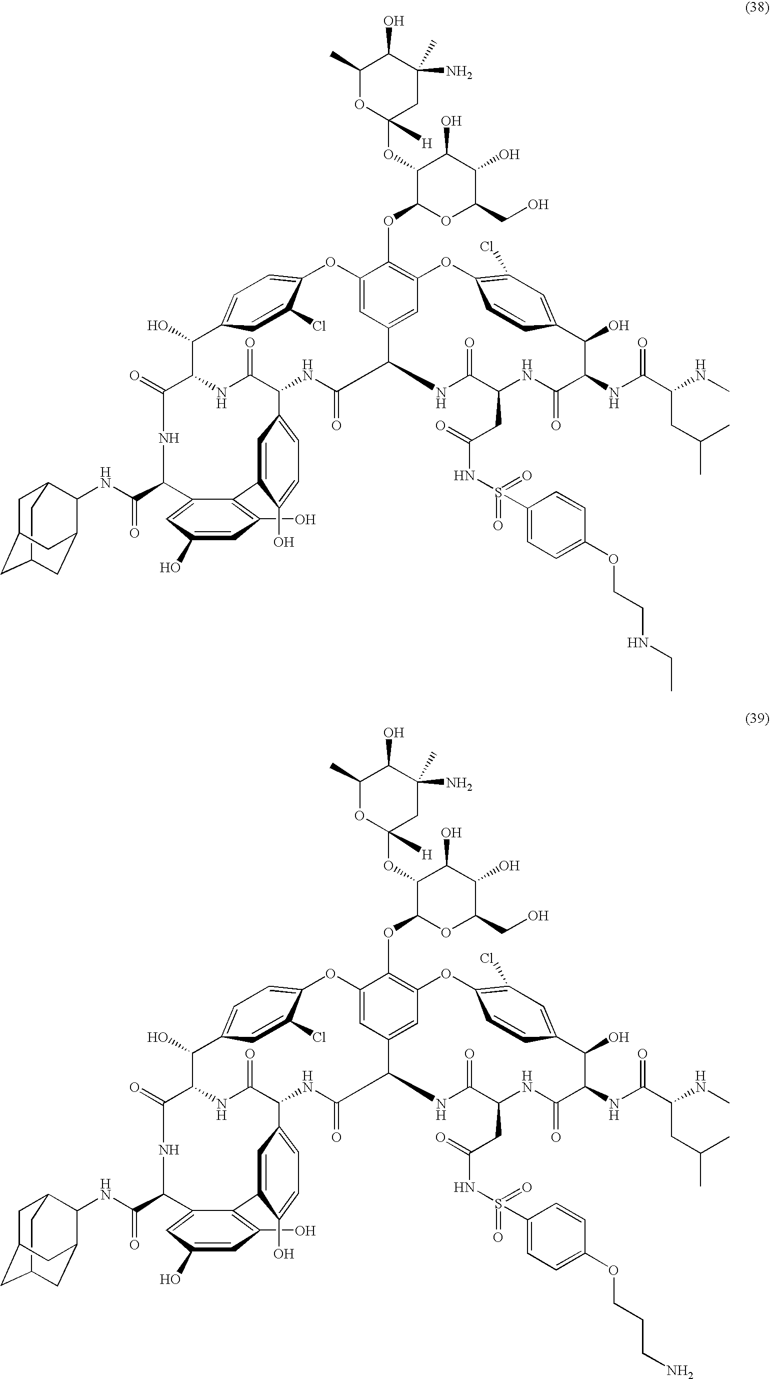

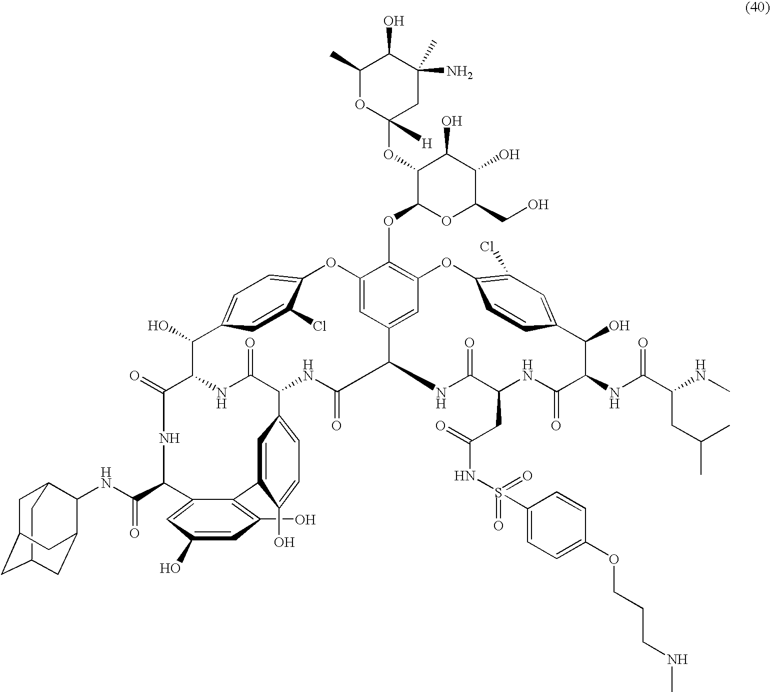

Following the experimental procedure as described in Example 51, for the preparation of Compound (26), replacing p-pentyloxybenzenesulfonyl chloride with various benzensulfonyl chloride or properly hydroxyl or amino protected benzensulfonyl chloride, various Compound (26) analogs are obtained. These Compound (26) analogs are subjected to the similar procedure as described in Example 52 for the preparation of Compound (27), (followed by deprotection for hydroxyl or amino protected compounds), Compounds (28-33) and Compound (36) were prepared and Compound (34), Compound (35), Compound (37), Compound (38), Compound (39) and Compound (40), are obtained.

### Example 54

### Synthesis of Compound (41)

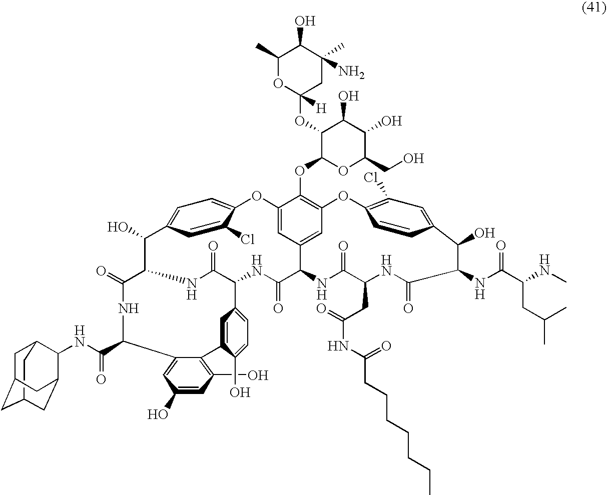

To a solution of octanoic acid (0.5 g) in THF (6 mL) was slowly added CDI (0.7 mL, 1.3 eq.) at 0° C. After the reaction mixture was stirred at room temperature for 2 hr, a solution of Compound (25) (0.55 g) in DMF (7 mL) was added at ambient temperature. The resulting mixture was stirred at rt for an additional 2 hr. The analytical HPLC monitoring showed the reaction completed. The reaction mixture was poured into ether. The formed crude Fmoc-acylamide product (600 mg) was collected by filtration and was dissolved into DMF (8 mL). Diethylamine (0.8 mL) was added at room temperature and the reaction mixture was stirred for 1.5 hr, monitored by analytical HPLC. The mixture was poured into ether. The formed solid was collected by filtration and applied onto preparative HPLC to give Compound (41) (50 mg). ESIMS-MS m/z at 1822.1 (100%), [M+CF3COO]⁻=1821.7, found: 1822.1; +MS m/z at 1709.8 (40%), 1140.0 (100%), 855.4 (60%), [M+H]⁺ 1709.7, Found: 1709.8.

### Example 55

### Synthesis of Compound (42)

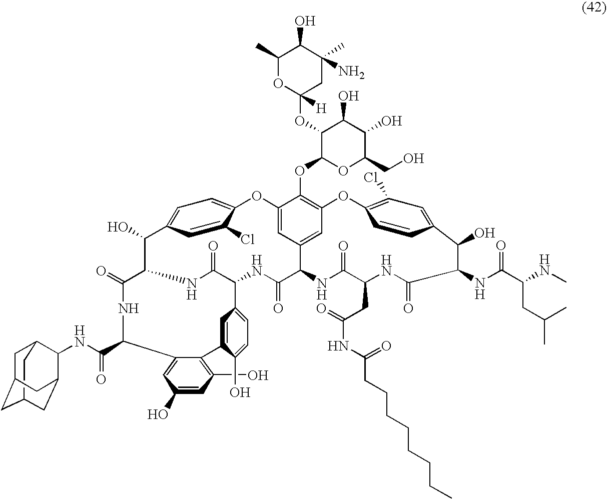

To a solution of n-nonanoic acid (0.6 g) in THF (6 mL) was slowly added CDI (0.76 mL, 1.4 eq.) at 0° C. After the reaction mixture was stirred at room temperature for 2 hr, a solution of Compound (25) (0.6 g) in DMF (7 mL) was added. The resulting mixture was stirred at room temperature for an additional 2 hr. The analytical HPLC monitoring showed the reaction completed. The reaction mixture was poured into ether. The formed crude Fmoc-acylamide product (620 mg) was collected by filtration and was dissolved into DMF (8 mL) and then was treated with 3 eq. of diethyl amine at room temperature for 1.5 h. The mixture was analyzed by analytical HPLC. On the completion of the reaction, the mixture was diluted with ether (30 ml). The forming solid was collected by filtration and then washed with ether, dried under vacuum and isolated by preparative HPLC to afford 40 mg of Compound (42) as a white powder. ESIMS-MS m/z at 1835.8 (100%), [M+CF3COO]⁻=1835.7, found: 1835.8; +MS m/z at 1723.7 (100%), 1149.4 (80%), 862.3 (30%), [M+H]⁺ 1723.7, Found: 1723.7.

### Example 56

### Synthesis of Compound (43)

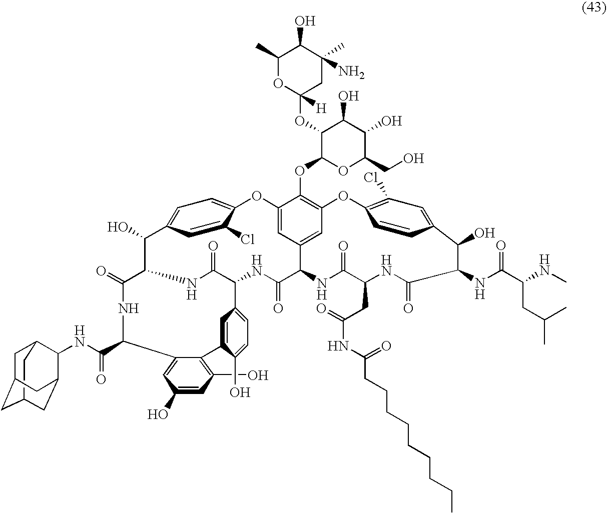

A solution of decanoic acid (0.8 g) in THF (8 ml) was treated with CDI (1.0 ml) at 0° C. The mixture was stirred at room temperature for 2 h. The resulting mixture was added into a solution of Compound (25) (0.62 g) in DMF (7 ml) at room temperature. The mixture was analyzed by analytical HPLC. On the completion of the reaction, the mixture was diluted with ether (40 ml). The resulted solid was collected by filtration to afford 640 mg of crude Fmoc-acylamide product as a white powder. De-protection of the Fmoc group by diethylamine led to Compound (43). 45 mg of Compound (43) was isolated by preparative RP-HPLC as a white powder. ESIMS-MS m/z at 1849.9 (100%), [M+CF3COO]⁻=1849.7, found: 1849.9; +MS m/z at 1737.7 (100%), 1158.6 (50%), 869.4 (20%), [M+H]⁺ 1737.7, Found: 1737.7.

### Example 57

### Synthesis of Compounds 44, 45, 46, 47, 48, 49, 50, 51, 52, 53 and 54

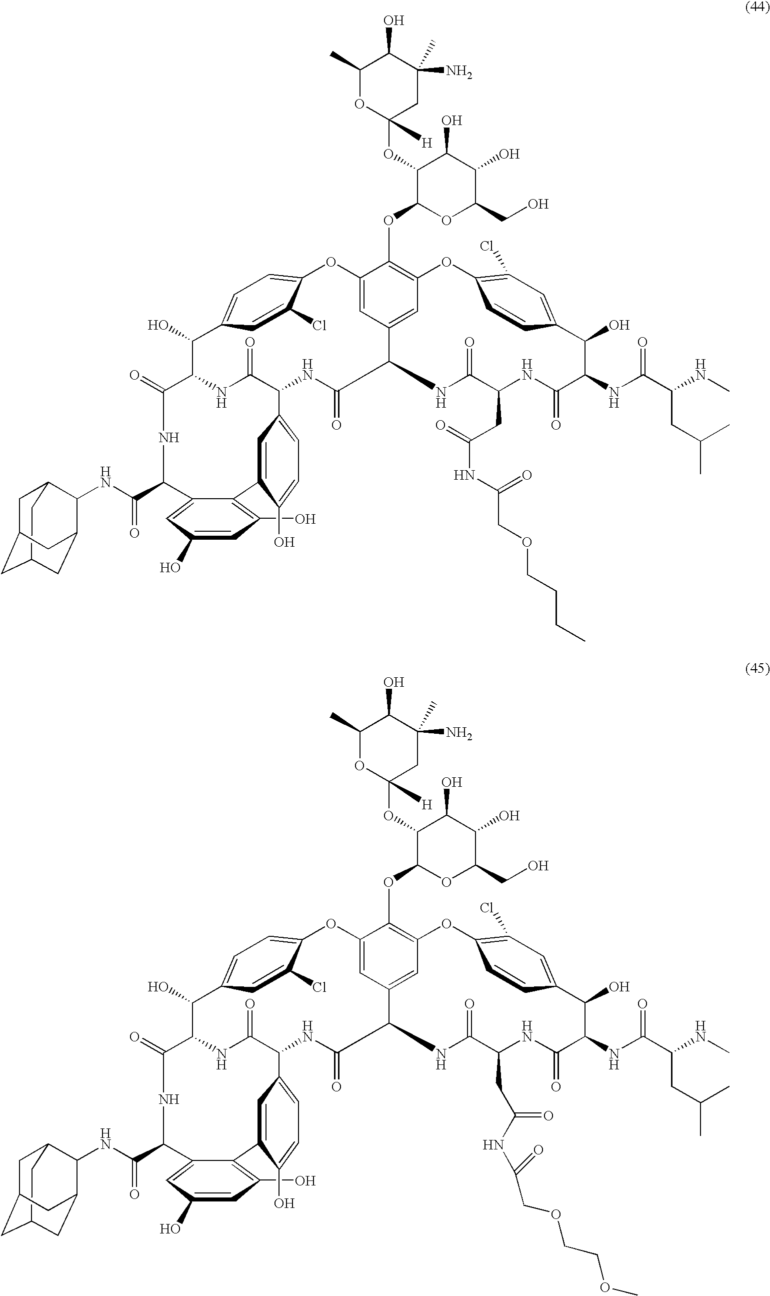

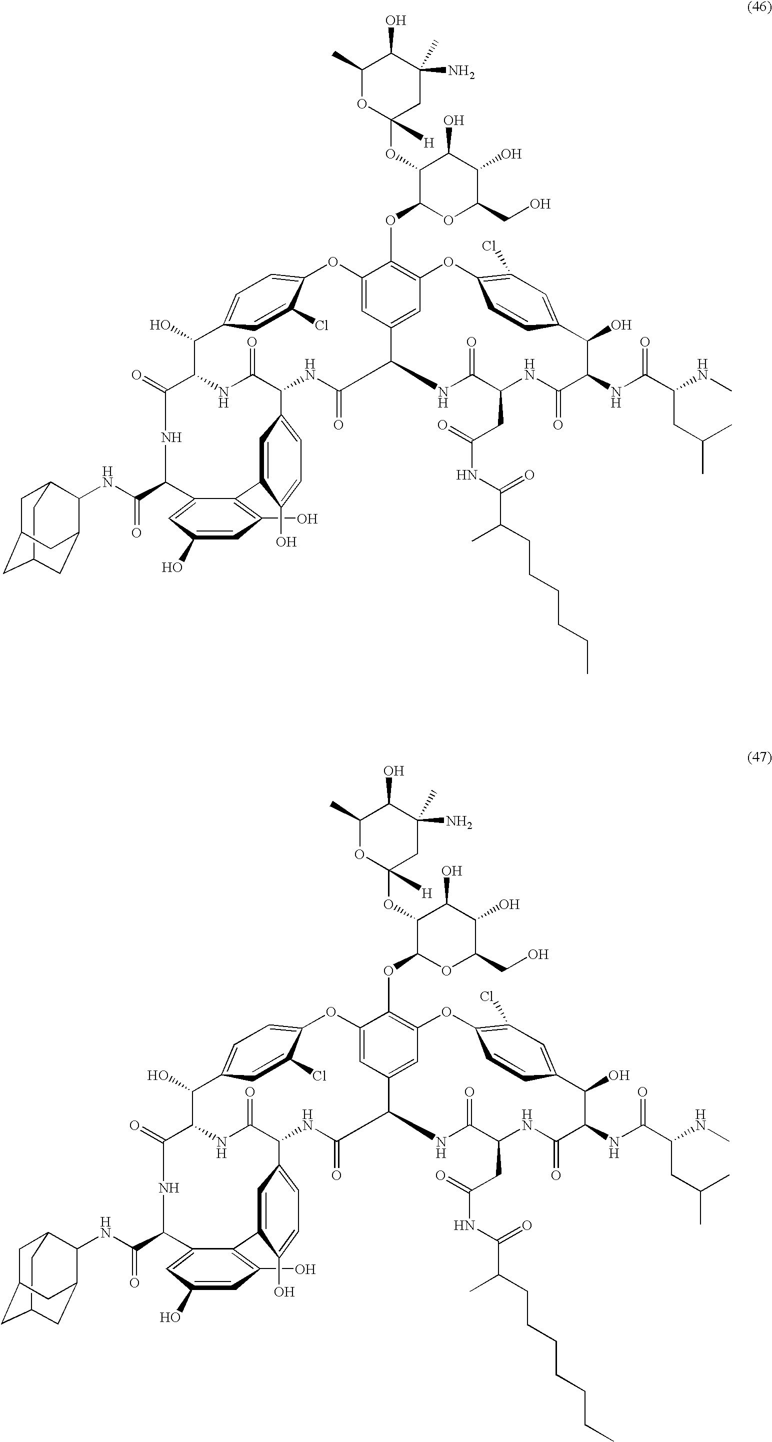

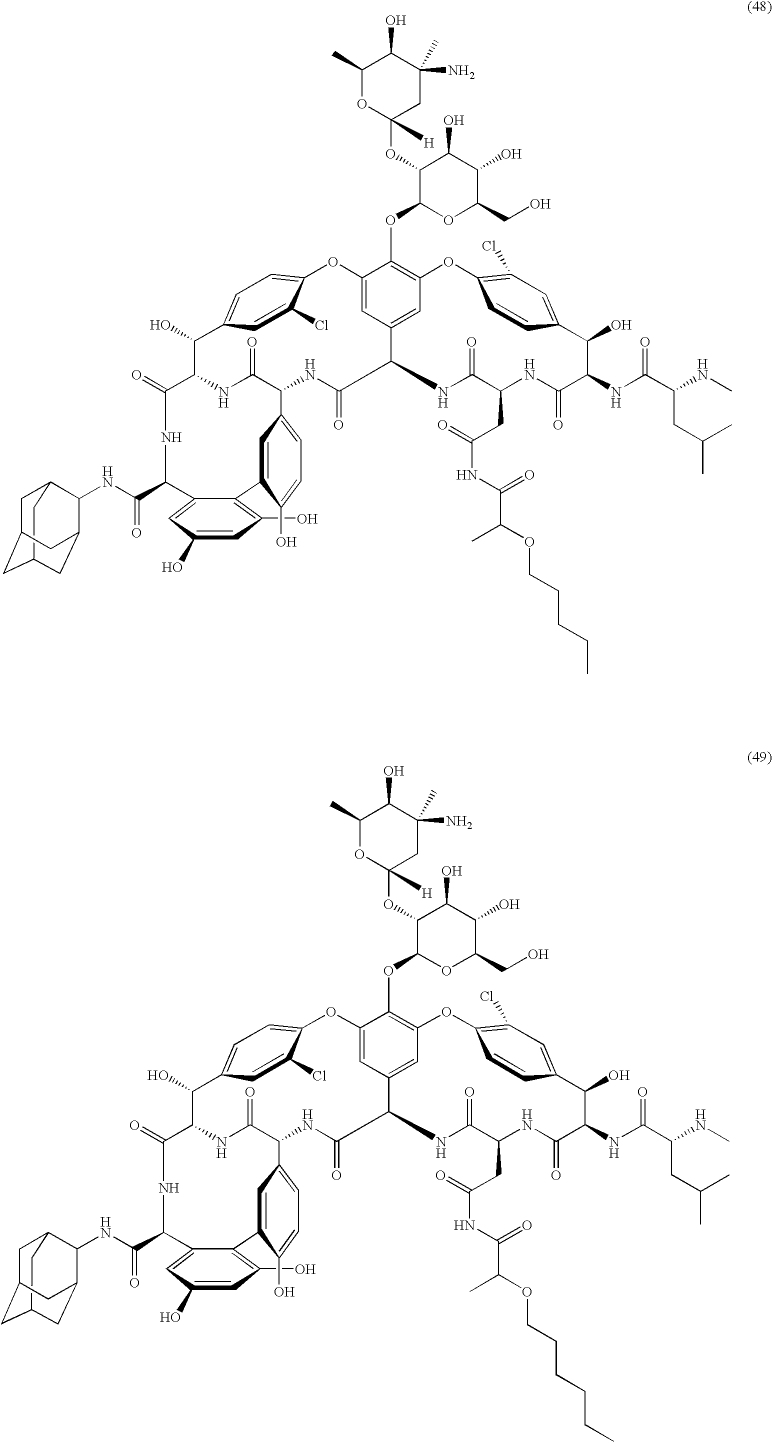

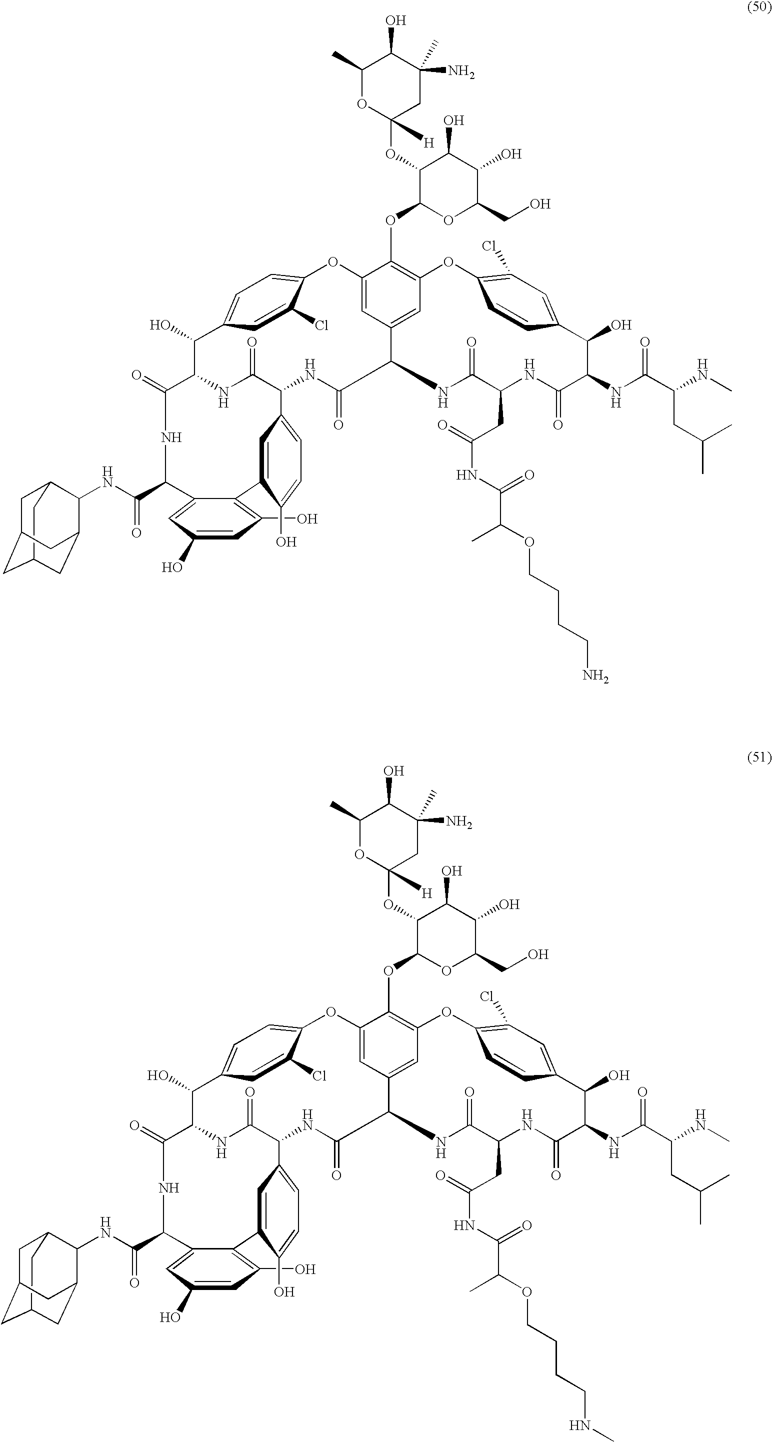

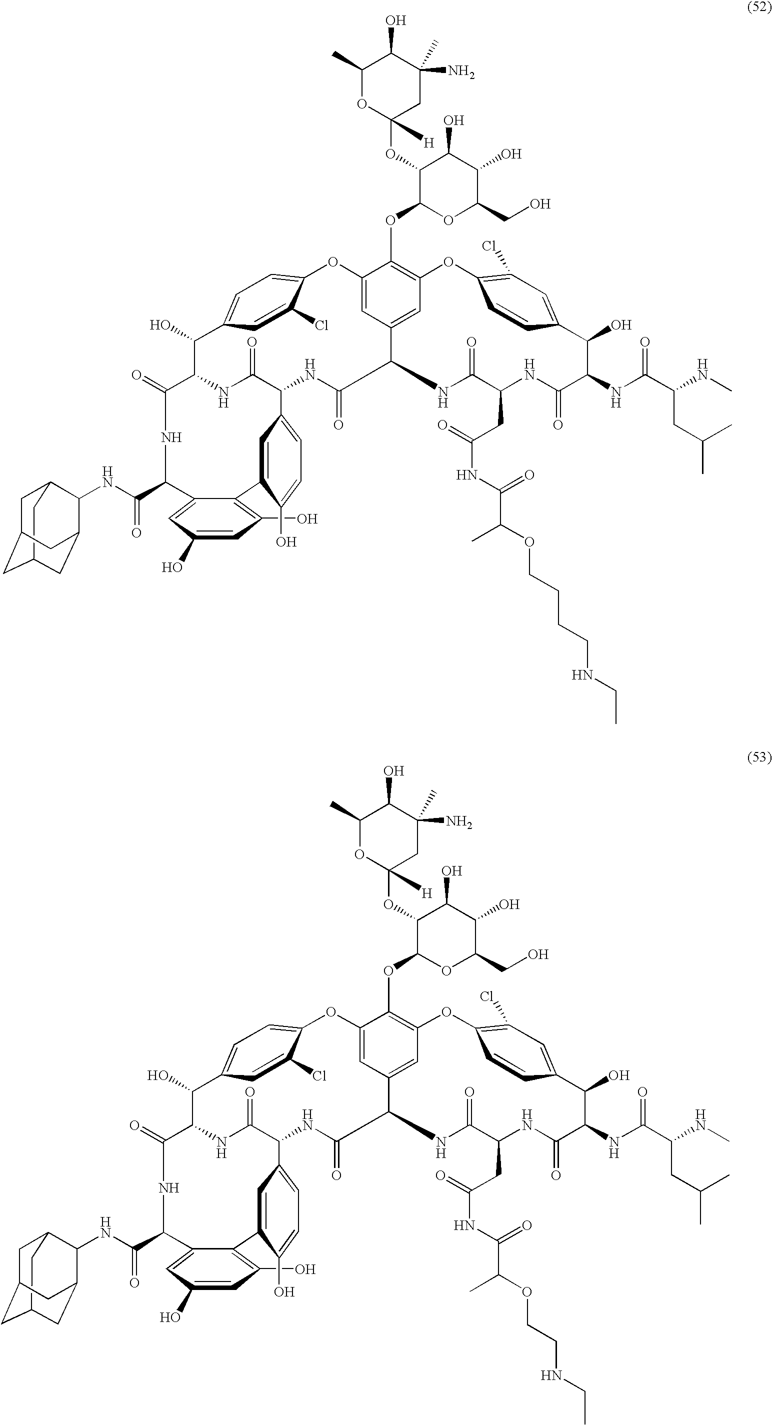

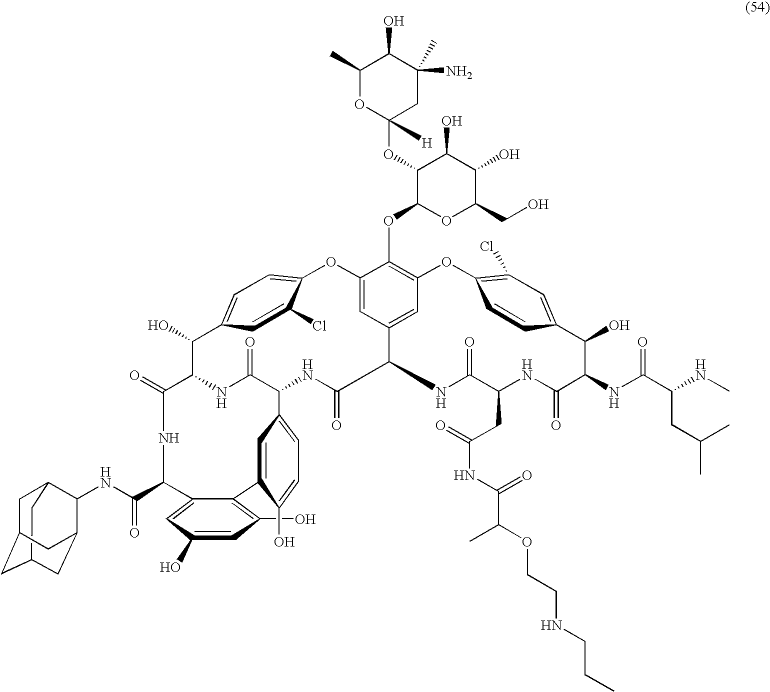

Following the experimental procedure as described in Example 54, for the preparation of Compound (41), replacing octanoic acid with various carboxylic acid or properly hydroxyl or amino protected carboxylic acid, various Fmoc-Compound (41) analogs are obtained. These Fmoc-Compound (41) analogs are subjected to the similar deprotection procedure as described in Example 54 (followed by deprotection for hydroxyl or amino protected compounds), Compound (44) and Compound (46) were prepared and Compound (45), Compound (47), Compound (48), Compound (49), Compound (50), Compound (51), Compound (52), Compound (53) and Compound (54), are made.

### Example 58

### Synthesis of Compounds 55, 56, 57, 58, 59, 60, 61, 62, 63, 64, 65, 66, 67, 68, and 69

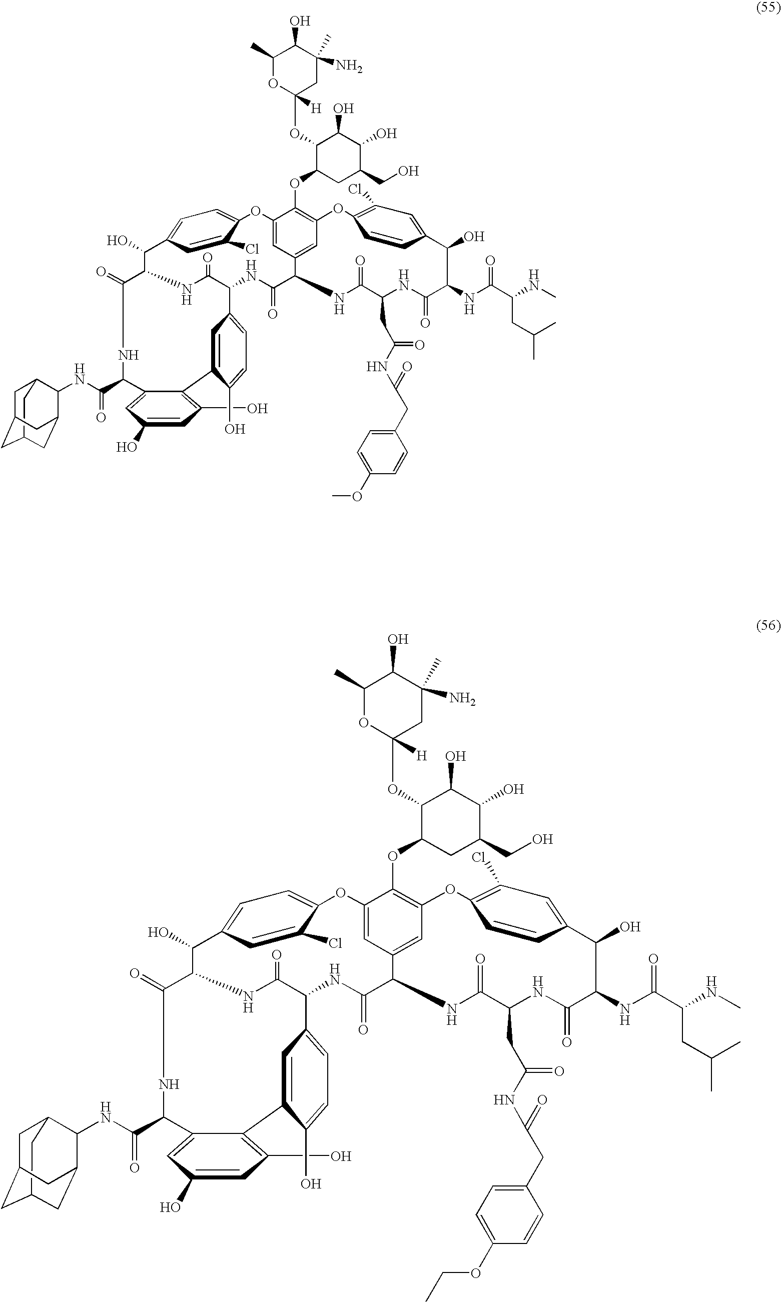

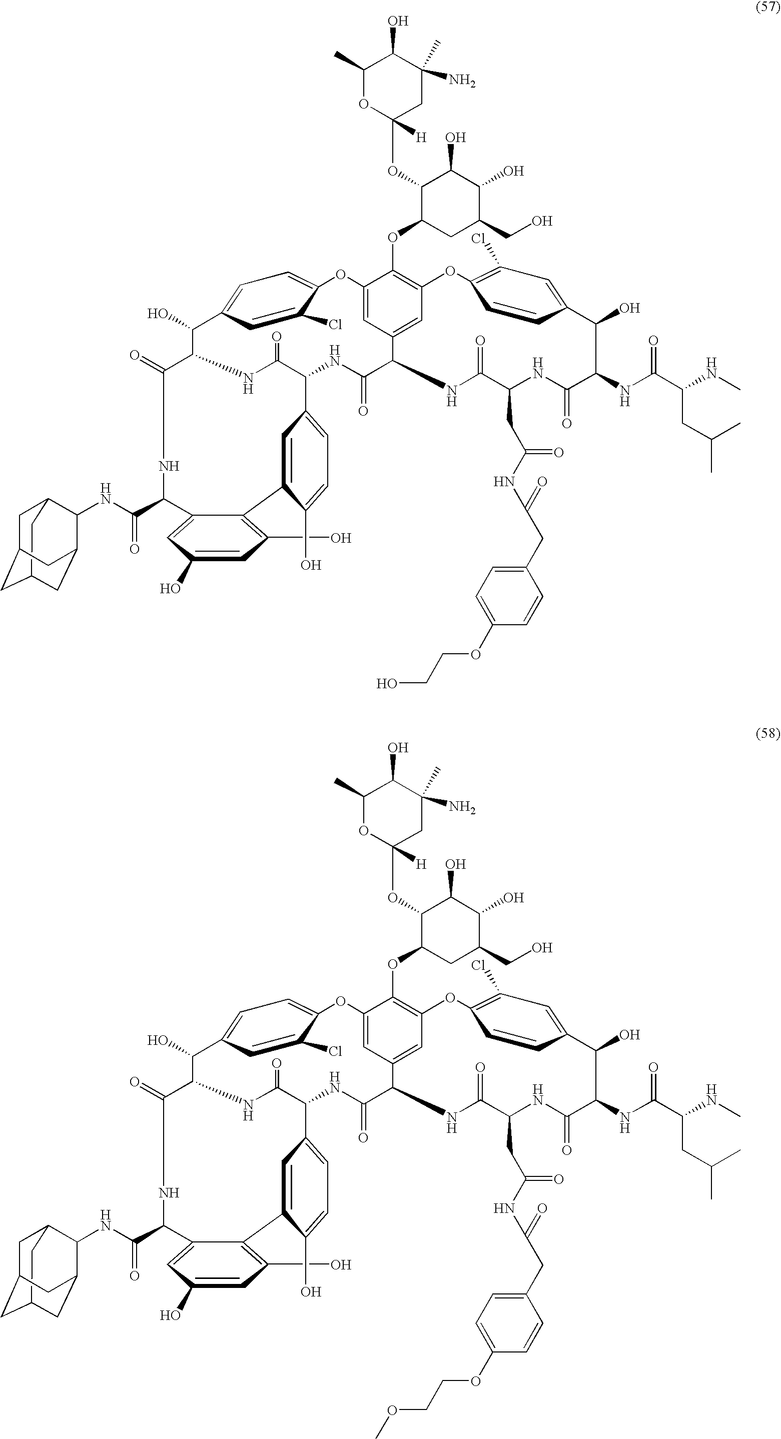

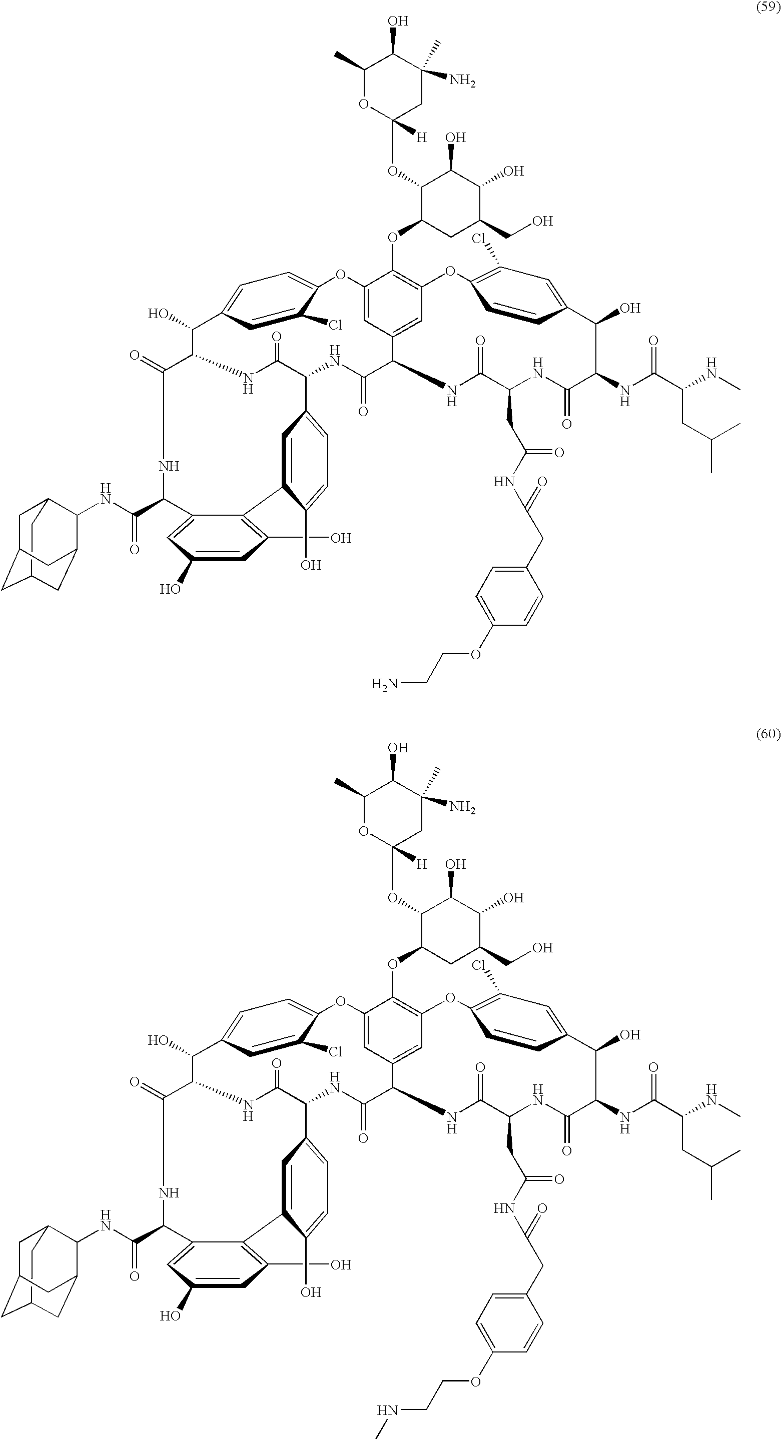

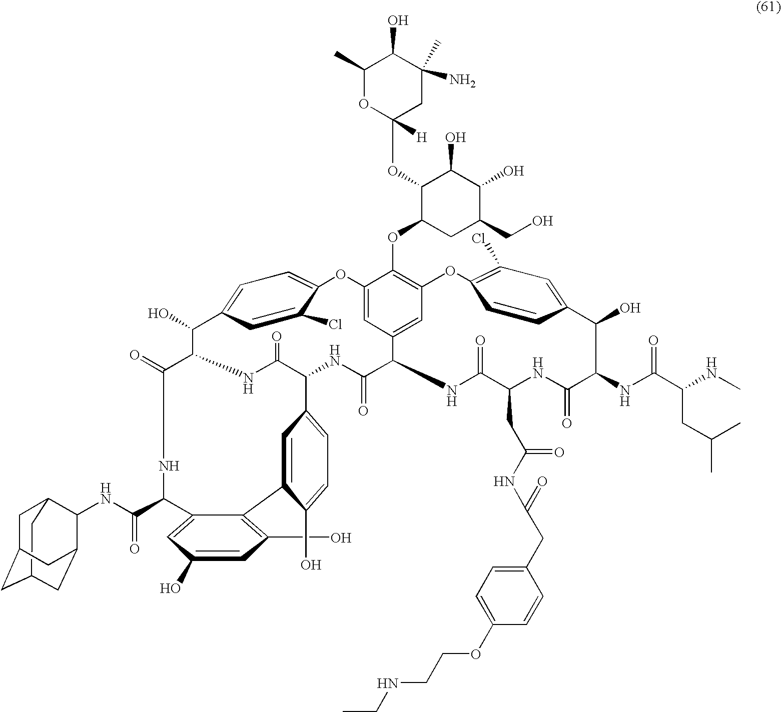

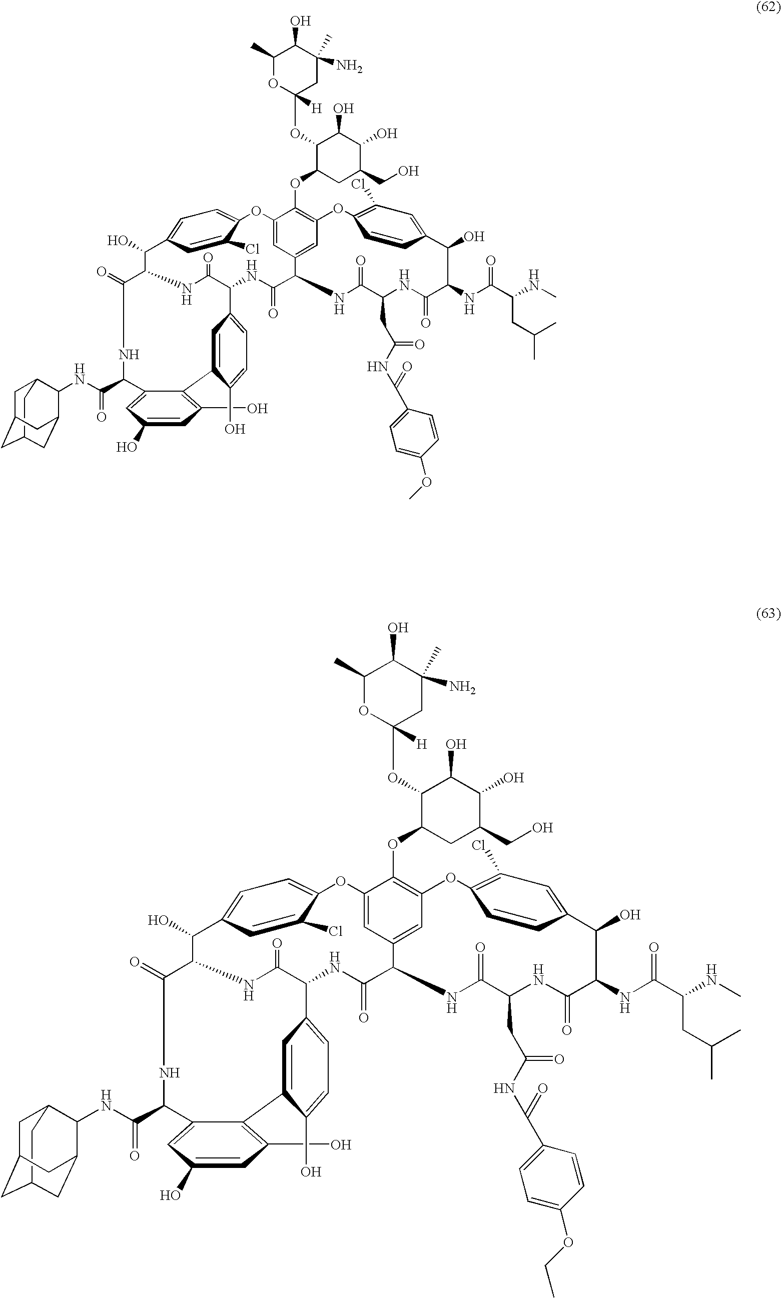

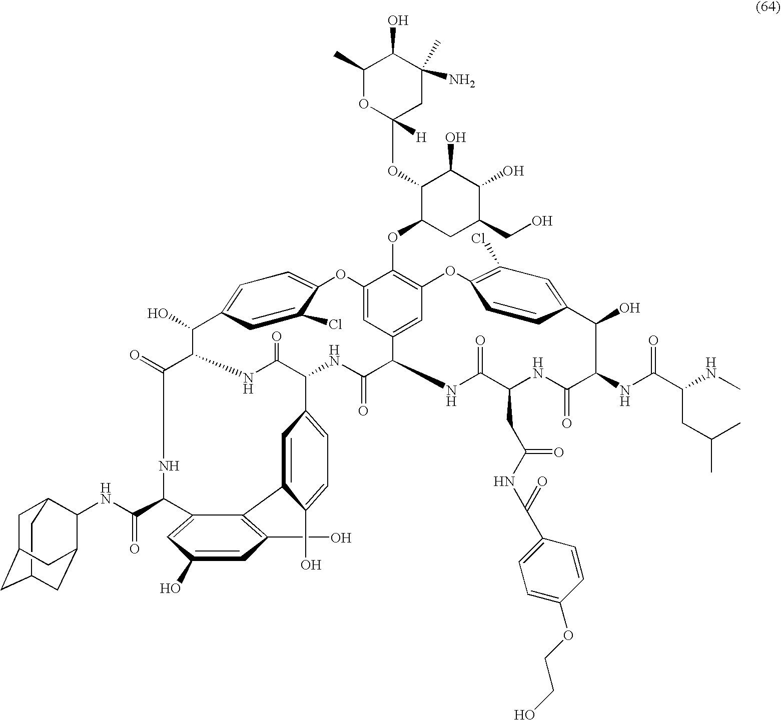

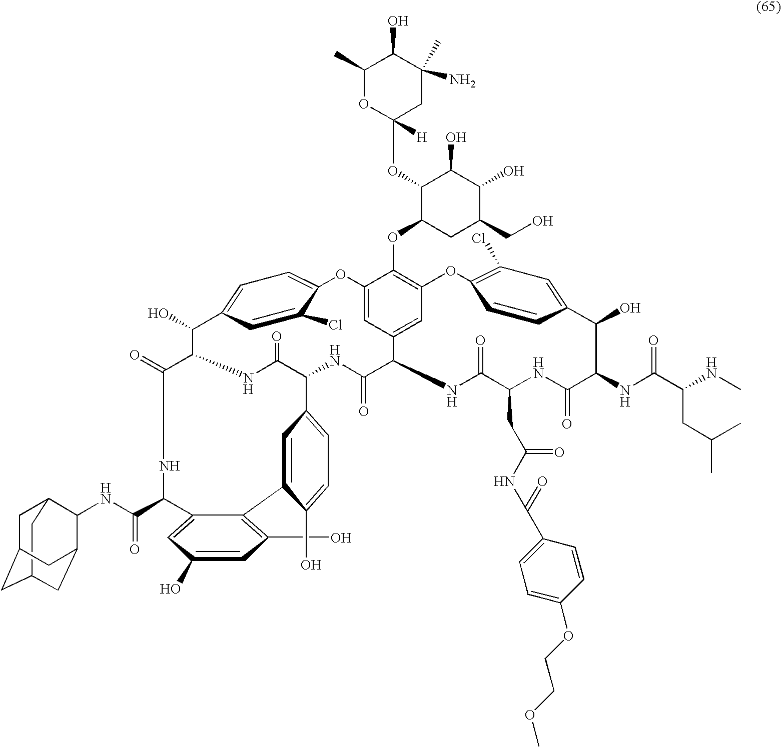

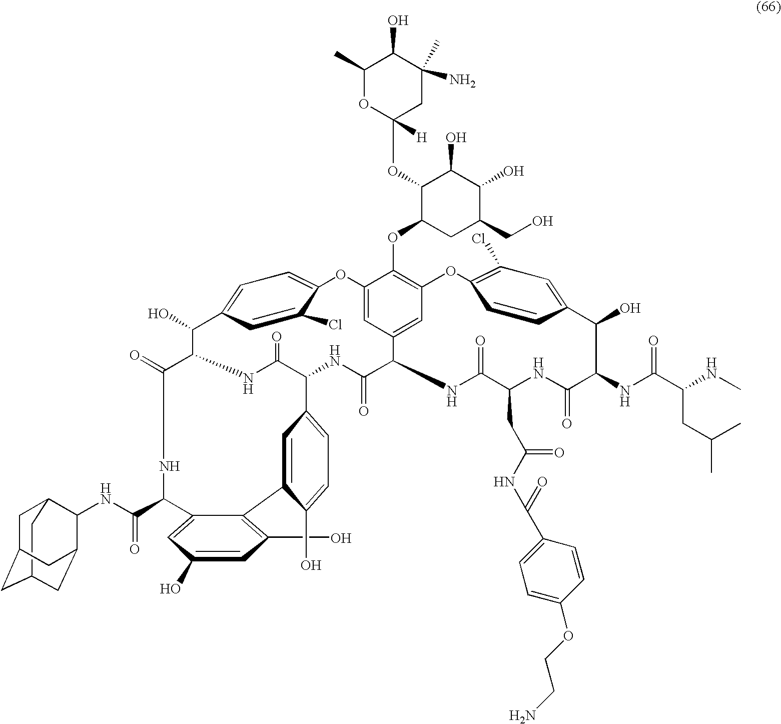

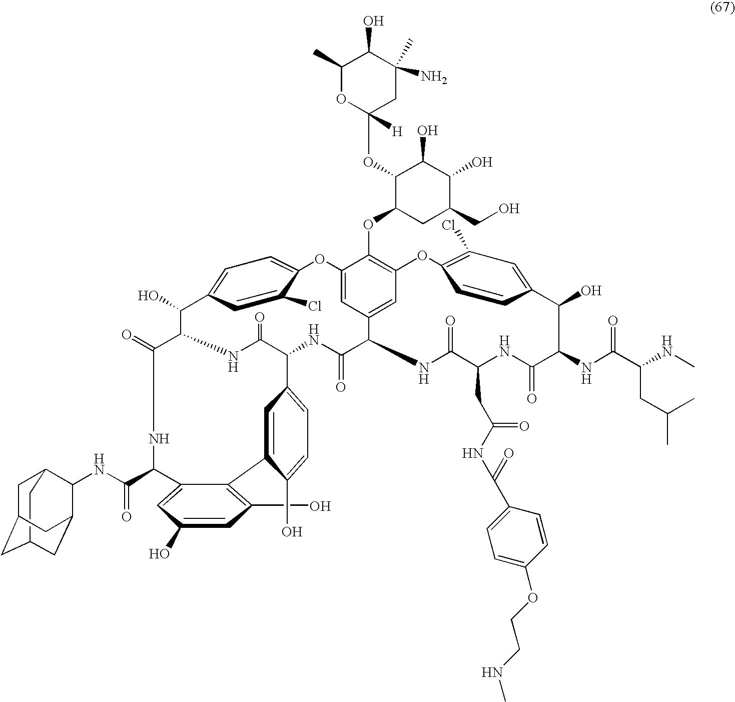

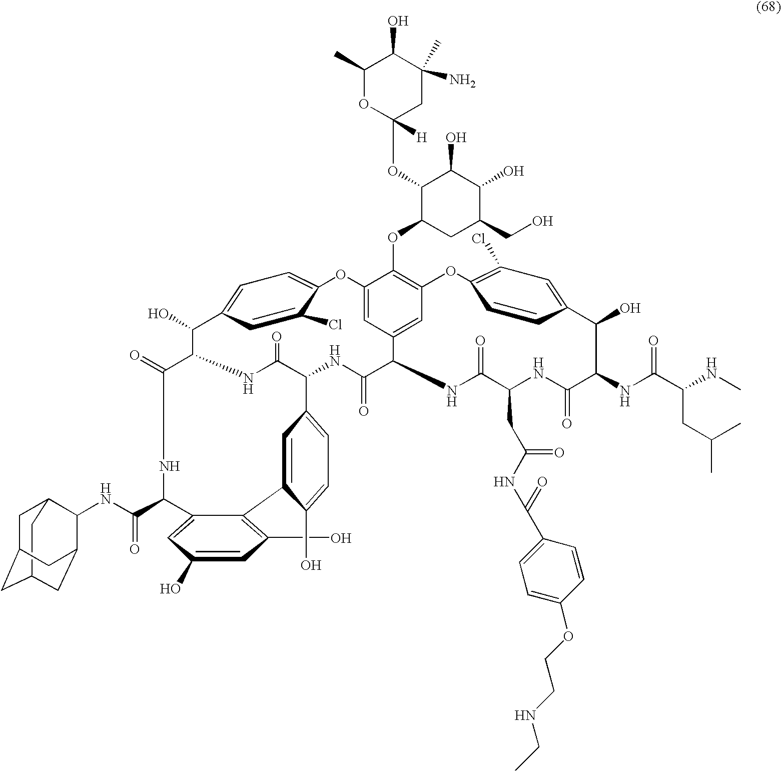

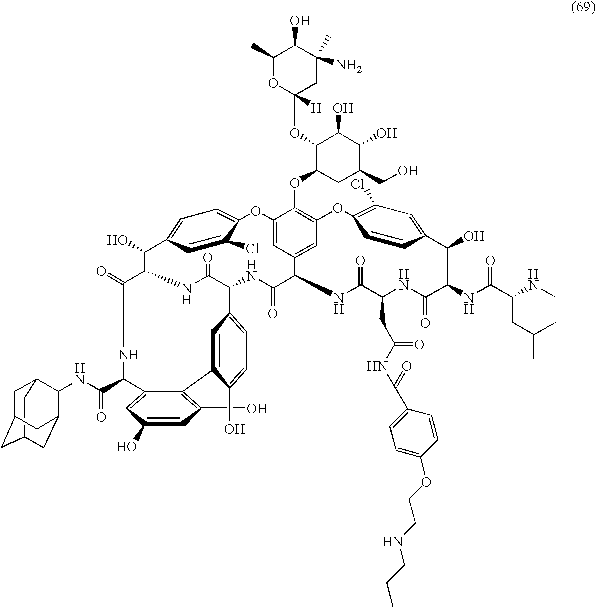

Following the experimental procedure as described in Example 54, for the preparation of Compound (41), replacing octanoic acid with various carboxylic acids or properly hydroxyl or amino protected carboxylic acids, various Fmoc-Compound (41) analogs are obtained. These Fmoc-Compound (4) analogs are subjected to the similar deprotection procedure as described in Example 54 (followed by deprotection for hydroxyl or amino protected compounds), Compound (62), Compound (63), Compound (65), Compound (66), and Compound (67) were prepared and Compound (55), Compound (56), Compound (57), Compound (58), Compound (59), Compound (60), Compound (61), Compound (68) and Compound (69) are made.

### Example 59

### Synthesis of Compound (70)

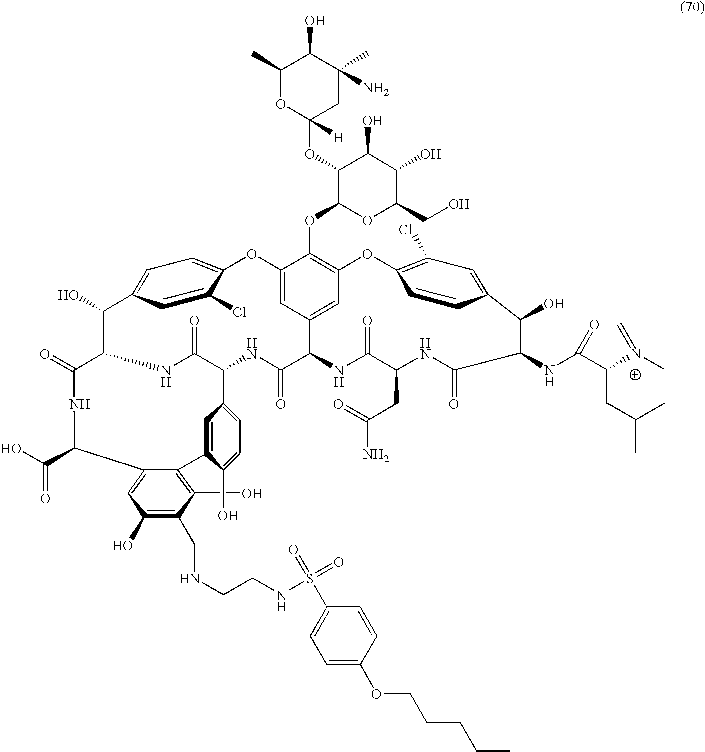

To a solution of a mixture of N-(2-aminoethyl)-4-(pentyloxy)benzenesulfonamide (29 mg) and vancomycin hydrochloride (145 mg) in acetonitrile (5 mL) and water (5 mL) was added 37% aqueous formaldehyde (0.2 g) and acetic acid (64 mg) at room temperature. The reaction mixture was stirred for an additional 20 hr at room temperature. The volatile solvents were removed under reduced pressure. The formed solid was applied on preparative HPLC to give Compound (70) (20 mg) as white powder. ESIMS-MS m/z at 1872.6 (100%), [M+CF3COO]⁻=1872.7, found: 1872.6; +MS m/z at 1760.4 (40%), 1174.1 (40%), 880.7 (100%), [M+H]⁺ 1760.7, Found: 1760.4.

### Example 60

### Synthesis of Compound (71)

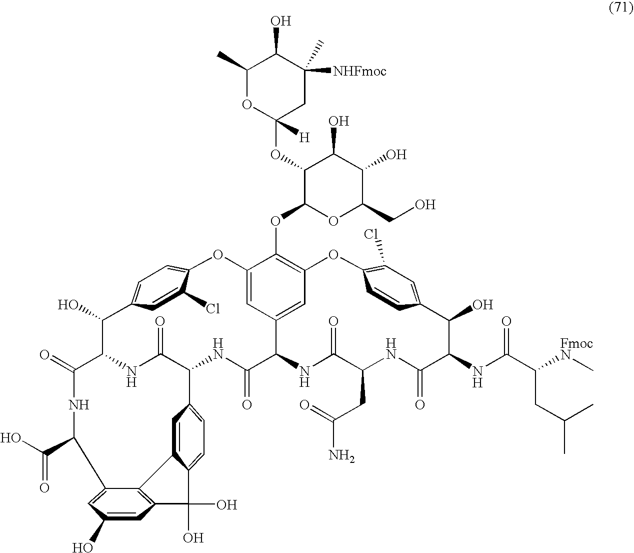

Using a procedure similar to the preparation of Compound (25), as in Example 50 and replacing Compound (21) with vancomycin hydrochloride, Compound (71) is made.

### Example 61

### Synthesis of Compound (72)

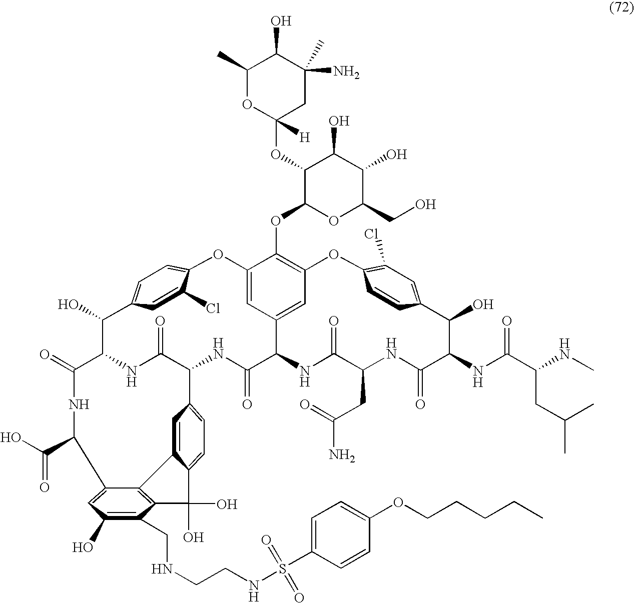

To a solution of mixture of N-(2-aminoethyl)-4-(pentyloxy)benzenesulfonamide (151 mg, 0.53 mmol) and Compound (a) (1 g, 0.53 mmol) in acetonitrile (30 mL) and water (30 mL) was added 37% aqueous formaldehyde (1.2 g, 14.8 mmol) and acetic acid (640 mg, 10.7 mmol) at room temperature. The reaction mixture was stirred for an additional 20 hr at room temperature. The volatile solvents were removed under reduced pressure. The formed solid was collected by filtration and washed with EtOAc. The crude product was dissolved into DMF (5 mL). After diethylamine (22 mg) was added, the reaction mixture was stirred at room temperature for 40 minutes and then was poured into ether (20 mL). The formed solid was applied on preparative HPLC to give Compound (72) (25 mg) as white powder. ESIMS-MS m/z at 1861.1 (100%), [M+CF3COO]⁻=1860.7, found: 1861.1; +MS m/z at 1748.9 (60%), 1165.8 (40%), 874.9 (100%), [M+H]⁺ 1748.7, Found: 1748.9.

### Example 62

### Synthesis of Compound (73)

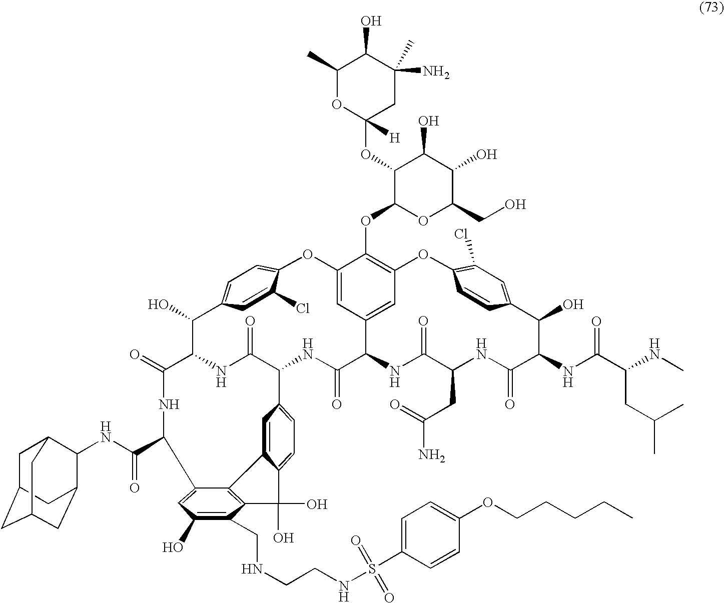

To a solution of Compound (25) (1 g, 0.40 mmol) and N-(2-aminoethyl)-4-(pentyloxy)benzenesulfonamide (120 mg, 0.40 mmol) in acetonitrile (25 mL) and water (25 mL) was added 37% aqueous formaldehyde (1 g, 12 mmol) and acetic acid (400 mg, 6.7 mmol) at room temperature. The reaction mixture was stirred for an additional 20 hr at room temperature. The volatile solvents were removed under reduced pressure. The formed solid was collected by filtration and washed with EtOAc. The crude product was dissolved into DMF (5 mL). After diethylamine (22 mg) was added, the reaction mixture was stirred at room temperature for 40 minutes and then was poured into ether (20 mL). The formed solid was applied on preparative HPLC to give Compound (73) (35 mg, 18%) as white powder. ESIMS-MS m/z at 1994.1 (100%), [M+CF3COO]⁻=1993.9, found: 1994.1; +MS m/z at 1882.0 (20%), 1254.7 (30%), 941.5 (100%), [M+H]⁺ 1881.9, Found: 1882.0.

### Example 63

### Synthesis (74)

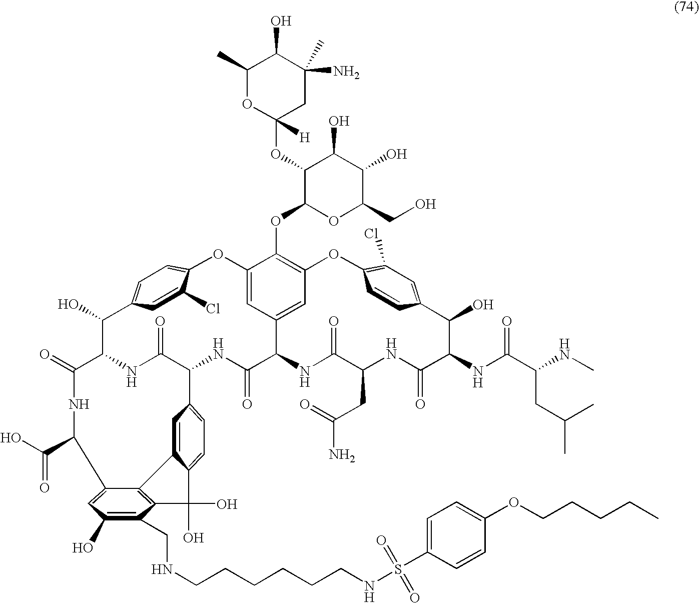

To a solution of Compound (71) (0.95 g) and N-(6-aminohexyl)-4-(pentyloxy)benzenesulfonamide (170 mg) in acetonitrile (40 mL) and water (20 mL) was added 37% aqueous formaldehyde (1 g) and acetic acid (1 mL) at rt. The reaction mixture was stirred for an additional 75 hr at room temperature. The volatile solvents were removed under reduced pressure. The formed solid was collected by filtration and washed with EtOAc. The crude product was dissolved into DMF (10 mL). After diethylamine (0.7 mL) was added, the reaction mixture was stirred at room temperature for 1 hr and then was poured into ether (35 mL). The formed solid was applied onto preparative HPLC to give Compound (74) (55 mg) as white powder. ESIMS-MS m/z at 1917.1 (100%), [M+CF3COO]⁻=1916.8, found: 1917.1; +MS m/z at 902.9 (100%), 1203.5 (40%), 1804.9 (40%), [M+H]⁺ 1804.8, Found: 1804.9.

### Example 64

### Synthesis of Compound (75)

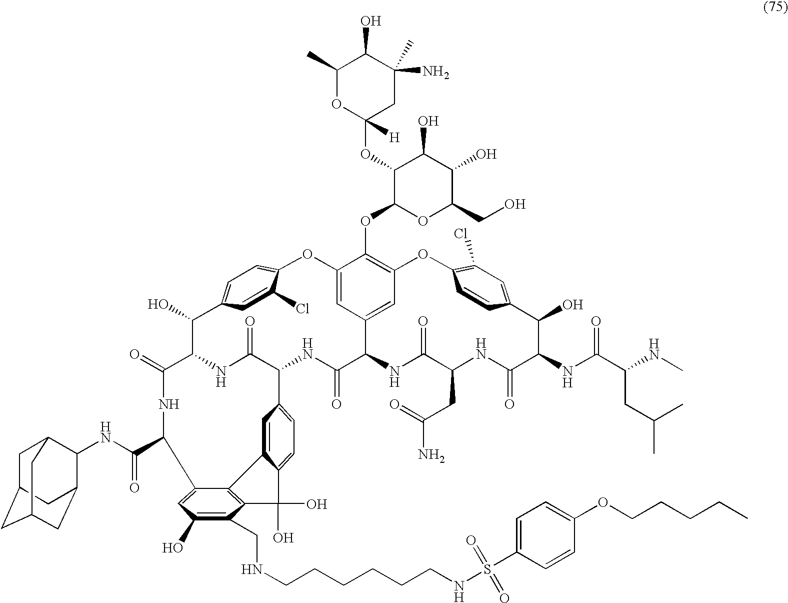

To a solution of Compound (25) (1 g) and N-(6-aminohexyl)-4-(pentyloxy)benzenesulfonamide (170 mg) in acetonitrile (40 mL) and water (20 mL) was added 37% aqueous formaldehyde (1 g) and acetic acid (1 mL) at room temperature. The reaction mixture was stirred for an additional 75 hr at room temperature. The volatile solvents were removed under reduced pressure. The formed solid was collected by filtration and washed with EtOAc. The crude product was dissolved into DMF (10 mL). After diethylamine (1 mL) was added, the reaction mixture was stirred at room temperature for 1 hr and then was poured into ether (35 mL). The formed solid was applied onto preparative HPLC to give Compound (75) (25 mg) as white powder. ESIMS-MS m/z at 2050.2 (100%), [M+CF3COO]⁻=2050.0, found: 2050.2; +MS m/z at 969.5 (100%), 1292.2 (20%), 1938.1 (15%), [M+H]⁺=1938.0, Found: 1938.1

### Example 65

### Synthesis of Compounds 76, 77, 78, 79, 80, 81, 82, 83, 84 and 85

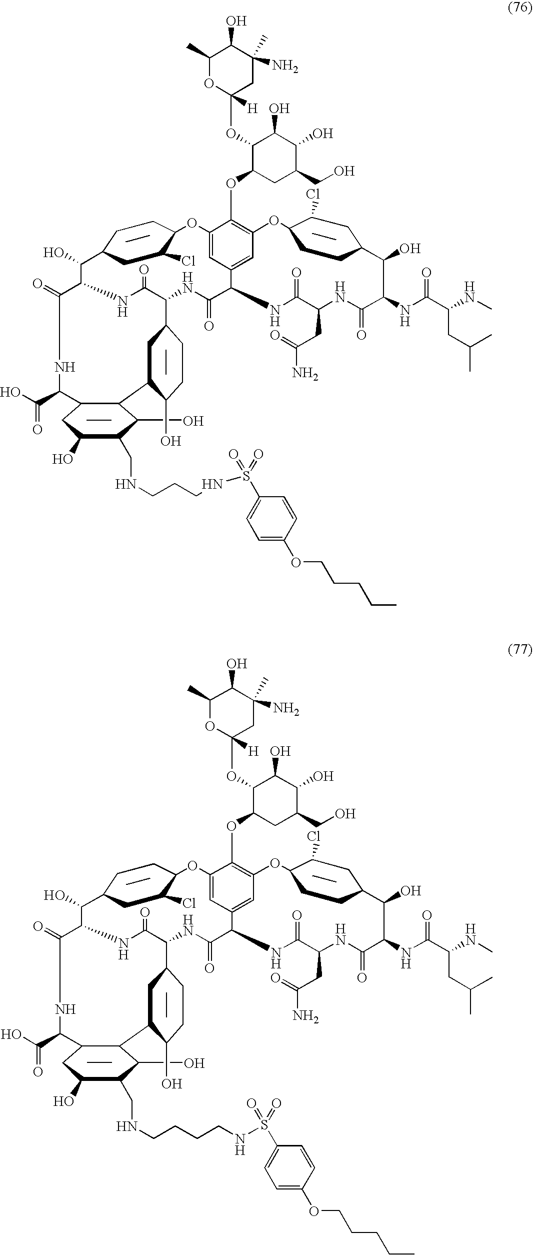

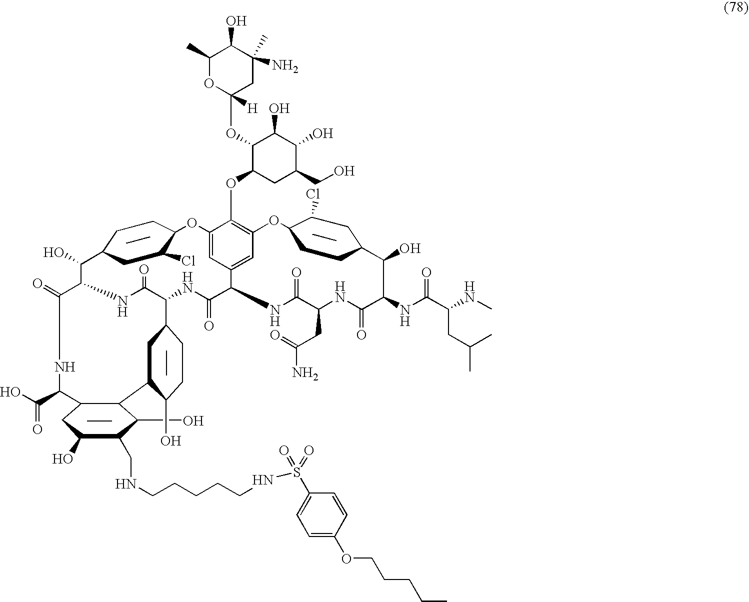

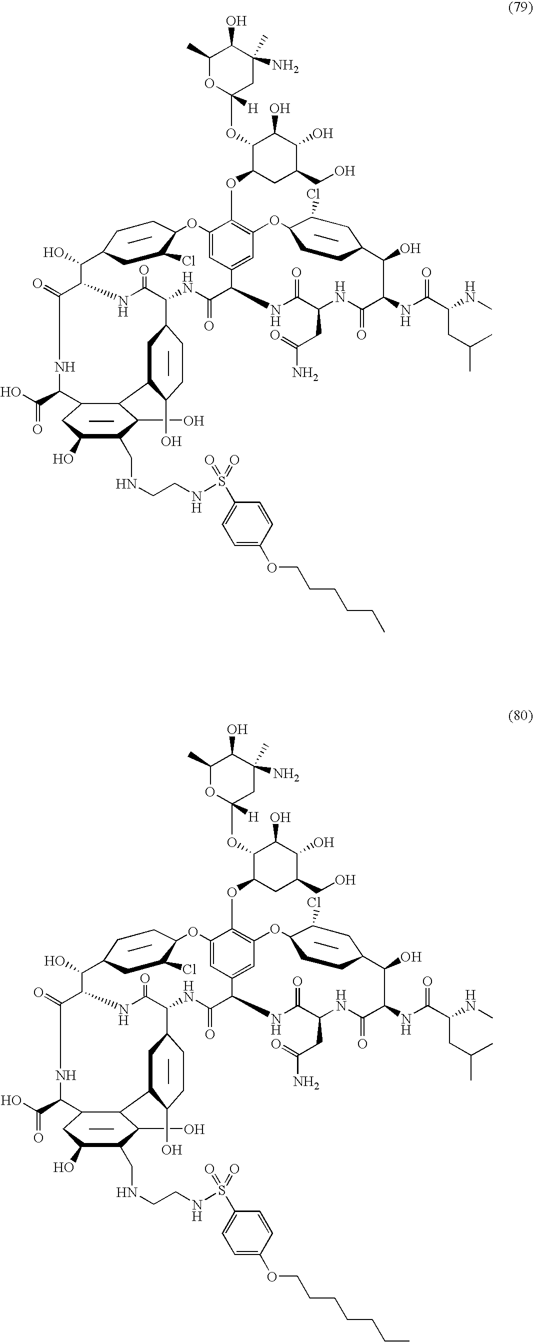

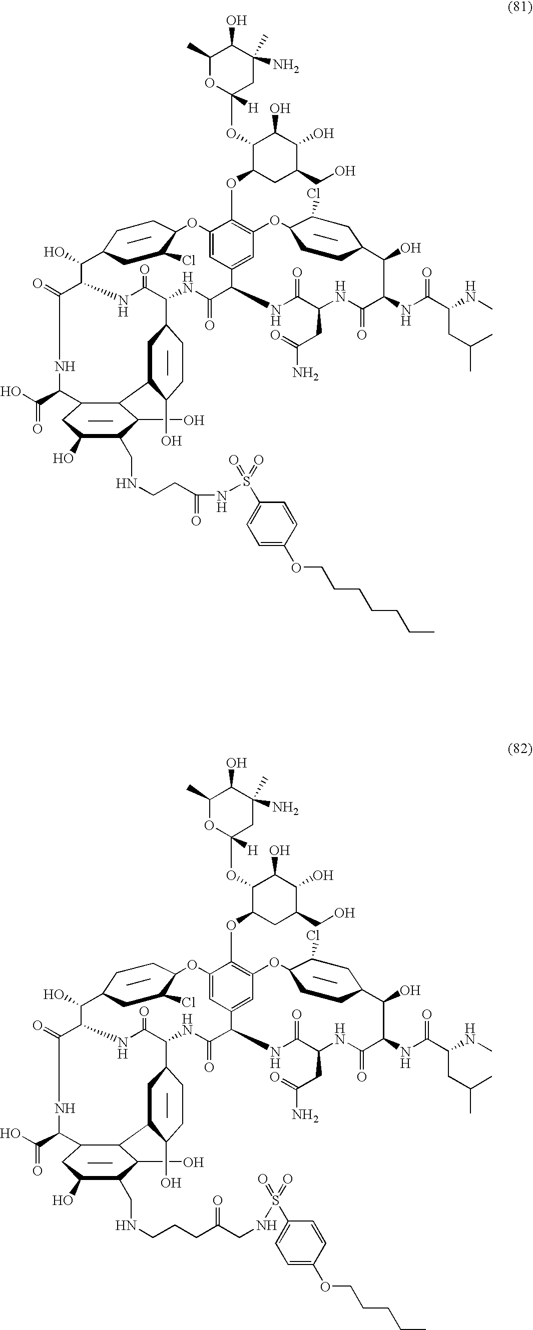

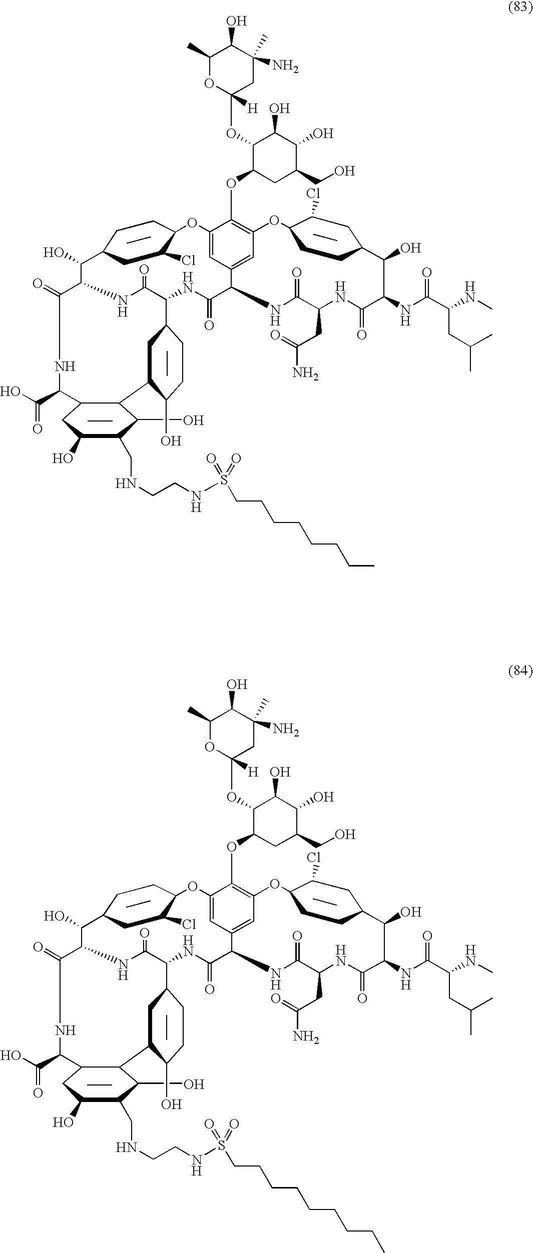

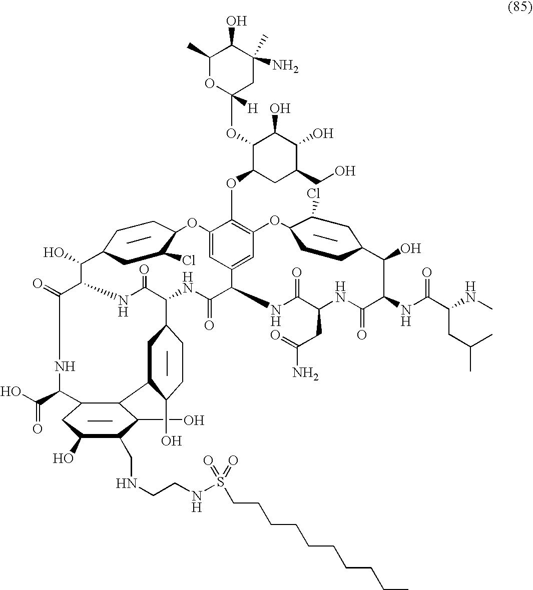

Using a procedure similar to the preparation of Compound (74), as in Example 63 and replacing N-(6-aminohexyl)-4-(pentyloxy)benzenesulfonamide with various appropriate amino-sulfonamide derivatives, Compounds (76-85) are made.

### Example 66

### Synthesis of Compounds 86, 87, 88, 89, 90, 91, 92 and 93

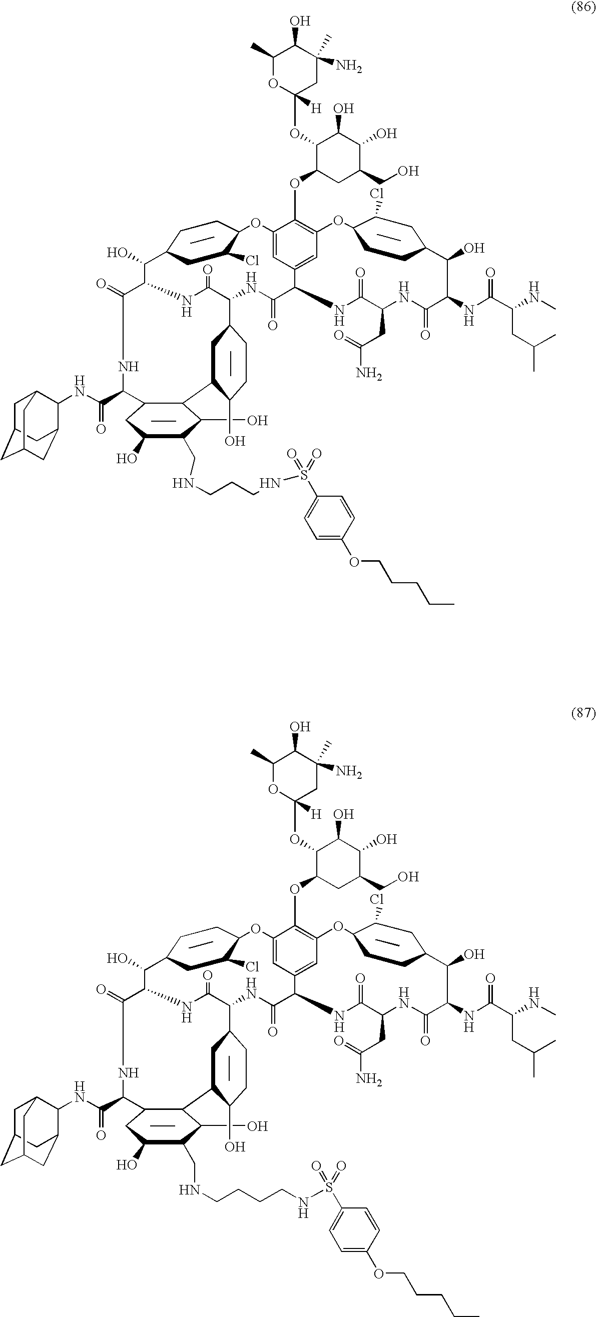

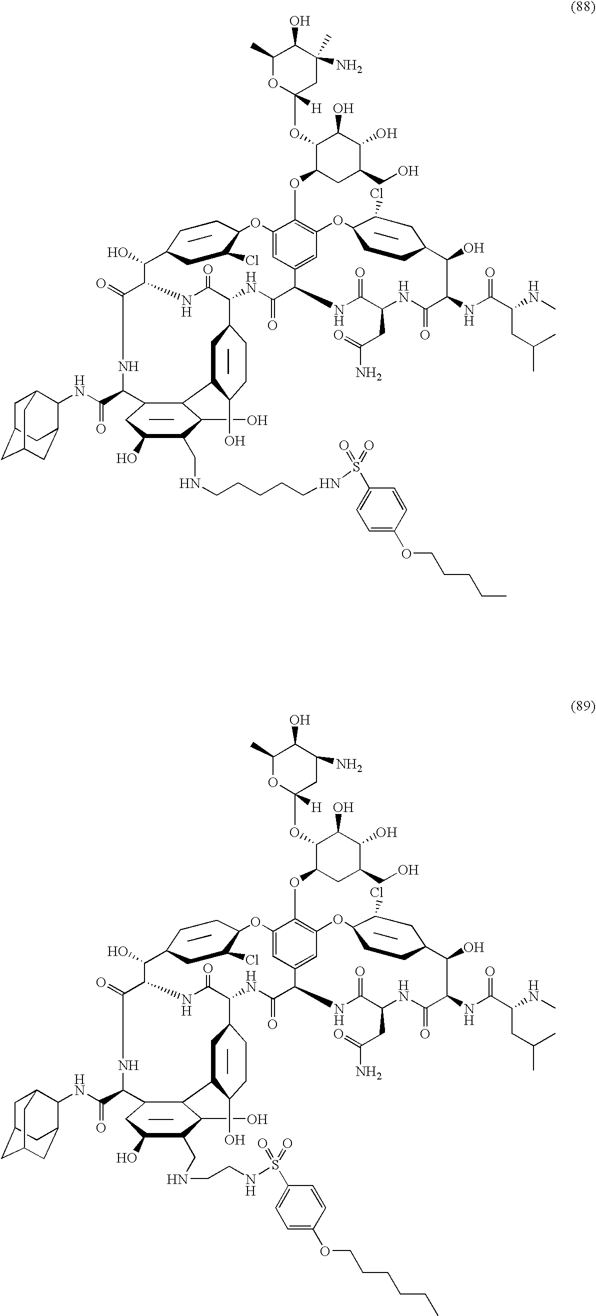

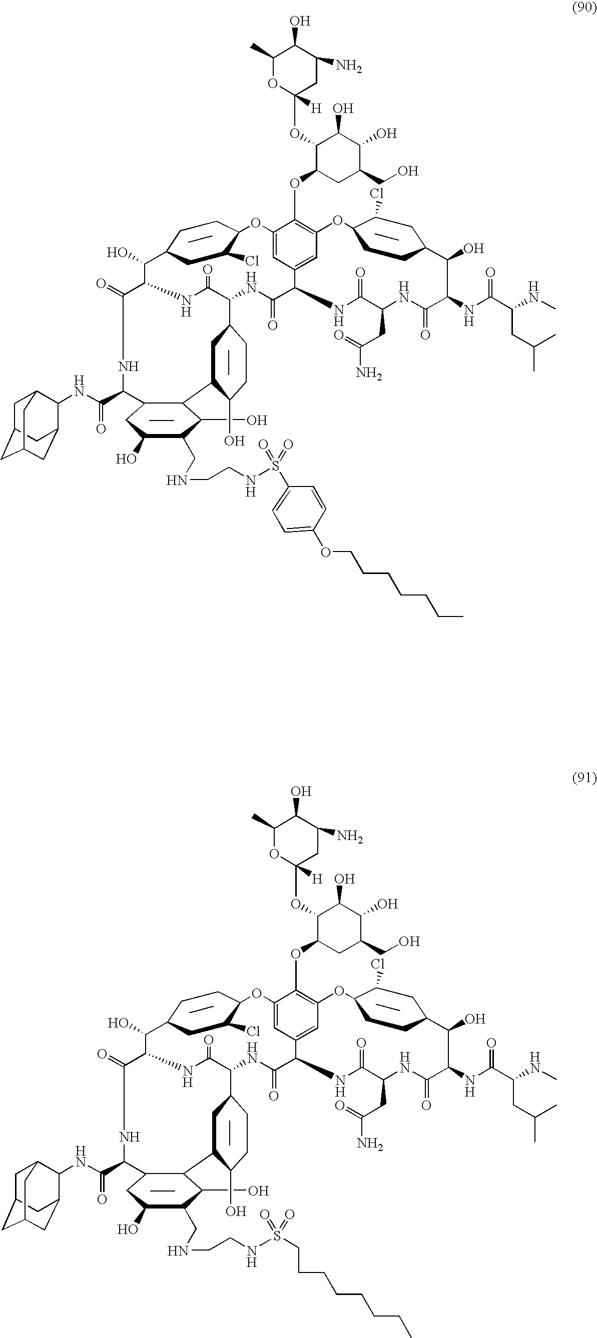

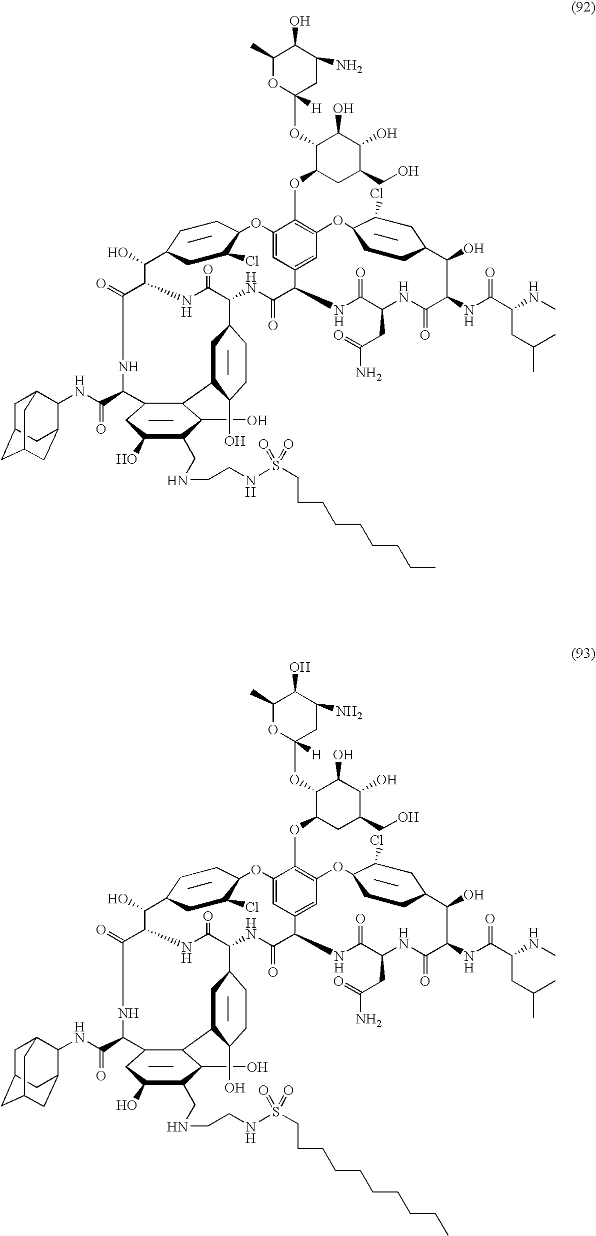

Using a procedure similar to the preparation of Compound (75), as in Example 64 and replacing N-(6-aminohexyl)-4-(pentyloxy)benzenesulfonamide with various appropriate amino-sulfonamide derivatives, Compounds (86-93) are made.

### Example 67

### Synthesis of Compound (94)

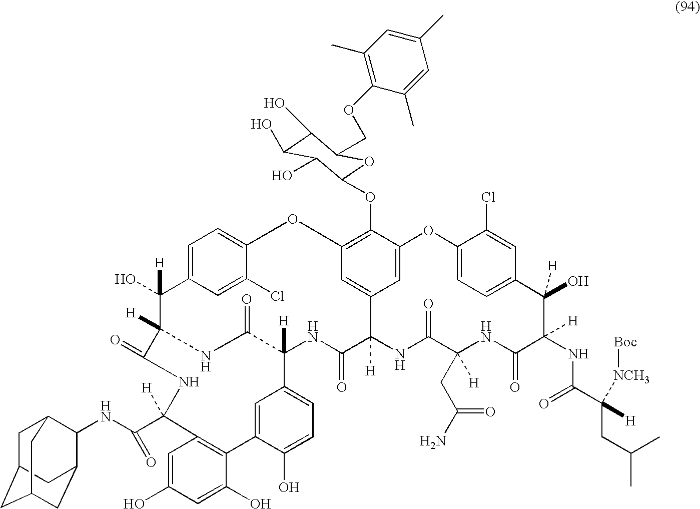

Compound (11) (1 g, 0.649 mmol) was azeotroped with toluene 3 times and then dissolved in anhydrous pyridine. Mesitylenesulfonyl chloride (426 mg, 1.95 mmol) in 1 ml of anhydrous pyridine was added in to the solution dropwise at 0° C., and the mixture was kept stirring for 2 hour. The reaction mixture was poured into water and filtered. The solid was purified by flashing normal phase column (MeOH/DCM=1/10˜1/5) to give Compound (94) as a white solid (500 mg, yield=5 0%). LC-MS (ESI): 1620 (M⁺+1-Boc).

### Example 68

### Synthesis of Compound (95)

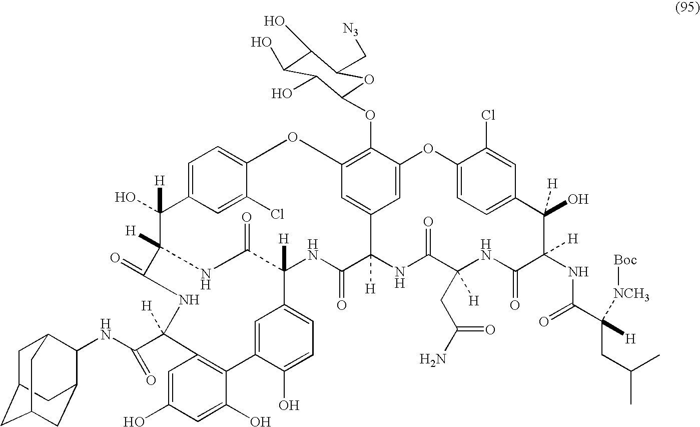

A solution of Compound (94) (1 g, 0.581 mmol) and sodium azide (377 mg, 5.81 mmol, 10 eq.) in anhydrous DMF was heated to 70° C. overnight. The reaction mixture was cooled and added to water. The solid was filtered, washed with water, and purified by flashing normal phase column (MeOH/DCM=1/12˜1/9) to give Compound (95) as a pale yellow solid (500 mg, yield=50%). LC-MS (ESI): 1463 (M⁺+1-Boc).

### Example 69

### Synthesis of Compound (96)

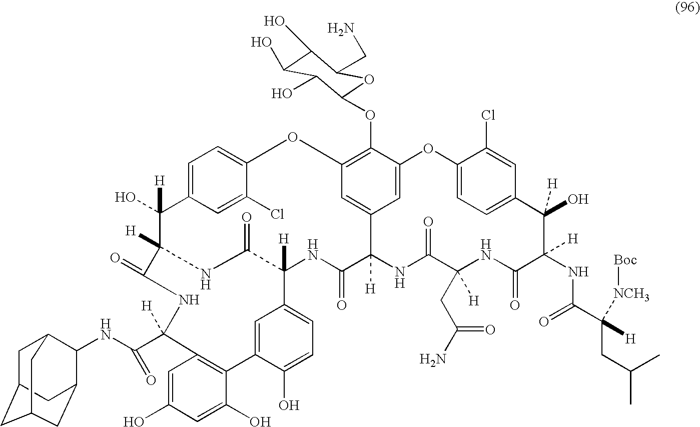

To a solution of Compound (95) (1 g, 0.639 mmol) in 5 ml THF containing a few drops of water was added n-Bu₃P (905 mg, 4.47 mmol). The mixture was heated to reflux overnight, then cooled to room temperature, and poured into water. The solid was filtered, washed with water, and purified by flashing reverse phase column (MeCN/H₂O=1/9˜1/3) to afford Compound (96) as a pale yellow solid (100 mg, yield=10%). LC-MS (ESI): 1537 (M⁺+1).

### Example 70

### Synthesis of Compound (97)

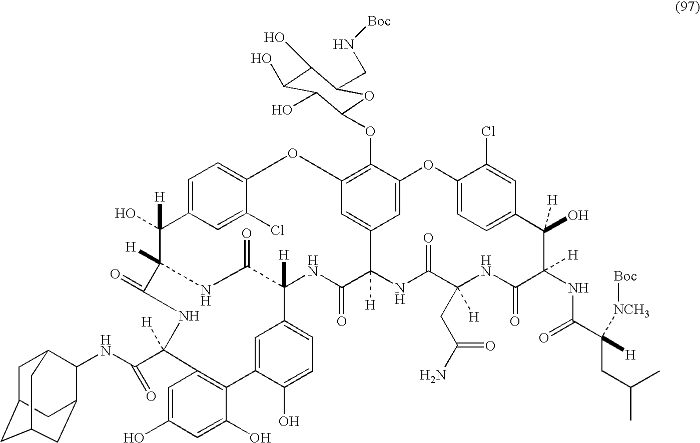

To a solution of Compound (96) (380 mg) in 2 ml of THF and 10 drops of water were added (Boc)₂O (1.05 eq) and TEA (2.0 eq). The mixture was stirred at room temperature for several hours. Check completion by HPLC-MS. The solvent was evaporated to afford Compound (97).

### Example 71

### Synthesis of Compound (98)

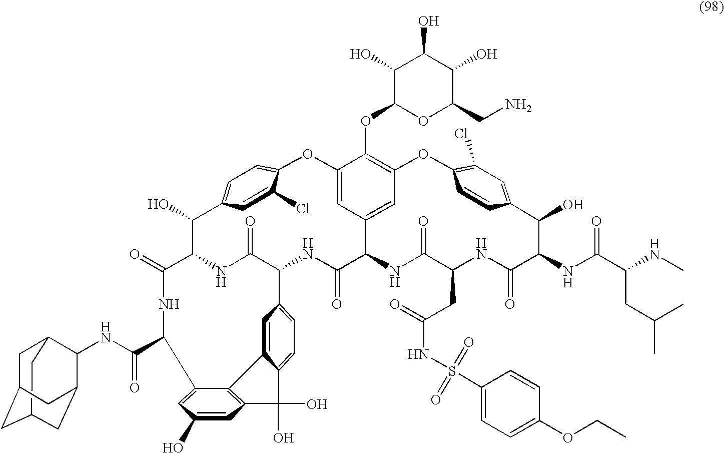

Compound (97) (100 mg) was azeotroped with toluene for three times. It was then dissolved in 1 ml of dry DMF. Triethylamine (18.5 mg, 3.0 eq) in 1 ml of dry DMF was added under argon atmosphere with ice bath followed by addition of 4-ethoxybenzene-1-sulfonyl chloride (27 mg, 2.0 eq) in 1 ml of dry DMF. The mixture was stirred at room temperature overnight. Check completion by HPLC-MS. The reaction was quenched by adding water, and then filtered. The cake was washed three times by water. The crude was purified by Prep-HPLC to afford Boc-Compound (98) intermediate. A solution of the intermediate in 2 ml of TFA/DCM (1/1) was stirred for about 1 h with ice-bath. Check completion by HPLC-MS. The solvent was removed under reduced pressure at 0° C. The residue was washed with ether and filtered to give the expected Compound (98).

### Example 72

### Synthesis of Compounds 99, 100, 101, 102, 103, and 104

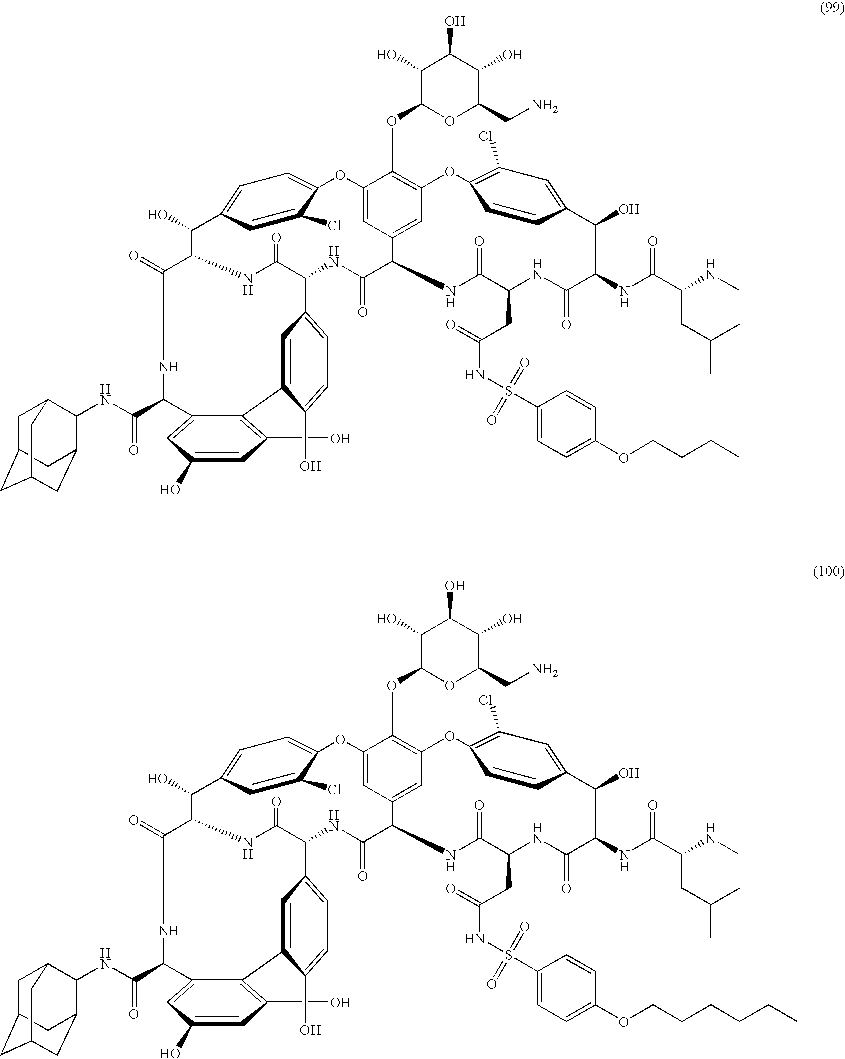

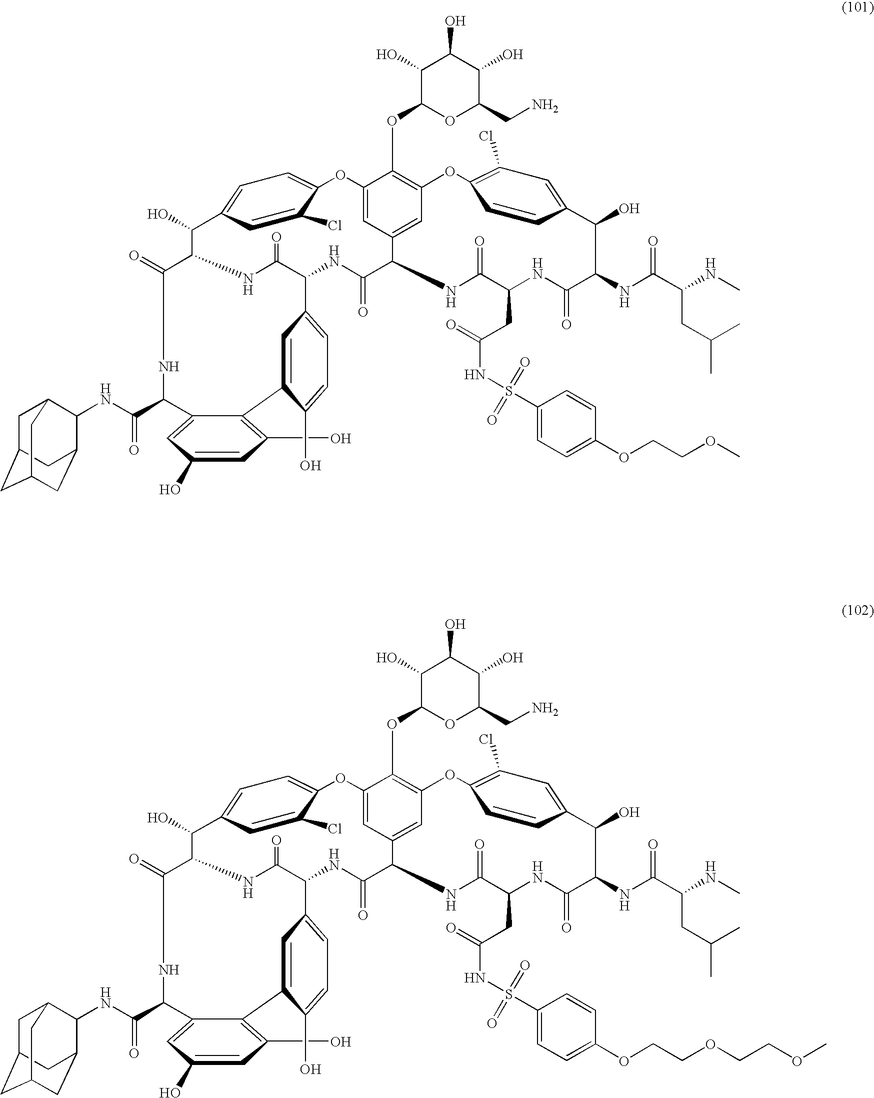

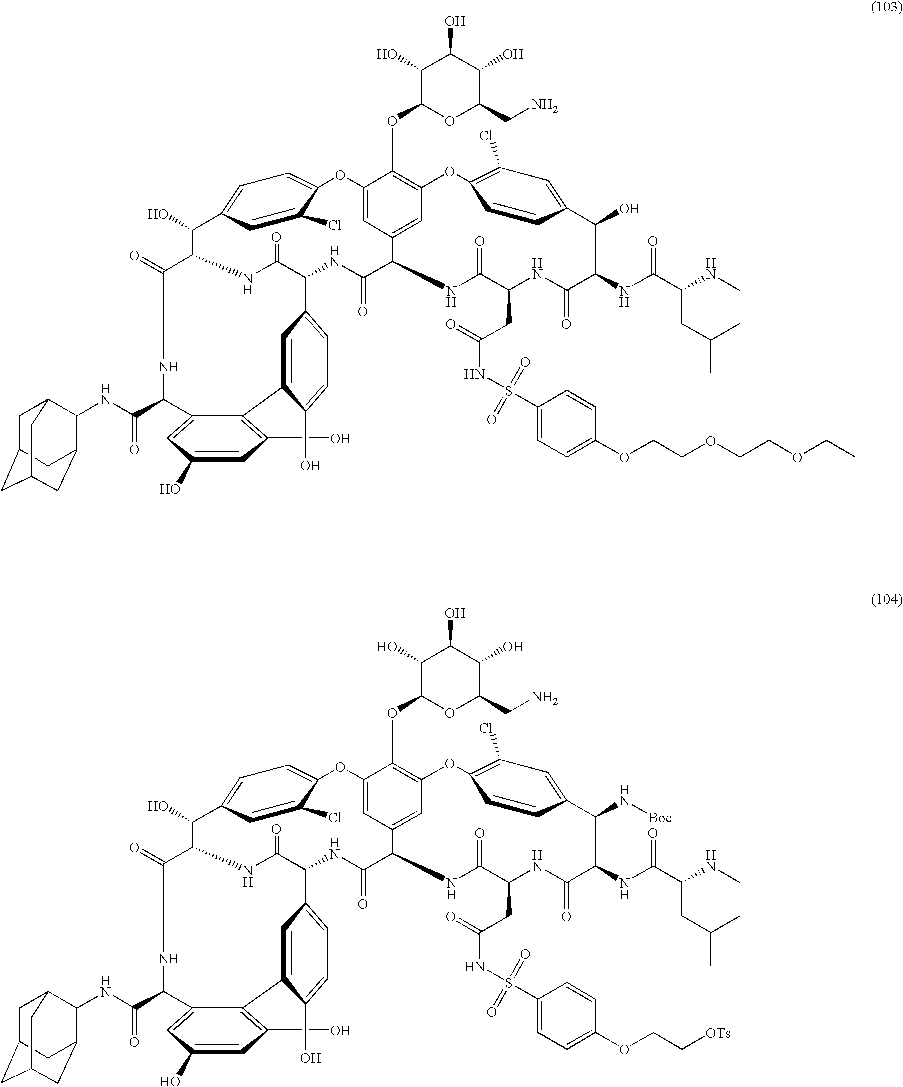

Using a procedure similar to the preparation of Compound (98), as in Example 71 and replacing 4-ethoxybenzene-1-sulfonyl chloride with various appropriate sulfonyl chloride derivatives, Compounds (99-103) were prepared. Without carrying the deprotection of nitrogen protecting Boc group, compound (104) can also be made.

### Example 73

### Synthesis of Compounds 105, 106 and 107

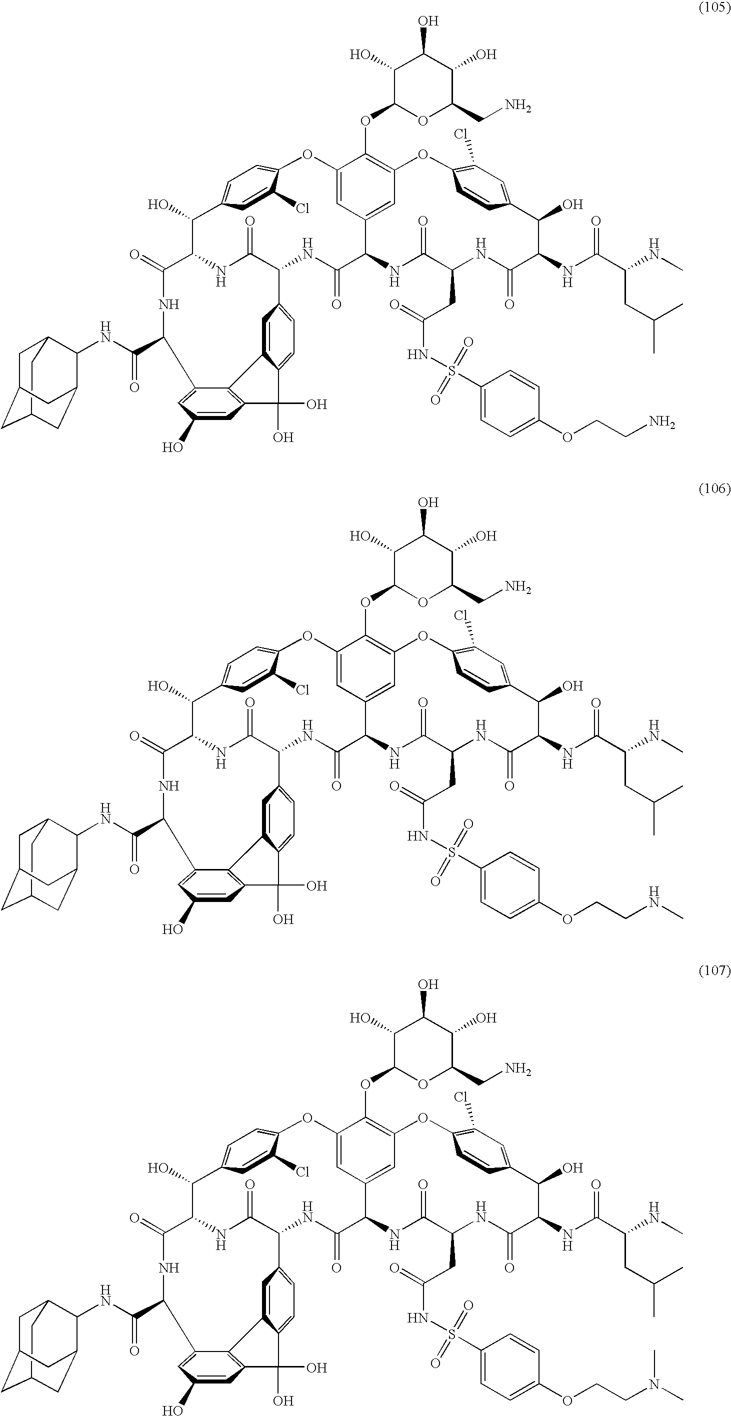

To a solution of 200 mg of Compound (104) in DMF (2 ml) was added 3 mole equivalent of methanolic solution of ammonia, methylamine or dimethyl amine. The solution was stirred at room temperature for 4 hr. to give the Boc-amino intermediates. A solution of the intermediates in 2 ml of TFA/DCM (1/1) was stirred for about 1 h with ice-bath. Check completion by HPLC-MS. The solvent was removed under reduced pressure at 0° C. The residue was washed with ether and filtered giving the expected Compounds (105), (106) and (107).

### Example 74

### Synthesis of Compounds 108, 109, 110 and 111

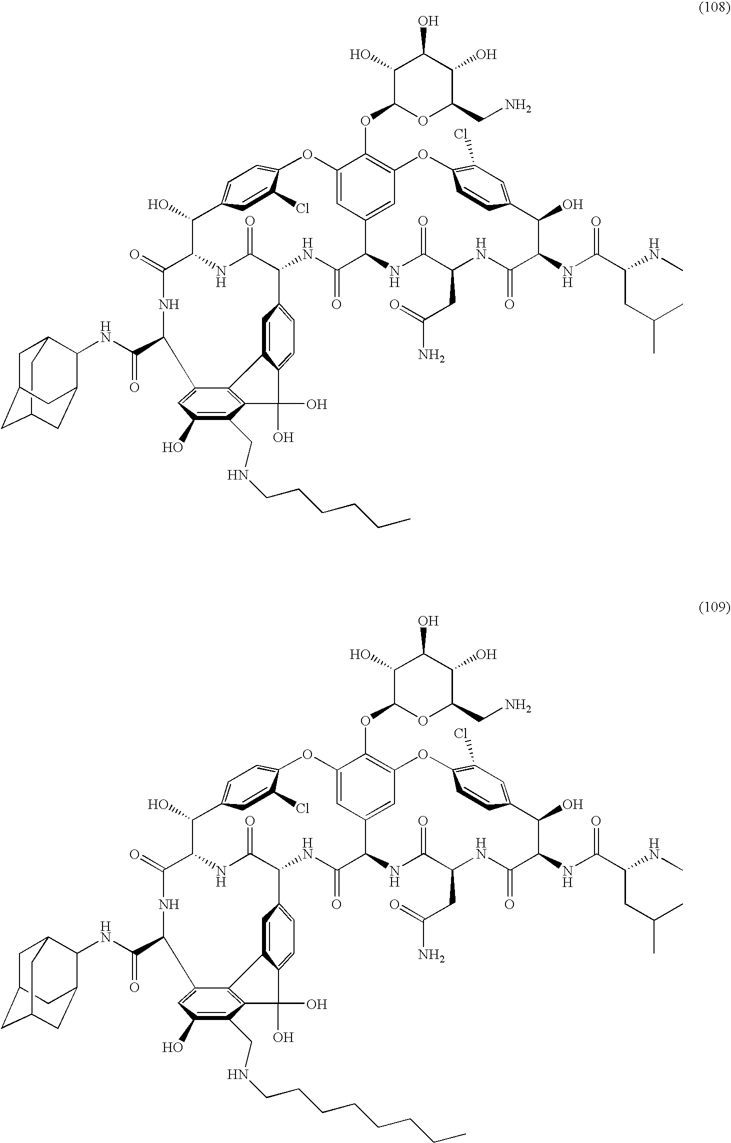

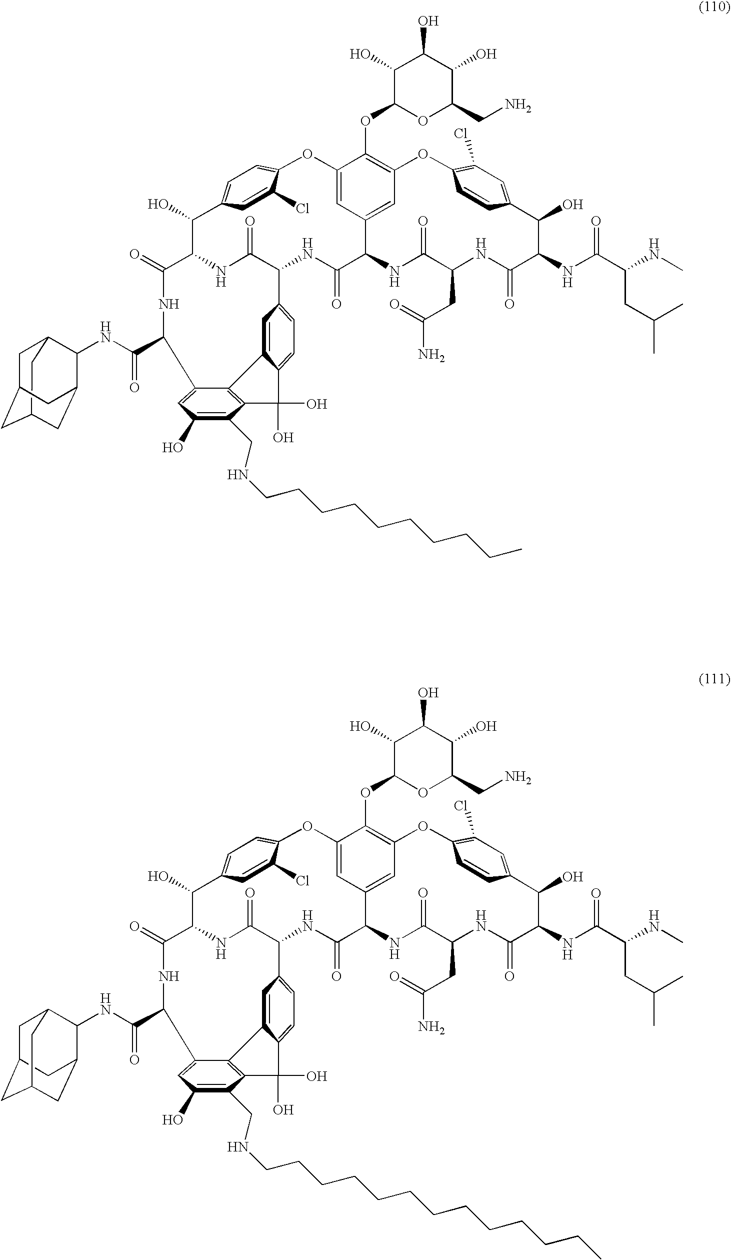

Using a procedure similar to the preparation of Compound (75) as in Example 64 and replacing N-(6-aminohexyl)-4-(pentyloxy)benzenesulfonamide with various appropriate amines, and Compound (25) with Compound (97), the nitrogen protection Boc-Compounds (108-111) are prepared. A solution of these Boc intermediates in 2 ml of TFA/DCM (1/1) was stirred for about 1 h with ice-bath. Check completion by HPLC-MS. The solvent was removed under reduced pressure at 0° C. The residue was washed with ether and filtered to give the expected Compounds (108), (109), (110) and (111).

### Example 75

### Synthesis of Compounds 112, 113 and 114

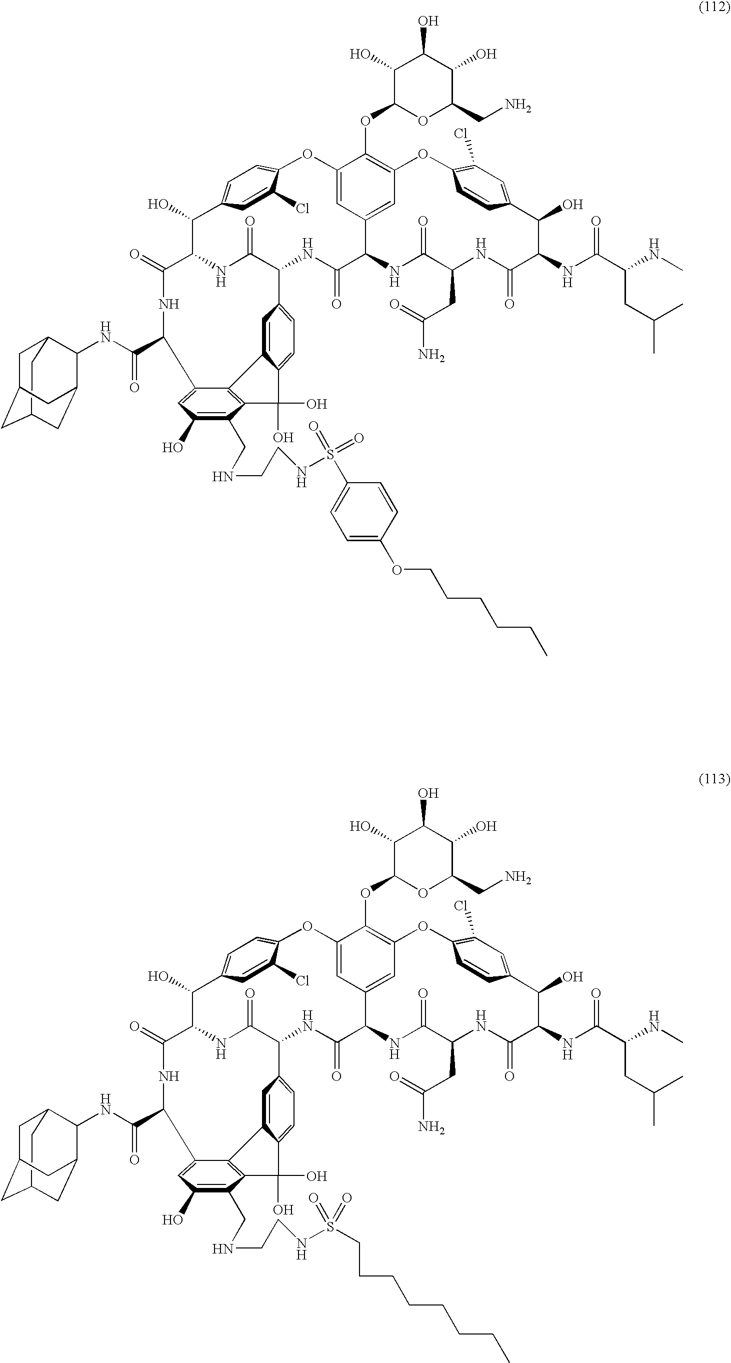

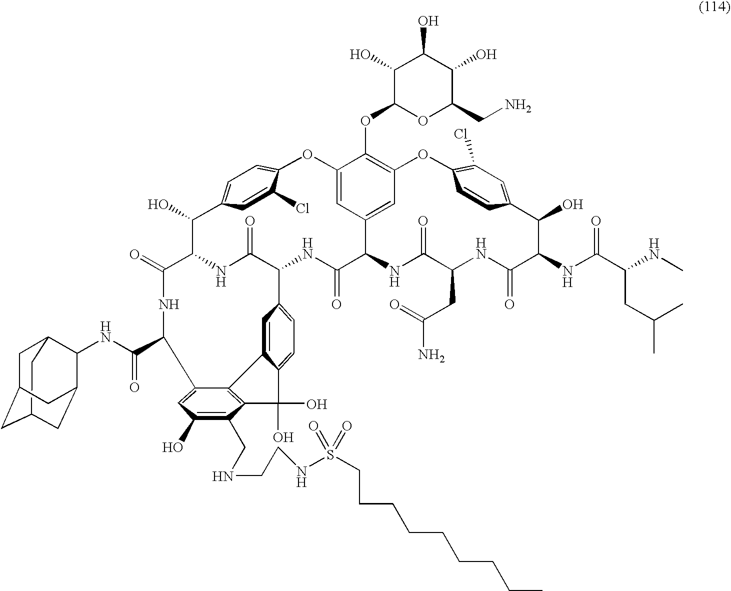

Using a procedure similar to the preparation of Compound (75), as in Example 64 and replacing Compound (25) with Compound (97) and also N-(6-aminohexyl)-4-(pentyloxy)benzenesulfonamide with various appropriate amino-sulfonamides, the nitrogen protection Boc-Compounds (112-114) are prepared. A solution of these Boc intermediates in 2 ml of TFA/DCM (1/1) was stirred for about 1 h with ice-bath. Check completion by HPLC-MS. The solvent was removed under reduced pressure at 0° C. The residue was washed with ether and filtered to give the expected Compounds (112), (113), and (114) and prepared.

### Example 76

### Synthesis of Compounds 115, 116, 117, 118, 119, 120, 121, 122, and 123

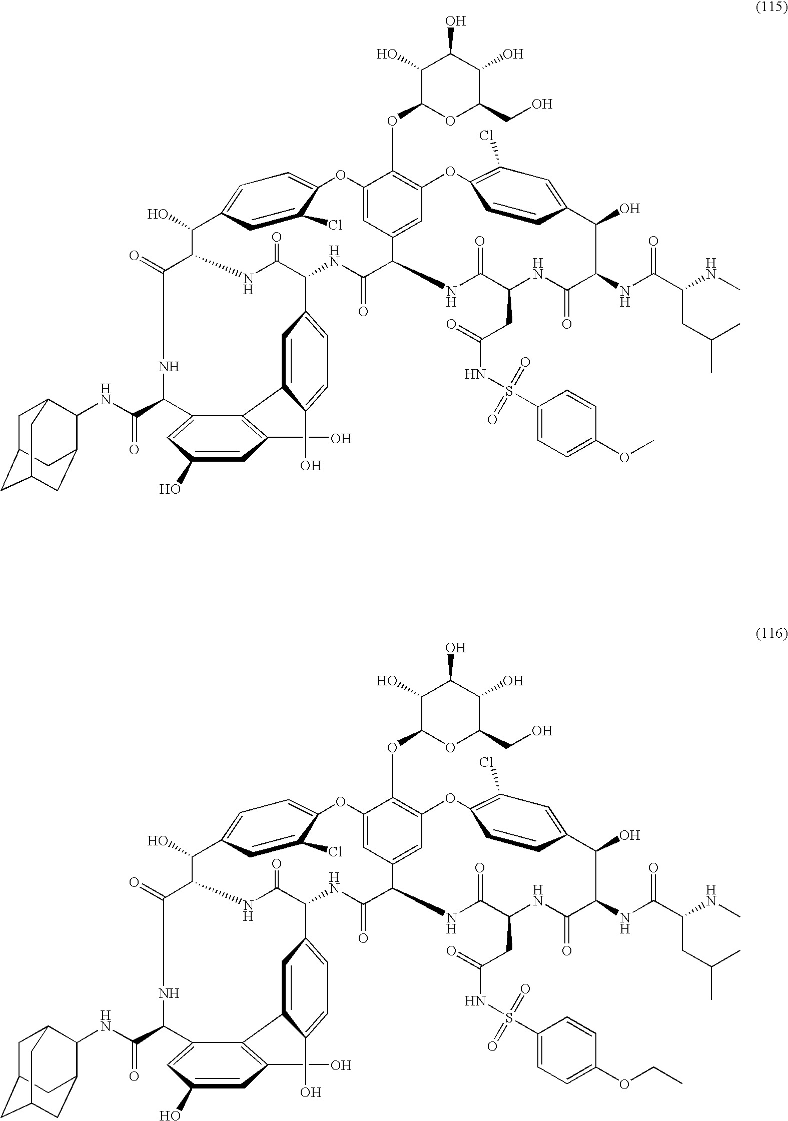

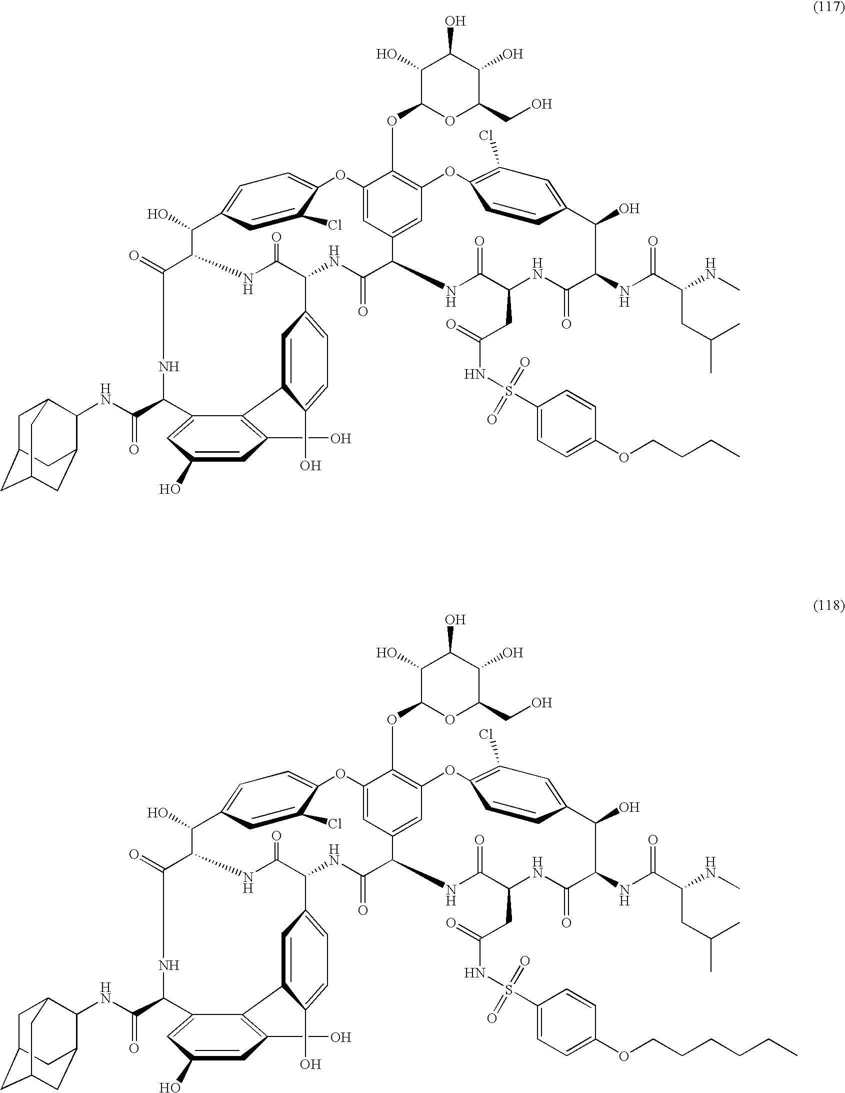

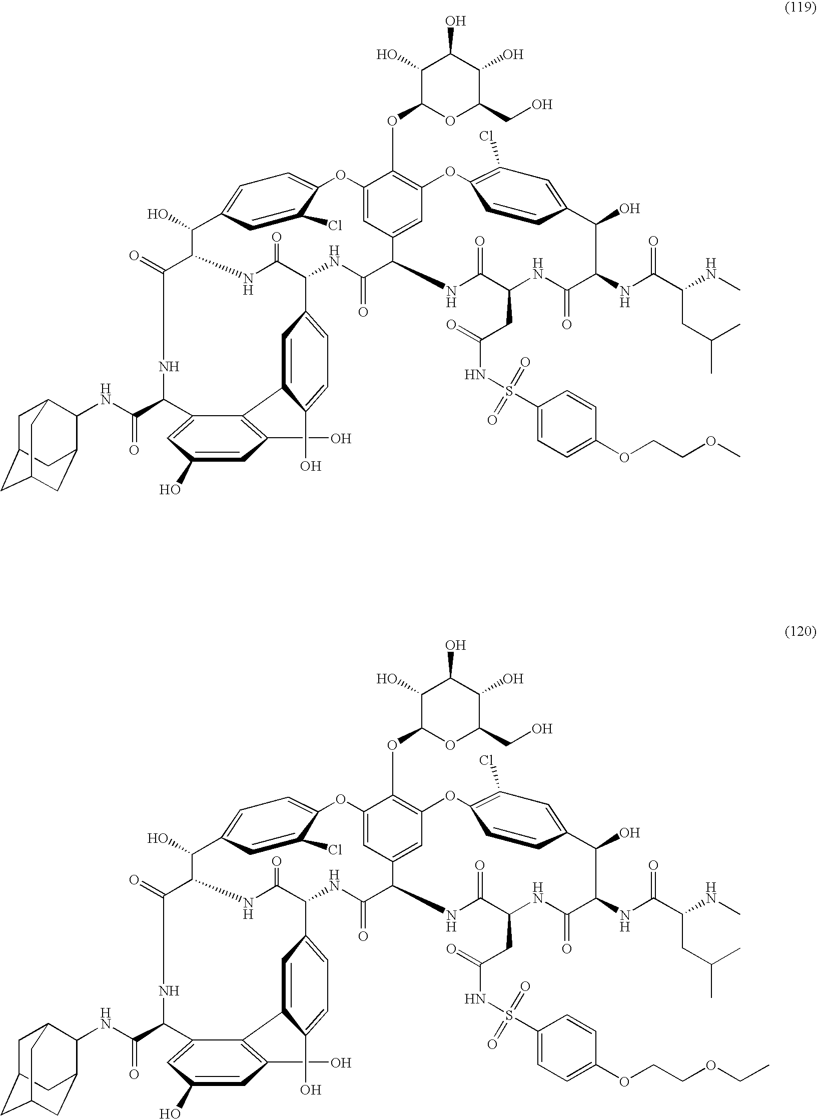

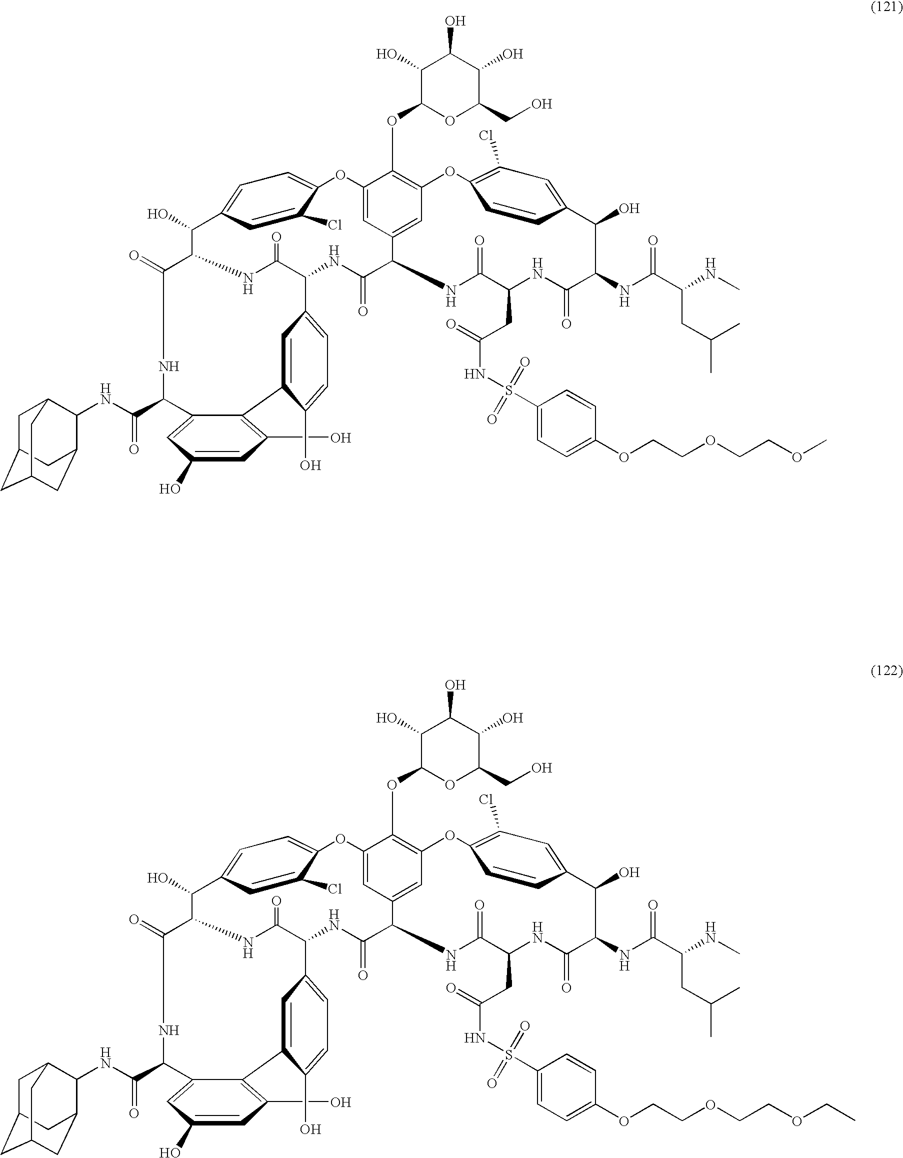

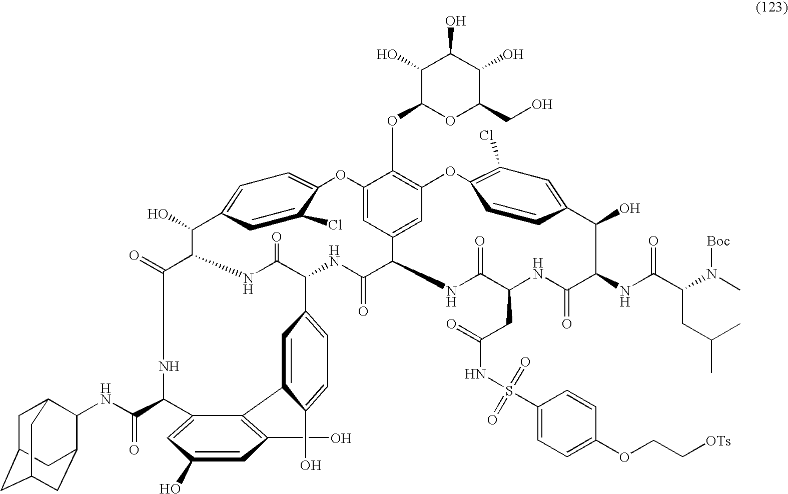

Using a procedure similar to the preparation of Compound (98), as in Example 71 and replacing compound (97) with Compound (11) and 4-ethoxybenzene-1-sulfonyl chloride with various appropriate benzene-1-sulfonyl chloride derivatives, Compound (117), and Compound (118) were prepared and Compound (117), Compound (116), Compound (119), Compound (120), Compound (121), and Compound (122) are made. Without carrying the deprotection of nitrogen protecting Boc group, compound (123) can also be made.

### Example 77

### Synthesis of Compounds 124, 125 and 126

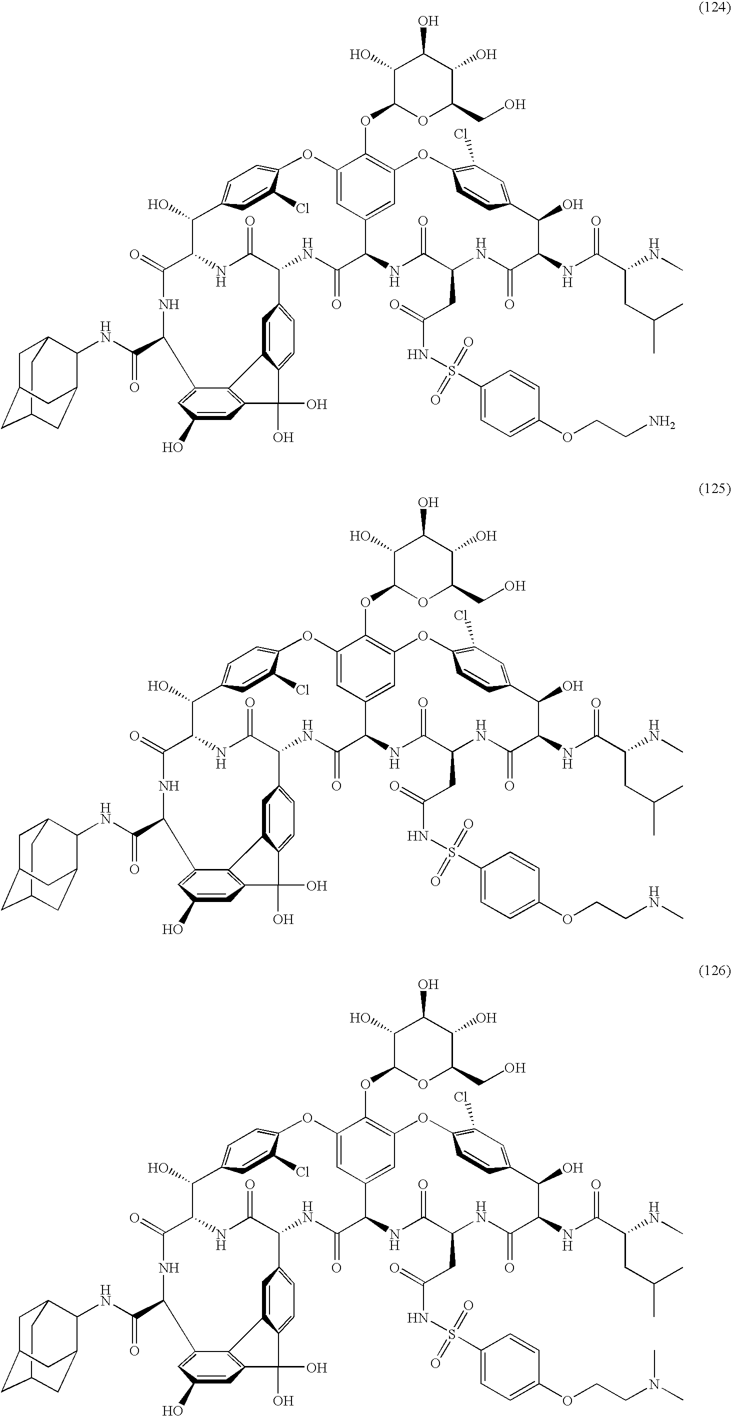

To a solution of 200 mg of Compound (123) in DMF (2 ml) is added 3 mole equivalent of methanolic solution of ammonia, methylamine or dimethyl amine. It is then stirred at room temperature at for 4 hr. to give the Boc-amino intermediates. A solution of the intermediates in 2 ml of TFA/DCM (1/1) was stirred for about 1 h with ice-bath. Check completion by HPLC-MS. The solvent was removed under reduced pressure at 0° C. The residue was washed with ether and filtered to give Compounds (124), (125) and (126).

### Example 78

### Synthesis of Compound (127)

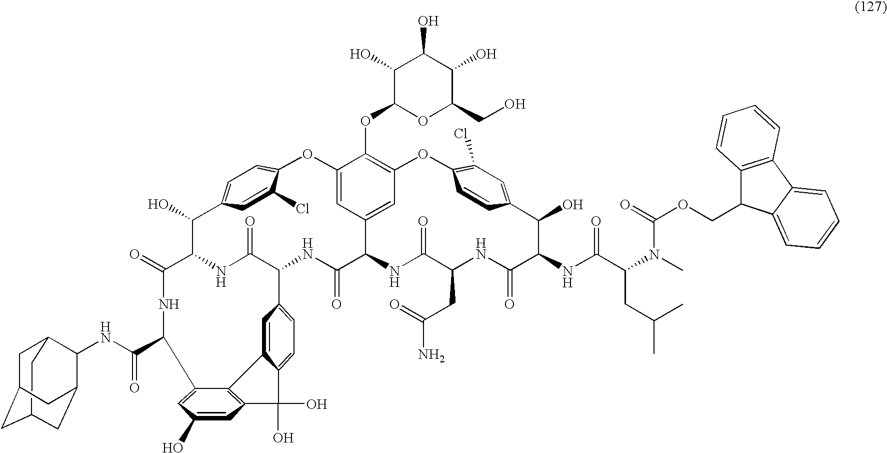

To a solution of Compound (1) (14.30 g, 10.95 mmol) and 2-aminoadamantane hydrochloride (16.42 mmol, 1.5 eq.) in DMSO (100 mL) was added DIPEA (5.66 g, 4 eq.) and HATU (4.17 g, 1.0 eq.) at rt with stirring. The reaction was monitored by analytical HPLC. After stirred at room temperature overnight, the reaction mixture was poured into CH₂Cl₂ (300 mL) with stirring. The formed solid was collected by filtration under reduced pressure and washed with ether (3×100 mL) to provide a crude product, Compound (17) (9.40 g), which was used into the next step without further purification. The crude product Compound (17) (9.38 g) was dissolved into a mixture of THF (70 mL) and H₂O (50 mL). Fmoc-OSu (2.20 g, 6.52 mmol) and NaHCO₃ (0.55 g, 6.52 mmol) was added into the solution. After stirred at room temperature for 2 hr, the solvents was removed away by rotary evaporator. The residue was purified through flash column chromatography on silica gel (eluent: MeOH/CH₂Cl₂=15/1→12/1→10/1→8/1) to provide Compound (127) (5.0 g) as a white powder. ESI-MS m/z, [M+Na]⁺=1684.5, Found: 1684.5; [M−H]⁺=1660.7, Found: 1660.5.

### Example 79

### Synthesis of Compounds 128, 129, 130, 131, 132, 133, 134, 135, 136, 137, 138, and 139

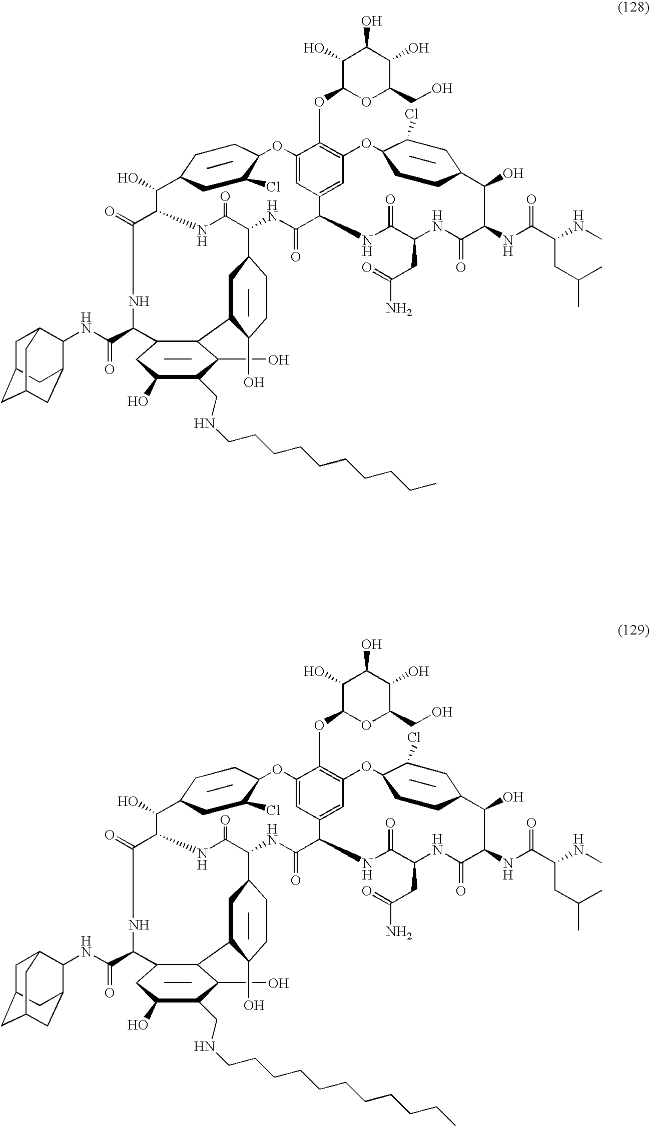

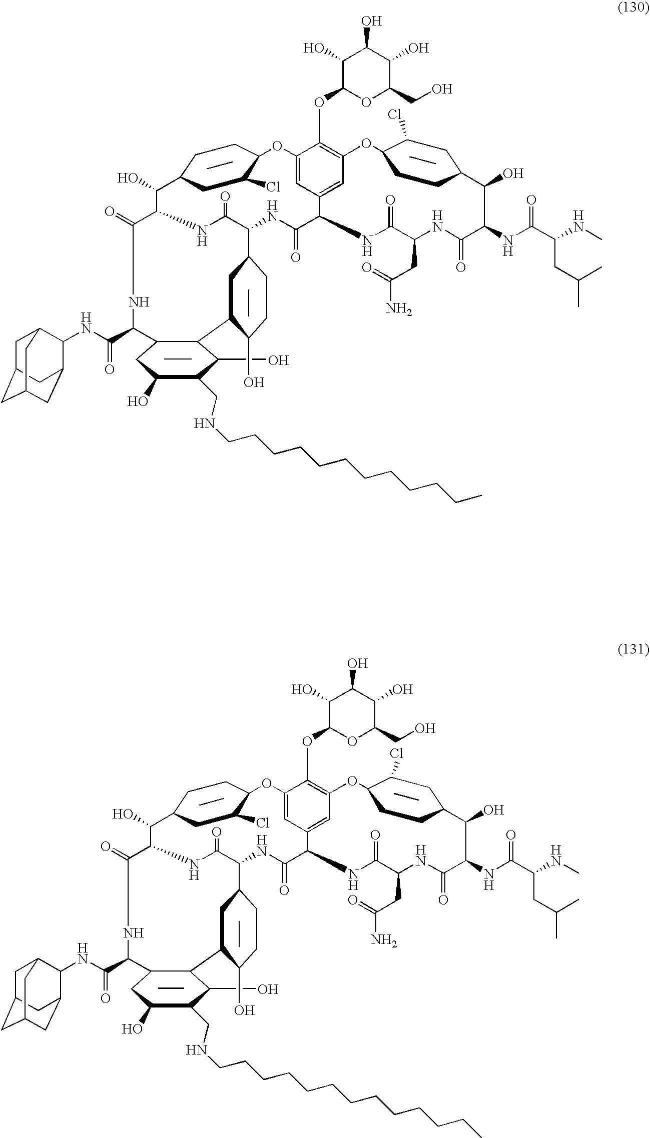

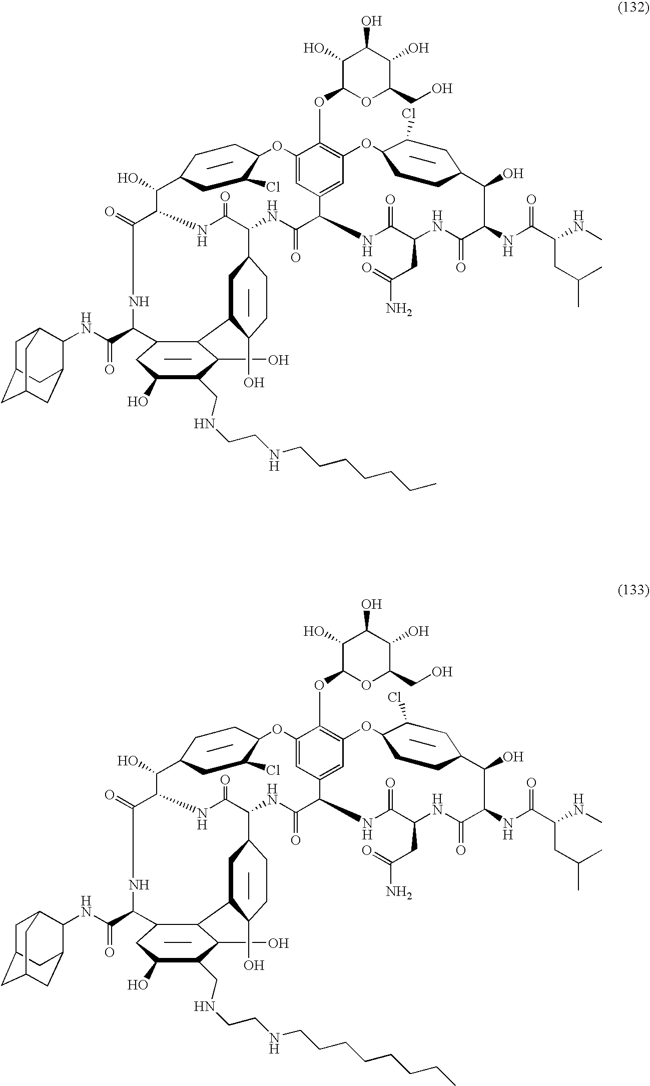

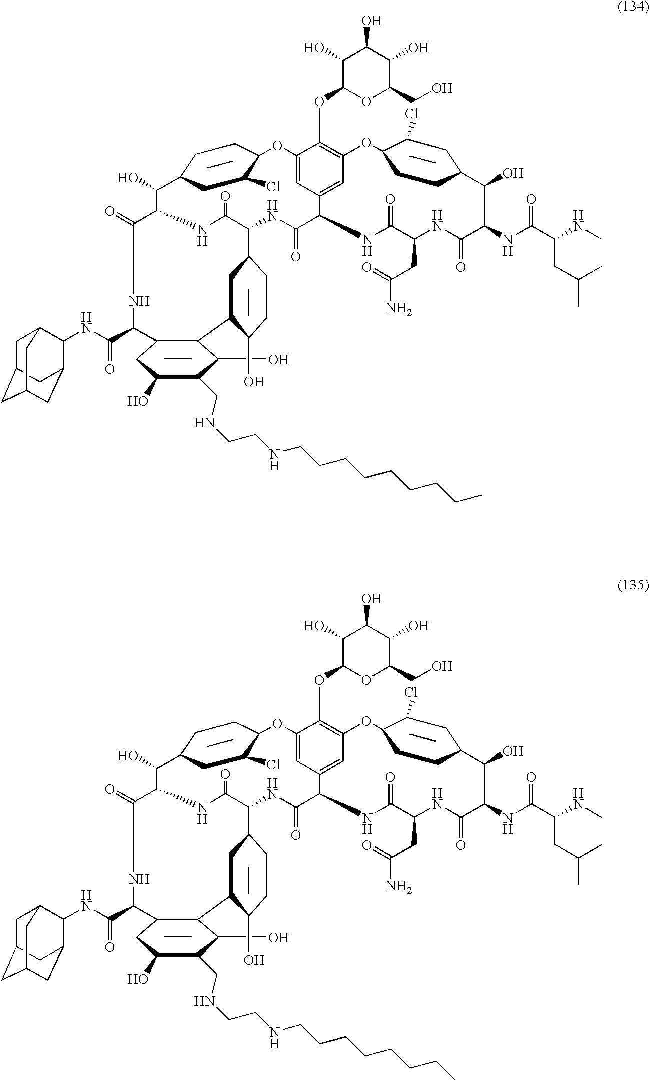

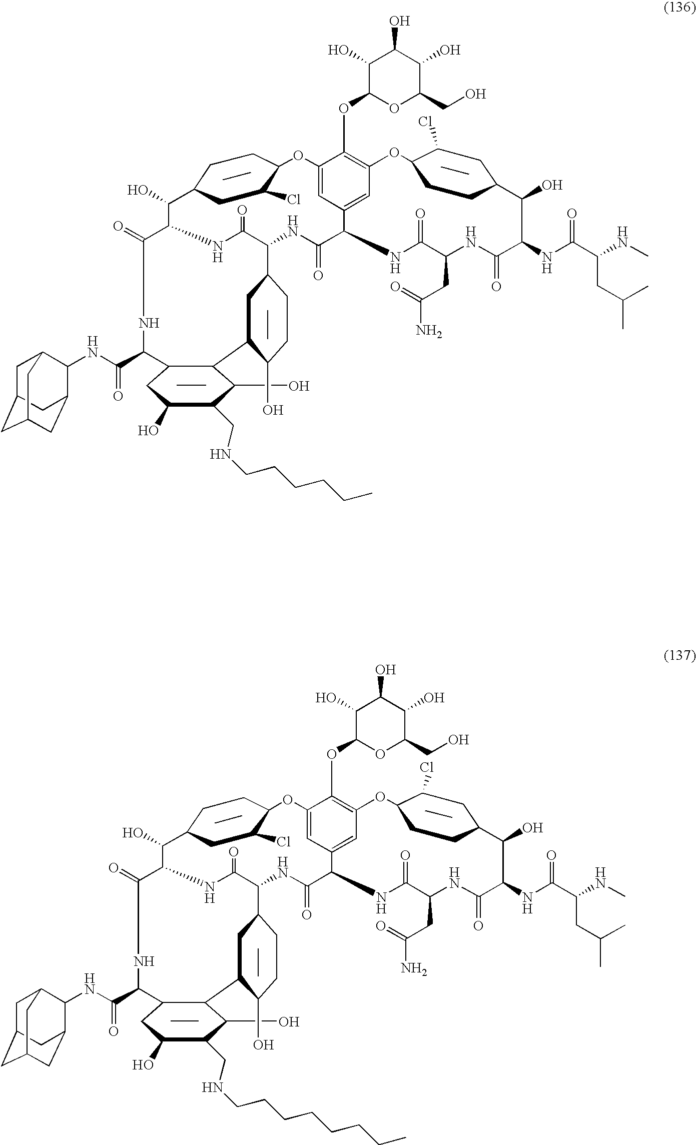

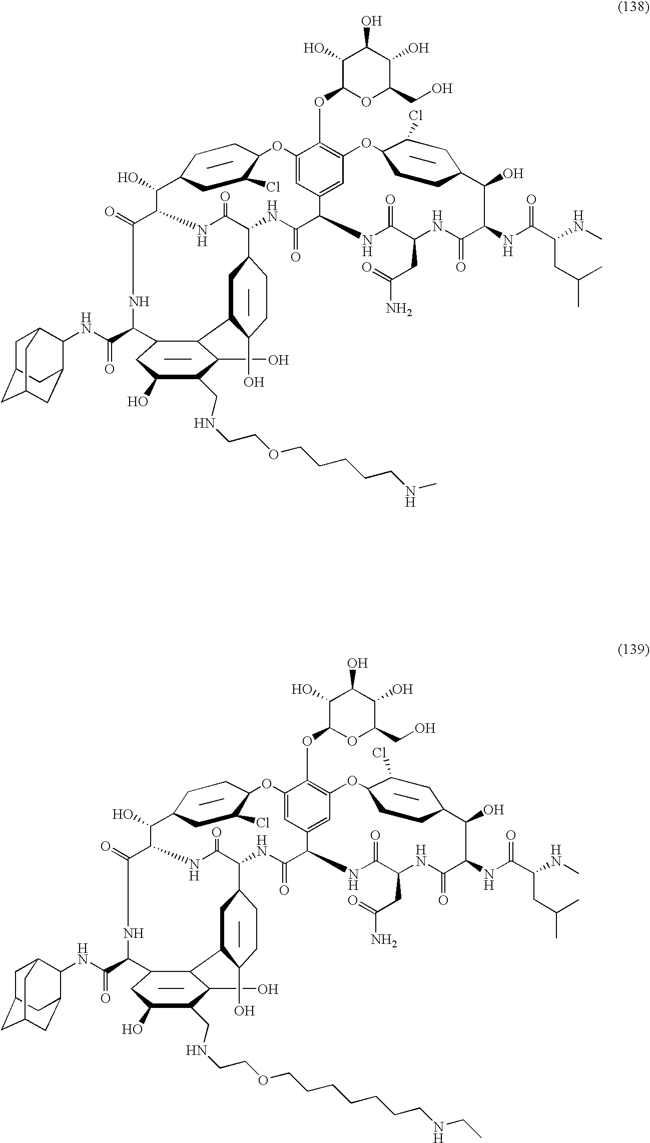

Using a procedure similar to the preparation of Compound (75), as in Example 64 and replacing Compound (25) with Compound (127) as well as N-(6-aminohexyl)-4-(pentyloxy)benzenesulfonamide with various appropriate amines (when there is an extra amino group, the secondary amino group is protected by Fmoc group) derivatives, Compounds (128-139) are made.

### Example 80

### Synthesis of Compound (140)

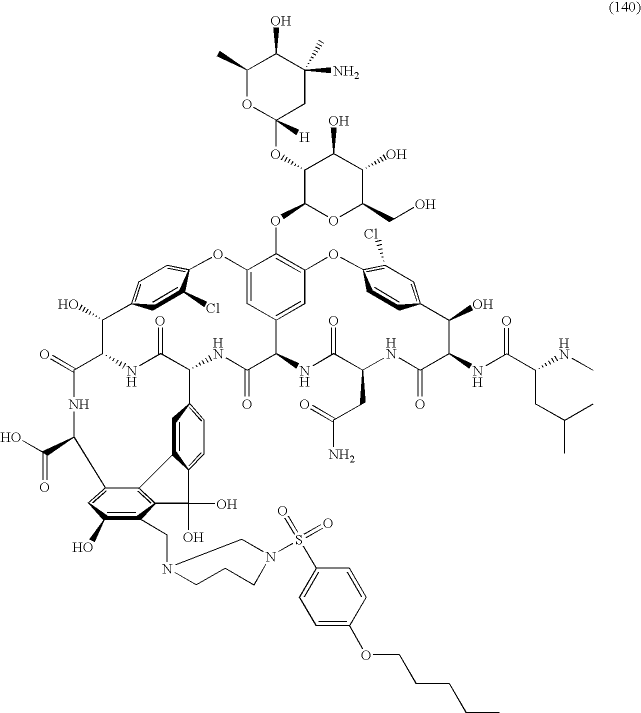

To a solution of Compound (71) (1 g, 0.53 mmol) in CH₃CN (30 mL) and H₂O (20 mL) was added N-(3-aminopropyl)-4(pentyloxy)benzenesulfonamide (200 mg, 0.67 mmol), aq. HCHO (37%, 1 g, 23 eq.) and HOAc (200 mL) at rt with stirring. The reaction was monitored by analytical HPLC in conjunction with ESI-MS. After stirred at rt for 48 hr, the solvents was removed by rotary evaporator. The resulting white solid was collected by filtration under reduced pressure, washed with EtOAc (2×20 mL) and dried under vacuum to give a Fmoc-product (768 mg) (ESI-MS m/z, [M+H]⁺=2218.2, Found: 2218.8; [M+CF3CO2]⁻=2331.2, Found: 2331.1) The product was directly treated with diethylamine (70 mg, 0.95 mmol, 3 eq.) in dry DMF (10 mL) at rt for 50 min. The reaction mixture was poured into ether. The resulting solid was collected by filtration under reduced pressure and washed with ether. The solid was isolated by reversed phase preparative HPLC (C18, 250×22 mm, ACN—H₂O containing 0.03% TFA, 10 ml/min) to give Compound (140) as a white solid. ESI-MS m/z, [M+H]⁺=1774.7, Found: 1774.5; [M+CF3CO2]⁻=1886.7, Found: 1886.7.

### Example 81

### Synthesis of Compound (141)

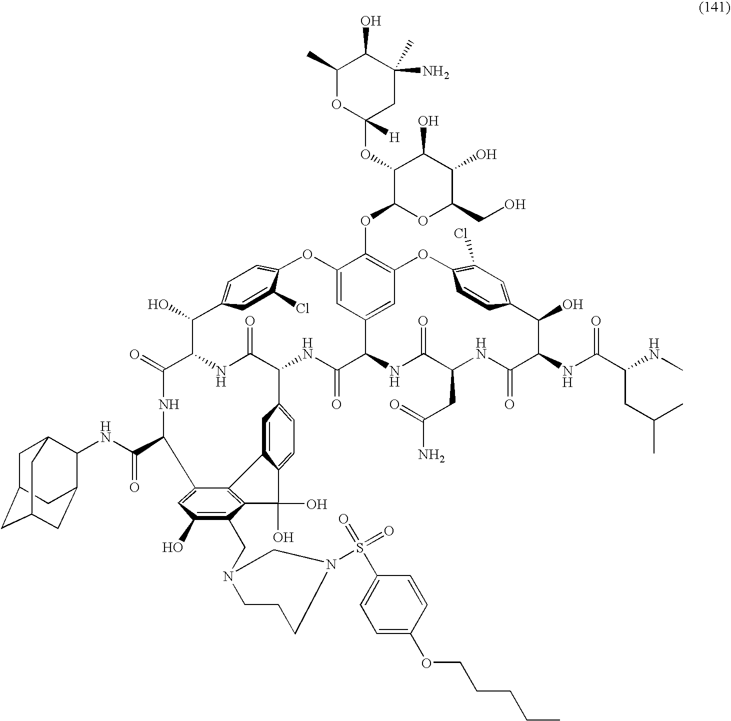

Using a procedure similar to the preparation of Compound (140), as in Example 80 and replacing Compound (71) with Compound (25), Compounds (141) was made.

### Example 82

### Synthesis of Compound (142)

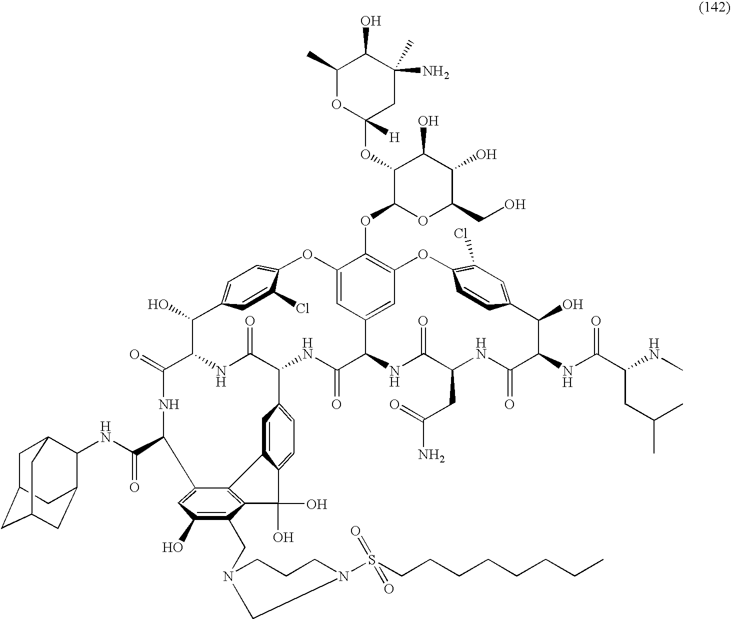

Using a procedure similar to the preparation of Compound (75), as in Example 64 and replacing N-(6-aminohexyl)-4-(pentyloxy)benzenesulfonamide with N-(2-aminoethyl)octane-1-sulfonamide, Compounds (142) is prepared.

### Example 83

### Synthesis of Compound (143)

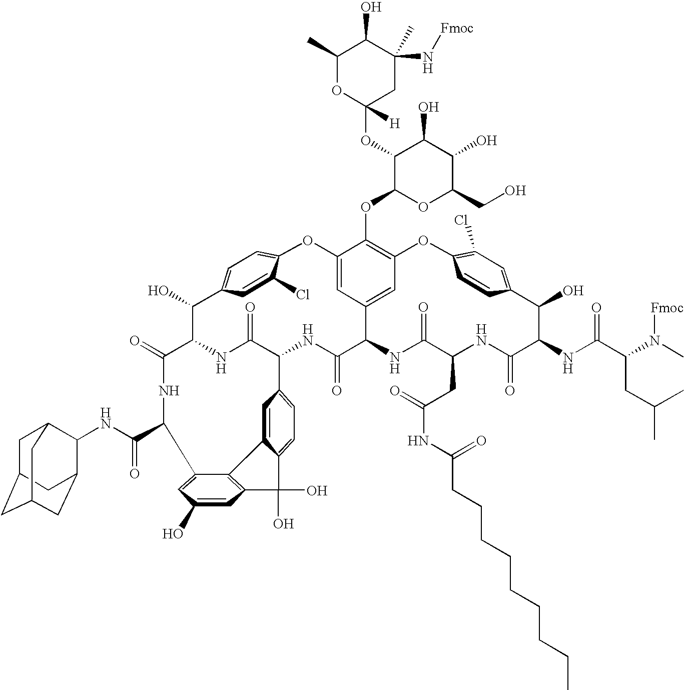

A solution of decanoic acid (0.8 g) in THF (8 ml) was treated with CDI (1.0 ml) at 0° C. The mixture was stirred at room temperature for 2 h. The resulting mixture was added into a solution of Compound (25) (0.62 g) in DMF (7 ml) at room temperature. The mixture was analyzed by analytical HPLC. On the completion of the reaction, the mixture was diluted with ether (40 ml). The resulted solid was collected by filtration to afford 640 mg of Fmoc-acylamide product Compound (143) as a white powder.

### Example 84

### Synthesis of Compound (144)

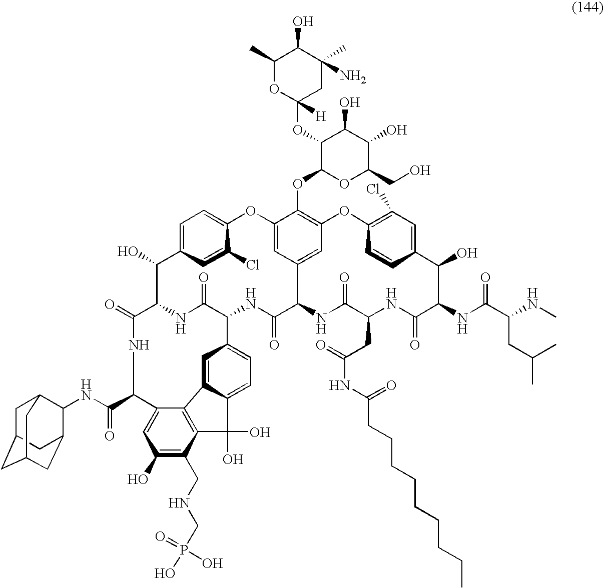

Using a procedure similar to the preparation of Compound (75), as in Example 64 and replacing Compound (25) with Compound (143) and replacing N-(6-aminohexyl)-4-(pentyloxy)benzenesulfonamide with aminomethylphosphonic acid, Compounds (144) is prepared.

### Example 85

### Synthesis of Compound (145)

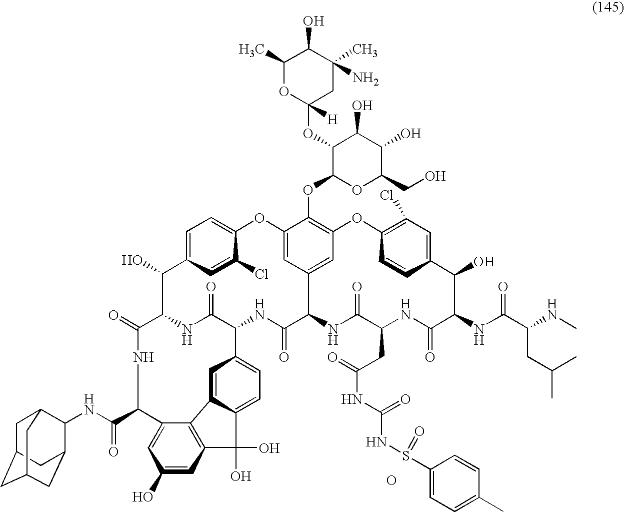

To a solution of Compound (25) (200 mg, 0.1 mmol) in dry DMF (1 mL) was added dropwise p-toluenesulfonyl isocyanate through a syringe at room temperature with stirring. The reaction was analytical HPLC in conjunction with ESI-MS. After stirred overnight at room temperature, the reaction mixture was poured into ether. The formed solid (about 210 mg) was collected by filtration under reduced pressure, which proved to be a mixture of multiple addition products. The mixture was directly treated with diethylamine (73 mg, 1 mmol, 10 eq.) in dry DMF (1 mL) at room temperature overnight. The reaction mixture was poured into ether. The resulting solid was collected by filtration under reduced pressure and washed with ether. The solid was isolated by reversed phase preparative HPLC(C18, 250×22 mm, ACN—H₂O containing 0.03% TFA, 10 ml/min) to give Compound (145) (6.02 mg, 3.4%) as a white solid. ESI-MS m/z, [M+H]⁺=1780.7, Found: 1780.7; [M−H]⁺=1777.7, Found: 1777.1.

### Example 86

### Synthesis of Compound (146)

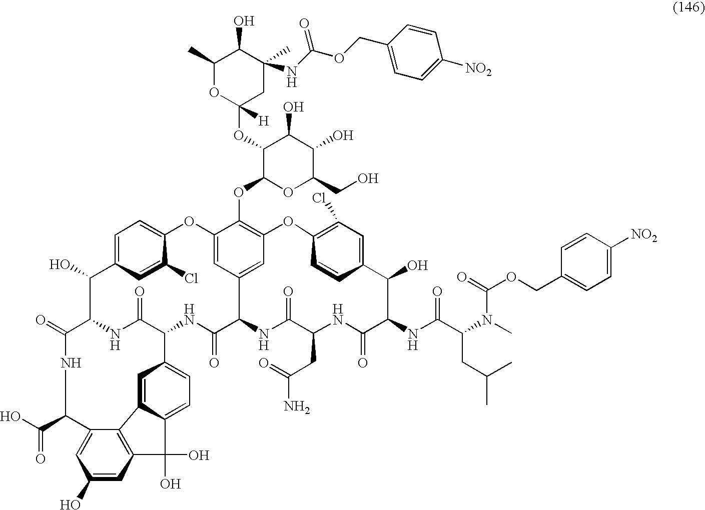

To a mixture solution of vancomycin hydrochloride (100.0 g, 67.3 mmol) and NaHCO₃ (28.3 g, 336.9 mmol) in THF (700 ml) and water (500 ml) was added a solution of pNZ—OSu (56.2 g, 191.2 mmol) in THF (200 ml) with stirring at 0° C. for 1 h. The reaction mixture was stirred at room temperature for 2 hr. and the organic layer was separated and the volatile was removed under reduce pressure. The resulting solid was collected by filtration under vacuum and washed with EtOAc and ether, dried under vacuum at 40° C. giving 130 g of compound (146) as a solid. ESI-MS: m/z: calcd for C₉₂H₁₁₆C₁₂N₁₄O₂₇ [M+H]+ 1921.89; Found: 1921.5 (33.1%), 1281.1 (28.5%), 961.1 (100%); [M+CF₃COO]−2033.5; Found: 2033.6 (100%).

### Example 87

### Synthesis of Compound (147)

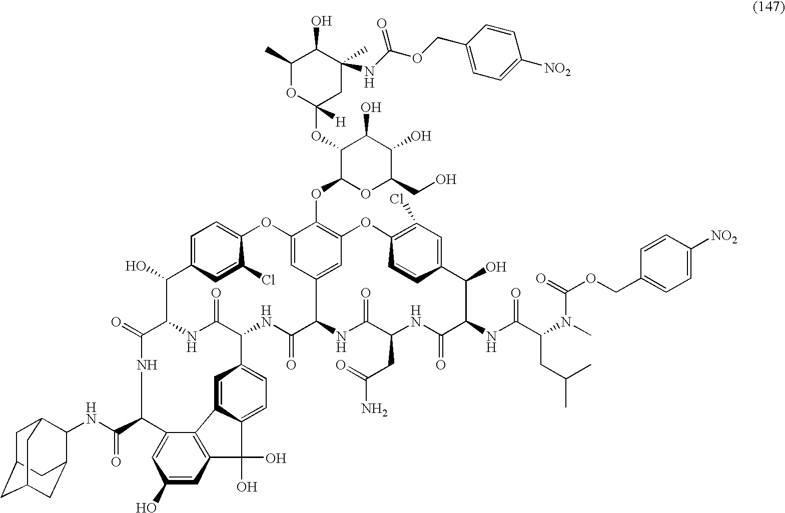

To a solution of compound (146) from the previous experiment (130 g) in DMSO (1000 ml) was added 2-adamantylamine hydrochloride (24.3 g, 129.5 mmol), DIPEA (46.47 g, 360.2 mmol) and HATU (54.69 g, 143.8 mmol) with stirring at room temperature. The reaction mixture was stirred overnight. Analytical HPLC showed the reaction completed. The reaction mixture was poured into ice-water (2000 ml). A precipitate was formed and collected by filtration. The solid was purified by column chromatography (silica gel, CH₃OH-DCM=1:9-1:5). The collected fraction was condensed to provide compound (147) (76 g, 58.2% yield from vancomycin hydrochloride) as white powder. ESI-MS: m/z: calcd for C₉₂H₁₀₀C₁₂N₁₂O₃₁ [M+H]+ 1941.75; Found: 1941.8 (100%); [M+CF₃COO]−2053.75; Found: 2053.8 (100%).

### Example 88

### Synthesis of Compound (148)

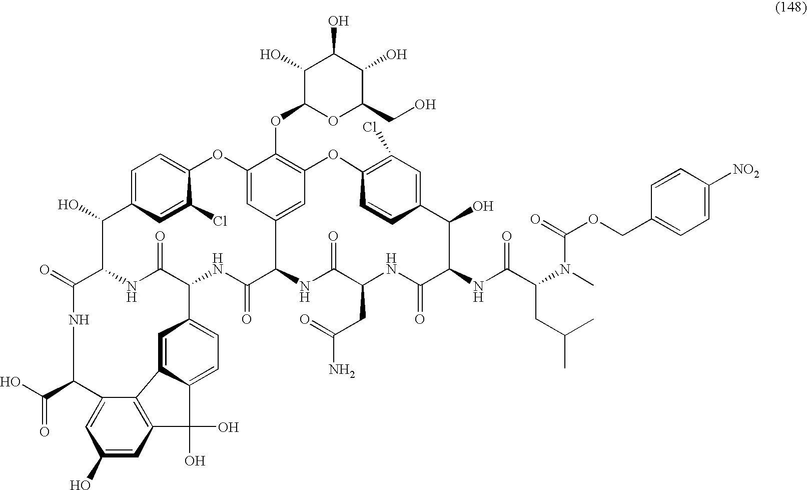

Using a procedure similar to the preparation of Compound (146) as in Example 86 and replacing vancomycin hydrochloride with Compound (1), Compound (148) was prepared.

### Example 89

### Synthesis of Compound (149)

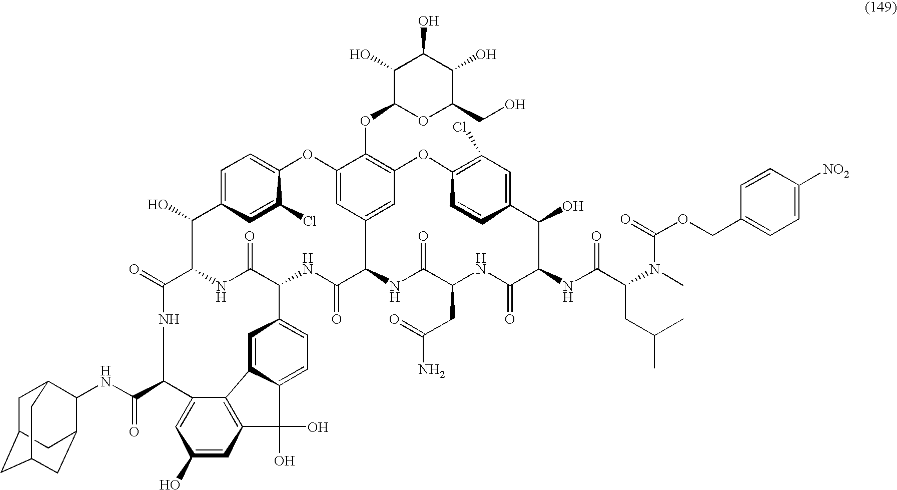

Using a procedure similar to the preparation of Compound (147) as in Example 87 and replacing Compound (146) with Compound (148), Compound (149) was made.

### Example 90

### Synthesis of Compound (150)

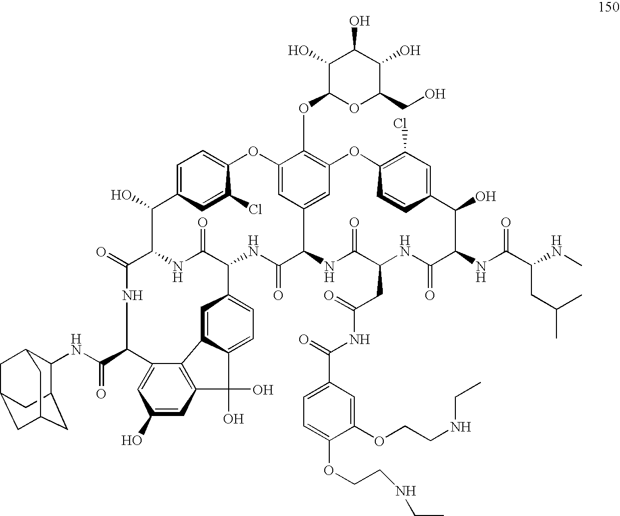

To a solution of 3,4-bis(2-(ethyl((4-nitrobenzyloxy)carbonyl)amino)ethoxy)benzoic acid (0.8 g) in DMF (15 ml) was slowly added DIC (0.5 ml) at 40° C. After the reaction mixture was stirred at 40° C. for 3 hr, Compound (149) (0.8 g) and DMAP (0.2 g) was added at this temperature. The resulting mixture was stirred at 40° C. overnight. The analytical HPLC monitoring showed the reaction completed. The solvent was removed under reduce pressure and ether was added. The formed crude product (1 g) was collected by filtration and dried under vacuum. The crude solid was dissolved in DMF (20 ml) was poured into a buffer solution of (20 ml) DMF-H₂O (3/2) containing N-methylmorpholine (0.68 g) and acetic acid (0.28 g) (pH 6.0). The resulting biphasic reaction mixture was hydrogenated over 5% Pd/C (0.5 g) at RT for overnight under 1 atm. The reaction was monitored by analytical HPLC. The reaction mixture was filtered and washed with DMF. The filtrate was concentrated and the residue was solidified with Et₂O. The solid was collected by filtration and purified by RP-HPLC to provide Compound (150) (30 mg). ESI-MS: m/z: calcd for C₈₄H₉₉Cl₂N₁₁O₂₄ [M+H]+ 1718.65; Found: 1718.4 (29.7%), 1145.9 (14.8%), 859.7 (100%); [M+CF3COO]−1830.65; Found: 1830.5 (100%).

### Example 91

### Synthesis of Compound (151)

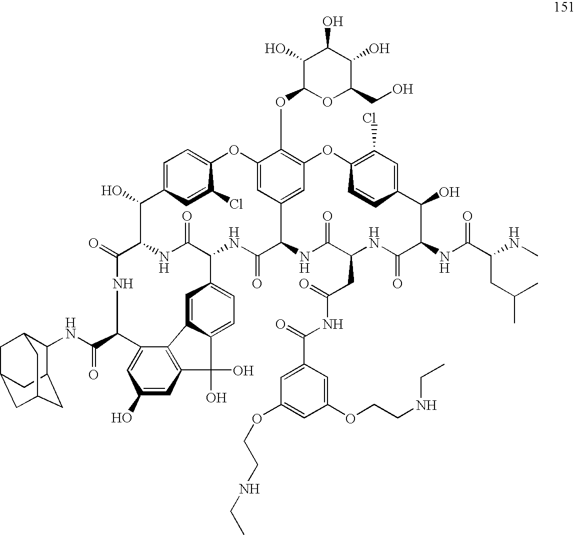

Using a procedure similar to the preparation of Compound (150), as in Example 90 and replacing 3,4-bis(2-(ethyl((4-nitrobenzyloxy)carbonyl)amino)ethoxy)benzoic acid with 3,5-bis(2-(ethyl((4-nitrobenzyloxy)carbonyl)amino)ethoxy)benzoic acid, Compounds (151) was made. Compounds (151) ESI-MS: m/z: calcd for C₈₄H₉₉Cl₂N₁₁O₂₄ [M+H]+ 1718.65; Found: 1718.5 (32.9%), 1145.9 (11.4%), 859.2 (100%); [M+CF3COO]−1830.65; Found: 1830.5 (100%).

### Example 92

### Synthesis of Compound (152)

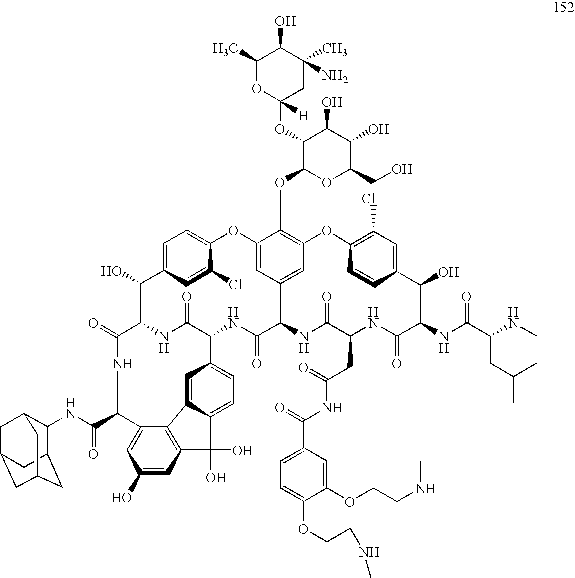

To a solution of 3,4-bis(2-(methyl((4-nitrobenzyloxy)carbonyl)amino)ethoxy)benzoic acid (3.3 g) in THF (60 ml) was slowly added DIC (945 mg) at RT. After the reaction mixture was stirred at room temperature for 2 hr, a solution of Compounds (25) (1.2 g) in DMF (7 ml) was added at room temperature. The resulting mixture was stirred at 80-90° C. overnight. The reaction was monitored by analytical HPLC. The reaction mixture was poured into MTBE. The formed crude product was collected by filtration and was dissolved into DMF (8 ml). Diethylamine (280 mg) was added at room temperature and the reaction mixture was stirred for 2 hr. The reaction was monitored by analytical HPLC. The solvent was removed under reduce pressure and ether was added. The formed solid was collected by filtration. The crude solid dissolved in DMF (30 ml) was poured into a buffer (80 ml) of DMF-H₂O (3/2) containing N-methylmorpholine (2.72 g) and acetic acid (1.12 g) (pH 6.0). The resulting biphasic reaction mixture was hydrogenated over 5% Pd/C (0.8 g) at room temperature for overnight under 1 atm. The reaction was monitored by analytical HPLC. The reaction mixture was filtered and washed with DMF. The filtrate was concentrated and the residue was solidified with Et₂O. The solid was collected by filtration and purified by RP-HPLC to provide Compounds (152) (35 mg). ESI-MS: m/z: calcd for C₈₉H₁₀₈Cl₂N₁₂O₂₆ [M+H]+ 1833.78; Found: 1733.5 (30%), 917.3 (100%); [M+CF3COO]−1945.78; Found: 1945.6 (95.1%), 1943.6 (100%).

### Example 93

### Synthesis of Compound (153)

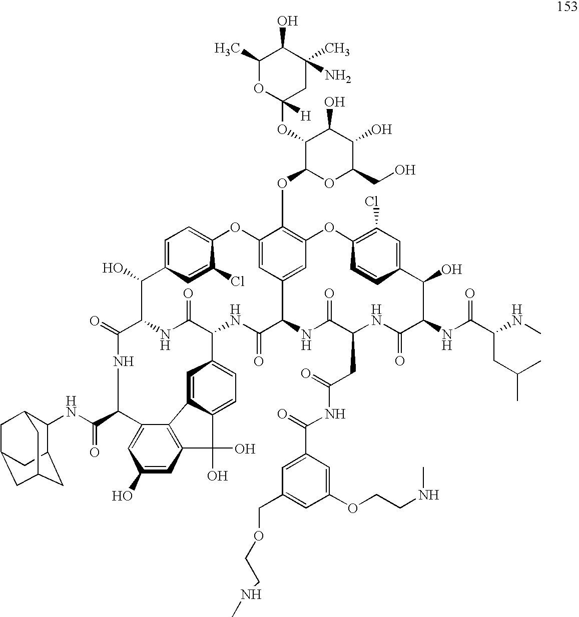

Using a procedure similar to the preparation of Compound (152), as in Example 92 and replacing 3,4-bis(2-(methyl((4-nitrobenzyloxy)carbonyl)amino)ethoxy)benzoic acid with 3,5-bis(2-(methyl((4-nitrobenzyloxy)carbonyl)amino)ethoxy)benzoic acid, Compounds (153) was made. ESI-MS: Compounds (153) m/z: calcd for C₈₉H₁₀₈Cl₂N₁₂O₂₆ [M+H]+ 1833.78; Found: 1733.5 (44.2%), 1222.6 (37.2%), 917.3 (100%); [M+CF3COO]−1945.78; Found: 1945.7 (100%).

### Example 94

### Synthesis of Compound (154)

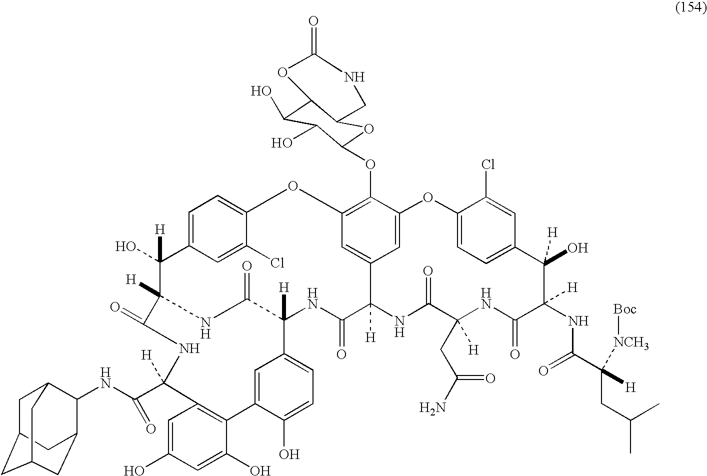

To a solution of Compound (96) (100 mg) in 3 ml of DMF was added CDI (15.8 mg, 1.5 eq) and TEA (19.7 mg, 3 eq). The mixture was stirred at 50° C. for 3 hours. Check completion by HPLC-MS. Then the solvent was removed under reduced pressure. The residue was purified by Prep-HPLC to afford Compound (154) (68 mg, yield=68%). LC-MS: 1563.5 (M+1).

### Example 95

### Synthesis of Compounds (155), (156), (157), and (158)

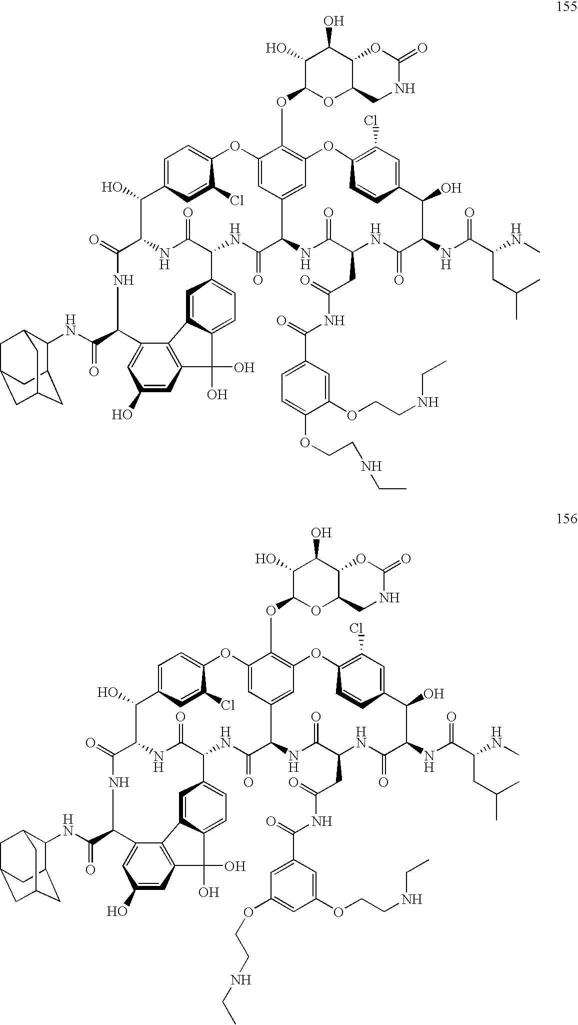

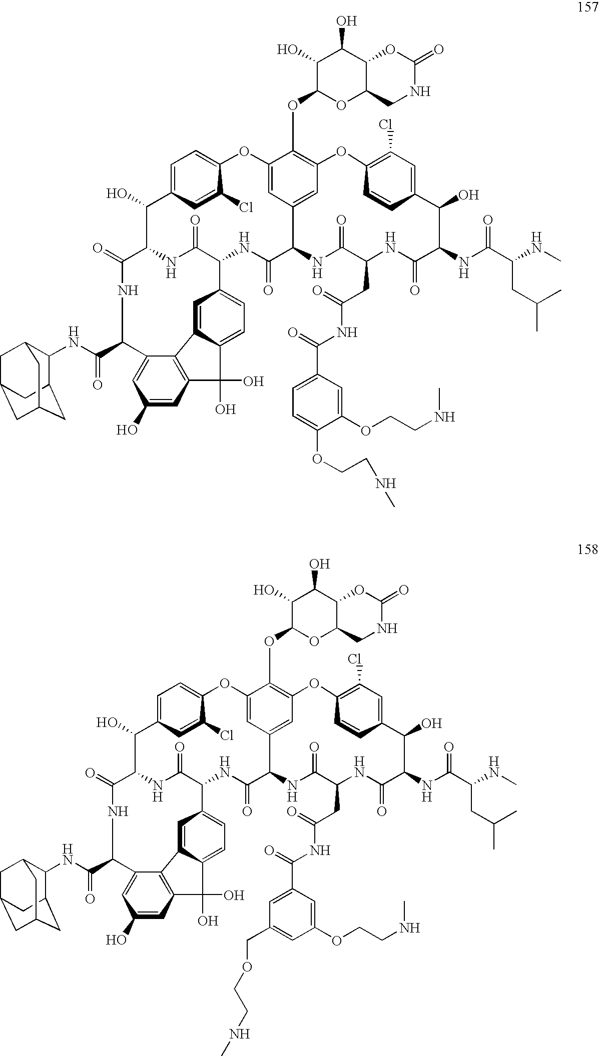

Using a procedure similar to the preparation of Compound (150), as in Example 90 and replacing Compound (149) with Compound (154) and either using 3,4-bis(2-(ethyl((4-nitrobenzyloxy)carbonyl)amino)ethoxy)benzoic acid or replacing it with 3,5-bis(2-(ethyl((4-nitrobenzyloxy)carbonyl)amino)ethoxy)benzoic acid or other similar benzoic acids, and dissolving the final product in 1;2 mixture of TFA and methylene dichloride and worked up after stirring for 6 hours Compounds (155), (156), (157), and (158), are made.

### Example 96

### Synthesis of Compounds (159), 160), (161), (162), and (163)

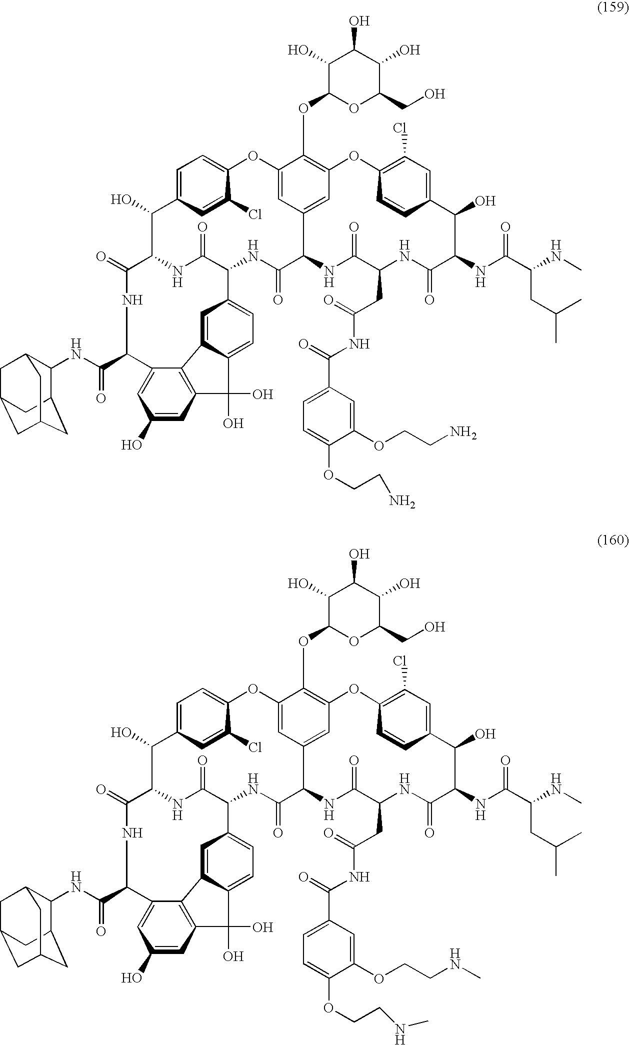

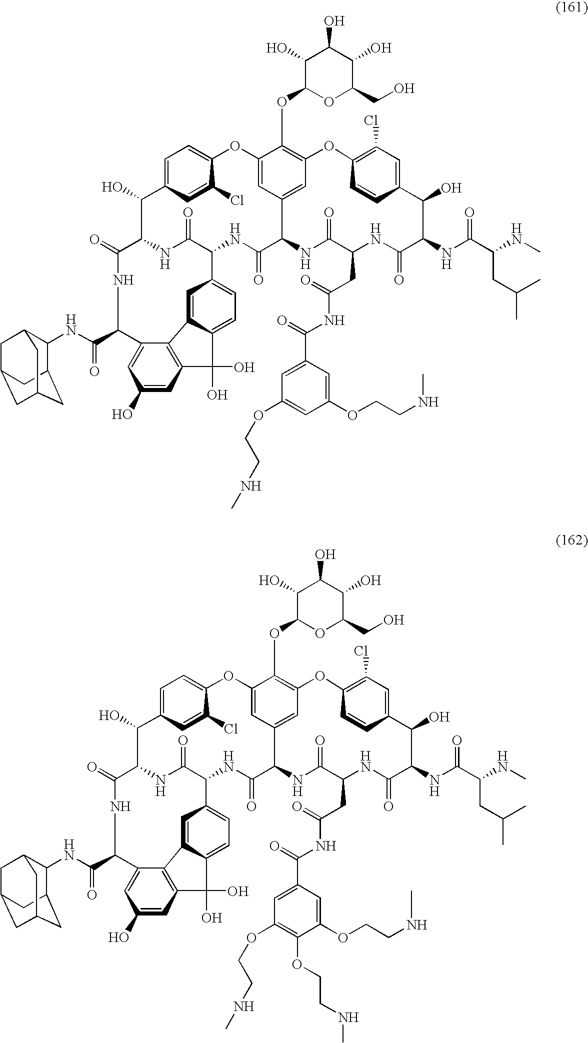

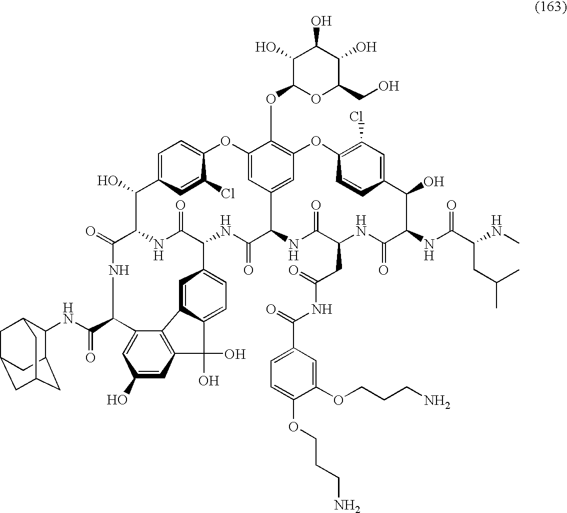

Using a procedure similar to the preparation of Compound (150), as in Example 90 and replacing 3,4-bis(2-(ethyl((4-nitrobenzyloxy)carbonyl)amino)ethoxy)benzoic acid with various similar protected substituted benzoic acids, (159), 160), (161) (162), and (163) were made.

### Example 97

### Synthesis of Compounds (164), 165) and (166)

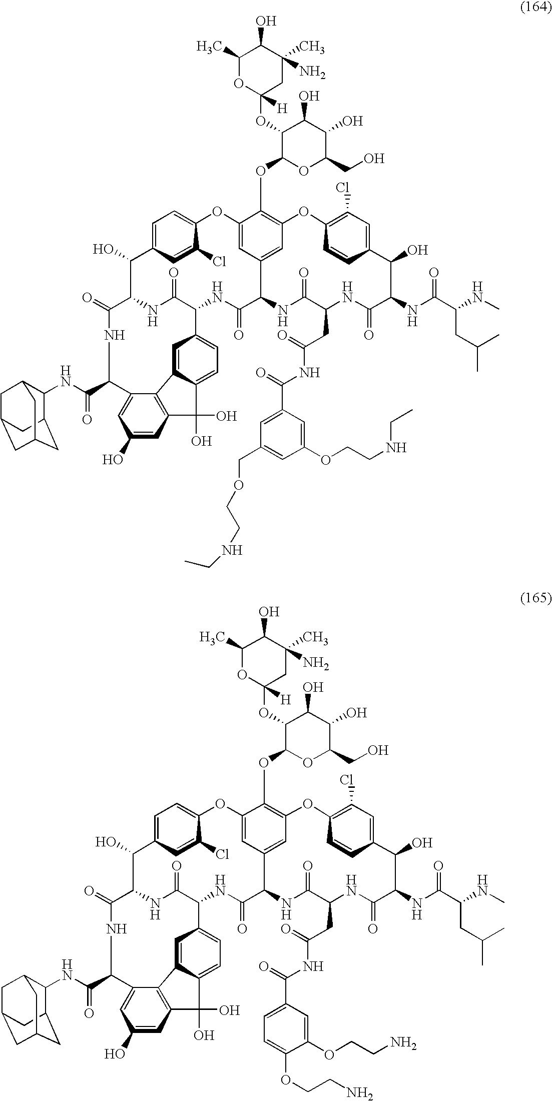

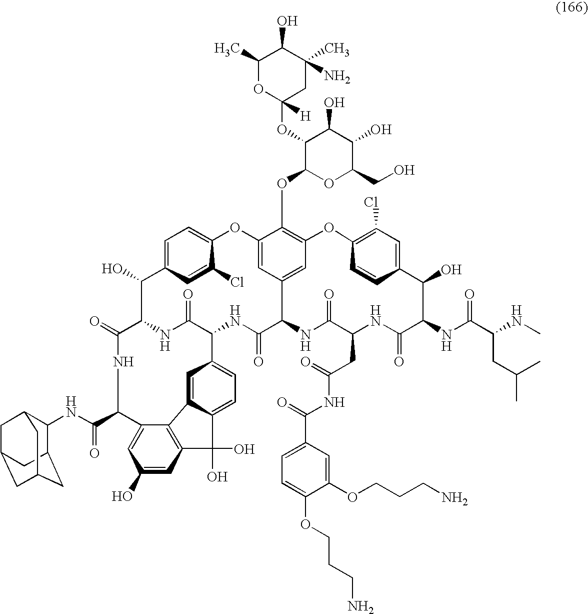

Using a procedure similar to the preparation of Compound (152), as in Example 92 and replacing 3,4-bis(2-(methyl((4-nitrobenzyloxy)carbonyl)amino)ethoxy)benzoic acid with various similar protected substituted benzoic acids, Compounds (164), 165) and (166) were made.

### Antibacterial Evaluation

Antibacterial activity in vitro is investigated by broth microdilution method in Meuller-Hinton broth as recommended by NCCLS. All strains tested are clinical isolates either sensitive or resistant to natural glycopeptides. MIC values were determined using the CLSI-recommended broth microdilution procedure (Clinical and Laboratory Standards Institute, Methods for Dilution Antimicrobial Susceptibility Tests for Bacteria That Grow Aerobically; Approved Standard-Seventh Edition). Automated liquid handlers (Multidrop 384, Labsystems, Helsinki, Finland; Biomek 2000 and Multimek 96, Beckman Coulter, Fullerton Calif.) were used to conduct serial dilutions and liquid transfers. MIC data for representative glycopeptides derivatives made and described in this application are summarized in Tables 1 and 2. The MIC value for vancomycin is given for comparison. The abbreviations for organisms tested are as follow: SA 100—Staphylococcus aureus 100 (ATCC 29213); SA 757—Staphylococcus aureus 757 (MRSA); SA 2012—Staphylococcus aureus 2012 (VISA); SE 835—Staphylococcus epidermidis 835; SE 831—Staphylococcus epidermidis 831 (MRSE); EFc 101—Enterococcus faecalis 101 (ATCC 29212); EFc 848—Enterococcus faecalis 848 (VRE, Van A); EFcm 800—Enterococcus faecium 800; EFcm 752—Enterococcus faecium 752 (VRE, Van A); SPNE 1195—Streptococcus pneumoniae 1195 (ATCC 49619); SPY 712—Streptococcus pyogenes 712.

### Biological Data

Most of the glycopeptides derivatives in Tables 1 and 2 are very potent and have activity against Streptococcus pneumoniae and MRSA, clinical important pathoges. Many derivatives have activity against vancomycin resistant bacteria such as VISA (vancomycin intermediate-resistant Staphylococcus aureus), and vancomycin resistant enterococci.

TABLE 1

| | SA | SA | SA | SE | SE | E FC | E FC | E FCM | E FCM | S PNE | S PYO |
|---|---|---|---|---|---|---|---|---|---|---|---|
| Compound | 100 | 757 | 2012 | 835 | 831 | 101 | 848 | 800 | 752 | 1195 | 712 |
| 30 | A | A | D | B | A | A | C | A | B | A | A |
| 42 | A | A | C | A | A | A | C | A | A | A | A |
| 43 | A | B | C | A | A | A | C | A | A | A | A |
| 103 | B | A | C | A | A | A | D | A | C | B | A |
| 102 | A | A | B | A | A | A | D | A | B | A | A |
| 101 | A | A | A | A | A | A | C | A | A | A | A |
| 99 | A | A | B | A | A | A | B | A | A | B | A |
| 31 | B | B | D | A | A | A | D | A | B | A | A |
| 32 | C | C | D | B | A | A | D | A | B | A | A |
| 33 | C | C | E | A | A | A | D | A | B | A | A |
| 117 | A | A | D | A | A | A | D | A | C | A | A |
| 118 | D | C | G | C | C | B | D | A | C | A | A |
| 44 | B | B | D | A | A | B | G | A | G | A | A |
| Vancomycin | 1 | 1 | 8 | 4 | 2 | 4 | >64 | 1 | >64 | 0.25 | 0.5 |

TABLE 2

| | SA | SA | SA | SE | SE | E FC | E FC | E FCM | E FCM | S PNE | S PYO |
|---|---|---|---|---|---|---|---|---|---|---|---|
| Compound | 100 | 757 | 2012 | 835 | 831 | 101 | 848 | 800 | 752 | 1195 | 712 |
| 106 | C | C | C | A | A | B | E | A | C | C | A |
| 63 | A | A | D | A | A | A | E | A | C | A | A |
| 62 | A | A | D | A | A | A | E | A | B | A | A |
| 46 | A | A | D | A | A | A | D | A | A | A | A |
| 65 | B | A | D | B | A | A | E | A | C | A | A |
| 67 | A | A | C | C | A | A | E | A | C | NR | A |
| 163 | C | C | C | C | A | C | G | B | E | B | A |
| 166 | A | A | C | B | A | A | G | A | D | A | A |
| 165 | A | A | C | B | A | A | G | A | D | A | A |
| 160 | C | C | C | B | A | B | G | A | E | B | A |
| 162 | D | C | D | B | A | C | G | B | F | C | A |
| 152 | A | A | C | A | A | A | G | A | E | A | A |
| 159 | B | B | B | B | A | B | G | A | E | B | A |
| 153 | A | A | B | A | A | A | G | A | G | A | A |
| 164 | A | A | C | A | A | A | G | A | F | A | A |
| Vancomycin | 1 | 2 | 4 | 2 | 2 | 4 | >64 | 1 | >64 | 0.25 | 0.5 |

MIC values determined in the presence of 0.002% Polysorbate 80.

MIC (μg/mL)

### 0.01<A≦0.5

### 0.5<B≦1.0

### 1.0<C≦2.0

### 2.0<D≦4.0

### 4.0<E≦8.0

### 8.0<F≦16.0

### 16.0<G

Clinical Trial of the Safety and Efficacy of Compounds of Formula (I)-(V) in Patients with C. difficile-Associated Diarrhea

Purpose: This study aims to determine the safety and efficacy of glycopeptide compounds presented herein for the treatment of symptoms of C. difficile-associated diarrhea and lowering the risk of repeat episodes of diarrhea. The compounds are evaluated in comparison to current standard antibiotic treatment, so all patients will receive active medication. All study-related care is provided including doctor visits, physical exams, laboratory tests and study medication. Total length of participation is approximately 10 weeks.

Patients: Eligible subjects will be men and women 18 years and older.

### Criteria:

Inclusion Criteria:

Be at least 18 years old;

Have active mild to moderate C. difficile-Associated Diarrhea (CDAD);

Be able to tolerate oral medication;

Not be pregnant or breast-feeding; and

Sign and date an informed consent form.

Study Design: This is a randomized, double-blind, active control study of the efficacy, safety, and tolerability of a compound of Formula (I)-(V) in patients with C. difficile-associated diarrhea.

Clinical Trial Comparing a Compound of Formula (I)-(V) with Vancomycin for the Treatment of MRSA Osteomyleitis

Purpose: This study aims to determine the efficacy of glycopeptide compounds presented herein as compared to vancomycin for the treatment of methicillin-resistant Staphylococcus aureus (MRSA) osteomyelitis.

Patients: Eligible subjects will be men and women 18 years and older.

### Criteria:

Inclusion Criteria:

Culture-proven MRSA, obtained in operating room or sterile biopsy procedure from bone site. The infection and sampling site is either within the bone or a deep soft-tissue site that is contiguous with bone; OR radiographic abnormality consistent with osteomyelitis in conjunction with a positive blood culture for MRSA;

Surgical debridement of infection site, as needed;

Subject is capable of providing written informed consent; and

Subject capable of receiving outpatient parenteral therapy for 12 weeks.

Exclusion Criteria:

Hypersensitivity to a compound of Formula (I)-(V) or vancomycin;

S. aureus resistant to a compound of Formula (I)-(V) or vancomycin;

Osteomyelitis that develops directly from a chronic, open wound;

Polymicrobial culture (the only exception is if coagulase-negative staphylococcus is present in the culture and the clinical assessment is that it is a contaminant);

Subject has a positive pregnancy test at study enrollment;

Baseline renal or hepatic insufficiency that would preclude administration of study drugs;

Active injection drug use without safe conditions to administer intravenous antibiotics for 3 months; and

Anticipated use of antibiotics for greater than 14 days for an infection other than osteomyelitis.

Study Design: This is a randomized, open-label, active control, efficacy trial comparing vancomycin with a compound of Formula (I)-(V) for the treatment of MRSA Osteomyelitis.

### Clinical Trial Evaluating a Compound of Formula (I)-(V) in Selected Serious Infections Caused by Vancomycin-Resistant Enterococcus (VRE)

Purpose: This study aims to determine the safety and efficacy of a compound of Formula (I)-(V) in the treatment of selected serious infections caused by VRE.

Patients: Eligible subjects will be men and women 18 years and older.

### Criteria:

Inclusion Criteria:

Isolation of one of the following multi-antibiotic resistant bacteria: vancomycin-resistant Enterococcus faecium, vancomycin-resistant Enterococcus faecalis alone or as part of a polymicrobial infection; and

Have a confirmed diagnosis of a serious infection (eg, bacteremia [unless due to an excluded infection], complicated intra-abdominal infection, complicated skin and skin structure infection, or pneumonia) requiring administration of intravenous (IV) antibiotic therapy.

Exclusion Criteria:

Subjects with any concomitant condition or taking any concomitant medication that, in the opinion of the investigator, could preclude an evaluation of a response or make it unlikely that the contemplated course of therapy or follow-up assessment will be completed or that will substantially increase the risk associated with the subject's participation in this study

Anticipated length of antibiotic therapy less than 7 days

Study Design: This is a randomized, double-blind, safety and efficacy study of a compound of Formula (I)-(V) in the treatment of selected serious infections caused by VRE.

Although the foregoing embodiments have been described in some detail for purposes of clarity of understanding, it will be apparent that in some embodiments, certain changes and modifications are practiced within the scope of the appended claims. The present embodiments are to be considered as illustrative and not restrictive, and the aspects described herein are not to be limited to the details given herein, but in some embodiments are modified within the scope and equivalents of the appended claims.

What is claimed is:

1. A compound having a structure selected from the group consisting of Formulas (I-V): 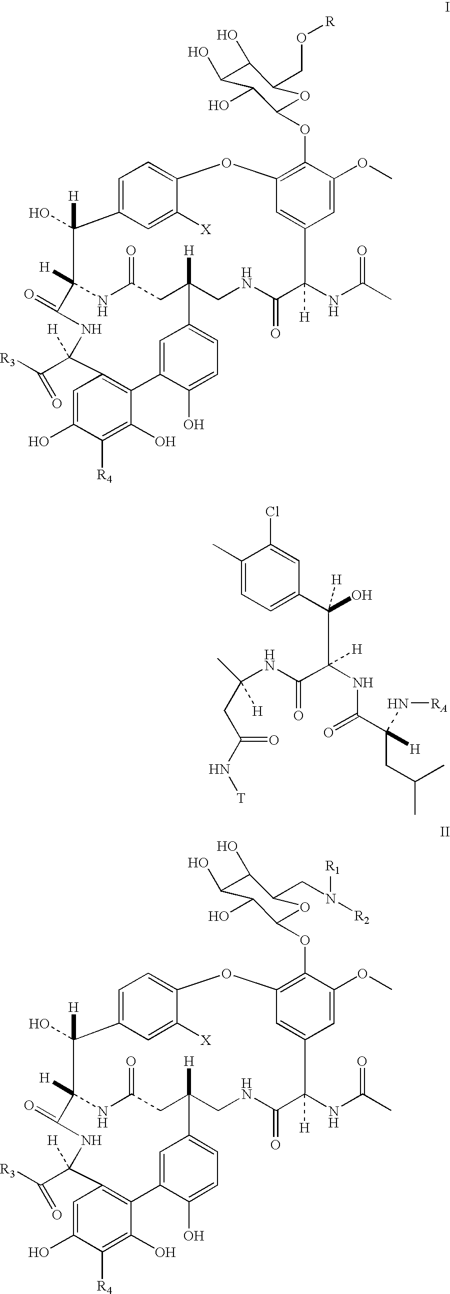 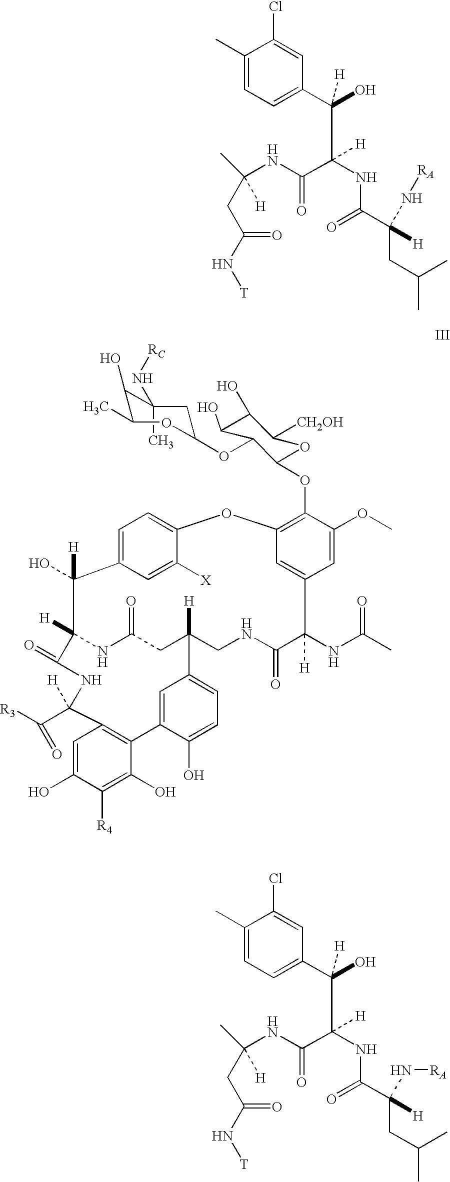 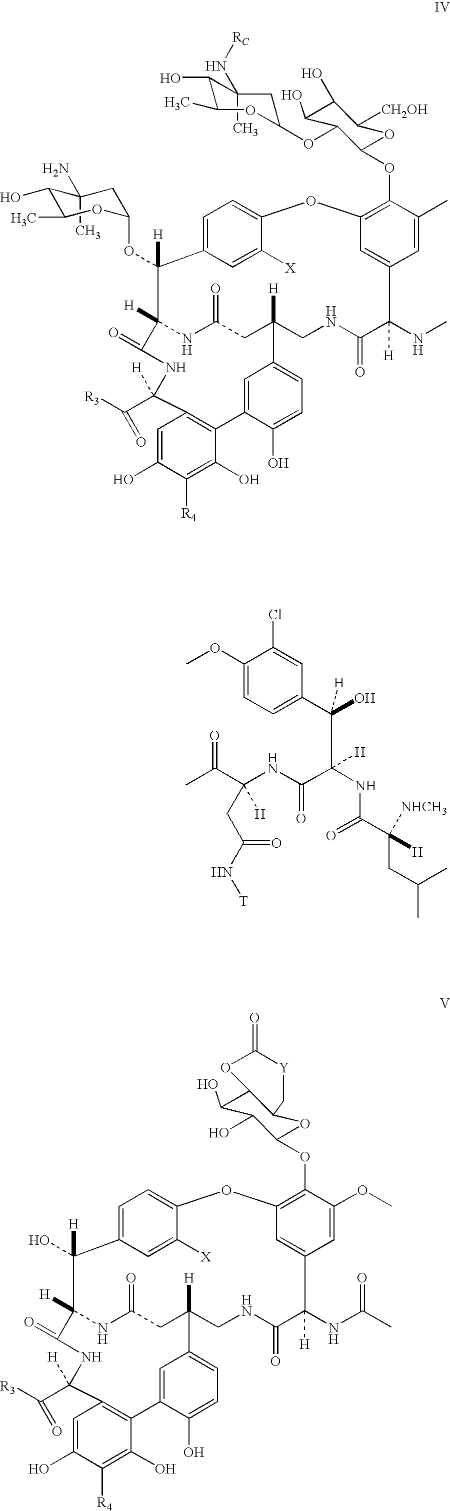 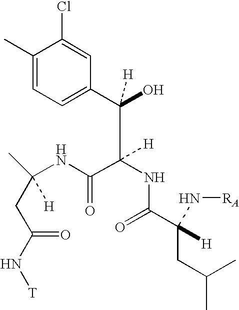
wherein,
R_{A} is selected from the group consisting of
a) hydrogen,
b) methyl,
c) C₂-C₁₂-alkyl;
R₁ and R₂ are each independently selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
or
R₁ and R₂ taken together with the atom to which they are attached form a substituted heteroaryl or 3-10 membered heterocycloalkyl ring which optionally contain one or two hetero functionalities selected from the group consisting of —O—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, —S—, and S(O)ₙ— wherein n is 1 or 2 and the 3-10 membered heterocycloalkyl ring is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) C₁-C₃-haloalkyl,
(h) C₁-C₃-alkoxy-C₁-C₃-alkyl,
and
k) C(═O)R₇,
l) C(═O)CHR₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
or
R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) C₁-C₃-haloalkyl,
(h) C₁-C₃-alkoxy-C₁-C₃-alkyl;
R₇ is selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C₁-C₁₂-alkylamino,
l) amino,
m) amino-cycloalkyl;
X is selected from the group consisting of
(1) hydrogen,
(2) chlorine;
Y is selected from the group consisting of
(1) oxygen,
(2) NR₁;
T is selected from the group consisting of
(1) —SO₂R_{B},
(2) —COR_{B},
(3) —CONHSO₂R_{B};
R is selected from the group consisting of
(1) hydrogen,
(2) cycloalkyl,
(3) cycloalkenyl,
(4) C₁-C₁₂-alkyl,
(5) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) —COOR₅ wherein R₅ is hydrogen or loweralkyl,
(f) —C(O)NR₅R₆ wherein R₆ is hydrogen or loweralkyl,
(g) amino,
(h) —NR₅R₆,
or
R₅ and R₆ taken together with the atom to which they are attached form a 3-10 membered heterocycloalkyl ring which is optionally substituted with one or more substituents independently selected from the group consisting of
(i) halogen.
(ii) hydroxy,
(iii) C₁-C₃-alkoxy,
(iv) C₁-C₃-alkoxy-C₁-C₃-alkoxy,
(v) oxo,
(vi) C₁-C₁₂-alkyl,
(vii) C₁-C₁₂haloalkyl,
and
(viii) C₁-C₃-alkoxy-C₁-C₁₂-alkyl,
(i) aryl,
(j) substituted aryl,
(k) heteroaryl,
(l) substituted heteroaryl,
(m) mercapto,
(n) C₁-C₁₂-thioalkoxy,
(6) C(═O)OR₁₁, wherein R₁₁ is hydrogen, loweralkyl, substituted loweralkyl, aryl, substituted aryl, heteroaryl or substituted heteroaryl,
(7) C(═O)NR₁₁R₁₂, wherein R₁₁ is as previously defined and R₁₂ is hydrogen, loweralkyl, substituted loweralkyl, aryl, substituted aryl, heteroaryl or substituted heteroaryl,
or
R₁₁ and R₁₂ together with the atom to which they are attached form a 3-10 membered heterocycloalkyl ring, which is optionally substituted with one or more substituents independently selected from the group consisting of
(a) halogen,
(b) hydroxy,
(c) C₁-C₃-alkoxy,
(d) C₁-C₃-alkoxy-C₁-C₃-alkoxy,
(e) oxo,
(f) C₁-C₁₂-alkyl,
(g) substituted loweralkyl,
(h) C₁-C₁₂haloalkyl,
(i) amino,
(j) alkylamino,
(k) dialkylamino
and
(l) C₁-C₃-alkoxy-C₁-C₁₂-alkyl,
or
R and its connected oxygen atom taken together is halogen;
R₃ is selected from the group consisting of
(1) OH,
(2) 1-adamantanamino,
(3) 2-adamantanamino,
(4) 3-amino-1-adamantanamino,
(5) 1-amino-3-adamantanamino,
(6) 3-loweralkylamino-1-adamantanamino,
(7) 1-loweralkylamino-3-adamantanamino,
(8) amino
(9) NR₁₃R₁₄ wherein R₁₃ and R₁₄ are each independently selected from the group consisting of hydrogen, loweralkyl, substituted loweralkyl, cycloalkyl, substituted cycloalkyl, aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy or
R₁₃ and R₁₄ together with the atom to which they are attached form a 3-10 membered heterocycloalkyl ring, which is optionally substituted with one or more substituents independently selected from the group consisting of
(a) halogen.
(b) hydroxy,
(c) C₁-C₃-alkoxy,
(d) C₁-C₃-alkoxy-C₁-C₃-alkoxy,
(e) oxo,
(f) C₁-C₁₂-alkyl,
(g) substituted loweralkyl,
(h) C₁-C₁₂haloalkyl,
(i) amino,
(j) alkylamino,
(k) dialkylamino
and
(l) C₁-C₃-alkoxy-C₁-C₁₂-alkyl;
R₄ is selected from the group consisting of
(1) CH₂NH—CHR₁₅—(CH₂)ₘ—NHSO₂R_{B}, wherein m is 1 to 6 and R₁₅ is H or loweralkyl,
(2) CH₂NH—CHR₁₅—(CH₂)ₚ—CONHSO₂R_{B}, wherein p is 0 to 6 and R₁₅ is H or loweralkyl,
(3) CH₂NH—CHR₁₅—(CH₂)ₚ—COOH, wherein p is 0 to 6 and R₁₅ is H or loweralkyl,
(4) CH₂NR_{D}—CHR₁₅—(CH₂)_{q}—NR_{E}SO₂R_{B}, wherein q is 2 to 4 and R₁₅ is H or loweralkyl, R_{D} and R_{E} together represents —CH₂—,
(5) H,
(6) CH₂NHCH₂PO₃H₂,
(7) aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy;
R_{B} is selected from the group consisting of
a) aryl,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) heteroaryl,
j) heterocycloalkyl,
k) aryl substituted with one or more substituents selected from the group consisting of
(a) halogen,
(b) hydroxy,
(c) C₁-C₁₂-alkoxy,
(d) C₁-C₆-alkoxy-C₁-C₆-alkoxy,
(e) amino,
(f) amino-C₁-C₆-alkoxy
(g) C₁-C₁₂-alkylamino,
(h) C₁-C₁₂-alkylamino-C₁-C₆-alkoxy,
(i) C₁-C₁₂-dialkylamino,
(j) C₁-C₁₂-dialkylamino-C₁-C₆-alkoxy,
(k) alkenyl,
(l) alkynyl,
(m) C₁-C₁₂-thioalkoxy,
(n) C₁-C₁₂-alkyl,
(o) C₁-C₁₂-substituted alkyl,
(p) C₁-C₁₂-alkoxy-morpholino,
(q) C₁-C₁₂-alkoxy-C₁-C₁₂-dialkoxyamino,
(r) C₁-C₁₂-alkoxy-NHSO₂ C₁-C₆-alkyl,
(s) C₁-C₁₂-alkoxy-NHCO C₁-C₆-alkyl;
l) heteroaryl substituted with one or more substituents selected from the group consisting of
(a) halogen,
(b) hydroxy,
(c) C₁-C₁₂-alkoxy,
(d) C₁-C₆-alkoxy-C₁-C₆-alkoxy,
(e) amino,
(f) amino-C₁-C₆-alkoxy
(g) C₁-C₁₂-alkylamino,
(h) C₁-C₁₂-alkylamino-C₁-C₆-alkoxy,
(i) C₁-C₁₂-dialkylamino,
(j) C₁-C₁₂-dialkylamino-C₁-C₆-alkoxy,
(k) alkenyl,
(l) alkynyl,
(m) C₁-C₁₂-thioalkoxy,
n) C₁-C₁₂-alkyl,
(o) C₁-C₁₂-substituted alkyl;
R_{C} is each independently selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C(═O)R₇,
l) C(═O)CHR₈NR₉R₁₀ wherein R₈, R₉ and R₁₀ are each independently selected from a group consisting of hydrogen, loweralkyl, substituted loweralkyl, aryl, substituted aryl, heteroaryl or substituted heteroaryl,
or
R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) C₁-C₃haloalkyl,
(h) C₁-C₃-alkoxy-C₁-C₃-alkyl;
or a pharmaceutically acceptable salt, ester, solvate, alkylated quaternary ammonium salt, stereoisomer, tautomer or prodrug thereof.

2. The compound of claim 1, wherein the compound has the Formula I 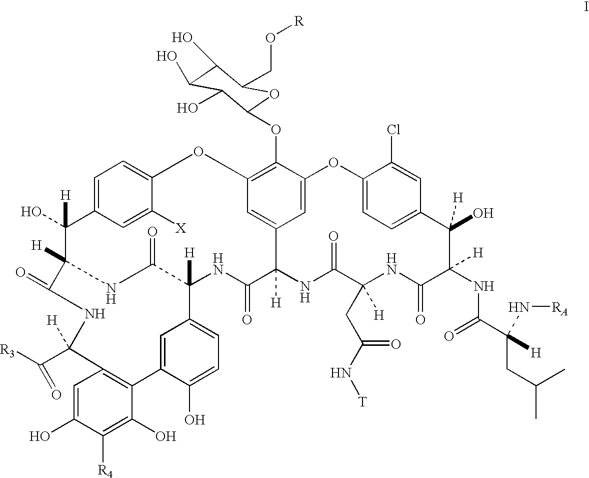
or a pharmaceutically acceptable salt, ester, solvate, alkylated quaternary ammonium salt, stereoisomer, tautomer or prodrug thereof.

3. The compound of claim 1, wherein the compound has the Formula II 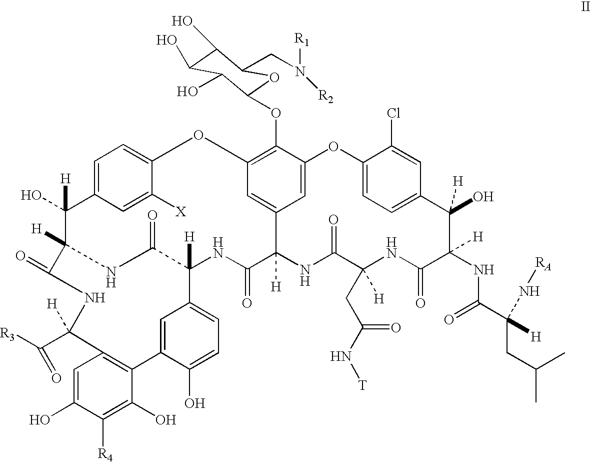
or a pharmaceutically acceptable salt, ester, solvate, alkylated quaternary ammonium salt, stereoisomer, tautomer or prodrug thereof.

4. The compound of claim 1, wherein the compound has the Formula III 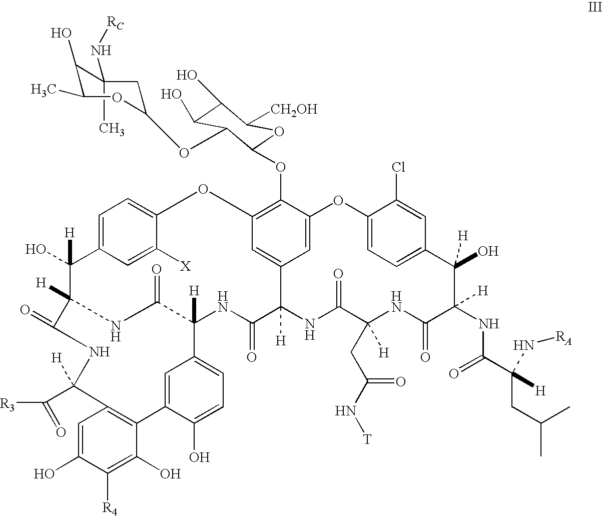
or a pharmaceutically acceptable salt, ester, solvate, alkylated quaternary ammonium salt, stereoisomer, tautomer or prodrug thereof.

5. The compound of claim 1, wherein the compound has the Formula IV 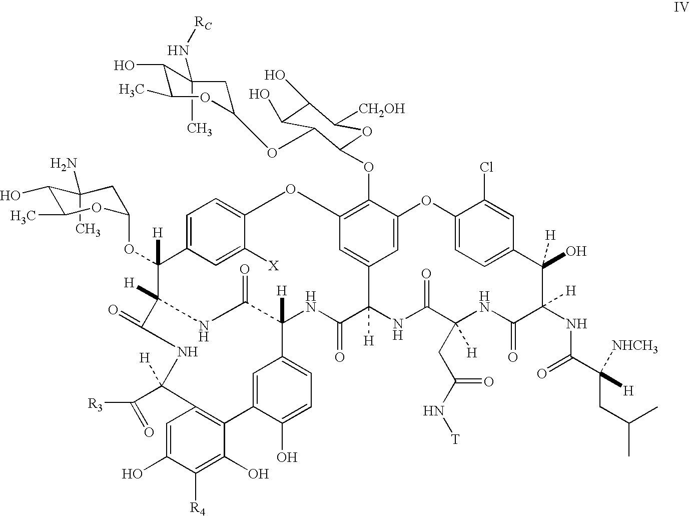
or a pharmaceutically acceptable salt, ester, solvate, alkylated quaternary ammonium salt, stereoisomer, tautomer or prodrug thereof.

6. The compound of claim 1, wherein the compound has the Formula V 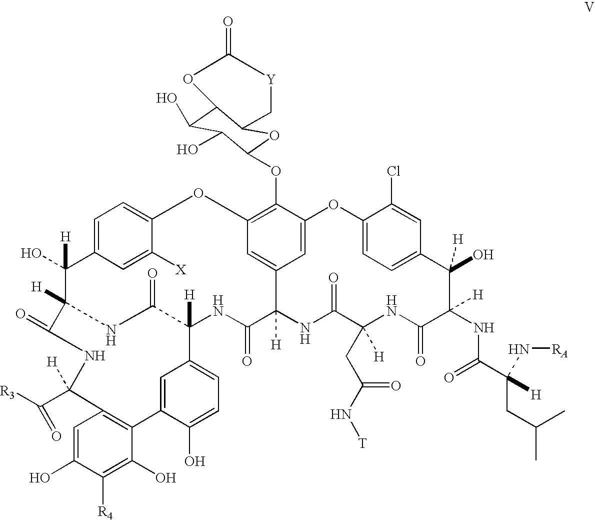
or a pharmaceutically acceptable salt, ester, solvate, alkylated quaternary ammonium salt, stereoisomer, tautomer or prodrug thereof.

7. The compound of claim 2, wherein R_{A} is hydrogen or methyl and R₄ is hydrogen.

8. The compound of claim 3, wherein R_{A} is hydrogen or methyl and R₄ is hydrogen.

9. The compound of claim 4, wherein R_{A} is hydrogen or methyl and R₄ is hydrogen.

10. The compound of claim 5, wherein X is hydrogen or chlorine and R₄ is hydrogen.

11. The compound of claim 6, wherein R_{A} is hydrogen or methyl and R₄ is hydrogen.

12. The compound of claim 1, wherein T is hydrogen and R₄ is CH₂NH—CHR₁₅—(CH₂)ₘ—NHSO₂R_{B}, wherein m is 1 to 6 and R₁₅ is H or loweralkyl.

13. The compound of claim 1, wherein R_{A} is hydrogen or methyl and T is —SO₂R_{B}.

14. The compound of claim 1, wherein R_{A} is hydrogen or methyl and T is —COR_{B}.

15. The compound of claim 1, wherein R_{A} is hydrogen or methyl and T is —CONHSO₂R_{B}.

16. The compound of claim 1 wherein R₃ is each selected from the group consisting of
(1) OH,
(2) 1-adamantanamino,
(3) 2-adamantanamino,
(4) 3-amino-1-adamantanamino,
(5) 1-amino-3-adamantanamino,
(6) 3-loweralkylamino-1-adamantanamino,
(7) 1-loweralkylamino-3-adamantanamino,
(8) amino
(9) NR₁₃R₁₄ wherein R₁₃ and R₁₄ are independently selected from the group consisting of hydrogen, loweralkyl, substituted loweralkyl, cycloalkyl, substituted cycloalkyl, aminoloweralkyl wherein the amino portion of the aminoloweralkyl group is further substituted with unsubstituted or substituted alkyl, alkenyl, cycloalkyl, cycloalkenyl, arylaryl, alkoxy, aryloxy, substituted alkoxy, and substituted aryloxy
or
R₁₃ and R₁₄ together with the atom to which they are attached form a 3-10 membered heterocycloalkyl ring, which is optionally substituted with one or more substituents independently selected from the group consisting of
(a) halogen,
(b) hydroxy,
(c) C₁-C₃-alkoxy,
(d) C₁-C₃-alkoxy-C₁-C₃-alkoxy,
(e) oxo,
(f) C₁-C₁₂-alkyl,
(g) substituted loweralkyl,
(h) halo-C₁-C₁₂-alkyl,
(i) amino,
(j) alkylamino,
(k) dialkylamino
and
(l) C₁-C₃-alkoxy-C₁-C₁₂-alkyl.

17. The compound of claim 1, wherein R_{B} is each selected from the group consisting of
a) aryl,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) heteroaryl,
j) heterocycloalkyl,
k) aryl substituted with one or more substituents selected from the group consisting of
(a) halogen,
(b) hydroxy,
(c) C₁-C₁₂-alkoxy,
(d) C₁-C₆-alkoxy-C₁-C₆-alkoxy,
(e) amino,
(f) amino-C₁-C₆-alkoxy,
(g) C₁-C₁₂-alkylamino,
(h) C₁-C₁₂-alkylamino-C₁-C₆-alkoxy,
(i) C₁-C₁₂-dialkylamino,
(j) C₁-C₁₂-dialkylamino-C₁-C₆-alkoxy,
(k) alkenyl,
(l) alkynyl,
(m) C₁-C₁₂-thioalkoxy,
(n) C₁-C₁₂-alkyl,
(o) C₁-C₁₂-substituted alkyl,
(p) C₁-C₁₂-alkoxy-morpholino,
(q) C₁-C₁₂-alkoxy-C₁-C₁₂-dialkoxyamino,
(r) C₁-C₁₂-alkoxy-NHSO₂ C₁-C₆-alkyl,
(s) C₁-C₁₂-alkoxy-NHCO C₁-C₆-alkyl;
l) heteroaryl substituted with one or more substituents selected from the group consisting of
(a) halogen,
(b) hydroxy,
(c) C₁-C₁₂-alkoxy,
(d) C₁-C₆-alkoxy-C₁-C₆-alkoxy,
(e) amino,
(f) amino-C₁-C₆-alkoxy,
(g) C₁-C₁₂-alkylamino,
(h) C₁-C₁₂-alkylamino-C₁-C₆-alkoxy,
(i) C₁-C₁₂-dialkylamino,
(j) C₁-C₁₂-dialkylamino-C₁-C₆-alkoxy,
(k) alkenyl,
(l) alkynyl,
(m) C₁-C₁₂-thioalkoxy,
(n) C₁-C₁₂-alkyl,
(o) C₁-C₁₂-substituted alkyl;

18. The compound of claim 7 wherein R is each selected from the group consisting of
(1) hydrogen,
(2) cycloalkyl,
(3) cycloalkenyl,
(4) C₁-C₁₂-alkyl,
(5) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) —COOR₅ wherein R₅ is hydrogen or loweralkyl,
(f) —C(O)NR₅R₆ wherein R₆ is hydrogen or loweralkyl,
(g) amino,
(h) —NR₅R₆,
or
R₅ and R₆ taken together with the atom to which they are attached form a 3-10 membered heterocycloalkyl ring which is optionally substituted with one or more substituents independently selected from the group consisting of
(i) halogen,
(ii) hydroxy,
(iii) C₁-C₃-alkoxy,
(iv) C₁-C₃-alkoxy-C₁-C₃-alkoxy,
(v) oxo,
(vi) C₁-C₁₂-alkyl,
(vii) C₁-C₁₂haloalkyl,
and
(viii) C₁-C₃-alkoxy-C₁-C₁₂-alkyl,
(i) aryl.
(j) substituted aryl,
(k) heteroaryl.
(l) substituted heteroaryl,
(m) mercapto,
(n) C₁-C₁₂-thioalkoxy,
(6) C(═O)OR₁₁, wherein R₁₁ is hydrogen, loweralkyl, substituted loweralkyl, aryl, substituted aryl, heteroaryl or substituted heteroaryl,
(7) C(═O)NR₁₁R₁₂, wherein R₁₂ is hydrogen, loweralkyl, substituted loweralkyl, aryl, substituted aryl, heteroaryl or substituted heteroaryl,
or
R₁₁ and R₁₂ together with the atom to which they are attached form a 3-10 membered heterocycloalkyl ring, which is optionally substituted with one or more substituents independently selected from the group consisting of
(a) halogen.
(b) hydroxy,
(c) C₁-C₃-alkoxy,
(d) C₁-C₃-alkoxy-C₁-C₃-alkoxy,
(e) oxo,
(f) C₁-C₁₂-alkyl,
(g) substituted loweralkyl,
(h) C₁-C₁₂haloalkyl,
(i) amino,
(j) alkylamino,
(k) dialkylamino
and
(l) C₁-C₃-alkoxy-C₁-C₁₂-alkyl,
or
R and its connected oxygen atom taken together is halogen.

19. The compound of any one of claims 9-10 and 12-17 wherein R_{C} is each independently selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C(═O)R₇,
l) C(═O)CHR₈NR₉R₁₀ wherein R₈, R₉ and R₁₀ are independently selected from a group consisting of hydrogen, loweralkyl, substituted loweralkyl, aryl, substituted aryl, heteroaryl or substituted heteroaryl,
or
R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) C₁-C₃haloalkyl,
(h) C₁-C₃-alkoxy-C₁-C₃-alkyl.

20. The compound of claim 8 wherein R₁ and R₂ are each independently selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
or
R₁ and R₂ taken together with the atom to which they are attached form a substituted heteroaryl or 3-10 membered heterocycloalkyl ring which optionally contains one or two hetero functionalities selected from the group consisting of —O—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, —S—, and S(O)ₙ— wherein n is 1 or 2 and the 3-10 membered heterocycloalkyl ring optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) C₁-C₃haloalkyl,
(h) C₁-C₃-alkoxy-C₁-C₃-alkyl,
and
k) C(═O)R₇,
l) C(═O)CHR₈NR₉R₁₀ wherein R₈, R₉ and R₁₀ are independently selected from a group consisting of hydrogen, loweralkyl, substituted loweralkyl, aryl, substituted aryl, heteroaryl or substituted heteroaryl,
or
R₉ and R₁₀ taken together with the atom to which they are attached form a 3-10 membered heterocycloalkyl ring which is optionally substituted with one or more substituents independently selected from the group consisting of
(a) halogen,
(b) hydroxyl,
(c) C₁-C₃-alkoxy,
(d) C₁-C₃-alkoxy-C₁-C₃-alkoxy,
(e) oxo,
(f) C₁-C₃-alkyl,
(g) C₁-C₃haloalkyl,
(h) C₁-C₃-alkoxy-C₁-C₃-alkyl;
wherein R₇ is selected from the group consisting of
a) hydrogen,
b) C₁-C₁₂-alkyl,
c) C₁-C₁₂-alkyl substituted with one or more substituents selected from the group consisting of
(a) halogen,
(b) hydroxy,
(c) C₁-C₁₂-alkoxy,
(d) C₁-C₃-alkoxy-C₁-C₃-alkoxy,
(e) amino,
(f) C₁-C₁₂-alkylamino,
(g) C₁-C₁₂-dialkylamino,
(h) alkenyl,
(i) alkynyl,
(j) C₁-C₁₂-thioalkoxy,
d) C₁-C₁₂-alkyl substituted with aryl,
e) C₁-C₁₂-alkyl substituted with substituted aryl,
f) C₁-C₁₂-alkyl substituted with heteroaryl,
g) C₁-C₁₂-alkyl substituted with substituted heteroaryl,
h) cycloalkyl,
i) cycloalkenyl,
j) heterocycloalkyl,
k) C₁-C₁₂-alkylamino,
l) amino,
m) amino-cycloalkyl;

21. A compound having the formula selected from the structure formulae consisting

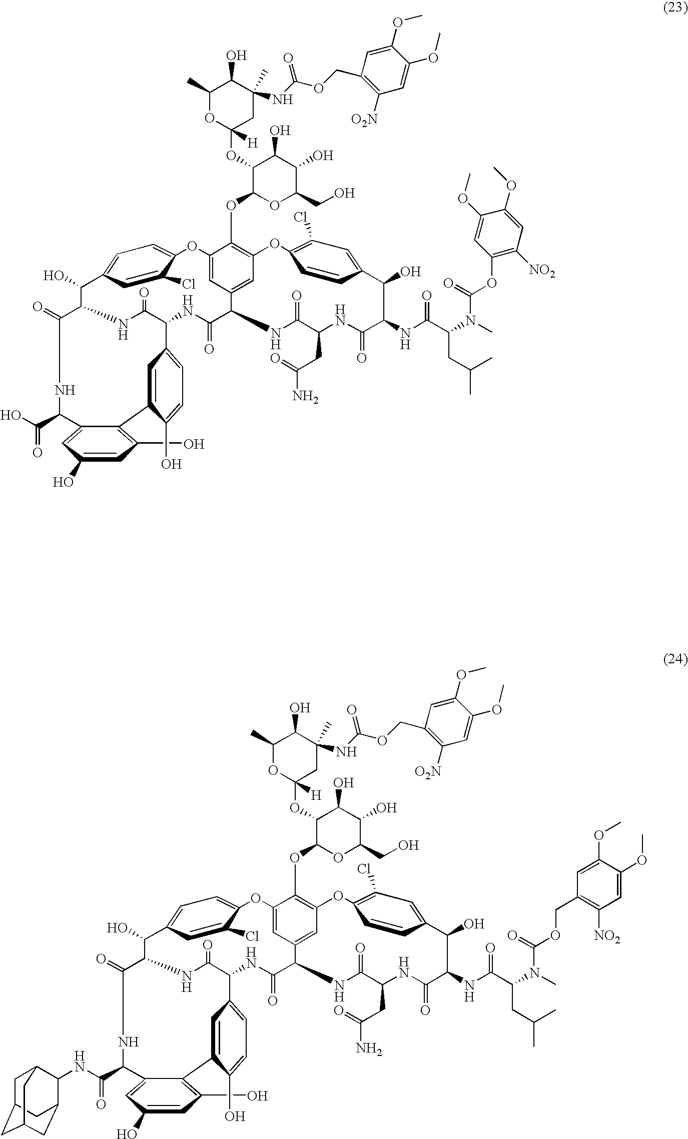

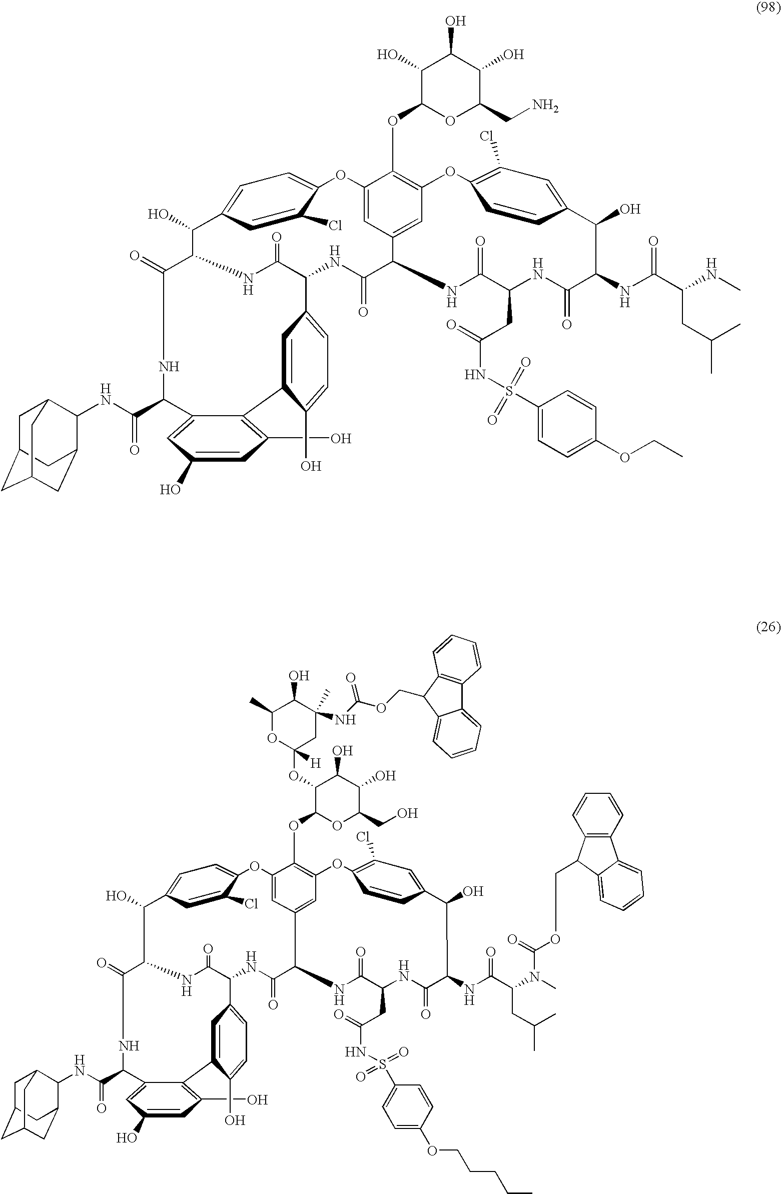

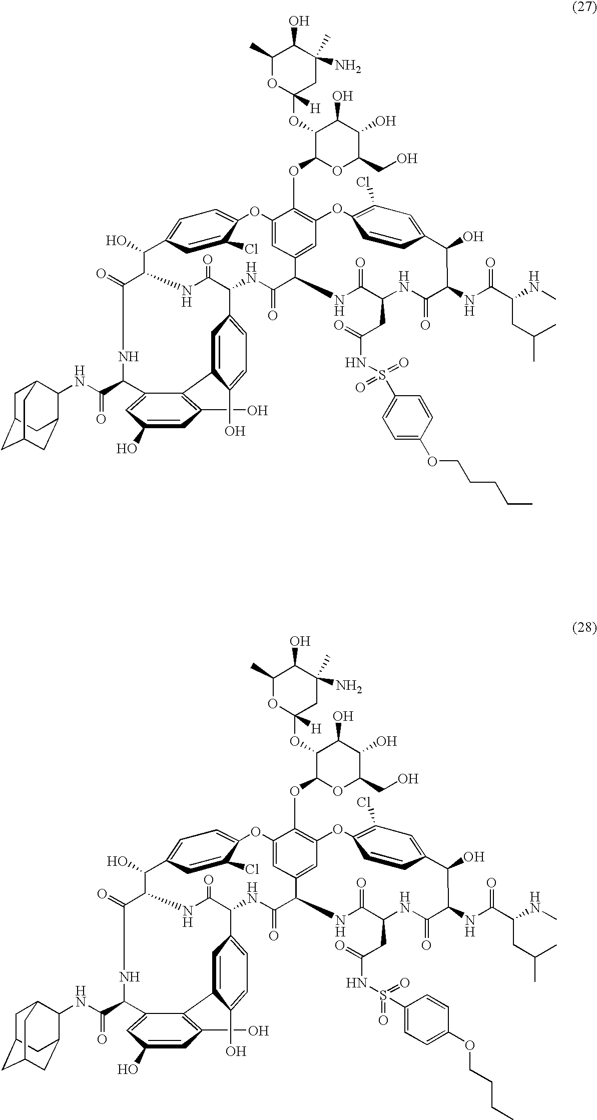

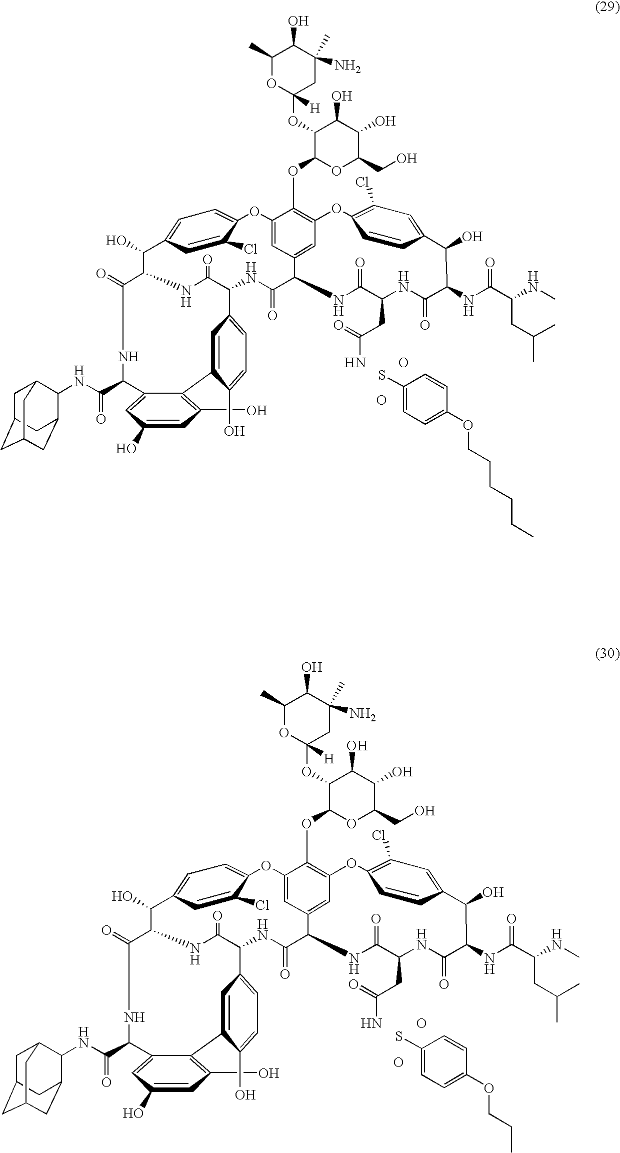

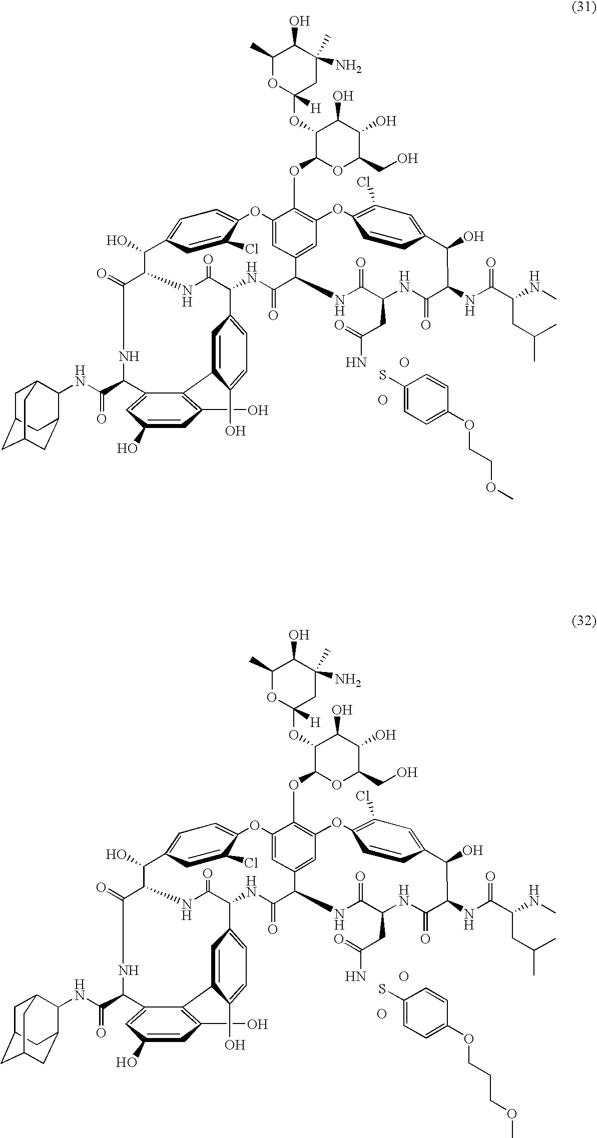

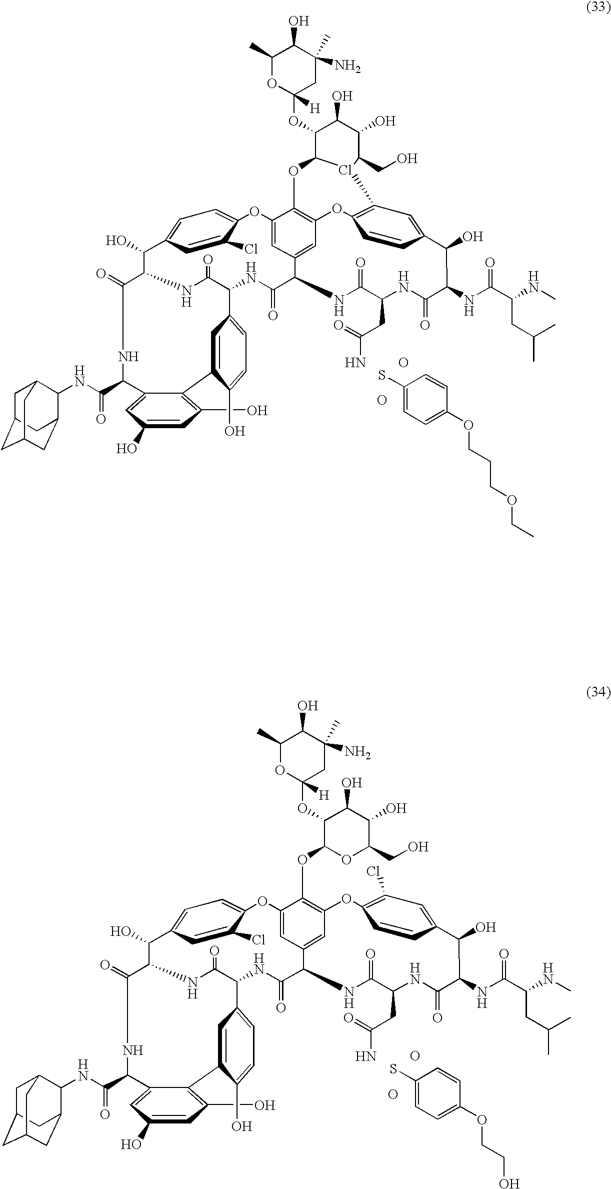

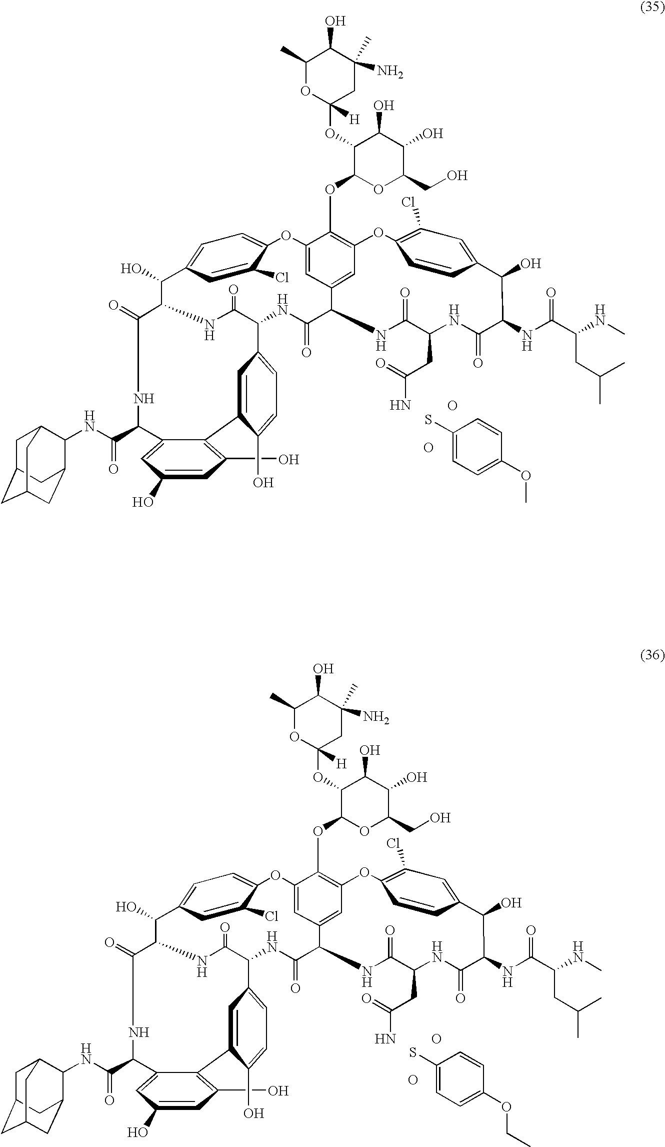

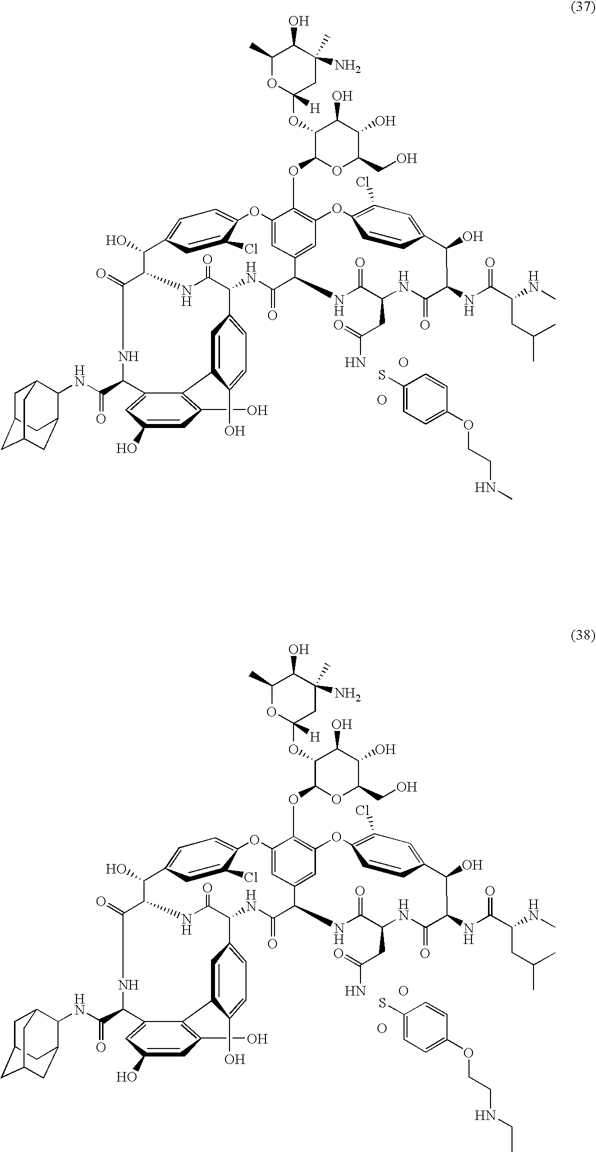

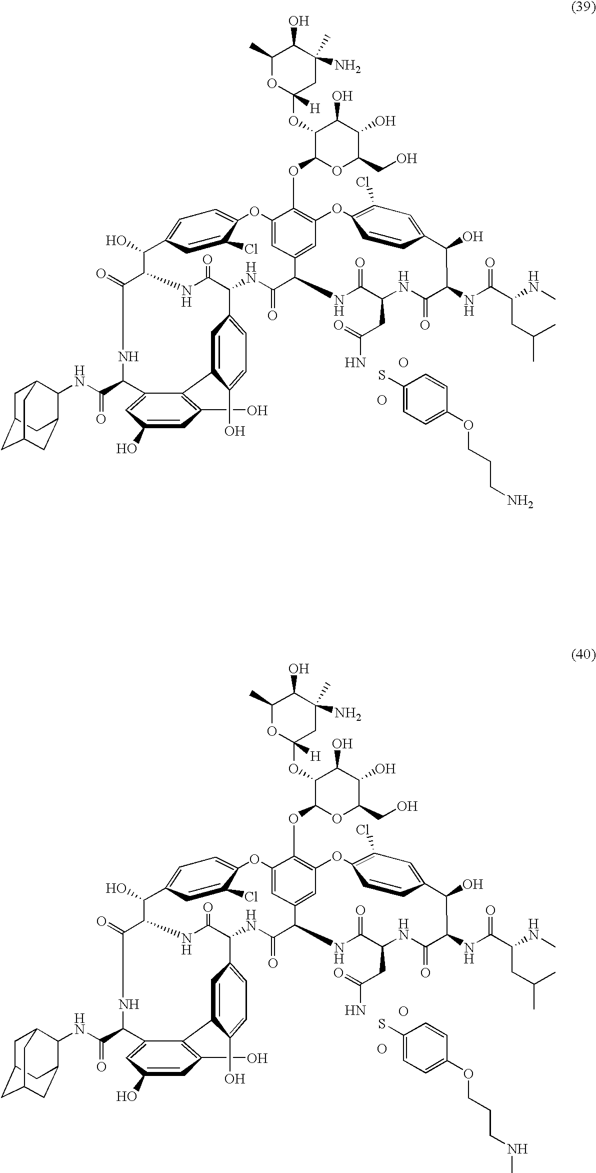

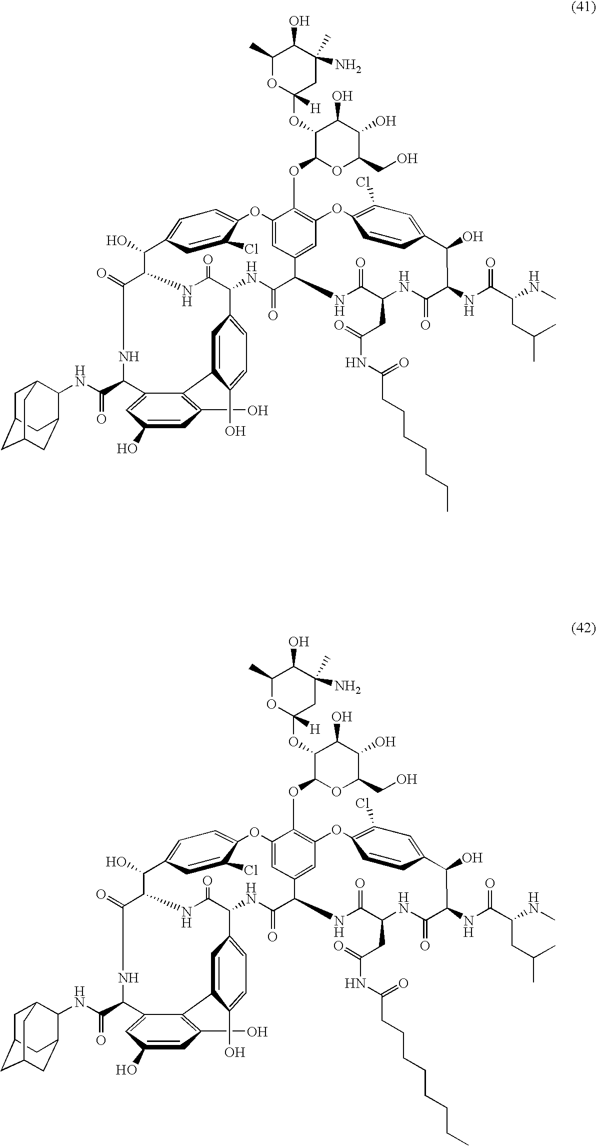

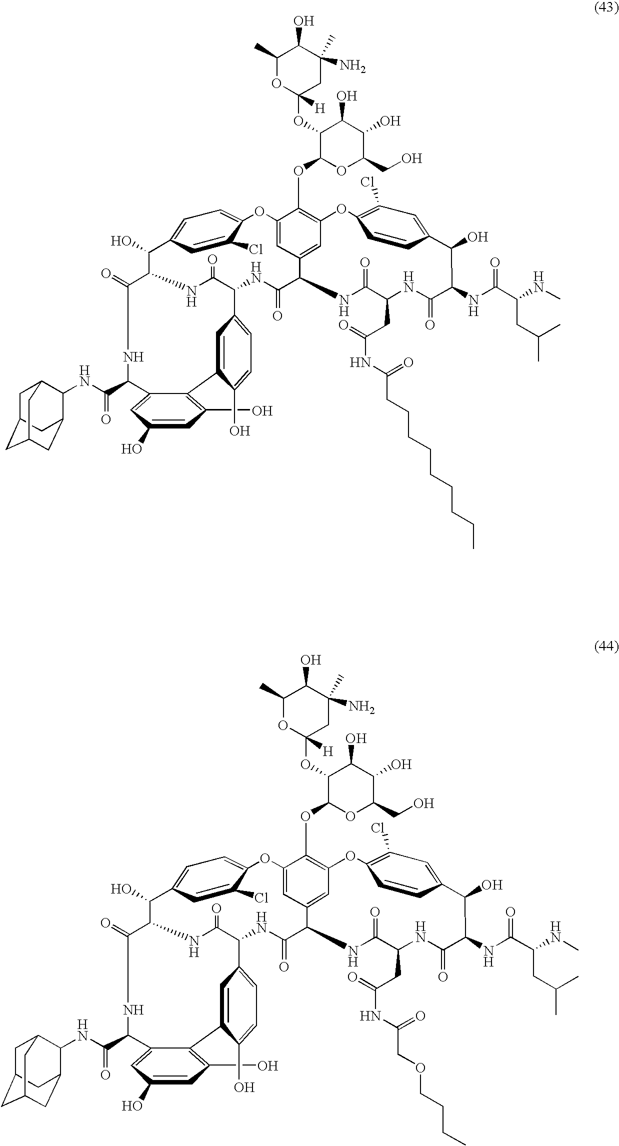

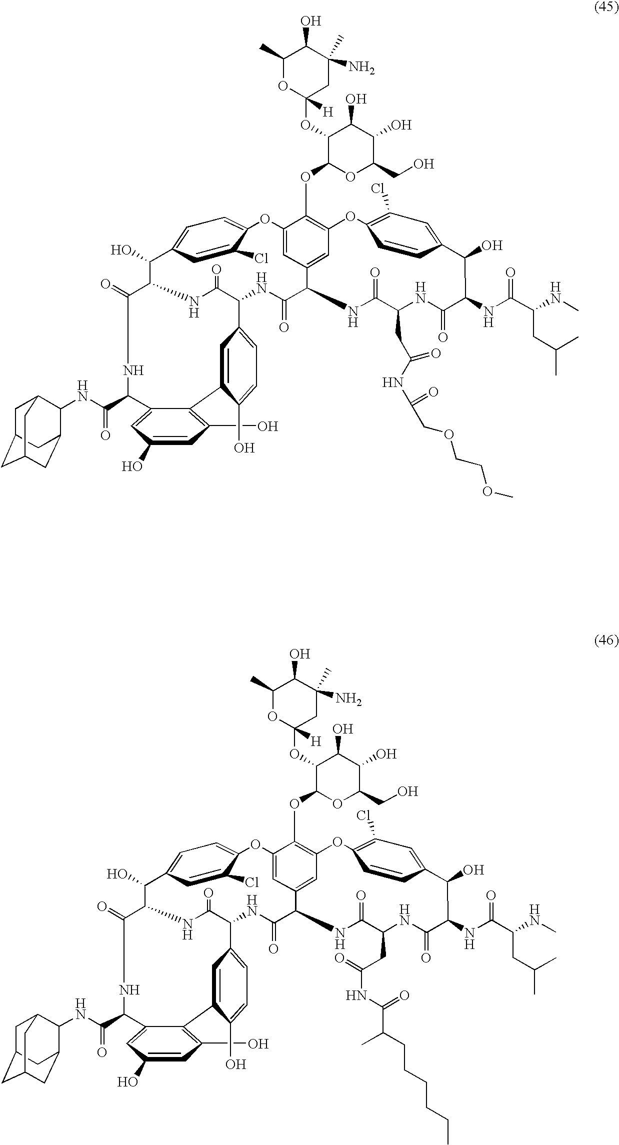

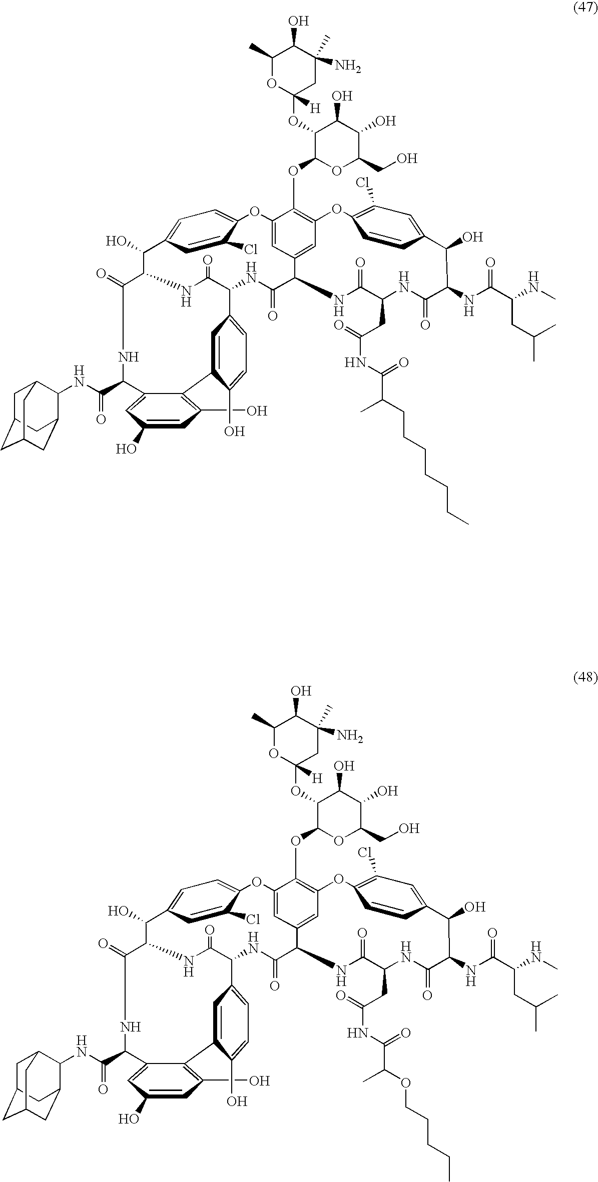

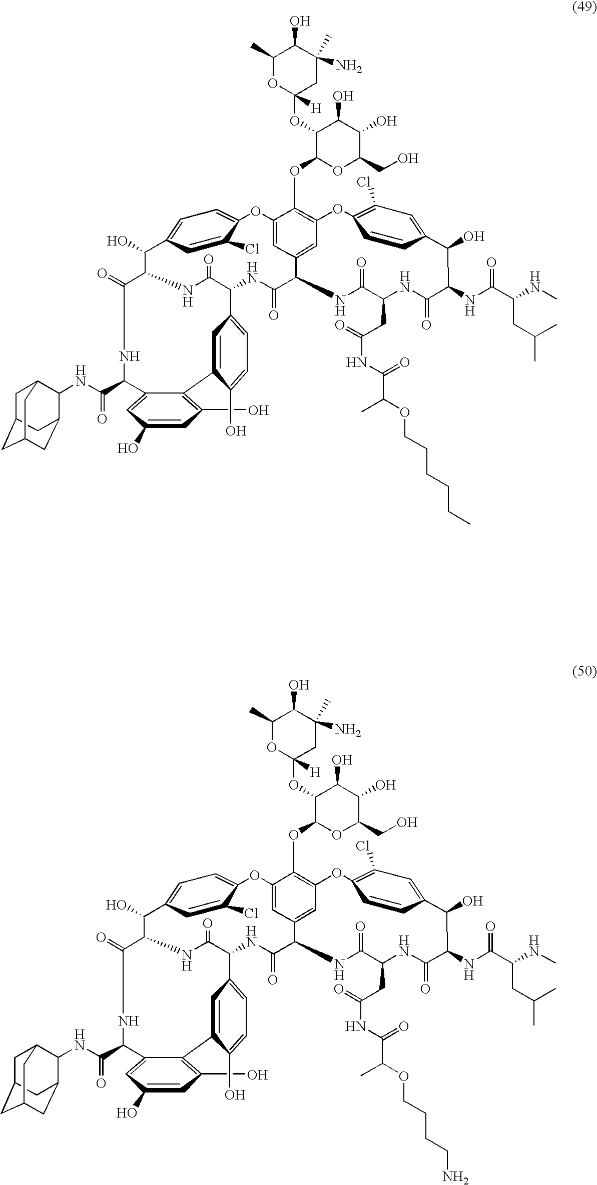

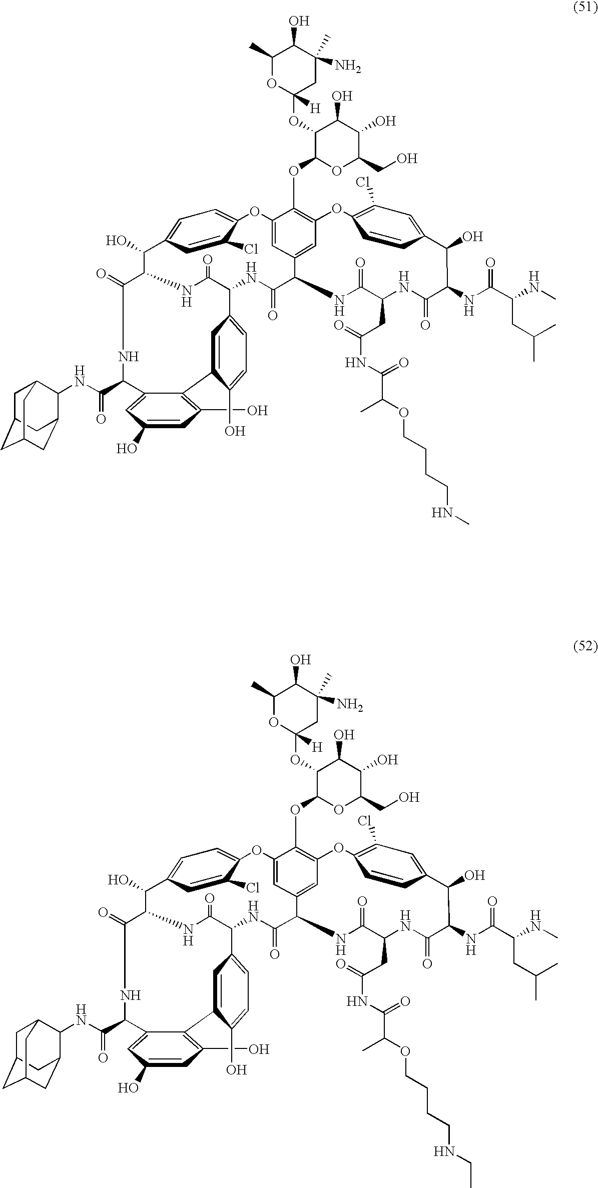

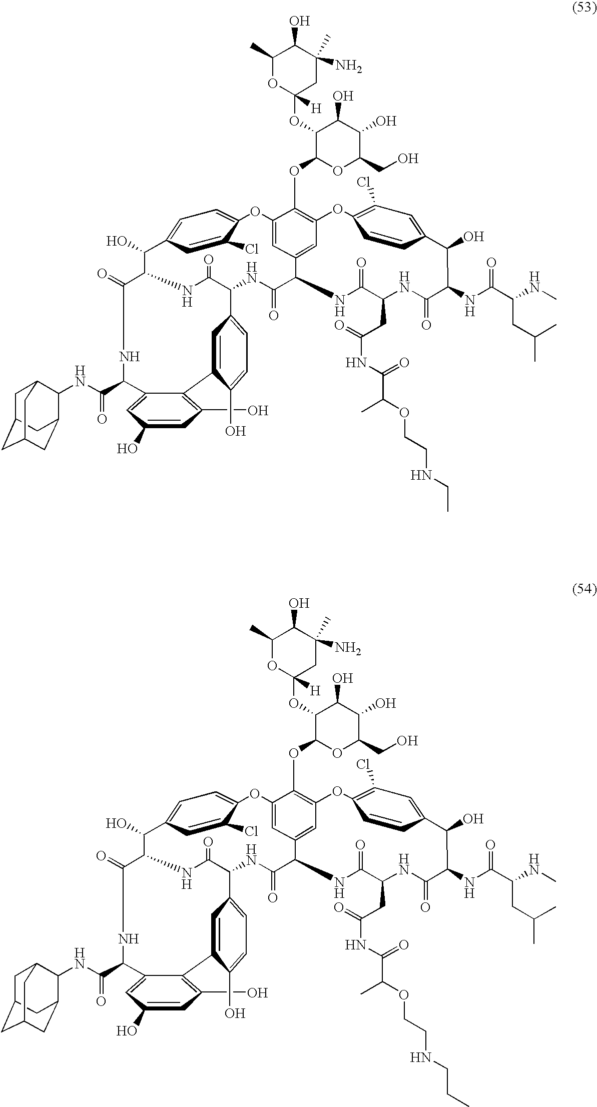

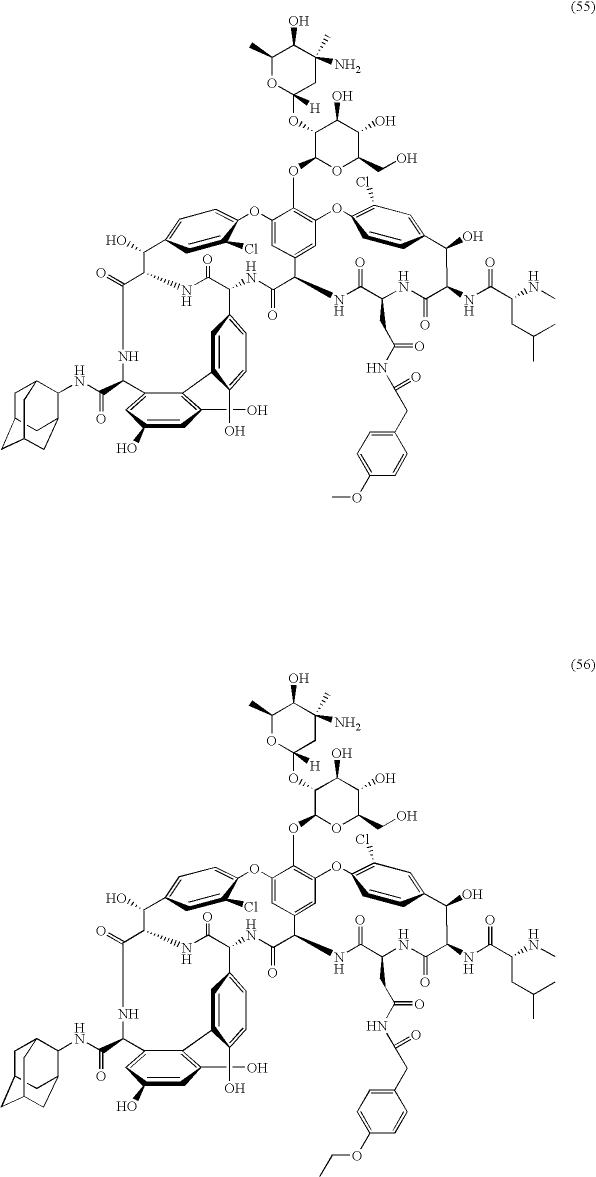

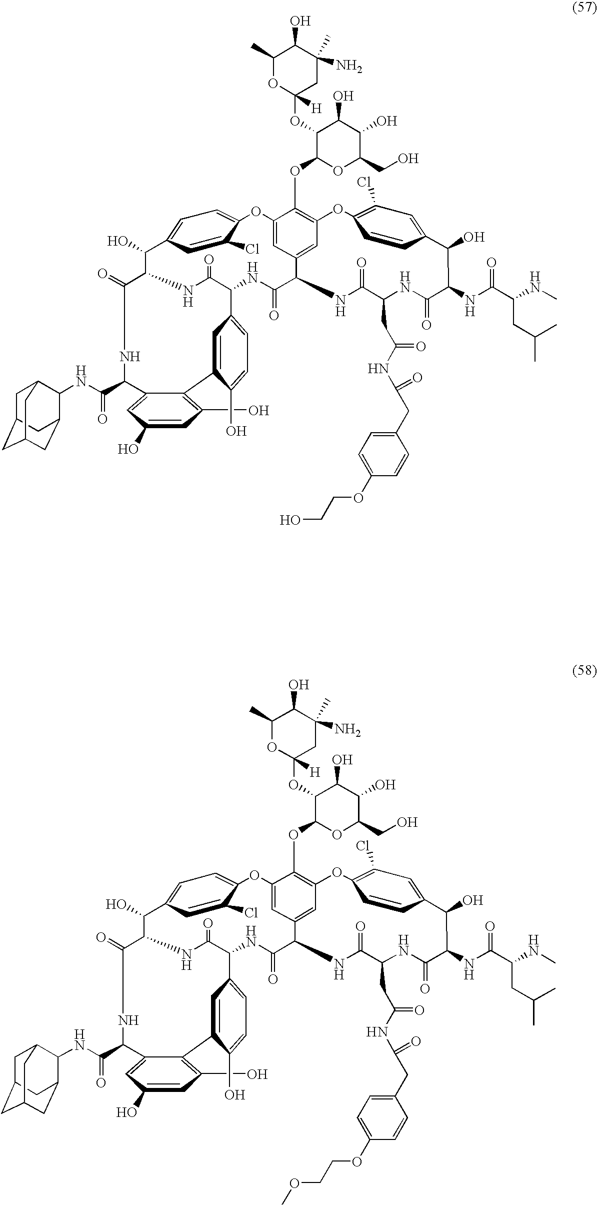

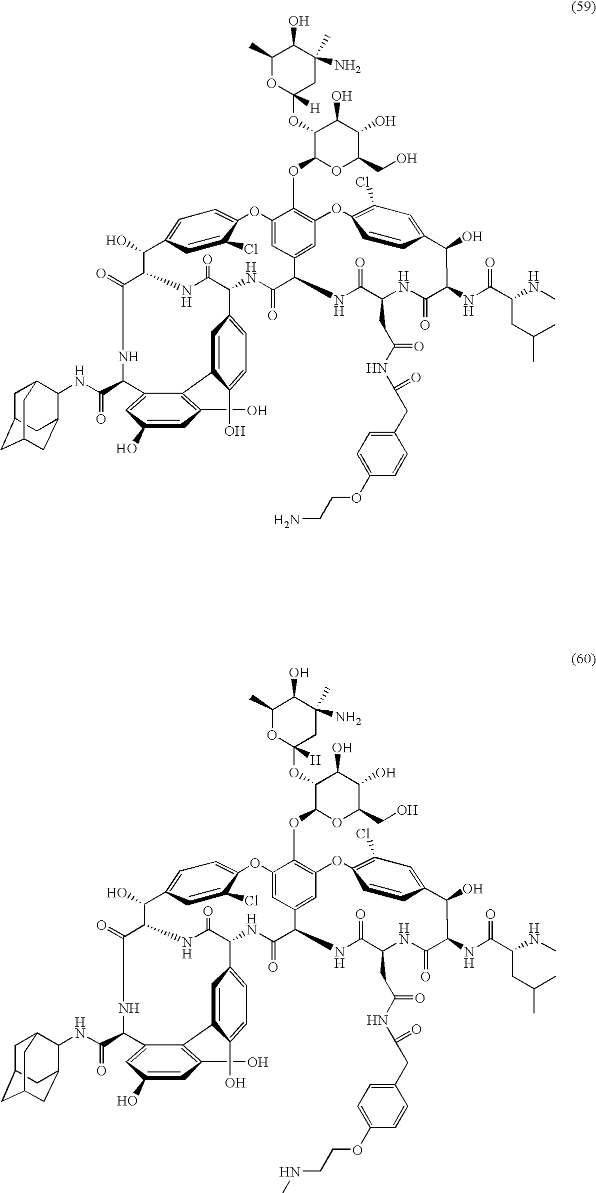

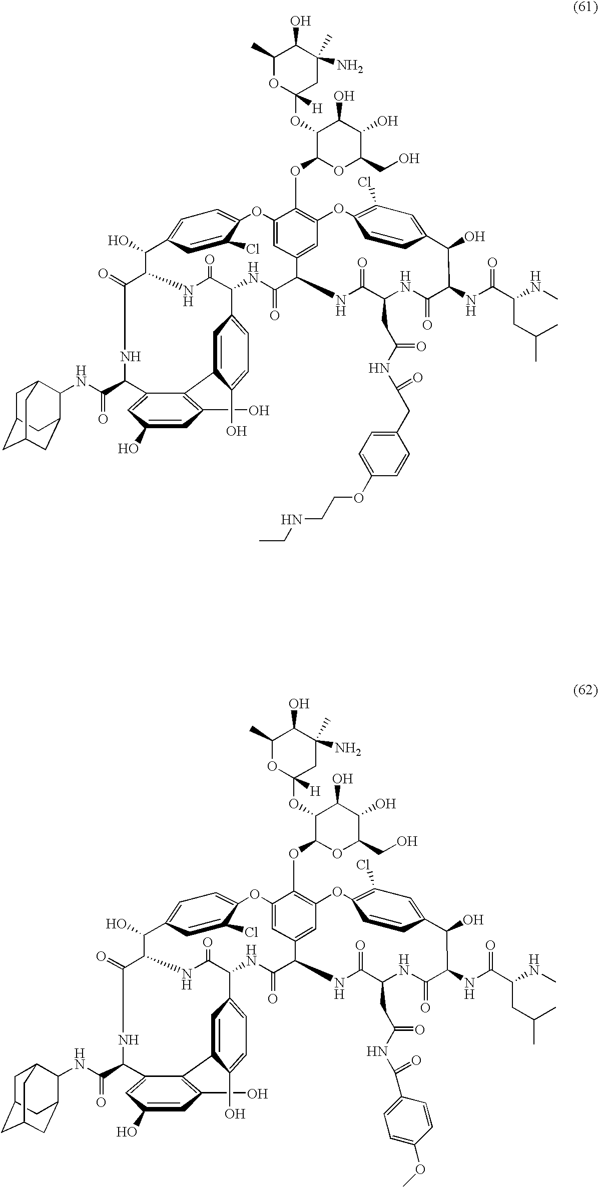

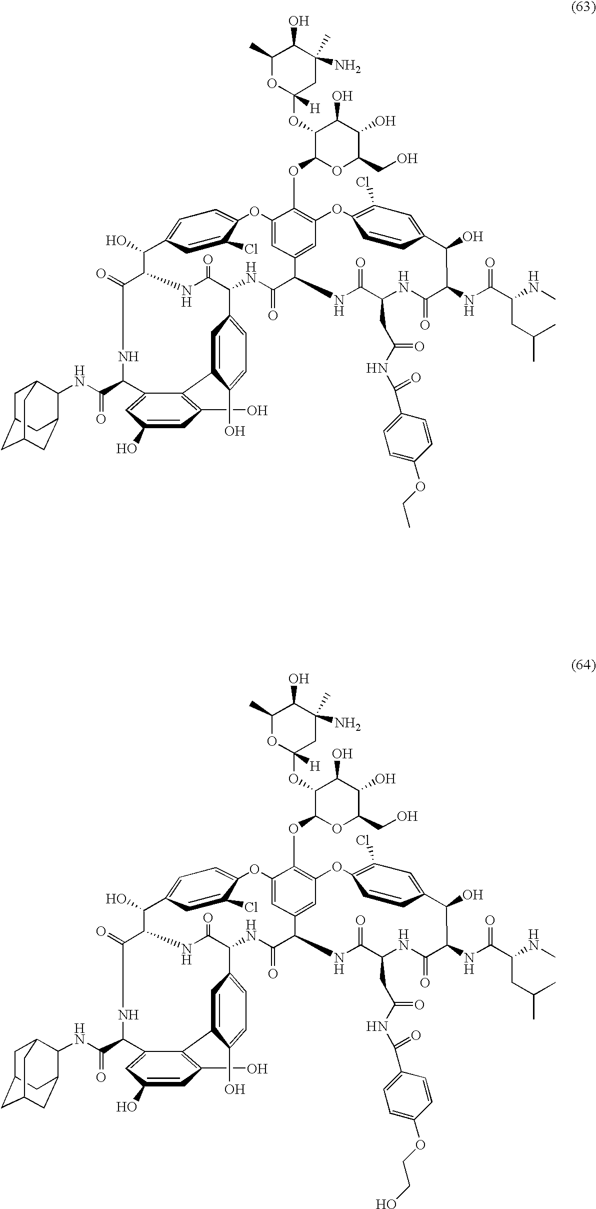

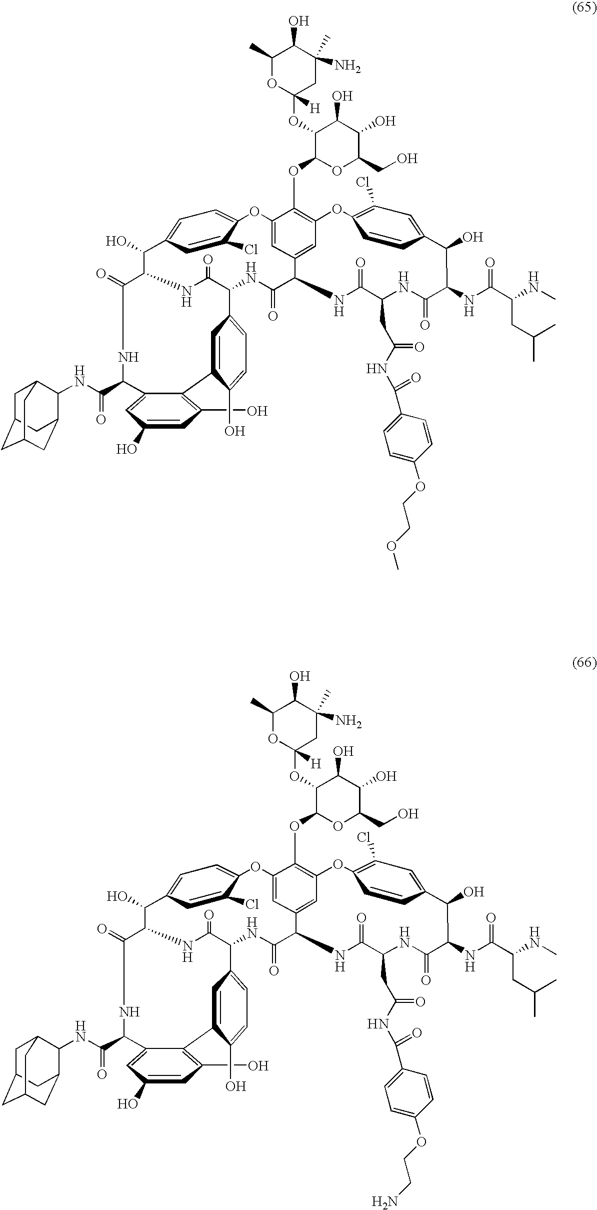

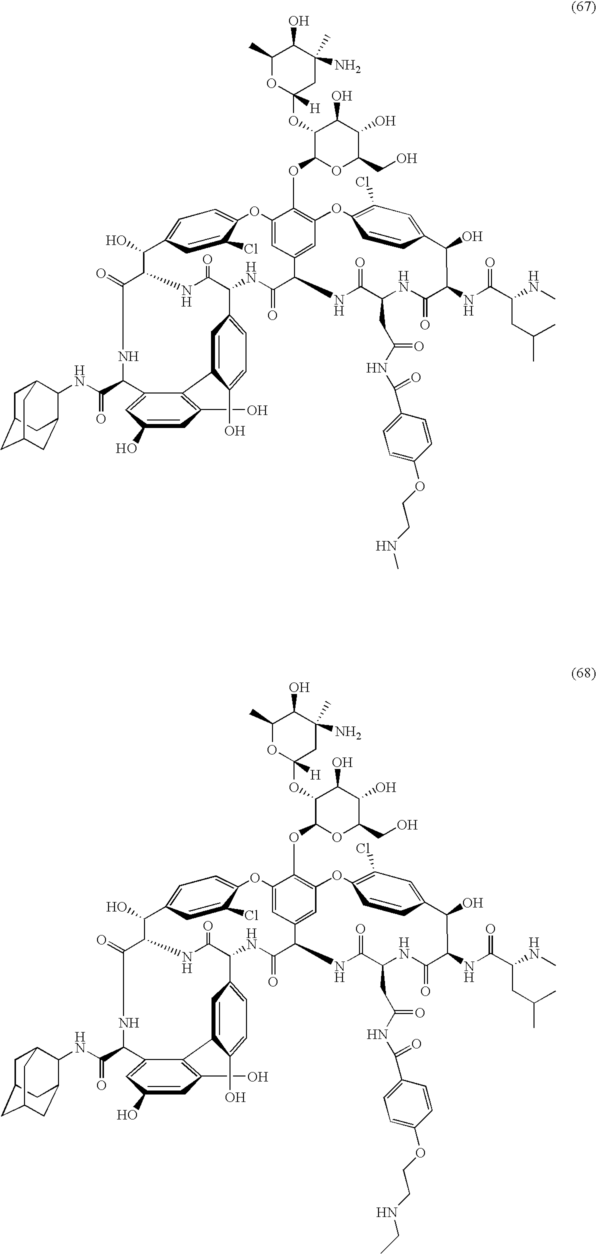

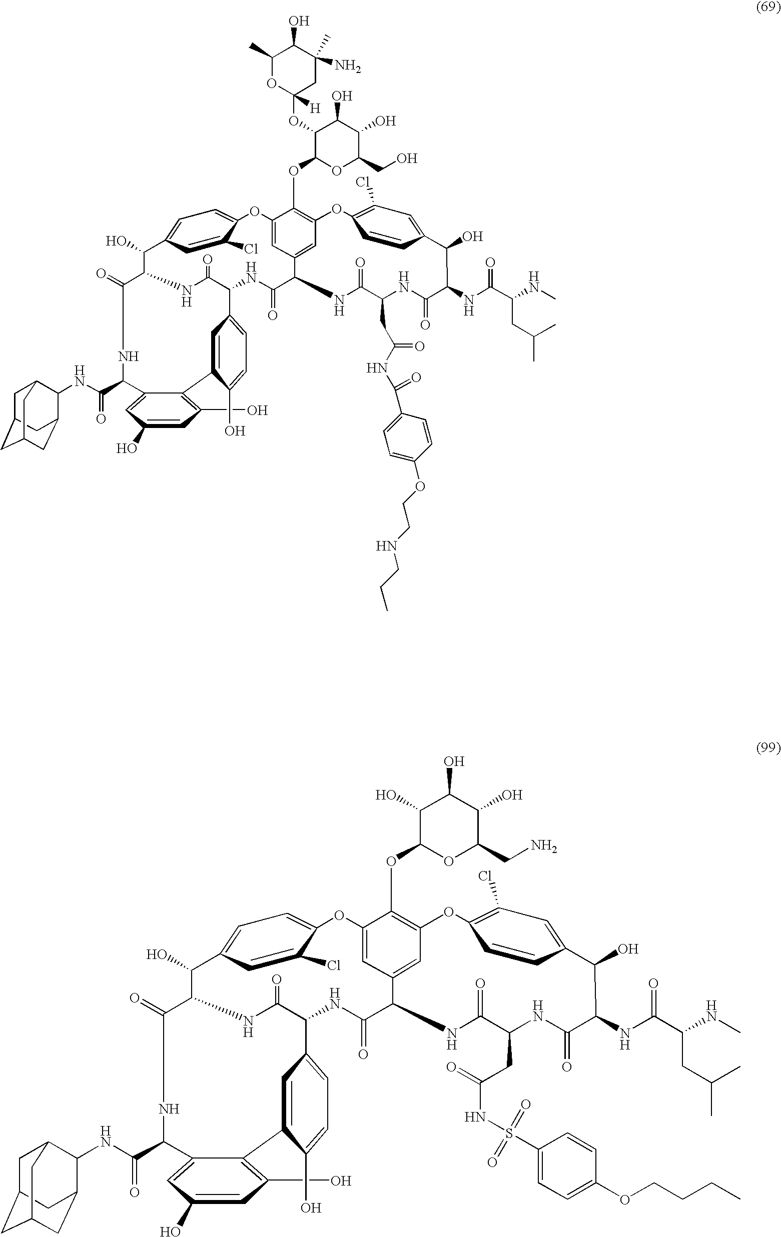

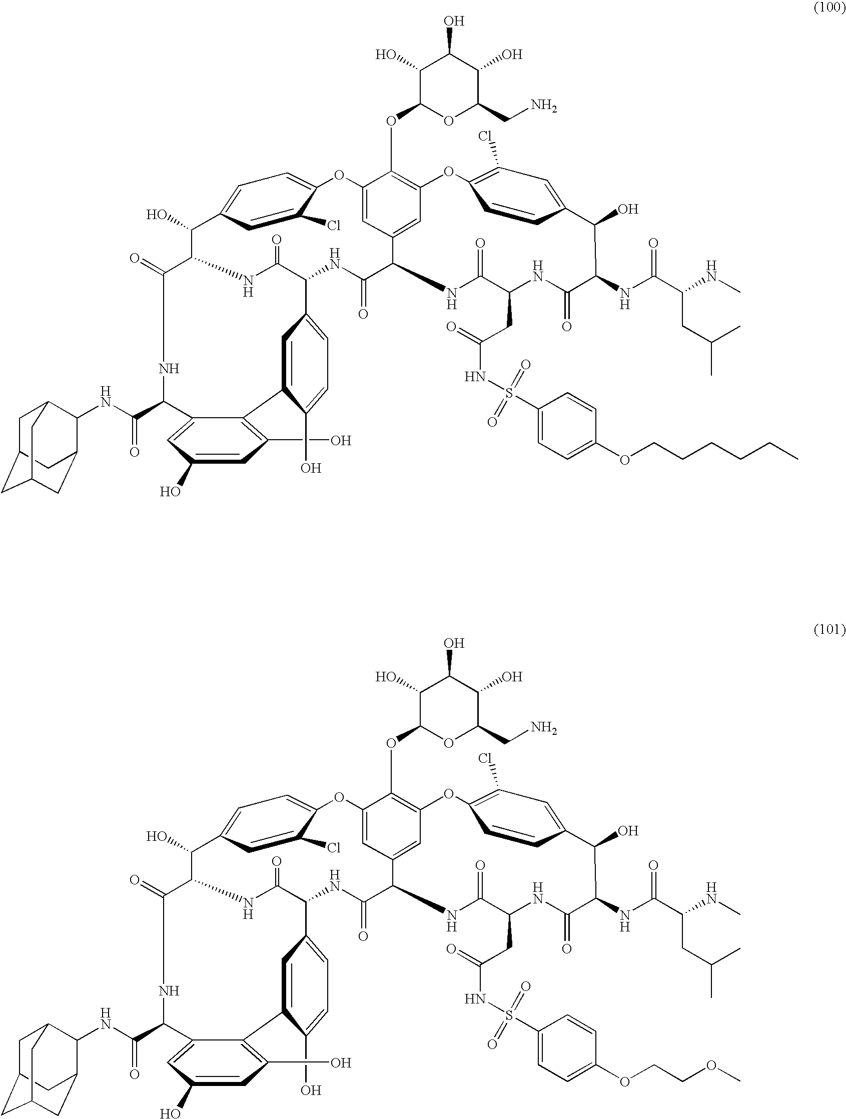

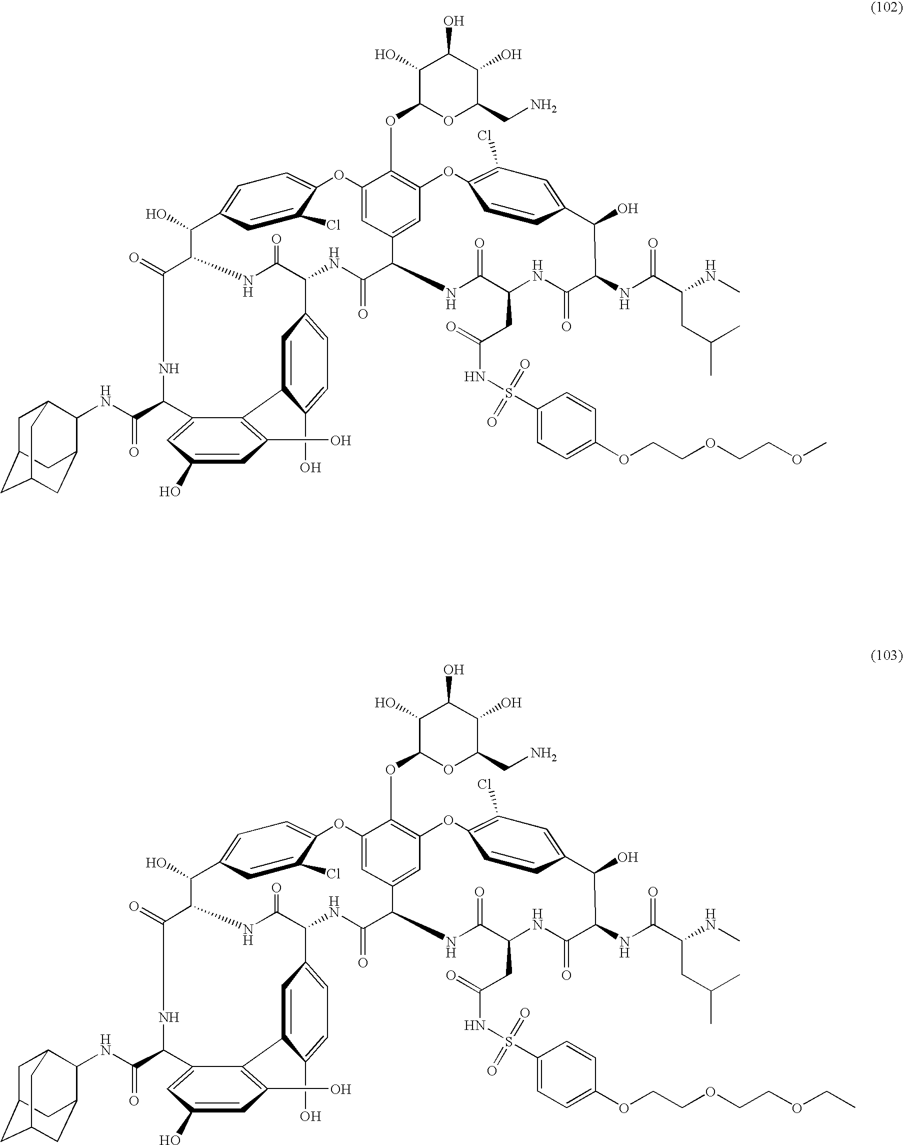

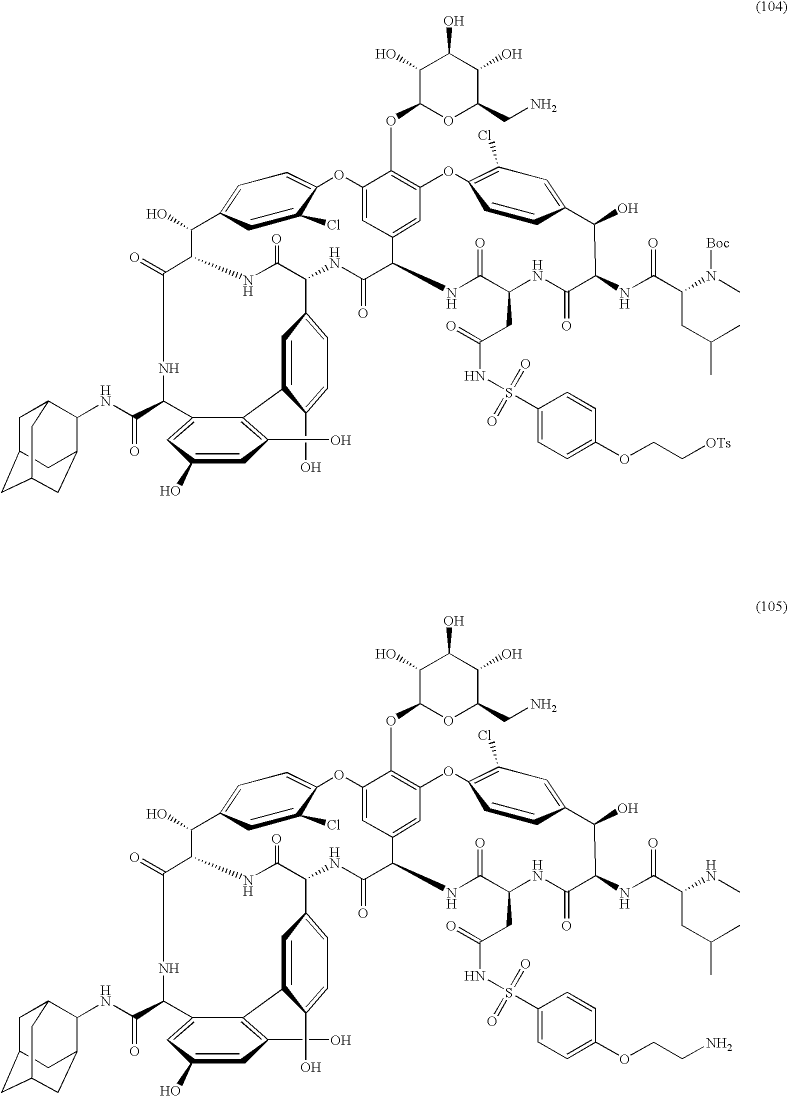

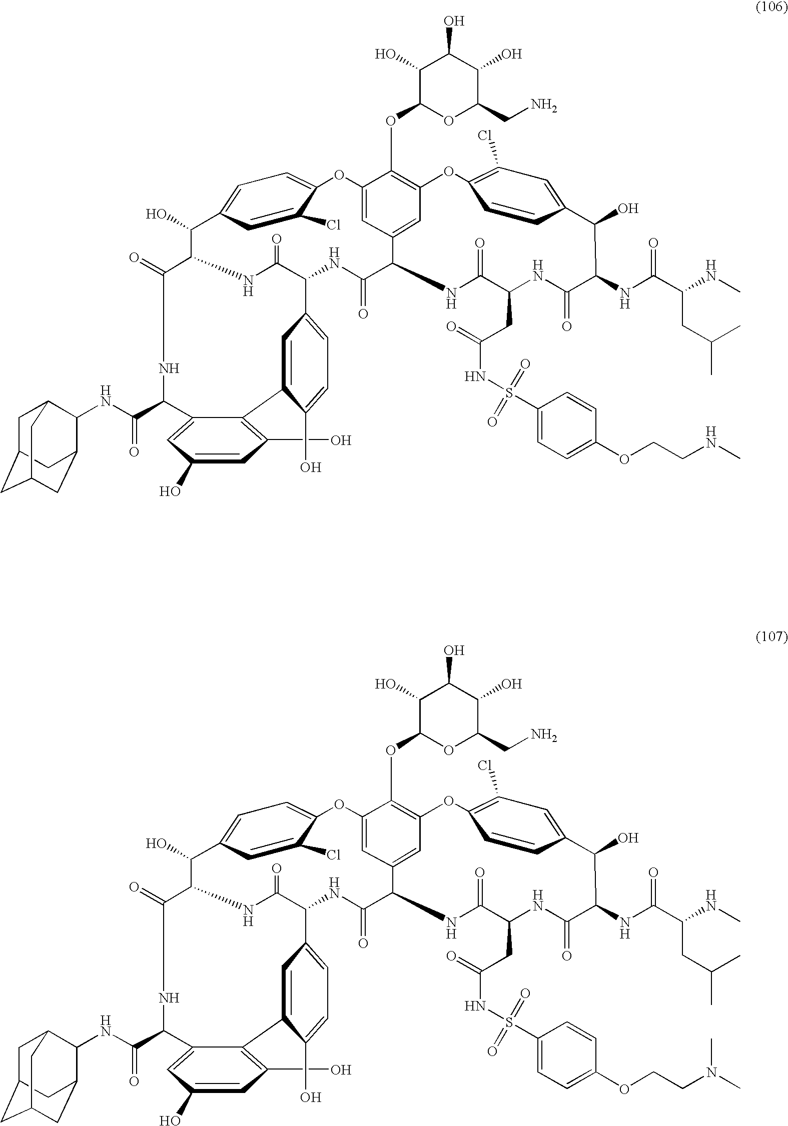

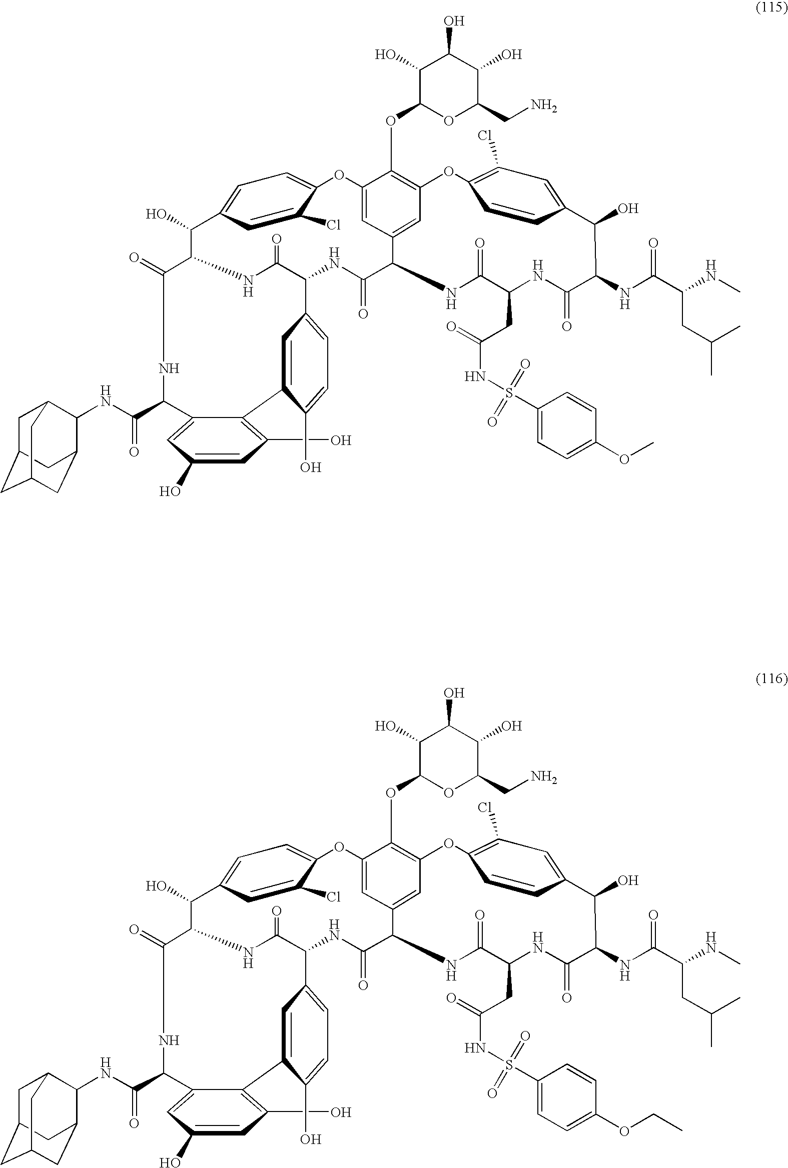

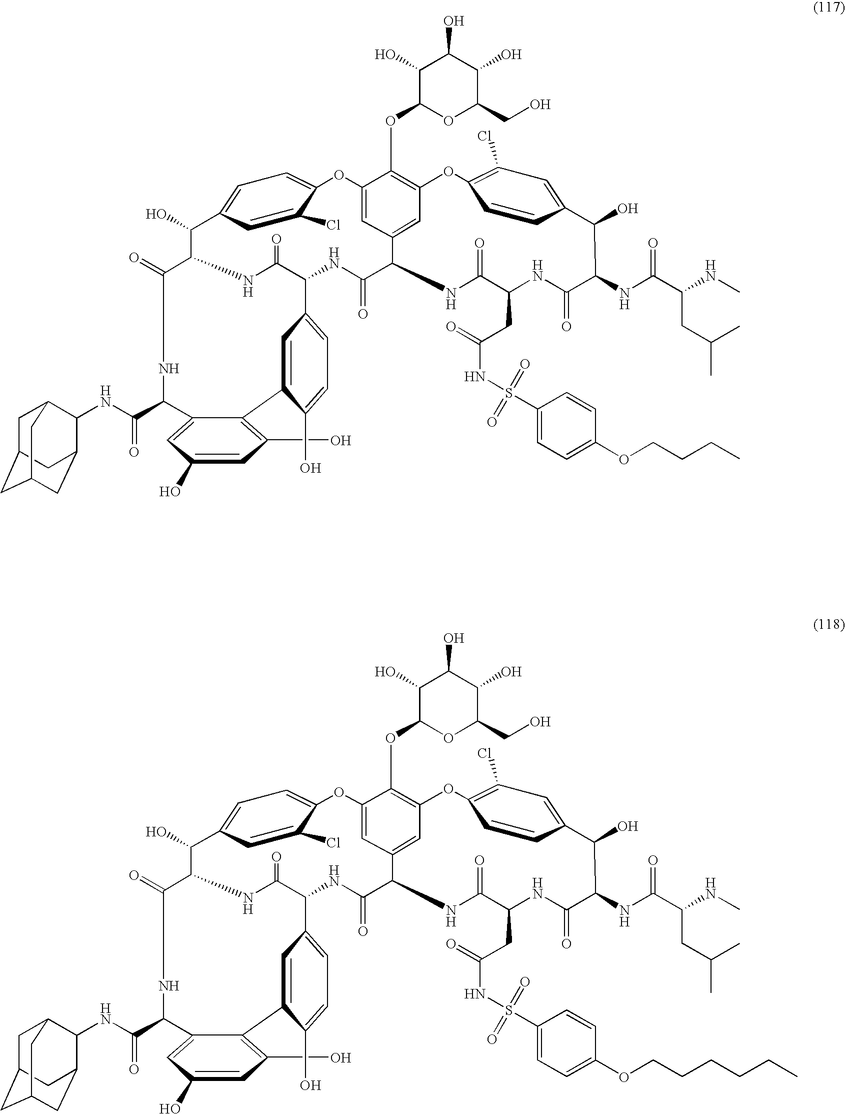

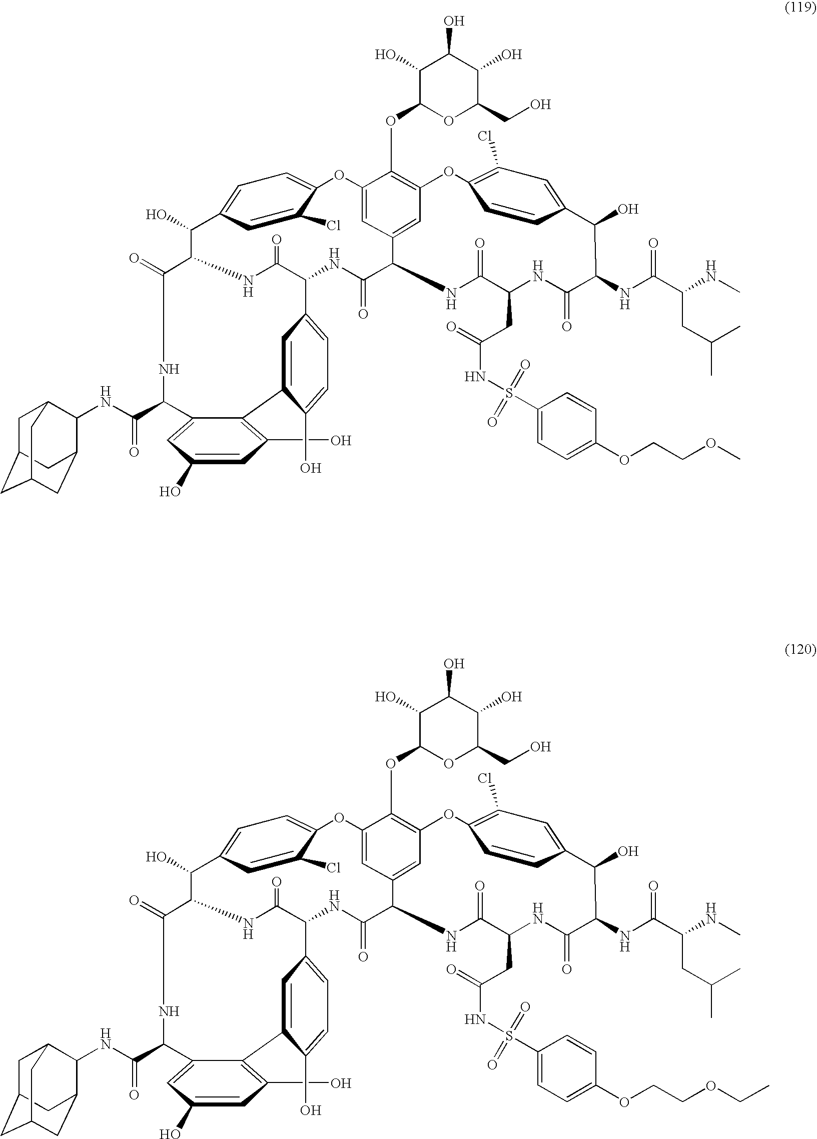

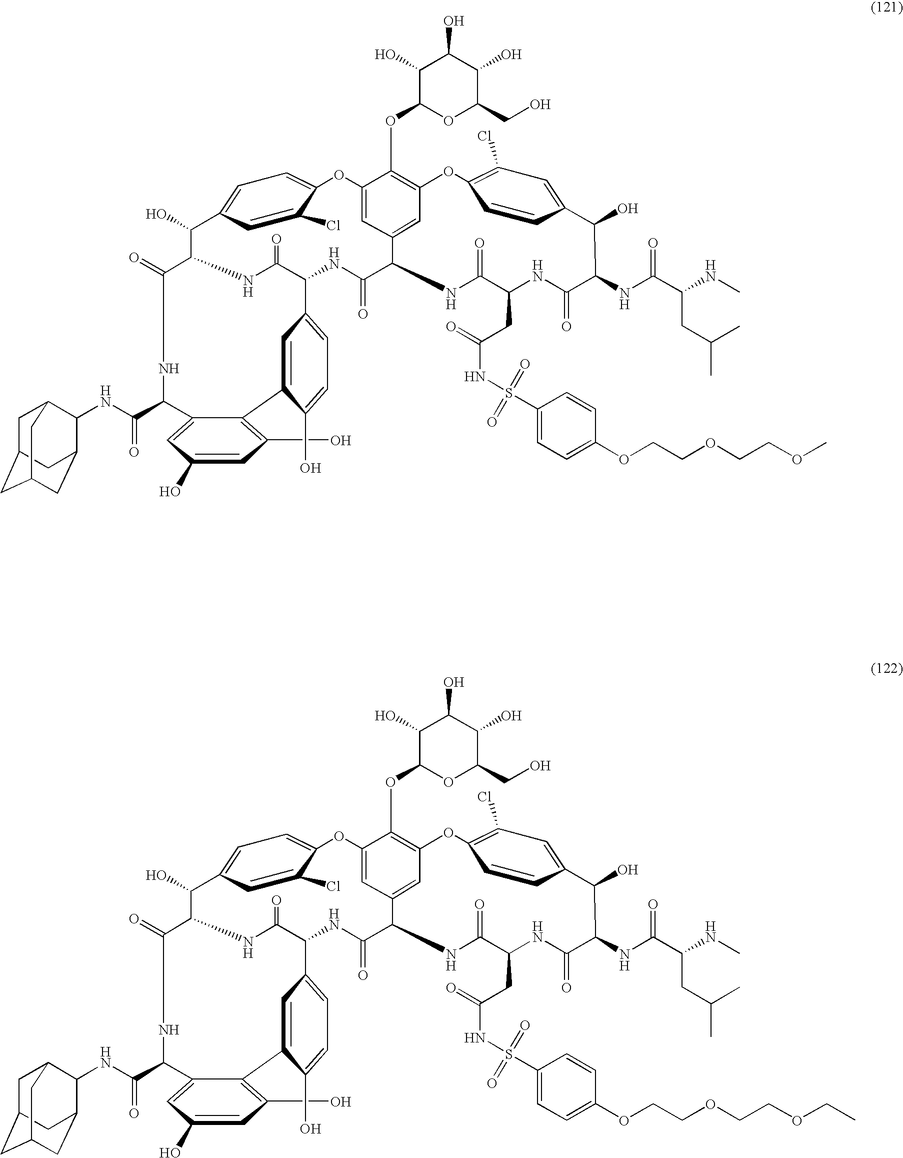

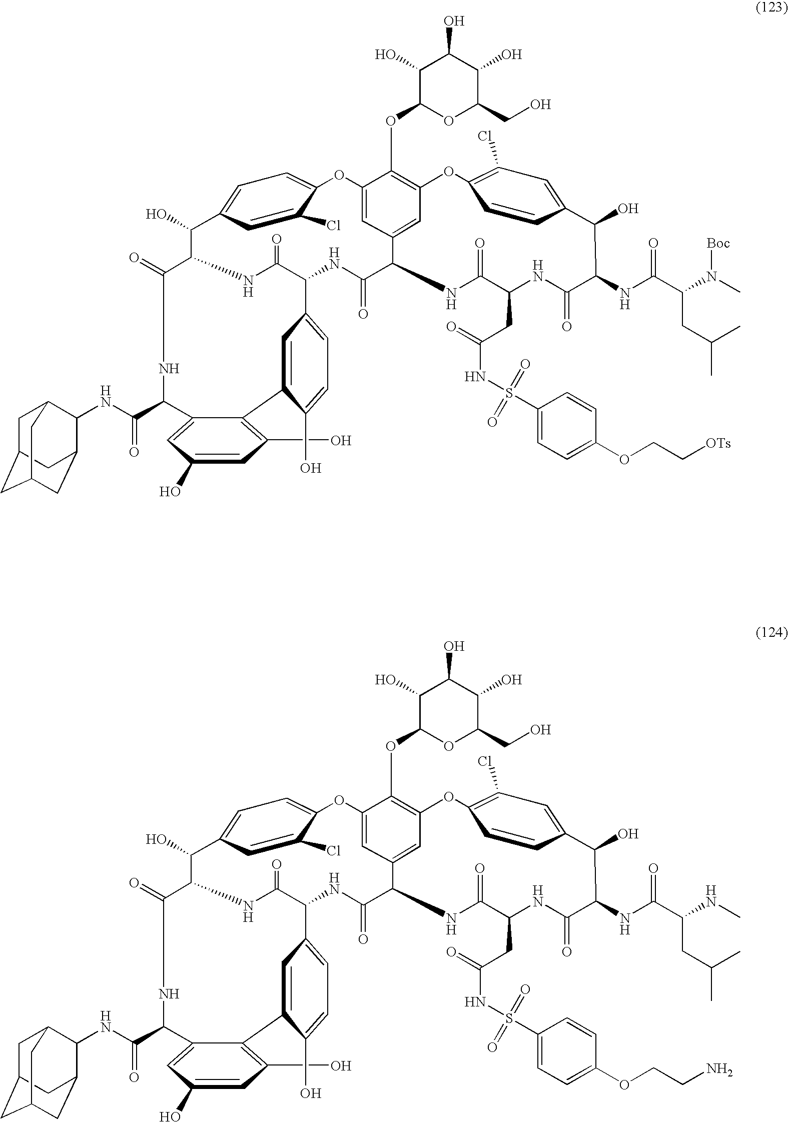

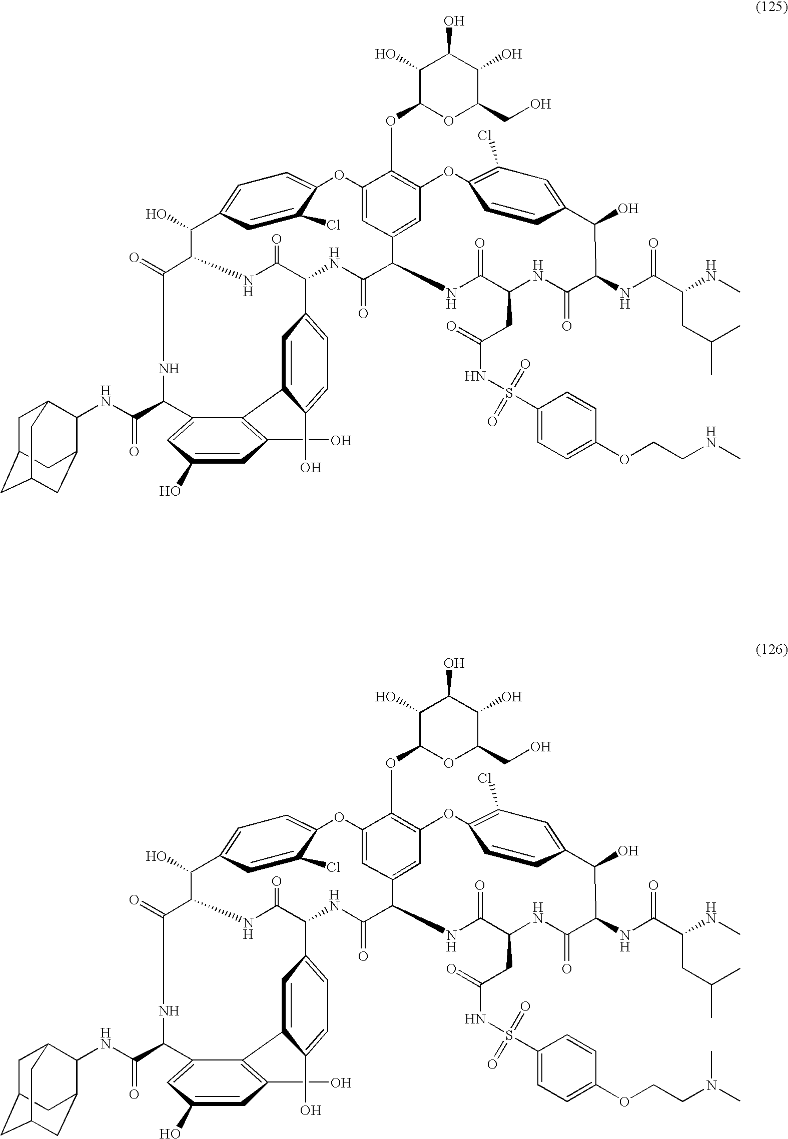

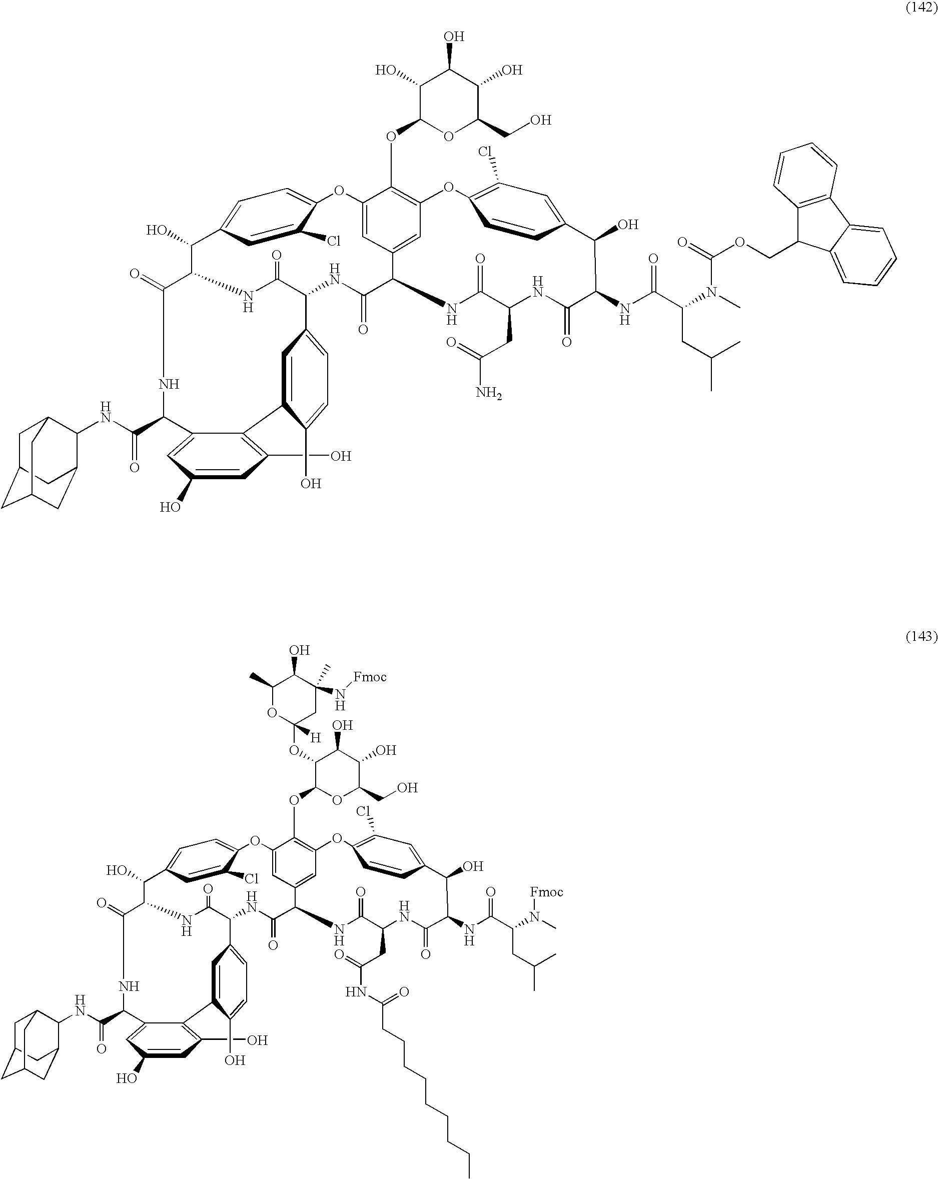

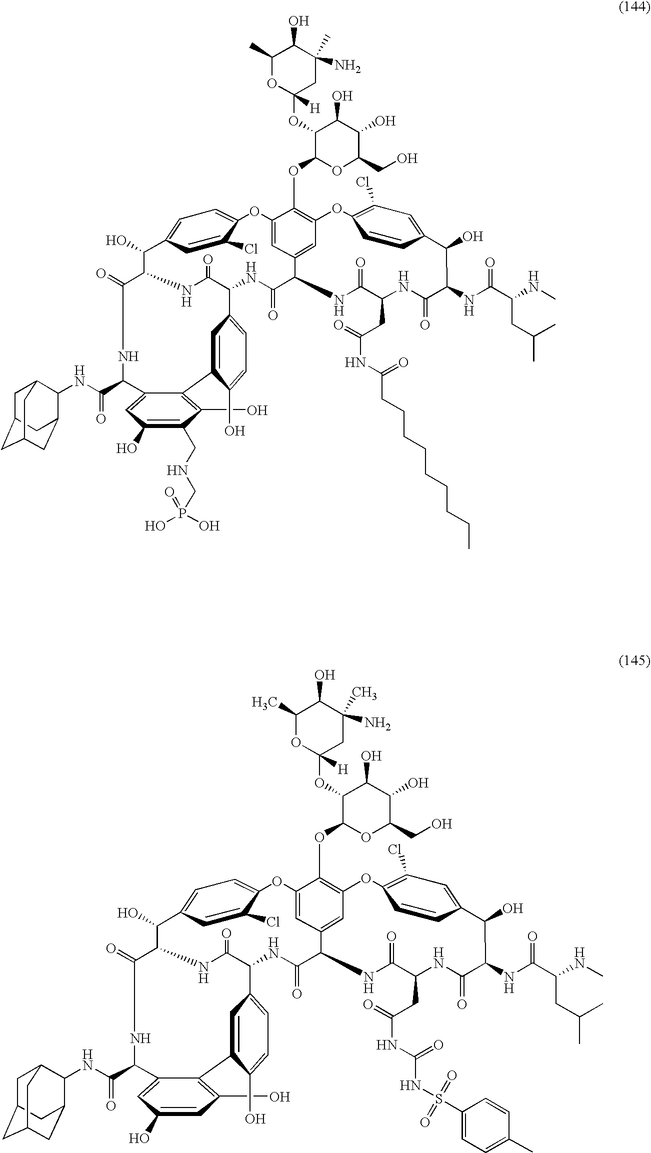

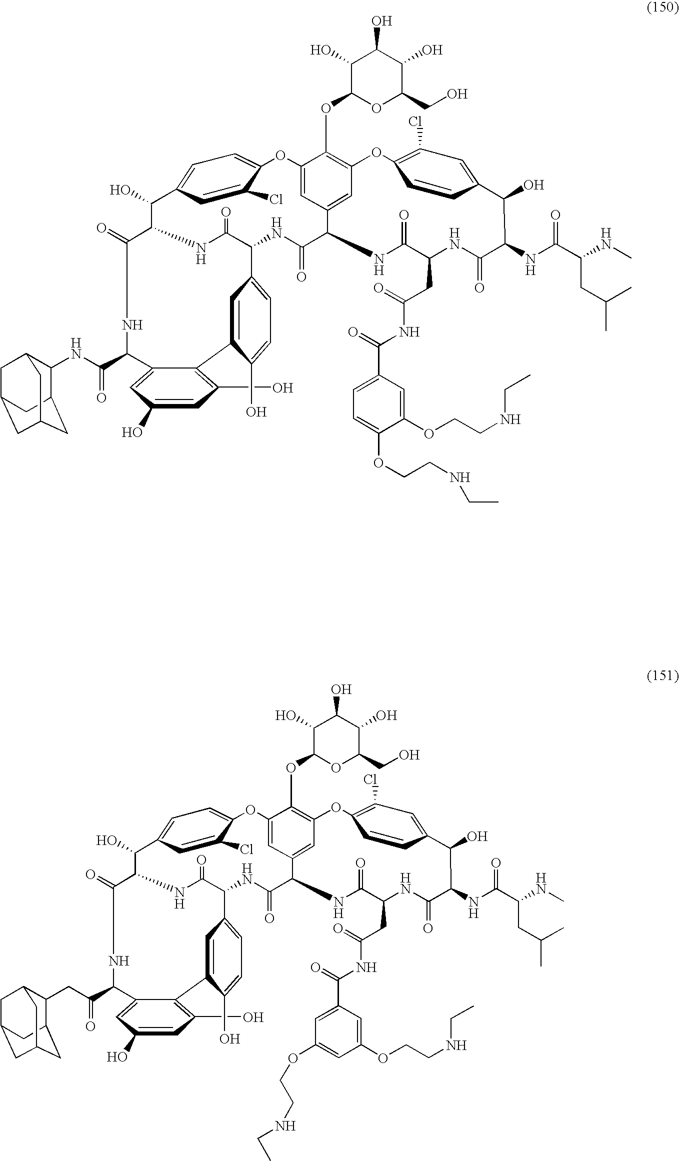

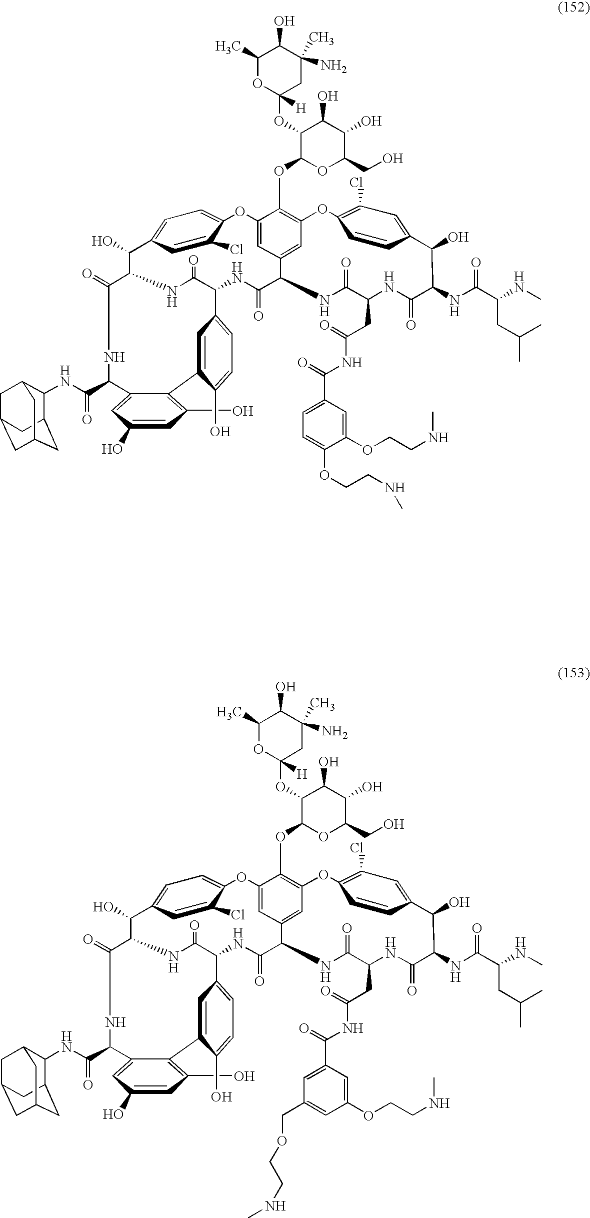

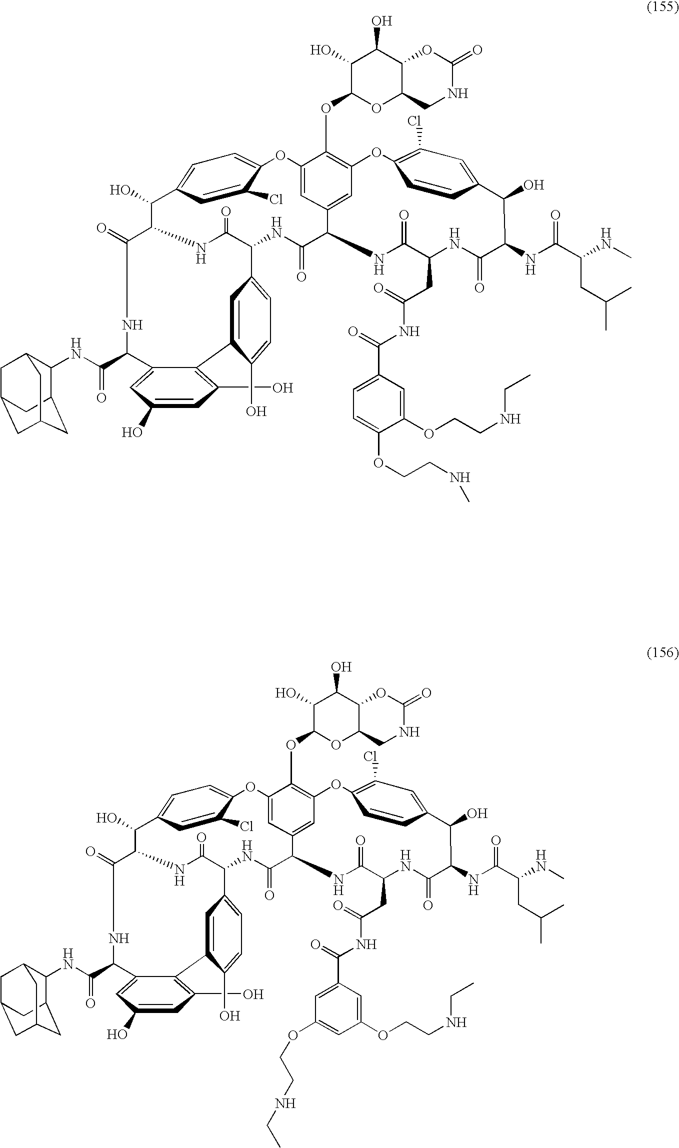

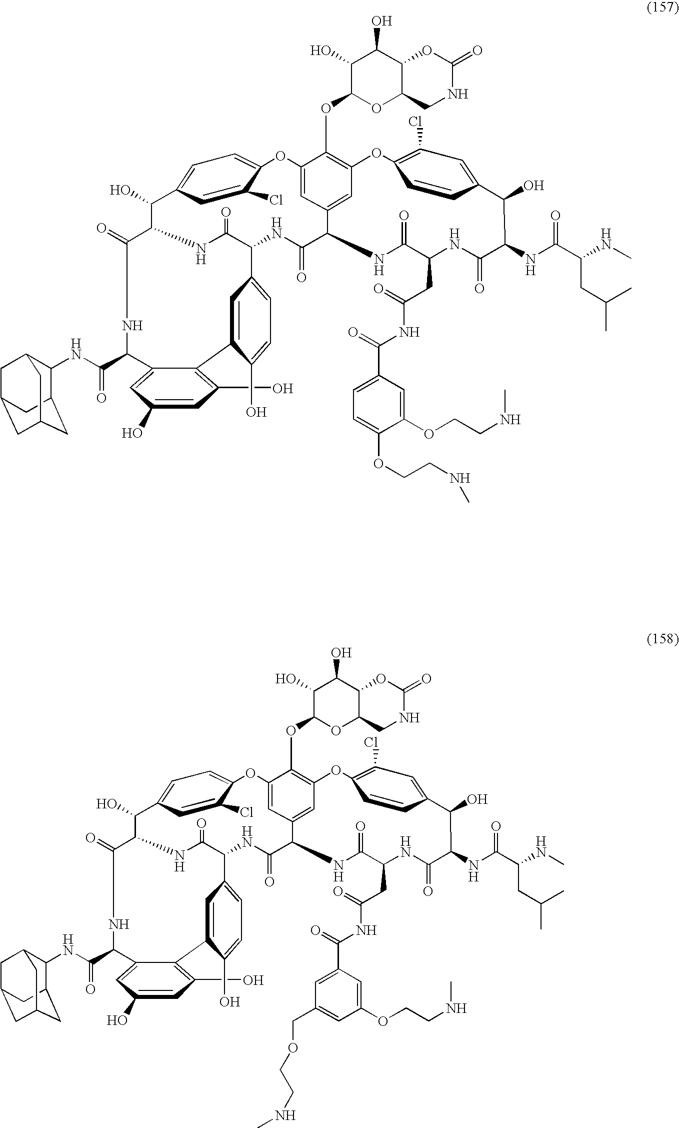

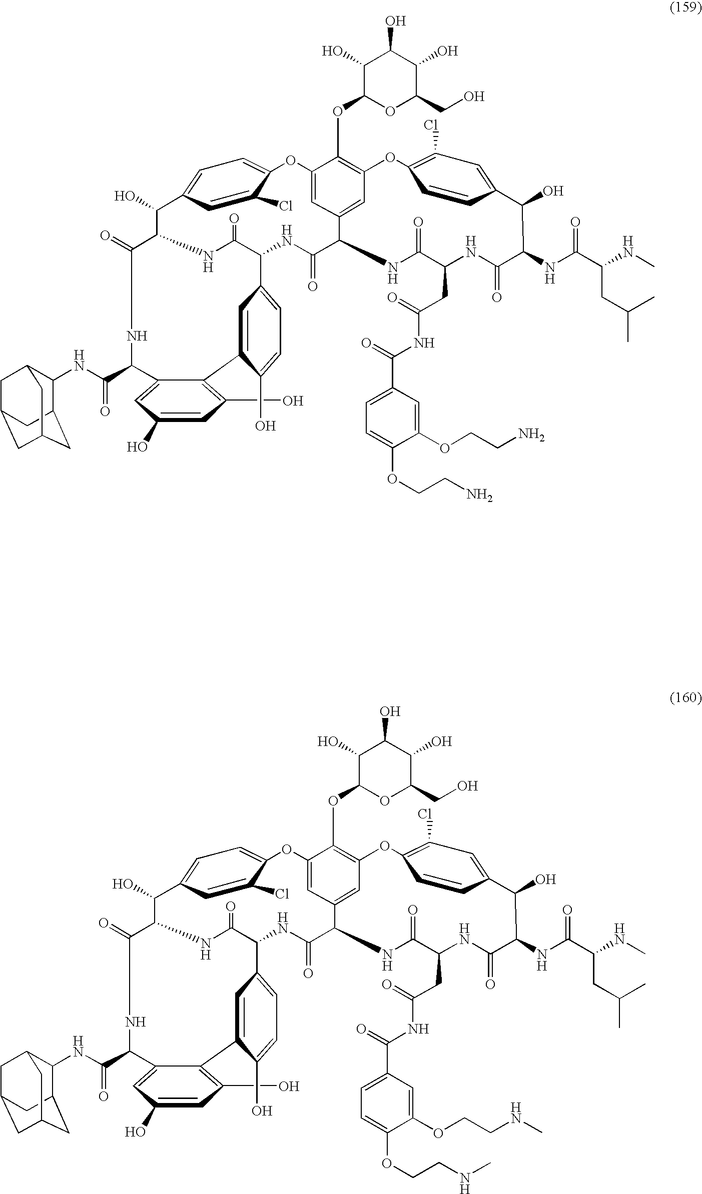

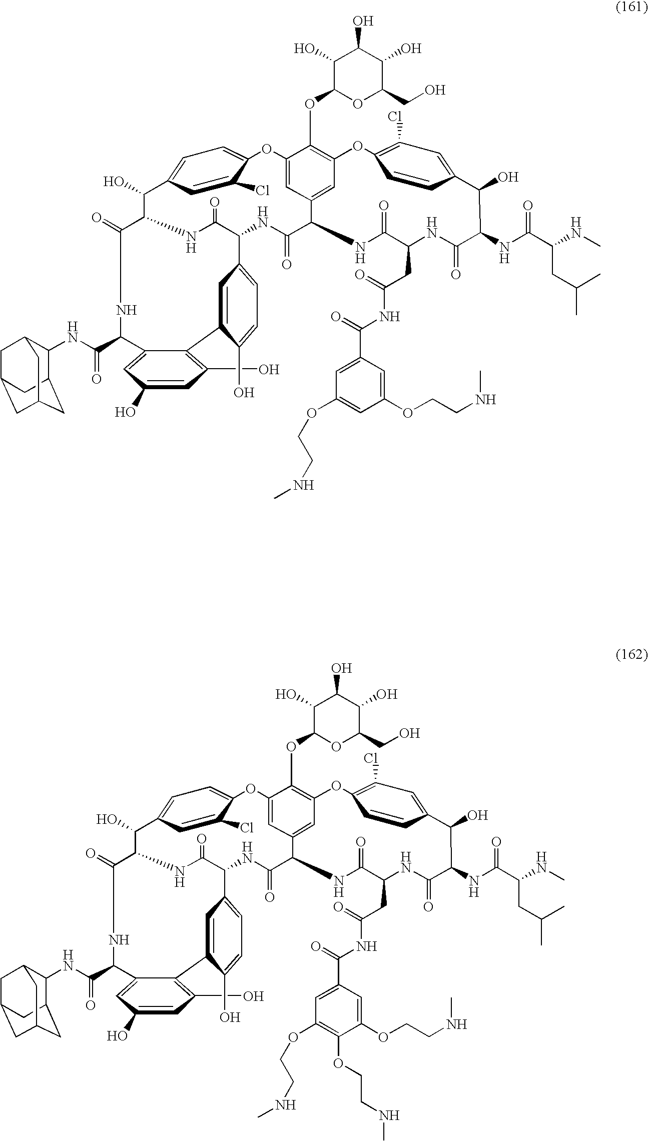

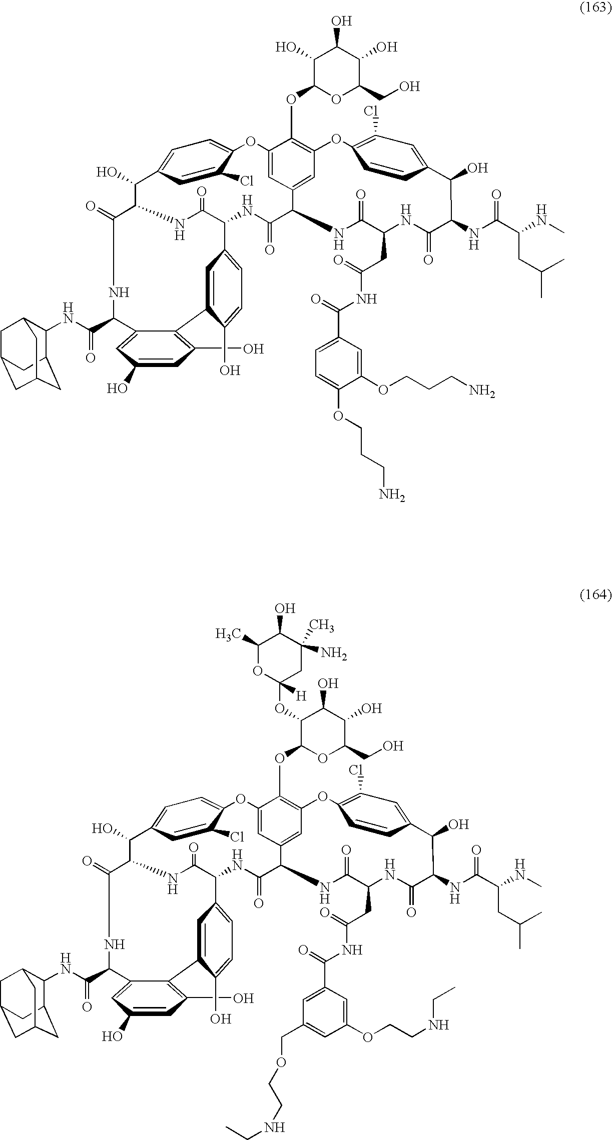

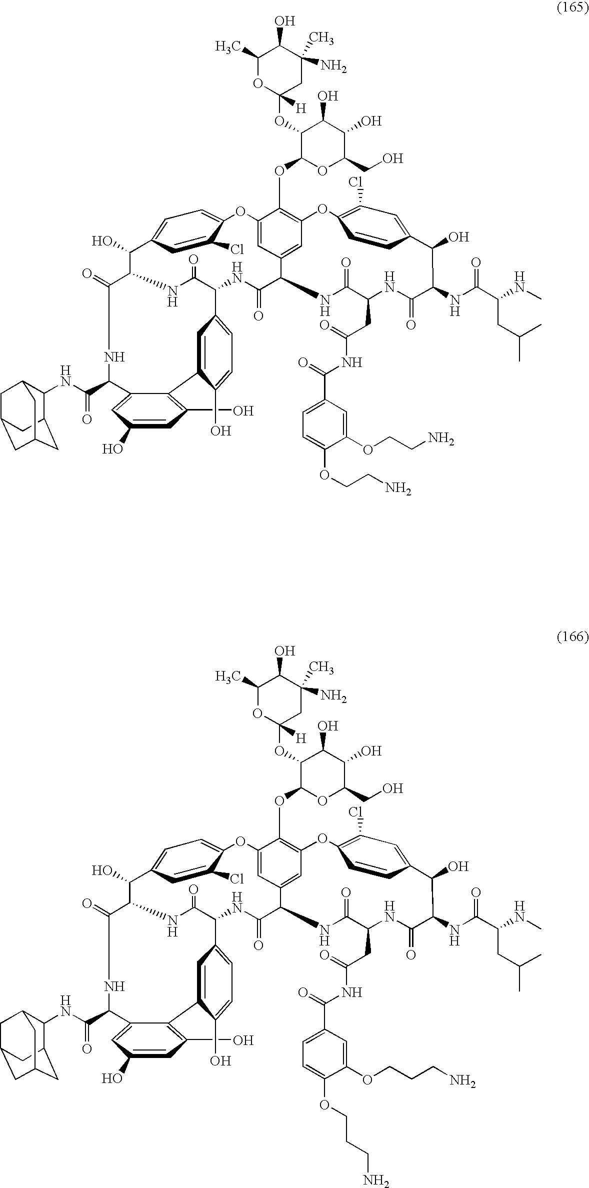

22. A pharmaceutical composition comprising a therapeutically effective amount of a compound of claim 1, together with a pharmaceutically acceptable carrier or excipient thereof.

23. A method of treating a mammal in need of such treatment comprising administering to the mammal an antibacterial effective amount of a compound of claim 1 together with a pharmaceutically acceptable carrier or excipient thereof.

24. A method of making a compound of Formulas I-V, comprising:
modifying a compound from the group consisting of Formulas i, ii, iii, iv and v, 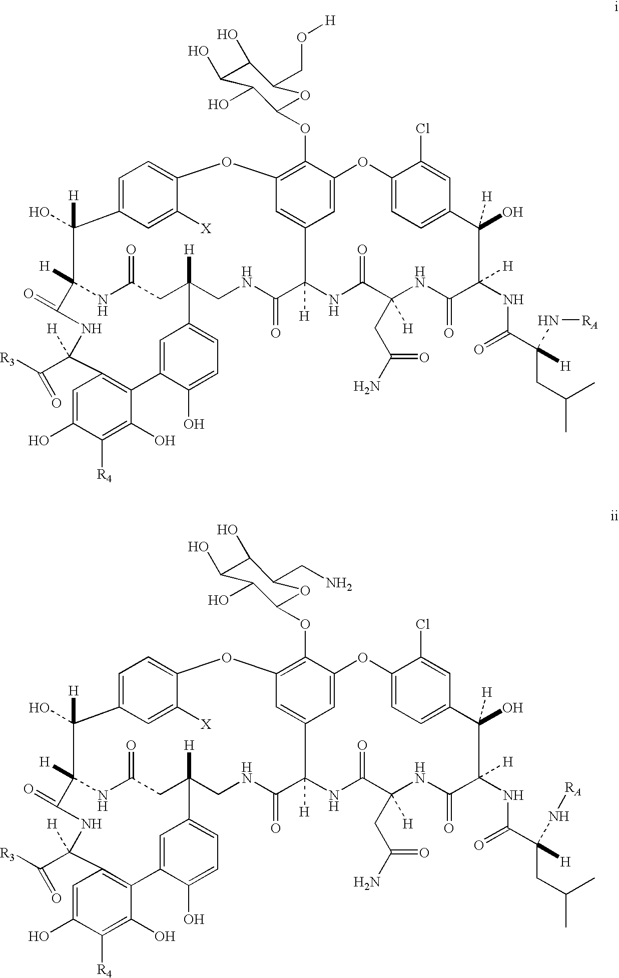 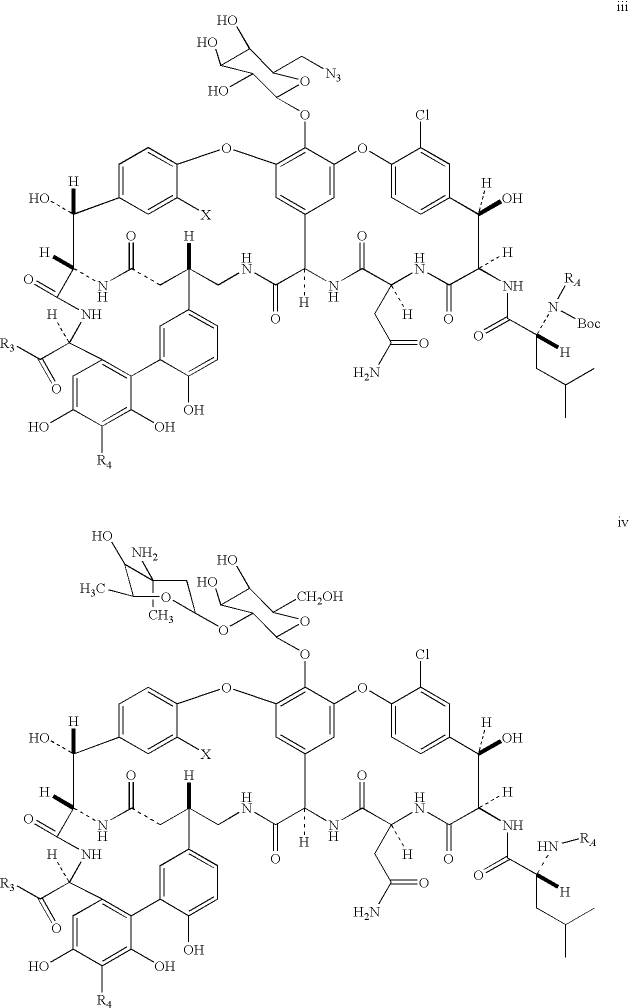 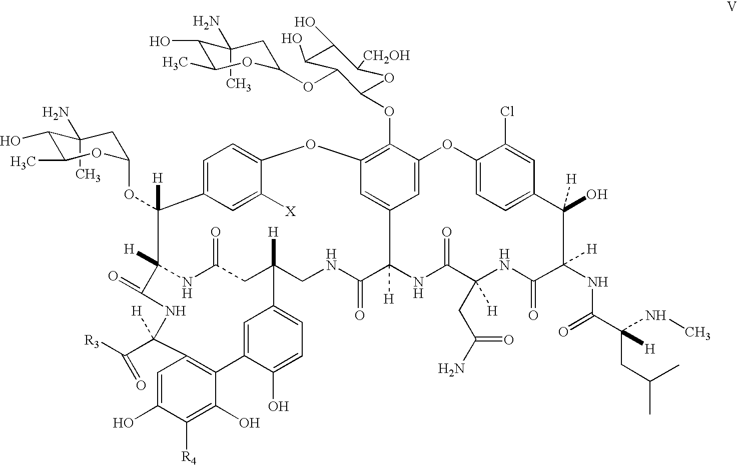
wherein R_{A} is hydrogen or methyl, X is chlorine or hydrogen, R₃ is OH or alkoxy, 2-adamantanamino, or loweralkylamino as defined herein, R₄ is hydrogen or protected CH₂NHCH₂PO₃H₂, or Boc-aminoloweralkyl as defined herein, by a technique selected from the group consisting of,
(a) protecting the amino group with 9-fluorenylmethoxycarbonyl (Fmoc) or tert-butoxycarbonyl (Boc), or other appropriate nitrogen protecting groups.
(b) acylating the primary amide group of the 3^{rd} amino acid asparagine with an R_{B}SO₂Cl, R_{B}COOH with a coupling reagent, or R_{B}SO₂—NCO group in the presence of a base such as triethylamine and the like,
(c) removing the amino protecting group wherein the Boc protecting group is removed with mild acid such as trifluoroacetic acid and Fmoc group is removed with a base such as diethylamine and the like,
(d) if the R₃ is alkoxy, removing the alkoxy group by mild base or acid hydrolysis to give the carboxylic acid derivative,
(e) reducing the azide function to an amine,
(f) alkylating the primary alcohol of the mono-sugar or the amino substituent on the amino-substituted sugar moiety of the 4^{th} amino acid of the compound with an alkyl halide having the structure R-J where J is a halogen, R₁-J where J is a halogen, R₂-J where J is a halogen or R_{C}-J where J is a halogen,
(g) acylating the primary alcohol of the mono-sugar or the amino substituent on the amino-substituted sugar moiety of the 4^{th} amino acid of the compound with an acyl group having the structure C(═O)R₇,
(h) acylating the primary alcohol of the mono-sugar or the amino substituent on the amino-substituted sugar moiety of the 4^{th} amino acid of the compound with an acyl group having the structure C(═O)CHR₈NR₉R₁₀,
(i) reacting the amino substituent on the amino-substituted sugar moiety of the 4^{th} amino acid of the compound with an aldehyde or ketone followed by reductive amination of the resulting imine,
(j) conversing the acid moiety on the macrocyclic ring of the compound with substituted amide as defined by R₃,
(k) phosgene reaction on primary alcohol or primary amine of the mono-sugar moiety of the 4^{th} amino acid of the compound with the adjacent hydroxyl group,
(l) Mannich reaction on the 7^{th} amino acid of the compound where R₄ is hydrogen with NH₂—CHR₁₅—(CH₂)ₘ—NHSO₂R_{B}, NHR_{D}—CHR₁₅—(CH₂)_{q}—NR_{E}SO₂R_{B}, or NH₂—CHR₁₅—(CH₂)ₚ—CONHSO₂R_{B} in the presence of aqueous formaldehyde in acetonitrile and water or other suitable organic solvent,
(m) a combination of (a), (b) and (c),
(n) a combination of (a), (b), (c) and (d),
(o) a combination of (a), (b), (d), (j) and (c),
(p) a combination of (a), (b), (f), and (c),
(q) a combination of (a), (b), (g) and (c),
(r) a combination of (a), (b), (h) and (c),
(s) a combination of (a), (b), (i) and (c),
(t) a combination of (a), (b), (e) and (c),
(u) a combination of (a), (b), (e), (d) and (c),
(v) a combination of (a), (b), (d), (j), (e) and (c),
(w) a combination of (a), (b), (d), (e) and (c),
(x) a combination of (a), (b), (d), (j), (e), (f) and (c),
(y) a combination of (a), (b), (d), (j), (e), (g) and (c),
(z) a combination of (a), (b), (d), (j), (e), (h) and (c),
(aa) a combination of (a), (b), (d), (j), (e), (i) and (c),
(bb) a combination of (a), (b), (d), (e), (f) and (c),
(cc) a combination of (a), (b), (d), (e), (g) and (c),
(dd) a combination of (a), (b), (d), (e), (h) and (c),
(ee) a combination of (a), (b), (d), (e), (i) and (c),
(ff) a combination of (a), (b), (k), and (c),
(gg) a combination of (a), (b), (k), (d), (j) and (c),
(hh) a combination of (a), (b), (e), (k), and (c),
(ii) a combination of (a), (b), (e), (k), (d), (j) and (c),
(jj) a combination of (a), (l), and (c),
(kk) a combination of (a), (j), (l), and (c),
(ll) a combination of (j), (a), (l), and (c),
to form a compound having a formula selected from the group consisting of: 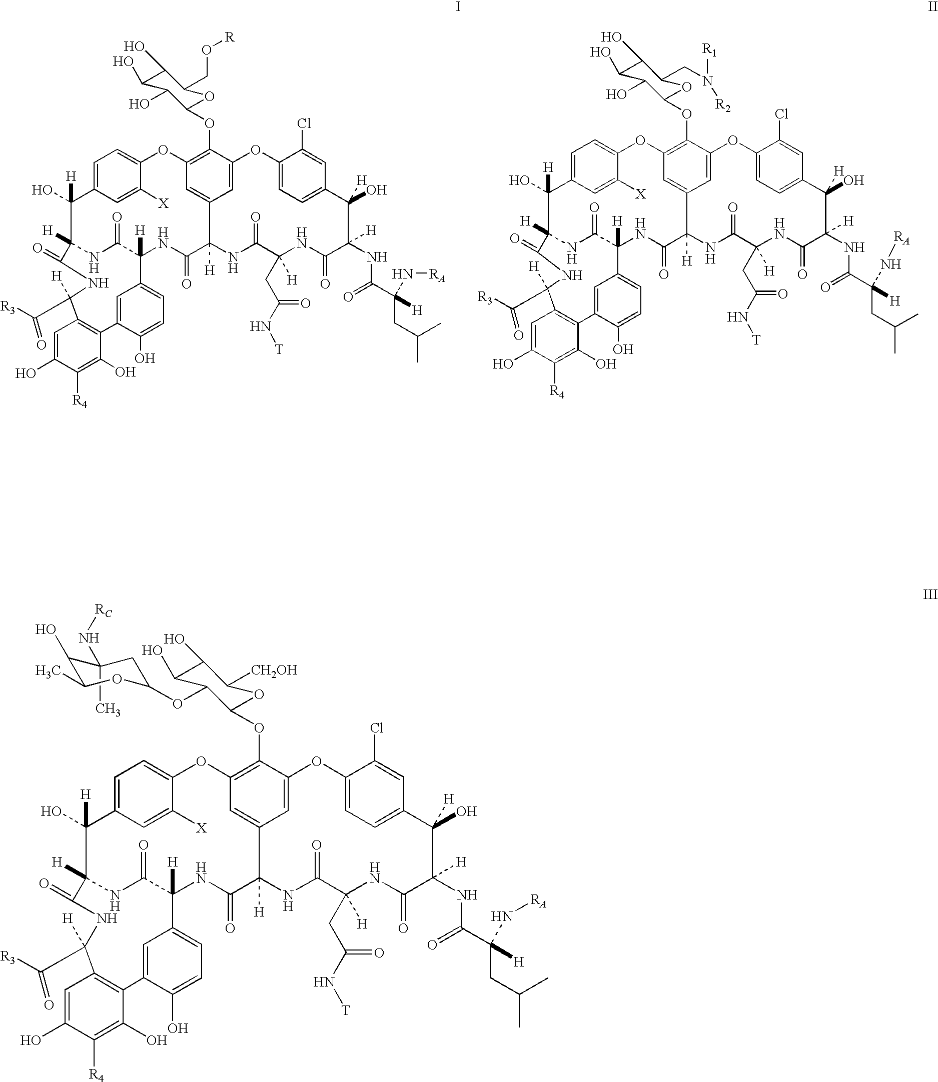 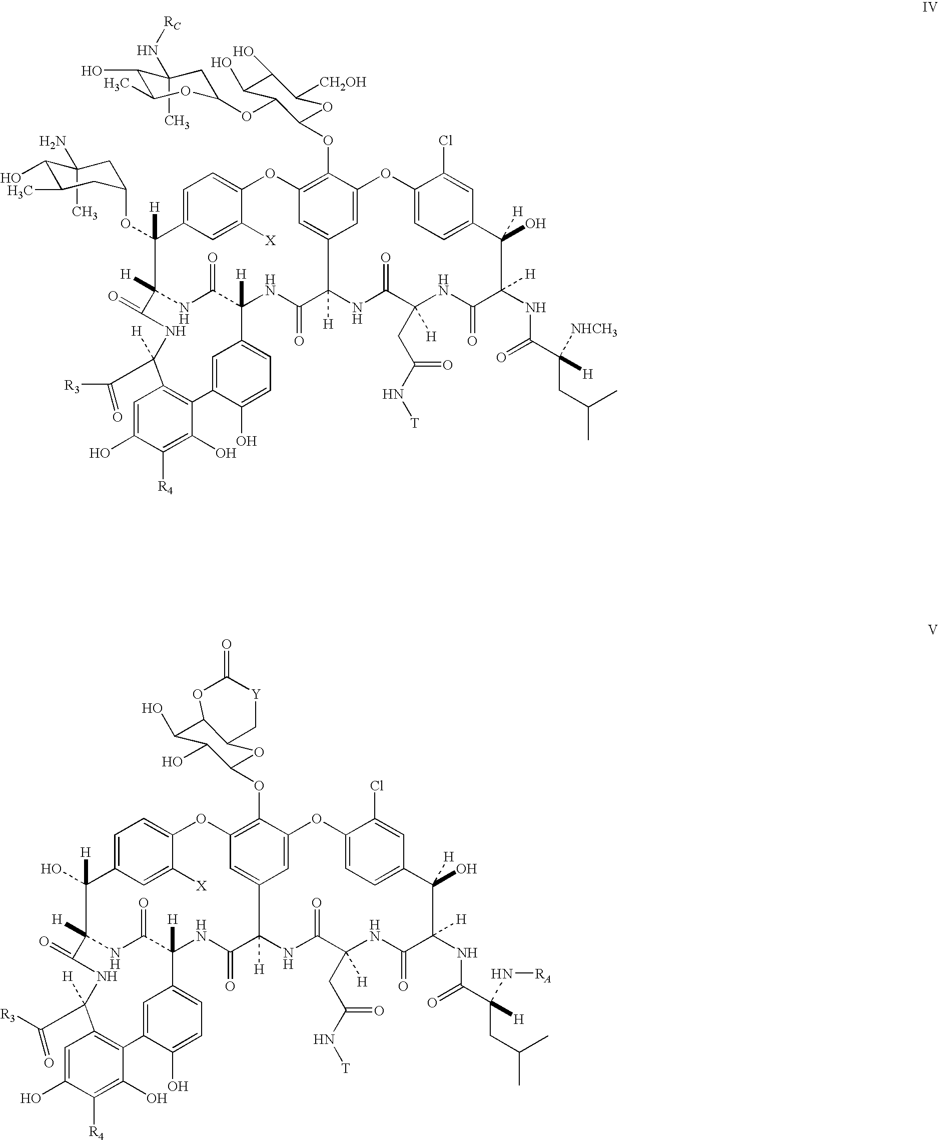
wherein R, R₁, R₂, R₃, R₄, R_{A}, X, Y, and T are as defined herein.
